(19) United States
(12) Patent Application Publication
(10) Pub. No.: US 2021/0308171 A1
(43) Pub. Date: Oct. 7, 2021

(54) METHODS FOR COMBINATORIAL SCREENING AND USE OF THERAPEUTIC TARGETS THEREOF

(71) Applicants: THE BROAD INSTITUTE, INC., CAMBRIDGE, MA (US); THE GENERAL HOSPITAL CORPORATION, BOSTON, MA (US)

(72) Inventors: Bradley E. BERNSTEIN, Boston, MA (US); John G. DOENCH, Cambridge, MA (US); Fadi J. NAJM, Boston, MA (US)

(21) Appl. No.: 17/266,538

(22) PCT Filed: Aug. 7, 2019
(86) PCT No.: PCT/US2019/045558

§ 371 Date: Feb. 5, 2021

Related U.S. Application Data
(60) Provisional application No. 62/715,779, filed on Aug. 7, 2018.
(60) Provisional application No. 62/880,579, filed on Jul. 30, 2019.

(51) Int. Cl. A61K 31/713; A61K 35/17; C12N 9/22; A61K 45/06; C12N 15/11; G01N 33/50; A61P 35/02

(52) U.S. Cl. CPC A61K 31/713; A61K 35/17; C12N 9/22; C40B 30/06; C12N 15/111; G01N 33/5023; A61P 35/02; A61K 45/06

(57) ABSTRACT

CRISPR-Cas9 has enabled a new generation of screening strategies to interrogate gene function. However, redundant genes and the complexity of functional gene networks can confound single gene knockout approaches. Furthermore, simple addition of two or more sgRNAs has shown only modest targeting efficacy in screening approaches. The present invention relates to combined orthogonal CRISPR-derived components to maximize gene targeting activity with minimal cross-talk and interference. The present invention also relates to efficient S. aureus Cas9 sgRNA design rules, which were paired with S. pyogenes Cas9 sgRNA design rules to achieve dual target gene inactivation in a high fraction of cells. Applicants developed a lentiviral vector and cloning strategy to generate high complexity pooled dual-knockout libraries and show that screening these libraries can identify combinatorial phenotypes, including synthetic lethal gene pairs across multiple cell types. The gene pairs can be targeted therapeutically and Applicants disclose therapeutically effective combination therapies.

### CROSS-REFERENCE TO RELATED APPLICATIONS

This application claims the benefit of U.S. Provisional Application Nos. 62/715,779, filed Aug. 7, 2018 and 62/880,579, filed Jul. 30, 2019. The entire contents of the above-identified applications are hereby fully incorporated herein by reference.

### STATEMENT REGARDING FEDERALLY SPONSORED RESEARCH

This invention was made with government support under Grant Nos. CA216873 and CA224536 awarded by the National Institutes of Health. The government has certain rights in the invention.

### REFERENCE TO AN ELECTRONIC SEQUENCE LISTING

The contents of the electronic sequence listing (BROD-2600WP_ST25.txt”; Size is 8,549,577 bytes and it was created on Jul. 26, 2019) is herein incorporated by reference in its entirety.

### TECHNICAL FIELD

The subject matter disclosed herein is generally directed to compositions and methods for combinatorial screening of phenotypic interactions between a set of target sequences using orthogonal CRISPR enzymes and therapeutic targets identified.

### BACKGROUND

Mapping the functional relationships between genes is a critical step towards understanding how disease states arise from gene dysfunction¹⁻³. In yeast, high-throughput methods have enabled the creation of genetic networks, with 23 million double mutants identifying nearly 1 million interactions⁴. Network complexity is orders of magnitude greater in human cells, with ˜10-fold more pairwise combinations of protein-coding genes and thousands of distinct cell types in which to examine interactions.

RNAi and CRISPR technologies can simultaneously perturb two or more genes, and thus represent a promising approach to uncover genetic interactions^{2,5}. Initial combinatorial CRISPR screens⁶ were performed using lentiviral constructs. However, repetitive elements in lentiviral vectors, including the U6 promoter, lead to high levels of recombination and decrease combinatorial screen efficiency⁷⁻¹⁰. Two efforts to achieve combinatorial CRISPR screens employed orthologous U6 promoters, from mouse and human^{7,8}, although another study found that multiple copies of the S. pyogenes tracrRNA sequence were likewise prone to recombination¹¹. Finally, because Cpf1 enzymes process their own transcripts, they can deliver multiple sgRNAs from one transcript. However, the reported efficiency of multiple indels in the same cell is less than 10%¹², too low for screening applications. In all cases, loading distinct RNAs into a common effector enzyme may result in competition between individual perturbagens and decreased overall efficiency^{13,14.} These design challenges are accentuated when using lentivirus to deliver reagents at single-copy for large scale, pooled genetic screens.

Citation or identification of any document in this application is not an admission that such document is available as prior art to the present invention.

### SUMMARY

It is an objective of the present invention to provide for novel CRISPR-Cas9 screening strategies to interrogate gene function. Redundant genes and the complexity of functional gene networks can confound single gene knockout approaches. Furthermore, simple addition of two or more sgRNAs has shown only modest targeting efficacy in screening approaches. It is another objective of the present invention to provide for combined orthogonal CRISPR-derived components to maximize gene targeting activity with minimal cross-talk and interference. Additionally, it is another objective of the present invention to use machine learning to establish efficient S. aureus Cas9 sgRNA design rules and pair the rules with rules discovered for S. pyogenes Cas9 to achieve dual target gene inactivation in a high fraction of cells. It is another objective of the present invention to develop a lentiviral vector and cloning strategy to generate high complexity pooled dual-knockout libraries and show that screening these libraries can identify synthetic lethal gene pairs across multiple cell types, including genes in the MAPK pathway and anti-apoptotic genes. It is another objective of the present invention to provide for flexible manipulations using the orthogonal CRISPR screening strategy to allow interrogation of combinatorial knockout, activation, or repression screens.

The present inventors have in an unprecedented way adapted the use of the CRISPR/Cas system to interrogate combinatorial phenotypes using the “Big Papi” approach described herein. It is another objective of the present invention to provide therapeutic targets based on the combination of genes identified in the screens.

Preferred statements (features) and embodiments of this invention are set herein below. Each statement and embodiment of the invention so defined may be combined with any other statement and/or embodiments unless clearly indicated to the contrary. In particular, any feature indicated as being preferred or advantageous may be combined with any other feature or features or statements indicated as being preferred or advantageous. Hereto, the present invention is in particular captured by any one or any combination of one or more of the below statements and embodiments, with any other statement and/or embodiments.

In one aspect, the present invention provides for a method for treating cancer in a subject in need thereof comprising administering to the subject a therapeutically effective amount of a combination therapy comprising one or more agents targeting the expression, activity, substrate or products of WDR77 and BRD4. In certain embodiments, the cancer is Acute myeloid leukemia (AML) NUT (nuclear protein in testis) midline carcinoma, or multiple myeloma.

In another aspect, the present invention provides for a method for treating inflammation in a subject in need thereof comprising administering to the subject a therapeutically effective amount of a combination therapy comprising one or more agents targeting the expression, activity, substrate or products of WDR77 and BRD4. In certain embodiments, the inflammation is caused by an autoimmune disease. In certain embodiments, the inflammation is caused by a pathogen.

In another aspect, the present invention provides for a method for reactivation of HIV in a subject in need thereof comprising administering to the subject a therapeutically effective amount of a combination therapy comprising one or more agents targeting the expression, activity, substrate or products of WDR77 and BRD4.

In certain embodiments, the one or more agents targeting BRD4 is selected from the group consisting of AZD5153, PFI-1, CPI-203, CPI-0610, RVX-208, OTX015, I-BET151, I-BET762, I-BET-726, dBET1, ARV-771, ARV-825, BETd-260/ZBC260 and MZ1.

In another aspect, the present invention provides for a CD8+ T cell for use in adoptive cell transfer comprising a CD8+ T cell treated with a combination of one or more agents targeting the expression, activity, substrate or products of WDR77 and BRD4. The CD8+ T cell may be a CAR T cell. In certain embodiments, the one or more agents targeting BRD4 is selected from the group consisting of AZD5153, JQ1, PFI-1, CPI-203, CPI-0610, RVX-208, OTX015, I-BET151, I-BET762, I-BET-726, dBET1, ARV-771, ARV-825, BETd-260/ZBC260 and MZ1.

In another aspect, the present invention provides for a method for treating cancer in a subject in need thereof comprising administering to the subject a therapeutically effective amount of a combination therapy comprising one or more agents targeting the expression, activity, substrate or products of SETD6 and INO80. In certain embodiments, the cancer comprises an MLL fusion, such as Acute myeloid leukemia (AML).

In another aspect, the present invention provides for a method for treating cancer in a subject in need thereof comprising administering to the subject a therapeutically effective amount of a combination therapy comprising one or more agents targeting the expression, activity, substrate or products of KAT6B and CHD8. In certain embodiments, the cancer is Acute myeloid leukemia (AML).

In another aspect, the present invention provides for a method for treating cancer in a subject in need thereof comprising administering to the subject a therapeutically effective amount of a combination therapy comprising one or more agents targeting the expression, activity, substrate or products of ATRX and SMARCAL1. In certain embodiments, the cancer is Acute myeloid leukemia (AML).

In another aspect, the present invention provides for a method for treating cancer in a subject in need thereof comprising administering to the subject a therapeutically effective amount of a combination therapy comprising one or more agents targeting the expression, activity, substrate or products of MTA1 and MTA2. In certain embodiments, the cancer comprises Acute myeloid leukemia (AML). In certain embodiments, the cancer comprises a rearrangement in TEL or MLL.

In another aspect, the present invention provides for a method for treating cancer in a subject in need thereof comprising administering to the subject a therapeutically effective amount of a combination therapy comprising one or more agents targeting the expression, activity, substrate or products of HDAC1 and HDAC2. In certain embodiments, the cancer comprises Acute myeloid leukemia (AML). In certain embodiments, the cancer comprises a rearrangement in TEL or MLL.

In another aspect, the present invention provides for a method for treating cancer in a subject in need thereof comprising administering to the subject a therapeutically effective amount of a combination therapy comprising one or more agents targeting the expression, activity, substrate or products of CHD3 and HDAC2. In certain embodiments, the cancer comprises Acute myeloid leukemia (AML). In certain embodiments, the cancer does not comprise a rearrangement in TEL or MLL.

In another aspect, the present invention provides for a method for treating cancer in a subject in need thereof comprising administering to the subject a therapeutically effective amount of a combination therapy comprising one or more agents targeting the expression, activity, substrate or products of ING1 and ING2. In certain embodiments, the cancer comprises Acute myeloid leukemia (AML). In certain embodiments, the cancer comprises a rearrangement in TEL or MLL.

In another aspect, the present invention provides for a method for treating cancer in a subject in need thereof comprising administering to the subject a therapeutically effective amount of a combination therapy comprising one or more agents targeting the expression, activity, substrate or products of ASF and ASF1A. In certain embodiments, the cancer comprises Acute myeloid leukemia (AML). In certain embodiments, the cancer comprises a rearrangement in TEL or MLL.

In another aspect, the present invention provides for a method for treating cancer in a subject in need thereof comprising administering to the subject a therapeutically effective amount of a combination therapy comprising one or more agents targeting the expression, activity, substrate or products of ING4 and ING5. In certain embodiments, the cancer comprises Acute myeloid leukemia (AML). In certain embodiments, the cancer comprises a rearrangement in TEL or MLL.

In another aspect, the present invention provides for a personalized method for treating cancer comprising administering to a subject suffering from a cancer having a deficiency in function or expression or a mutation in either gene in a pair of genes selected from the group consisting of MTA1 and MTA2, HDAC1 and HDAC2, CHD3 and HDAC2, ING1 and ING2, ING4 and ING5, ASF1B and ASF1A, ARID4A and JADE2, ARID4A and SMYD1, ARID4A and SETD9, ATRX and HIRA, SLBP and HIRA, CREBBP and CARM1, ARID3A and RAD54L2, JMJD6 and WDR5, DPF2 and SMYD5, JMJD6 and MBD2, MSL3 and SRCAP, KMT2C and KMT2D, HDAC3 and SETD1B, KMT2A and KMT2B, KDM3B and KMT2D, SMARCA4 and SMARCA2, BRD8 and SMARCA1, WDR77 and BRD4, SETD6 and INO80, SMARCAL1 and ATRX, KAT6B and CHD8, ARID1B and ARID1A, WDR77 and HDAC6, WDR77 and KAT6B, KDM3B and ARID1A, KDM3B and CHD3, SETD2 and NSD1, MTA1 and DOT1L, KDM3B and BRD1, KDM4A and KAT6A, INO80 and CBX1, HDAC6 and EZH2, SMARCAL1 and HDAC8, KAT5 and CHAF1B, SUV39H1 and HDAC6, KDM3B and BRD4, KMT2B and BRD8, PRMT5 and KAT5, SIRT4 and CBX1, KAT6A and CHD6, WDR77 and DOT1L, KAT2B and EHMT1, KMT2E and KAT6A, KDM3B and DOT1L, KDM3B and KDM3A, CHD8 and BRD1, HIRA and ATRX, KDM5C and KDM3B, PRDM6 and KDM3B, KAT6B and KAT6A, SMARCB1 and KDM6A, MECP2 and KDM4B, KAT2A and HDAC5, SETD2 and KDM3B, RFWD2 and CHD6, SMARCB1 and ARID3C, SETMAR and BRD1, HDAC2 and DIDO1, HDAC2 and DNMT3B, KDM4D and BRD1, PRDM1 and HDAC8, SMARCA5 and KAT6A, and KMT2D and ARID1A a therapeutically effective amount of one or more agents targeting the expression, activity, substrate or products of the gene not having the deficiency or mutation. In certain embodiments, the cancer comprises Acute myeloid leukemia (AML). In certain embodiments, the cancer comprises a rearrangement in TEL or MLL.

In another aspect, the present invention provides for a method for treating cancer in a subject in need thereof comprising administering to the subject a therapeutically effective amount of one or more agents targeting a first gene and one or more agents targeting a second gene for one or more gene pairs, wherein said one or more gene pairs are selected from the group consisting of MTA1 and MTA2, HDAC1 and HDAC2, CHD3 and HDAC2, ING1 and ING2, ING4 and ING5, ASF1B and ASF1A, ARID4A and JADE2, ARID4A and SMYD1, ARID4A and SETD9, ATRX and HIRA, SLBP and HIRA, CREBBP and CARM1, ARID3A and RAD54L2, JMJD6 and WDR5, DPF2 and SMYD5, JMJD6 and MBD2, MSL3 and SRCAP, KMT2C and KMT2D, HDAC3 and SETD1B, KMT2A and KMT2B, KDM3B and KMT2D, SMARCA4 and SMARCA2, BRD8 and SMARCA1, WDR77 and BRD4, SETD6 and INO80, SMARCAL1 and ATRX, KAT6B and CHD8, ARID1B and ARID1A, WDR77 and HDAC6, WDR77 and KAT6B, KDM3B and ARID1A, KDM3B and CHD3, SETD2 and NSD1, MTA1 and DOT1L, KDM3B and BRD1, KDM4A and KAT6A, INO80 and CBX1, HDAC6 and EZH2, SMARCAL1 and HDAC8, KAT5 and CHAF1B, SUV39H1 and HDAC6, KDM3B and BRD4, KMT2B and BRD8, PRMT5 and KAT5, SIRT4 and CBX1, KAT6A and CHD6, WDR77 and DOT1L, KAT2B and EHMT1, KMT2E and KAT6A, KDM3B and DOT1L, KDM3B and KDM3A, CHD8 and BRD1, HIRA and ATRX, KDM5C and KDM3B, PRDM6 and KDM3B, KAT6B and KAT6A, SMARCB1 and KDM6A, MECP2 and KDM4B, KAT2A and HDAC5, SETD2 and KDM3B, RFWD2 and CHD6, SMARCB1 and ARID3C, SETMAR and BRD1, HDAC2 and DIDO1, HDAC2 and DNMT3B, KDM4D and BRD1, PRDM1 and HDAC8, SMARCA5 and KAT6A, and KMT2D and ARID1A, and wherein the one or more agents target the expression, activity, substrate or products of said first and second genes.

In another aspect, the present invention provides for a method for treating cancer in a subject in need thereof comprising administering to the subject a therapeutically effective amount of one or more agents targeting a gene selected from the group consisting of: MEAF6, SRCAP, WDR77, CHAF1B, TAF5, CSTF1, WDHD1, BRD4, DNMT1, WDR61, GTF3C2, PRMT5, RBBP5, HDAC3, TRIM24, CHD7, HIRA and SMC1A; or HDAC3, PRMT5, DNMT1 and TAF3; or BRD4, KMT2A and CHD7; or SMC2, SMC3, TAF1, WDR92, KDM2B and HUWE1, wherein the one or more agents target the expression, activity, substrate or products of said gene.

In certain embodiments, the one or more agents according to any embodiment herein comprises a small molecule inhibitor, small molecule degrader (e.g., PROTAC), genetic modifying agent, antibody, antibody fragment, antibody-like protein scaffold, aptamer, protein, or any combination thereof.

In certain embodiments, the one or more agents comprise a histone acetylation inhibitor, histone deacetylase (HDAC) inhibitor, histone lysine methylation inhibitor, histone lysine demethylation inhibitor, DNA methyltransferase (DNMT) inhibitor, inhibitor of acetylated histone binding proteins, inhibitor of methylated histone binding proteins, sirtuin inhibitor, protein arginine methyltransferase inhibitor or kinase inhibitor. In certain embodiments, the DNA methyltransferase (DNMT) inhibitor is selected from the group consisting of azacitidine (5-azacytidine), decitabine (5-aza-2′-deoxycytidine), EGCG (epigallocatechin-3-gallate), zebularine, hydralazine, and procainamide. In certain embodiments, the histone acetylation inhibitor is C646. In certain embodiments, the histone deacetylase (HDAC) inhibitor is selected from the group consisting of vorinostat, givinostat, panobinostat, belinostat, entinostat, CG-1521, romidepsin, ITF-A, ITF-B, valproic acid, OSU-HDAC-44, HC-toxin, magnesium valproate, plitidepsin, tasquinimod, sodium butyrate, mocetinostat, carbamazepine, SB939, CHR-2845, CHR-3996, JNJ-26481585, sodium phenylbutyrate, pivanex, abexinostat, resminostat, dacinostat, droxinostat, RGFP966 and trichostatin A (TSA). In certain embodiments, the histone lysine demethylation inhibitor is selected from the group consisting of pargyline, clorgyline, bizine, GSK2879552, GSK-J4, KDM5-C70, JIB-04, and tranylcypromine. In certain embodiments, the histone lysine methylation inhibitor is selected from the group consisting of EPZ-6438, GSK126, CPI-360, CPI-1205, CPI-0209, DZNep, GSK343, EI1, BIX-01294, UNC0638, EPZ004777, GSK343, UNC1999 and UNC0224. In certain embodiments, the inhibitor of acetylated histone binding proteins is selected from the group consisting of AZD5153, PFI-1, CPI-203, CPI-0610, RVX-208, OTX015, I-BET151, I-BET762, I-BET-726, dBET1, ARV-771, ARV-825, BETd-260/ZBC260 and MZ1. In certain embodiments, the inhibitor of methylated histone binding proteins is selected from the group consisting of UNC669 and UNC1215. In certain embodiments, the sirtuin inhibitor comprises nicotinamide.

In certain embodiments, the genetic modifying agent comprises a CRISPR system, shRNA, a zinc finger nuclease system, a TALEN, or a meganuclease. The CRISPR system may comprise a Cas13 system. The Cas13 system may comprise Cas13-ADAR.

In certain embodiments, the one or more agents target an active site. In certain embodiments, the cancer is Acute lymphoblastic leukemia (ALL) or Acute myeloid leukemia (AML). In certain embodiments, the one or more agents according to any embodiment herein are administered concurrently or sequentially. In certain embodiments, an additional cancer therapy is administered (e.g., chemotherapy, radiation, surgery, immunotherapy).

In another aspect, the present invention provides for a DNA construct comprising a sequence encoding two CRISPR guide sequences positioned in an inverted orientation to each other and flanked by convergent regulatory sequences, wherein each guide sequence is operably linked to the regulatory sequence flanking the guide sequence, wherein each guide sequences is specific for an orthogonal CRISPR enzyme, and wherein the regulatory sequences do not have 100% sequence identity to one another. In certain embodiments, each regulatory sequence is a RNA polymerase III (RNAP III) promoter. In certain embodiments, one RNAP III promoter comprises the U6 promoter and one RNAP III promoter comprises the H1 promoter. In certain embodiments, the orthogonal CRISPR enzymes comprise S. aureus Cas9 and S. pyogenes Cas9. In certain embodiments, the DNA construct further comprises a sequence encoding a CRISPR enzyme operably linked to a separate regulatory sequence. The CRISPR enzyme may be S. aureus Cas9 (e.g., because it is smaller). In certain embodiments, the DNA construct further comprises a sequence encoding at least one selectable marker. The at least one selectable marker may be an antibiotic resistance gene. The at least one selectable marker may be a fluorescent gene. Each guide sequence may further comprise a barcode sequence (e.g., to identify the guide sequence). In certain embodiments, one or more of the regulatory sequences are inducible. In certain embodiments, one or both of the guide sequences comprise an aptamer sequence (e.g., for recruitment of a functional domain). The aptamer sequence may comprise an MS2 aptamer. In certain embodiments, the DNA construct further comprises primer binding sequences flanking the guide sequences.

In another aspect, the present invention provides for a vector comprising a DNA construct according to any embodiment herein. The vector may be a viral vector. The viral vector may be a lentivirus, adeno associated virus (AAV) or adenovirus vector.

In another aspect, the present invention provides for a library for the combinatorial screening of phenotypic interactions between a set of target sequences comprising a plurality of vectors according to any embodiment herein, wherein the library comprises vectors comprising all possible pairwise combinations of guide sequences specific for the set of target sequences. The set of target sequences may comprise sequences targeting expression of at least two protein coding genes. In certain embodiments, at least one protein coding gene is selected from the group consisting of: genes in Table 1; or DNMT1, KDM5A, KDM5B, KDM5C, KDM5D, SETDB1, SETDB2, BAZ2A, BAZ2B, ASH1L, KMT2A, KMT2B, SUV39H1, SUV39H2, JARID2, KAT2A, KAT2B, CHD3, CHD4, CHD5, CHAF1A, ZMYND8, BRPF1, BRPF3, BRD1, MBD2, MBD3, MBD1, HDAC4, HDAC5, HDAC9, BRWD1, BRWD3, KDM2A, PHIP, PBRM1, CXXC1, SETMAR, EHMT1, EHMT2, ATAD2, ATAD2B, KMT2C, KMT2D, KMT2E, MGMT, WBSCR22, CARM1, KDM4A, KDM4B, KDM4C, KDM4D, KDM4E, ARID4A, ARID4B, PHF2, PHF8, SP140L, BPTF, BAZ1A, BAZ1B, KDM7A, TRIM24, TRIM33, TRIM66, KAT5, KAT6A, KAT6B, KATE, CHD1, CHD2, CHD6, CHD7, CHD8, CHD9, SMARCA2, SMARCA4, SMARCA1, SMARCA5, EPC1, EPC2, KDM1A, KDM1B, DNMT3A, DNMT3B, WHSC1, WHSC1L1, NSD1, ZMYND11, SHPRH, MBD4, MBD3L1, MBD3L2, MECP2, ASF1A, ASF1B, ELP3, ING1, ING2, ING3, ING4, ING5, SLBP, SAP30L, SAP30, HAT1, HDAC1, HDAC10, HDAC11, HDAC2, HDAC3, HDAC6, HDAC7, HDAC8, DOT1L, MEAF6, FBXW9, FBXL19, TAF5L, TAF5, WDHD1, WDR48, WDR5, WDR61, WDR77, WDR82, WDR92, CHAF1B, CSTF1, CORO2A, DDB2, ELP2, EED, GTF3C2, HIRA, KDM2B, MTA2, MTA3, MTA1, RBBP4, RBBP5, RBBP7, RFWD2, TET1, TET3, CBX1, CBX2, CBX3, CBX4, CBX5, CBX6, CBX7, CBX8, CDYL2, CDYL, CDY1, CDY1B, CDY2A, CDY2B, SIRT1, SIRT2, SIRT3, SIRT4, SIRT5, SIRT6, SIRT7, SMC1A, SMC1B, SMC2, SMC3, SMC4, PRDM1, PRDM11, PRDM14, PRDM16, PRDM2, PRDM6, PRDM9, SMYD1, SMYD2, SMYD3, SMYD4, SETD1A, SETD1B, SETD2, SETD3, SETD4, SETD5, SETD6, SETD9, SETD7, SMYD5, EZH1, EZH2, ARID1A, ARID1B, ARID2, ARID3A, ARID3B, ARID3C, ARID5A, ARID5B, CREBBP, EP300, SP100, SP140, TAF1L, TAF1, BRD2, BRD3, BRD4, BRD7, BRD8, BRD9, BRDT, CECR2, HR, JMJD1C, JMJD4, JMJD6, KDM3A, KDM3B, KDM6A, KDM6B, UTY, PHRF1, PHF1, PHF10, PHF12, PHF13, PHF14, PHF19, PHF21A, PHF21B, PHF23, PHF3, TAF3, AIRE, DIDO1, DPF1, DPF2, DPF3, INTS12, KAT7, MSL3, MTF2, METTL13, MORF4L1, PRMT1, PRMT2, PRMT5, PYGO1, PYGO2, RSF1, TRIM28, UHRF1, UHRF2, EP400, INO80, RAD54L, RAD54L2, SET, SMARCAL1, SMARCB1, SMARCAD1, SRCAP, TBP, TSPYL2, ATRX, CHD1L, IL411, JADE1, JADE2 and JADE3; or DOT1L, EZH2, EHMT1, EHMT2, SETD7, SMYD2, DNMT1, PRMT1, PRMT3, PRMT5, PRMT4, PRMT6, PRMT5, KDM1A, KDM6A, KDM6B, HDAC1, HDAC2, HDAC3, HDAC6, HDAC8, SIRT1, SIRT2, SIRT6, BAZ2A, BAZ2B, BRD4, BRD9/7, EP300, CECR2, SMARCA4, P300, CDK7, EED, SMYD3, BRPF1, KDM4A, KDM4B, KDM4C, KDM4D, KDM4E, KDM5A, KDM5B, KDM5C and KDM5D.

In certain embodiments, at least one protein coding gene comprises a protein domain selected from the group consisting of PF00439:Bromodomain, PF00145:C-5 cytosine-specific DNA methylase, PF02373:JmjC domain, hydroxylase, PF00385:Chromo (CHRromatin Organisation MOdifier) domain, PF00850:Histone deacetylase domain, PF01388:ARID/BRIGHT DNA binding domain, PF02375:jmjN domain, PF00856:SET domain, PF13508:Acetyltransferase (GNAT) domain, PF06466:PCAF (P300/CBP-associated factor)N-terminal domain, PF01853:MOZ/SAS family, PF11717:RNA binding activity-knot of a chromodomain, PF08241:Methyltransferase domain, PF13847:Methyltransferase domain, PF05185:PRMT5 arginine-N-methyltransferase, PF12047:Cytosine specific DNA methyltransferase replication foci domain, PF11531:Coactivator-associated arginine methyltransferase 1 N terminal, PF12589:Methyltransferase involved in Williams-Beuren syndrome, PF01035:6-O-methylguanine DNA methyltransferase, DNA binding domain, PF02870:6-O-methylguanine DNA methyltransferase, ribonuclease-like domain, PF00628:PHD-finger, PF05033:Pre-SET motif, PF00004:ATPase family associated with various cellular activities (AAA), PF02463:RecF/RecN/SMC N terminal domain, PF02146:Sir2 family, PF01426:BAH domain, PF02008:CXXC zinc finger domain, PF06464:DMAP1-binding Domain, PF00400:WD domain, G-beta repeat, PF08123:Histone methylation protein DOT1, PF09340:Histone acetyltransferase subunit NuA4, PF10394:Histone acetyl transferase HAT1 N-terminus, PF13867:Sin3 binding region of histone deacetylase complex subunit SAP30, PF12203:Glutamine rich N terminal domain of histone deacetylase 4, PF04729:ASF1 like histone chaperone, PF12998:Inhibitor of growth proteins N-terminal histone-binding, PF15247:Histone RNA hairpin-binding protein RNA-binding domain, PF00583:Acetyltransferase (GNAT) family, PF01429:Methyl-CpG binding domain, PF14048:C-terminal domain of methyl-CpG binding protein 2 and 3, PF00956:Nucleosome assembly protein (NAP), PF01593:Flavin containing amine oxidoreductase, PF06752:Enhancer of Polycomb C-terminus, PF10513:Enhancer of polycomb-like, PF12253:Chromatin assembly factor 1 subunit A, PF15539:CAF1 complex subunit p150, region binding to CAF1-p60 at C-term, PF15557:CAF1 complex subunit p150, region binding to PCNA, PF00176:SNF2 family N-terminal domain, PF09110:HAND and PF04855: SNF5/SMARCB1/INI1.

In certain embodiments, each pairwise combination of guide sequences comprises a guide sequence selected from SEQ ID NOS: 1-552 and a guide sequence selected from SEQ ID NOS: 553-1104. In certain embodiments, each pairwise combination of guide sequences comprises a guide sequence selected from the group consisting of SEQ ID NOS: 1105-23903 and a guide sequence selected from the group consisting of SEQ ID NOS: 23904-45515.

In another aspect, the present invention provides for a method of combinatorial screening of phenotypic interactions between a set of target sequences in a population of cells comprising: introducing a library according to any embodiment herein to a population of cells, wherein two orthogonal CRISPR enzymes are expressed in said cells; selecting for cells comprising a vector of the library; selecting for cells having a desired phenotype; and determining in the cells having the desired phenotype the enrichment or depletion of combinations of guide sequences as compared to the representation in the library introduced. In certain embodiments, selecting for cells comprising a vector of the library comprises treating the population of cells with an antibiotic. In certain embodiments, the phenotypic interaction is lethality, wherein combinations of guide sequences depleted in viable cells indicate lethal combinations. In certain embodiments, the method further comprises treating the population of cells with a drug, wherein the phenotypic interaction is sensitivity or resistance to the drug. In certain embodiments, the phenotypic interaction is differentiation, wherein combinations of guide sequences are detected in cells expressing a differentiation marker. In certain embodiments, the phenotypic interaction is modulation of a cell state, wherein combinations of guide sequences are detected in cells expressing a marker of the cell state.

In certain embodiments, the population of cells is a population of cancer cells. In certain embodiments, the population of cells is a population of stem cells. In certain embodiments, the population of cells is a population of immune cells. In certain embodiments, the method comprises screening for combinations of targets capable of altering the cell state in the immune cells. The cell state may be an effector or suppressive cell state.

In certain embodiments, the combinations of targets identified are used to treat autoimmunity. In certain embodiments, the combinations of targets are used to treat cancer. In certain embodiments, the combinations of targets are used to modulate cells for adoptive cell transfer (ACT).

In certain embodiments, the method further comprises prioritizing candidate drug targets comprising determining epistatic genes, pseudo-essential genes, essential genes, pseudo-synthetic lethal genes and synthetic lethal genes, wherein candidate drug targets comprise synthetic lethal gene pairs. In certain embodiments, determining epistatic genes, pseudo-essential genes, essential genes, pseudo-synthetic lethal genes and synthetic lethal genes comprises applying an algorithm to the pair wise combinations identified. In certain embodiments, the orthogonal CRISPR enzymes comprise a Cas9, dCas9, Cas12, dCas12, or dCas13. In certain embodiments, the dCas9 or dCas12 are fusion proteins comprising an activation or repression domain. In certain embodiments, one CRISPR enzyme activates a gene and one CRISPR enzyme inactivates a gene.

In another aspect, the present invention provides for a method for generating a library for the combinatorial screening of phenotypic interactions between a set of target sequences comprising: synthesizing a first set of oligonucleotides, each oligonucleotide comprising a guide sequence specific for a target sequence in the set of target sequences and specific for a first orthogonal CRISPR enzyme, wherein the oligonucleotides comprise a first non-palindromic hybridization sequence at the 3′ end and a site for cloning into a vector at the 5′end; synthesizing a second set oligonucleotides, each oligonucleotide comprising a guide sequence specific for a target sequence in the set of target sequences and specific for a second orthogonal CRISPR enzyme, wherein the oligonucleotides comprise a second hybridization sequence at the 3′ end of the sequence that is complementary to the first hybridization sequence and a site for cloning into a vector at the 5′end; hybridizing the first and second set of oligonucleotides; performing DNA extension using the hybridization region as priming sequences to generate a pool of dsDNA oligonucleotides comprising pairs of inverted guide sequences specific for orthogonal CRISPR enzymes, wherein all pairwise combinations of guide sequences from the first and second set of oligonucleotides is represented in the pool; joining the oligonucleotides from the pool of dsDNA oligonucleotides into a vector comprising two convergent regulatory sequences flanking a cloning site, wherein the two convergent regulatory sequences do not have 100% sequence identity to one another, and wherein the oligonucleotides are joined between the convergent regulatory sequences. In certain embodiments, the ends of the oligonucleotides comprise restriction enzyme sites and the vector comprises compatible restriction enzyme site(s) between the convergent regulatory sequences, whereby joining is by ligation of compatible restriction enzyme digested ends on the oligonucleotides and the vector. In certain embodiments, the ends of the oligonucleotides comprise homologous sequences configured for recombination and the vector comprises compatible homologous sequences between the convergent regulatory sequences, whereby joining is by recombination of the oligonucleotides into the vector. The convergent regulatory sequences may be RNA polymerase III (RNAP III) promoters. In certain embodiments, one RNAP III promoter comprises the U6 promoter and one RNAP III promoter comprises the H1 promoter. The orthogonal CRISPR enzymes may comprise S. aureus Cas9 and S. pyogenes Cas9. In certain embodiments, the vector may further comprise a sequence encoding a CRISPR enzyme operably linked to a regulatory sequence. The CRISPR enzyme may be S. aureus Cas9.

In another aspect, the present invention provides for a method for treating cancer comprising a mutation in the MAPK pathway in a subject in need thereof, said method comprising administering to the subject a pharmaceutical composition capable of inhibiting the expression or activity of MAPK1 and MAPK3.

In another aspect, the present invention provides for a method for treating cancer comprising a mutation in the MAPK pathway in a subject in need thereof, said method comprising administering to the subject a pharmaceutical composition capable of inhibiting the expression or activity of ERK1 and ERK2. In certain embodiments, the mutation in the MAPK pathway comprises BRAF V600E, KRAS G12S or NRAS Q61L.

In another aspect, the present invention provides for a method for treating cancer comprising a mutation in PIK3CA in a subject in need thereof, said method comprising administering to the subject a pharmaceutical composition capable of inhibiting the expression or activity of AKT1 and AKT2.

In another aspect, the present invention provides for a kit comprising vectors according to any of embodiment herein or a library according to any embodiment herein and instructions for use.

In another aspect, the present invention provides for a system for generating a library for combinatorial screening, comprising a vector comprising convergent RNA polymerase III (RNAP III) promoters flanking a cloning site configured for accepting an oligonucleotide comprising inverted CRISPR guide sequences, optionally, a restriction enzyme and buffers specific to the cloning site.

In another aspect, the present invention provides for a combination of one or more agents targeting a first gene and one or more agents targeting a second gene for use as a medicament, wherein said first and second genes are selected from the group consisting of MTA1 and MTA2, HDAC1 and HDAC2, CHD3 and HDAC2, ING1 and ING2, ING4 and ING5, ASF1B and ASF1A, ARID4A and JADE2, ARID4A and SMYD1, ARID4A and SETD9, ATRX and HIRA, SLBP and HIRA, CREBBP and CARM1, ARID3A and RAD54L2, JMJD6 and WDR5, DPF2 and SMYD5, JMJD6 and MBD2, MSL3 and SRCAP, KMT2C and KMT2D, HDAC3 and SETD1B, KMT2A and KMT2B, KDM3B and KMT2D, SMARCA4 and SMARCA2, BRD8 and SMARCA1, WDR77 and BRD4, SETD6 and INO80, SMARCAL1 and ATRX, KAT6B and CHD8, ARID1B and ARID1A, WDR77 and HDAC6, WDR77 and KAT6B, KDM3B and ARID1A, KDM3B and CHD3, SETD2 and NSD1, MTA1 and DOT1L, KDM3B and BRD1, KDM4A and KAT6A, INO80 and CBX1, HDAC6 and EZH2, SMARCAL1 and HDAC8, KAT5 and CHAF1B, SUV39H1 and HDAC6, KDM3B and BRD4, KMT2B and BRD8, PRMT5 and KAT5, SIRT4 and CBX1, KAT6A and CHD6, WDR77 and DOT1L, KAT2B and EHMT1, KMT2E and KAT6A, KDM3B and DOT1L, KDM3B and KDM3A, CHD8 and BRD1, HIRA and ATRX, KDM5C and KDM3B, PRDM6 and KDM3B, KAT6B and KAT6A, SMARCB1 and KDM6A, MECP2 and KDM4B, KAT2A and HDAC5, SETD2 and KDM3B, RFWD2 and CHD6, SMARCB1 and ARID3C, SETMAR and BRD1, HDAC2 and DIDO1, HDAC2 and DNMT3B, KDM4D and BRD1, PRDM1 and HDAC8, SMARCA5 and KAT6A, and KMT2D and ARID1A.

In another aspect, the present invention provides for a personalized method for selecting a cancer treatment comprising determining in a subject suffering from cancer a deficiency in function or expression or a mutation in one or more pairs of genes selected from the group consisting of MTA1 and MTA2, HDAC1 and HDAC2, CHD3 and HDAC2, ING1 and ING2, ING4 and ING5, ASF1B and ASF1A, ARID4A and JADE2, ARID4A and SMYD1, ARID4A and SETD9, ATRX and HIRA, SLBP and HIRA, CREBBP and CARM1, ARID3A and RAD54L2, JMJD6 and WDR5, DPF2 and SMYD5, JMJD6 and MBD2, MSL3 and SRCAP, KMT2C and KMT2D, HDAC3 and SETD1B, KMT2A and KMT2B, KDM3B and KMT2D, SMARCA4 and SMARCA2, BRD8 and SMARCA1, WDR77 and BRD4, SETD6 and INO80, SMARCAL1 and ATRX, KAT6B and CHD8, ARID1B and ARID1A, WDR77 and HDAC6, WDR77 and KAT6B, KDM3B and ARID1A, KDM3B and CHD3, SETD2 and NSD1, MTA1 and DOT1L, KDM3B and BRD1, KDM4A and KAT6A, INO80 and CBX1, HDAC6 and EZH2, SMARCAL1 and HDAC8, KAT5 and CHAF1B, SUV39H1 and HDAC6, KDM3B and BRD4, KMT2B and BRD8, PRMT5 and KAT5, SIRT4 and CBX1, KAT6A and CHD6, WDR77 and DOT1L, KAT2B and EHMT1, KMT2E and KAT6A, KDM3B and DOT1L, KDM3B and KDM3A, CHD8 and BRD1, HIRA and ATRX, KDM5C and KDM3B, PRDM6 and KDM3B, KAT6B and KAT6A, SMARCB1 and KDM6A, MECP2 and KDM4B, KAT2A and HDAC5, SETD2 and KDM3B, RFWD2 and CHD6, SMARCB1 and ARID3C, SETMAR and BRD1, HDAC2 and DIDO1, HDAC2 and DNMT3B, KDM4D and BRD1, PRDM1 and HDAC8, SMARCA5 and KAT6A, and KMT2D and ARID1A; and selecting a treatment targeting the gene without a deficiency in function or expression or a mutation if a gene pair has a deficiency in function or expression or a mutation in only one gene in the pair. In certain embodiments, the cancer comprises Acute myeloid leukemia (AML). In certain embodiments, the cancer comprises a rearrangement in TEL or MLL.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Development of a two Cas9 system for combinatorial screening. (a) Schematic of the dual sgRNA expressing lentiviral vector used in this study, pPapi, as well as the cloning scheme. Pools of oligos are annealed, extended, and ligated into the pPapi vector, and used in cells that already carry the pLX_311 vector expressing SpCas9. (b) Flow cytometry plots indicating double knockout efficiency with percentage of cells indicated in each quadrant. (c) Area-Under-the-Curve (AUC) analysis of library representation. Representation was evaluated for the pDNA library for the Big Papi and CDKO libraries. Plasmid DNA sequencing was not provided for CombiGEM or Shen-Mali libraries, so early time points of genomic DNA were used, which typically very tightly match distributions of pDNA for sgRNA libraries. A perfectly distributed library (ideal) is shown in black. Big Papi SynLet library: sequencing of plasmid DNA (pDNA); Shen-Mali: day 3 genomic DNA from HeLa cells; CombiGEM: day 5 genomic DNA; CDKO: pDNA; Paired linc: pDNA. Percentages indicate each library's representation at 90% cumulative reads, and AUC values are noted in the key.

FIG. 2—Development of SaCas9 on-target rules. (a) Performance of tiled libraries of all possible sgRNAs targeting the essential EEF2 gene, grouped by PAM sequence. The box represents the 25^{th}, 50^{th} and 75^{th} percentiles, whiskers show 10^{th} and 90^{th} percentiles. (b) Comparison of the activity of EEF2 sgRNAs targeting the same cut site using either SaCas9 (NNGRRT PAM) or SpCas9 (NGG PAM). (c) Spearman correlations of the activity of sgRNAs targeting essential genes across cell lines. (d) Single nucleotide features predictive of SaCas9 activity. Top 20% of sgRNA sequences were treated as highly active and a 20% versus 80% classification model was used to identify predictive features. The −log 10 p-values are plotted (two-sided Fisher's exact test). (e) Contribution of different groups of features to the gradient boosted regression tree model for SaCas9 activity. (f) Example performance of the model. Using a version of the model in which EEF2 sgRNAs were not used in the training, sgRNA activity score is plotted versus the measured value. (g) For the model version used in (f), the fraction of sgRNAs that led to at least 4-fold depletion, binned by predicted score. The number of sgRNAs in each bin is shown above the bar. (h) Increase in model performance as more genes are used in the training set, using Spearman correlation to compare the predicted activity score to the measured value. Error bars represent standard deviation across random draws of the training genes and the held-out test gene.

FIG. 3—Evaluation of synthetic lethal screens. (a) Schematic of the Big Papi screens performed with the SynLet library. (b) Comparison of log 2-fold-change for sgRNA pairs across biological replicates and cell lines for the Big Papi approach and other published screens. When multiple time points were assessed, each is shown as a point and the line segment represents the mean. CombiGEM: Day 20 compared to Day 15; Shen-Mali: Day 14, Day 21, and Day 28 compared to Day 3; CDKO: Day 14 compared to pDNA, drug library; Big Papi: Day 9, 11, or 21 compared to pDNA. (c) Example comparison of the activity of targeting sgRNAs in the U6 position when paired with different control sgRNAs in the H1 position for the Big Papi screening approach. This data demonstrates the correlation among subsets of distinct library constructs that all target the same genomic site. (d) Pearson correlations for all pairwise combinations of controls, as in panel (c), for both sgRNA positions for several screening approaches. The point indicates the mean, the error bars represent one standard deviation for the range of pairwise correlation values. The promoter expressing the targeting sgRNA labels the x-axis. CombiGEM (n=3 pairwise comparisons): sgRNAs paired with 3 ‘dummy’ controls. Shen-Mali (n=1): sgRNAs paired with the non-targeting sgRNAs #362 and #412 in the HeLa data. CDKO (n=3,081): sgRNAs paired with 79 ‘safe’ sgRNAs. Big Papi (n=28): sgRNAs paired with ‘6T’ and ‘HPRT intron’ controls in the Meljuso, day 21 data. (e) Assessment of the essentiality of individual genes with the Big Papi screening approach at day 21. The log 2-fold-change for all six targeting sgRNAs, three with SaCas9 and three with SpCas9, were averaged to produce a gene-level score.

FIG. 4—Synthetic lethal Big Papi screen. (a) Correlation between measured and expected log 2-fold-change values for combinatorial targeting. Data points above (red) and below (blue) 2 standard deviations are highlighted, representing buffering and synthetic lethal interactions, respectively. Data from Meljuso cells are plotted as a representative cell line. (b) Distribution of all false discovery rates determined for buffering and synthetic lethal interactions using either data from individual cell lines (1 line) or combining data from 5 lines. When 5 lines are combined, more pairs score with either low FDRs or with an FDR=1. (c) FDRs for synthetic lethal interactions for gene pairs within pre-defined groups at the day 21 time point. Results are shown from individual cell lines, all leave-one-out combinations, and the combination of all 6 lines. (d) Primary screening data showing the performance of sgRNAs for BCL2L1 and MCL1 when paired together or with 6T controls in Meljuso cells at day 21. Average is denoted with a line whereas each dot represents an sgRNA combination. Dotted line refers to 2 standard deviations (2SD) from the mean for individual sgRNAs paired with controls (black dots). P-values for depletion of the dual-targeting sets of sgRNA pairs are based on the Mann-Whitney test, **P<0.01; ***P<0.001; ****P<0.0001. (e) Comparisons of the estimated true positive rate to the calculated FDR for synthetic lethal and buffering interactions, using either individual cell lines or all leave-one-out combinations of 5 cell lines. (f) Estimation of the false negative rate based on analysis of same-gene buffering interactions, using either individual cell lines or all leave-one-out combinations of 5 cell lines, plotted against the FDR.

FIG. 5—Validation of synthetic lethal interactions. (a) Gene expression values from the Cancer Cell Line Encyclopedia. (b) Validation of genetic interactions with individual gene knockout combined with small molecules. Seven days after transduction with lentivirus expressing individual sgRNAs, cells were incubated with small molecules for three days before assaying viability by Cell Titer Glo. Points represent the average and whiskers represent the maximum and minimum of two replicate wells. (c) Validation of BCL2L1-MCL1 genetic interaction with combinations of small molecules. Cells were incubated with small molecules for three days before assaying viability by Cell Titer Glo (top). Bliss independence scores were then calculated (bottom). (d) Schematic of a competition experiment used to compare cell viability of single versus double knockout of BRCA1 and PARP1. EGFP is co-delivered with SpCas9 at a low MOI, followed by introduction of the pPapi vector, which contained SaCas9 and two sgRNAs targeting BRCA1 and PARP1 with SpCas9 and SaCas9, respectively (p083), or the reverse (p092). EGFP is thus a marker for SpCas9 delivery; EGFP+ cells are double knockouts while EGFP-cells only have knockout of the SaCas9-targeted gene. Controls, containing 6T in place of the sgRNA, were also included. (e) Fraction of EGFP+ cells over time for cells receiving the indicated vector, normalized to the population that received the 6T control construct. The pPapi vectors were infected in triplicate, and error bars represent the standard deviation of the three measurements.

FIG. 6—Apoptosis Big Papi screen. (a) Schematic of the screen design. (b) Genes targeted by the Apoptosis library and the viability effects caused by single gene knockout; fold change values are calculated relative to the pDNA pool for targeting sgRNAs paired with the 6T and HPRT intron controls. (c) FDRs for buffering interactions detected between pro- and anti-apoptotic genes in Meljuso and OVCAR8 cells as well as the combined data from both cell lines. (d) From the Cancer Cell Line Encyclopedia, expression levels of these genes in Meljuso cells. BAK1 was not assessed in the CCLE, indicated by an asterisk. (e) In Meljuso cells with single gene knockouts, comparison of resistance and sensitization phenotypes for two small molecules. The fold change values are calculated relative to the no drug arm for targeting sgRNAs paired with the 6T and HPRT intron controls. Genes of interest are colored and labeled. (f) Buffering interactions in Meljuso cells for combinations of multidomain apoptotic genes with BH3-only sensitizer genes in different growth conditions. Data from the three small molecules were combined for the final column. Heat map scale is the same as in panel c. (g) Buffering interactions in Meljuso cells for combinations of pro-apoptotic genes and caspase genes in standard growth conditions and the combined data from the three small molecules. Heat map scale is the same as in panel c.

FIG. 7—Big Papi screen with two Cas9 activities. (a) In addition to using either or both Cas9s as DNA endonucleases to inactivate genes, nuclease dead versions of Cas9 (dCas9) can be used with appended domains to manipulate DNA with multiple activities. (b) Schematic of the screen for the TsgOnco Big Papi library. (c) For the TsgOnco library in high attachment conditions in HAlE cells, comparison of the activity of CRISPRa sgRNAs when paired with control SaCas9 sgRNAs. (d) Comparison of the activity of CRISPR-knockout sgRNAs when paired with control dSpCas9-VPR sgRNAs in high attachment conditions. (e) Buffering interaction observed in HAlE cells, where knockout of TP53 protects the cells from loss of viability caused by overexpression of TP53. Data for both low and high attachment conditions are shown. P-values for depletion of the dual-targeting sets of sgRNA pairs are based on the Mann-Whitney test; significance labels: **P<0.01; ****P<0.0001. (f) Knockout of tumor suppressor genes, comparing viability upon TP53 overexpression to the average viability of all other CRISPRa target genes. Genes of interest are labeled and colored.

FIG. 8—Potential sources of inefficiency for single Cas9 systems, and their solutions in a two Cas9 system. (a) Single Cas9 system using two copies of the U6 promoter. Repetitive elements such as the S. pyogenes tracrRNA (Sp tr) and U6 promoter are prone to recombination. Use of one Cas9 also risks unequal targeting due to competitive association resulting from unequal sgRNA transcription rates, sgRNA stability, and/or sequence preferences of Cas9. (b) Single Cas9 system using two different promoters. Promoters are no longer prone to recombination, although SpCas9 trRNA sequences still have sequence overlap. Additionally, unequal transcription from different promoters may exacerbate competitive association for the same Cas9. (c) A two Cas9, two promoter system. Recombination at the plasmid and/or lentiviral stage is minimized since each Cas9 uses a distinct tracrRNA and each sgRNA is driven off a distinct promoter with minimal sequence overlap. Furthermore, a two-Cas9 system enables independent association of each sgRNA to its cognate Cas9, avoiding unequal targeting due to competition for a single Cas9 between sgRNAs with potentially unequal sgRNA transcription rates, sgRNA stability, and sequence preference for Cas9, especially in cases where Cas9 expression may be low.

FIG. 9—Dual sgRNA vectors targeting EGFP and CD81. (a) Flow cytometry plots of constructs assayed 7 days post-infection. A representative plot for live/dead gating using forward and side scatter is shown. From either promoter, targeting EGFP with SaCas9 achieved greater than 95% knockout efficiency when partnered with an SpCas9 sgRNA employing the other promoter. However, the EGFP-targeting SaCas9 sgRNA was rendered inactive from either promoter when partnered with another SaCas9 sgRNA. The same trend was observed, although with a smaller effect size, with an SpCas9 sgRNA targeting CD81. (b) For two constructs analyzed in (a), flow cytometry analysis 17 days post-infection. (c) PCR analysis of the lentiviral-integrated dual sgRNA expression cassettes from genomic DNA, using the same PCR primers and conditions as used to amplify libraries for sequencing analysis. A single product predominates when the construct contained one SpCas9 sgRNA and one SaCas9 sgRNA, whereas constructs with two SaCas9 sgRNAs or two SpCas9 sgRNAs showed diminished abundance of the full-size product and the appearance of multiple smaller products, suggesting that recombination contributes to decreased knockout efficiency. Constructs are numbered as in panel (a).

FIG. 10—Development of SaCas9 on-target rules. (a) Representation of all SaCas9 sgRNAs tested according to group and gene. sgRNAs are grouped based on whether the target gene is assayed by 6-thioguanine resistance, cell viability, or vemurafenib resistance. Control sgRNAs are also indicated. (b) Log 2 fold change of sgRNAs relative to their starting abundance in the plasmid DNA library in cell viability and resistance experiments. That knockout of NUDTS confers 6-thioguanine resistance in 293T cells but not A375 cell is expected based on previous results¹⁶. P-values: Kruskal-Wallis test with Dunn's multiple comparisons test for each gene relative to the set of non-targeting controls. Significance labels: ns, not significant; *P<0.05; **P<0.01; ***P<0.001; ****P<0.0001.

FIG. 11—Screening performance of SynLet library across cell lines. (a) Comparison of the effect of single gene knockout on cell viability as determined with SpCas9 versus SaCas9. Multiple sgRNAs per gene were averaged to generate a gene-level value. Pearson correlations are indicated. (b) Comparison of essentiality of individual genes across samples, plotting the average of the Log 2-fold-change values for SaCas9 and SpCas9. Data are the same as in FIG. 3e, with the addition of earlier time points when available.

FIG. 12—Analysis methodology for assessing genetic interactions. (a) Model for detecting genetic interactions by determining the delta Log 2-fold-change (ΔLFC), the deviation of the measured Log 2-fold-change from the expectation for two sgRNAs, as determined by their log 2-fold-change when paired with controls. (b) For each of the 6 sgRNAs (3 for each Cas9) for a given test gene, BCL2L1 in this example, the 96 partner sgRNAs are ranked by the ΔLFC calculation. These ranks are then collated by the identity of the partner gene and averaged. When data from multiple conditions (e.g. cell lines, small molecule treatments) were combined, the ΔLFC values were median-centered within each dataset, and then the test-partner analysis performed. (c) Average rank of partnered sgRNAs for the procedure described in (b). The null distribution was determined by performing the same calculations on 2,000 random shuffles of the sgRNA labels, allowing the calculation of a false discovery rate (FDR). Three outlier synthetic lethal gene pairs in Meljuso cells are highlighted. When information from multiple cell lines or small molecule treatments are combined, a new null distribution must be derived, as the ranked list of partner genes spans a larger range (e.g. for combining the SynLet data across all cell lines, there are 6×96=576 partner sgRNAs). (d) Comparison of the average ranks when the identities of test and partner genes are swapped. Pearson correlation is shown. Colored dots represent gene pairs of interest. Gray dots represent the special case where the test and partner genes are the same, which were excluded from the calculation of correlation. (e) Comparison of the average rank of partnered sgRNAs in Meljuso cells harvested at day 9 and day 21. Dots in gray are gene pairs where both Cas9s target the same gene. The blue and red dotted lines indicate ranks that correspond to a false discovery rate of 0.01 for synthetic lethal and buffering interactions, respectively.

FIG. 13—Comparisons of top hits across screening approaches. Dotted line refers to 2 standard deviations (2SD) from the mean for the set of all individual sgRNAs paired with controls. P-values for depletion of the dual-targeting sets of sgRNA pairs are based on the Mann-Whitney test; significance labels: ns, not significant; *P<0.05; **P<0.01; ***P<0.001; ****P<0.0001. Big Papi: Data from BCL2L1-MCL1 at Day 21 in Meljuso cells are repeated here from FIG. 4d for ease of comparison. Hits from other cell lines are shown; false discovery rates (FDRs) from Supplementary Table 4. CDKO: Data from two top hits in the primary screen. For BCL2L1-MCL1, data are shown when unfiltered or filtered by read count. Shen-Mali: top two hits from each cell line based on analysis provided in that publication. Log-fold-change values are the average for the day 14, 21, and 28 time points compared to the day 3 time point. CombiGEM: Data from comparison of Day 20 to Day 15 for two top hits highlighted in that publication.

FIG. 14—Analysis of same-gene interactions. These analyses examine the special case where both sgRNAs target the same gene. (a) For each cell line, the number of same-gene interactions that score with an FDR<0.25 for synthetic lethal and buffering interactions. (b) FDRs for the buffering and synthetic lethal interactions for individual cell lines, all leave-one-out iterations, and the combination of all 6 cell lines. Most genes engage in same-gene buffering interactions, indicating effective targeting, but some genes show a synthetic lethal interaction. In this case, each sgRNA is not fully effective in targeting the gene, and thus there is additional viability loss possible when two sgRNAs are used to target the gene.

FIG. 15—Additional validation of anti-apoptotic gene interactions. (a) As in FIG. 5b, cells were infected with individual sgRNAs and 7 days post-infection treated with small molecules over a range of doses. Cell viability was determined by Cell Titer Glo after 3 days. (b) As in FIG. 5c, cells were treated with combinations of small molecules and cell viability was determined by Cell Titer Glo after 3 days (top) and Bliss independence scores were calculated (bottom).

FIG. 16—Apoptosis library screening results. All pairwise buffering and synthetic lethal FDRs are shown for the combined OVCAR8 and Meljuso data in standard growth conditions.

FIG. 17—TsgOnco screening results. The log 2-fold-change values were first averaged across all pairs of sgRNAs targeting the same gene pairs, and then median-centered within each gene knockout.

FIG. 18—Rescue from TP53 overexpression. As in FIG. 7e, log 2-fold-change values for individual pairs of sgRNAs. P-values for depletion of the dual-targeting sets of sgRNA pairs are based on the Mann-Whitney test; significance labels: ns, not significant; *P<0.05; **P<0.01; ***P<0.001; ****P<0.0001.

FIG. 19—Subsampling of SynLet library. (a) Random draws of increasing numbers of sgRNAs were sampled and FDRs calculated. The dotted line indicates an FDR threshold of 0.01. 786O cells are not shown because no gene pairs scored with an FDR<0.01. Applicants found the largest single-step improvement in the number of detected interactions going from 3 to 4 total sgRNA combinations, when each gene in a pair is examined with at least 2 sgRNAs. (b) Total number of genes detected across the five cell lines with increasing numbers of sgRNAs. The points indicate the total at an FDR threshold of 0.01, and the whiskers indicate FDR thresholds of 0.005 and 0.02. Thus, the performance of the Big Papi system makes it feasible to reduce from 3 to 2 guides per gene, reducing the screen size by 2.25-fold (3²/2²) or alternatively to decrease the stringency of the analysis by requiring only a fraction of sgRNA pairs to display robust activity for hit identification in the primary screen, improving sensitivity.

FIG. 20—illustrates pPapi, U6-H1 region of the vector (SEQ ID NO:45,538).

FIG. 21—illustrates the PCR primers for sequencing deconvolution (SEQ ID NO:45,539 and SEQ ID NO:45,540).

FIG. 22—illustrates sgRNA sequences (SEQ ID NO:45,541-45,548).

FIG. 23—illustrates a schematic of disease relevant screening of a dual sgRNA pooled library in leukemia cell lines.

FIG. 24—illustrates a triage methodology for characterizing genes assayed in the combinatorial screen.

FIG. 25—illustrates synthetic lethal combinations (ARID1A;ARID1B). NT=non-targeting sgRNA. The combinations are shown for sgRNAs for both of the orthologous CRISPR enzymes.

FIG. 26—illustrates a schematic for follow-up validation of synthetic lethal combinations using a GFP vector.

FIG. 27—illustrates validation that ARID1A;ARID1B knockout impairs growth.

FIG. 28—illustrates that synthetic lethal genes rarely buffer in combinations and epistatic genes buffer lethal genes.

FIG. 29—illustrates buffering and that HDAC3 is a pseudo essential gene.

FIG. 30—illustrates buffering and that TAF3 (pseudo essential gene) knockout is rescued by NSD1/2 loss.

FIG. 31—illustrates buffering and that MLL (KMT2A) knockout is partially rescued by NSD1/2 loss.

FIG. 32—illustrates that the combination screening methodology can improve or predict responses from existing drugs or drug targets.

FIG. 33—illustrates that WDR77 and BRD4 are a synthetic lethal combination.

FIG. 34—illustrates that WDR77 KO sensitizes THP-1 cells to JQ1 treatment. JQ1 dose response+/−WDR77 KO. Octuplicate wells. Repeated with two sgRNAs.

FIG. 35—illustrates that WDR77 KO sensitizes THP-1 (AML MLL-AF9) cells to AZD5153 treatment.

FIG. 36—illustrates that WDR77 KO sensitizes MV4-11 (AML MLL-AF4) cells to AZD5153 treatment.

FIG. 37—illustrates that SETD6 and INO80 are a synthetic lethal combination.

FIG. 38—illustrates follow-up experiment validating synthetic lethality of SETD6 and INO80 in cancer cell lines. Cell lines THP-1 and Nomo-1 (MLL-AF9 fusion AML) and Reh (no MLL fusion) were utilized. Cells were transduced with combo CRISPR GFP lentivirus and fluorescence analyzed at two time points. NT=non-targeting guide. EEF2 is an essential gene.

FIG. 39—illustrates follow-up validation experiments for the indicated combinations. Fluorescence was analyzed at two time points (Day 3 and 21). NT=non-targeting guide. EEF2 is an essential gene.

FIG. 40—illustrates the screening result of TAF3 and PHF23 combination showing that PHF23 knockout buffers TAF3 essentiality.

FIG. 41—illustrates a singleton gene knockout data library screen in REH and THP-1 cells.

FIG. 42—Selection and characterization of chromatin regulators for combinatorial screening. a) Pie chart summarizing activities of the 268 selected chromatin regulator genes. These genes contained 374 protein family (PFAM) domains that were compiled into broad functional categories. b) Bar plot with deletion frequency of the 268 genes found in 10,967 TCGA samples. X-axis extends out to 35 deletions. Data were compiled from cbioportal. c) Top homozygous deletions of the 268 chromatin regulators in TCGA samples. Complexes and genes investigated further in this study are demarcated. d) Schematic representation of library cloning, lentiviral production, THP-1 or Reh cell transduction, and screening for viability.

FIG. 43—Essential singleton and combinatorial hits from the 300 k library screen. a,b) Histogram of singleton knockout data from the combinatorial screen in Reh (acute lymphocytic leukemia, a) and THP-1 (acute myeloid leukemia, b) cells. Hits below 2 standard deviations are highlighted. EEF2 is an essential gene used as a control. c,d) Volcano plot depicting the most likely synthetic lethal combinatorial hits from screening in Reh (c) and THP-1 (d). Data are an average of two replicates. Depletion score is an absolute measurement of loss of averaged gene pair data. Pi score is an interaction score accounting for the effects of each gene separately and measuring the additional effect by pairing the two genes.

FIG. 44—NuRD and SIN3A complex dependencies across eight leukemia lines. a) Schematic representation of the 8 k library generation from a selection of 39 genes, many hits from the 300 k combinatorial screen, and validation screening in Reh and THP-1 as well as six additional AML lines. b) Graphical representation of the canonical NuRD complex according to HUGO Gene Nomenclature Committee. c,d) Heatmaps of RNAseq data (c) and Avana knockout data (d) from the DepMap for tested NuRD complex members. e) Combinatorial knockout heatmap from the 8 k library screen. Pi score was supplemented with a z-score calculation to indicate confidence in heatmap. f,g) Heatmaps of RNAseq data (f) and Avana knockout data (g) from the DepMap for tested SIN3A complex members. h) Combinatorial knockout heatmap from the 8 k library screen for SIN3A complex members tested. i) Graphical representation of the canonical SIN3A complex according to HUGO Gene Nomenclature Committee. For all panels with Avana, 19q1 data were sourced. For all figures with RNAseq, 18Q1 data were sourced. “TEL-r” and “MLL-r” indicates a rearrangement in the TEL or MLL genes. “None” indicates neither a TEL or MLL rearrangement detected in the lines.

FIG. 45—ASF1 and KAT7 complex dependencies across eight leukemia lines. a,b) Heatmaps of RNAseq data (a) and Avana knockout data (b) from the DepMap for histone chaperone members. c) Combinatorial knockout heatmap from the 8 k library screen for ASF1A and ASF1B. d,e) Heatmaps of RNAseq data (d) and Avana knockout data (e) from the DepMap for KAT7 acetyltransferase complex. f) Combinatorial knockout heatmap from the 8 k library screen for ING4 and ING5. g) Graphical representation of the canonical KAT7 complex according to CORUM. For all panels with Avana, 19q1 data is sourced. For all figures with RNAseq, 18Q1 data is sourced. “TEL-r” and “MLL-r” indicates a rearrangement in the TEL or MLL genes. “None” indicates neither a TEL or MLL rearrangement detected in the lines.

FIG. 46—Characterization of 268 chromatin regulator genes. a) Gene ontology, molecular function. b) Homozygous deletion frequency in 881 CCLE samples. Inset: Pie chart depicting the percent of cell lines with 1 or greater deletions compared to no deletions. c) Histogram of the most frequently mutated chromatin regulator genes in blood CCLE sample. Relevant genes and complexes to this study are highlighted.

FIG. 47—Avana results of selected chromatin regulators. Heatmap with 234 tested of 268 chromatin regulators, with the top 22 pan-essential genes labeled.

FIG. 48—Cas9 ortholog performance comparison for the 40 k library screen. Two-tailed pearson correlation.

FIG. 49—Replicate correlation in the 40 k library screen. Two-tailed pearson correlation.

FIG. 50—40 k library screen data. a,b) Singleton knockout frequency distributions for Reh (a) and THP-1 (b) with hits below 2 standard deviations labeled. c,d) Example guide pair data for each cell line tested. ***P<0.001 e,f) Combinatorial data.

FIG. 51—Cas9 ortholog performance comparison for the 300 k library screen. Two-tailed pearson correlation.

FIG. 52—Replicate correlation in the 300 k library screen. Two-tailed pearson correlation.

FIG. 53—Singleton knockout data from the 300 k library screen correlated to Avana. a,b) Regression analysis of 300 k library screen dependency vs. Avana dependency in Reh (a) and THP-1 (b). 300 k library data were normalized such that the median log-2 fold change is 0 and the median absolute deviation is 1. c) Venn diagram of hits identified in 300 k library screen. Two-tailed pearson correlation.

FIG. 54—Tumor suppressor mutations found in the eight leukemia lines in this study. A selection of 129 tumor suppressors (Vogelstein) were analyzed for mutations. Data are from cbioportal and DepMap, with a detected mutation in either database selected for display.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +1-10% or less, +1-5% or less, +/−1% or less, and +1-0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to the following publication authored by the Applicants: Najm et al., Orthologous CRISPR-Cas9 enzymes for combinatorial genetic screens. Nat Biotechnol. 2018 February; 36(2):179-189. doi: 10.1038/nbt.4048. Epub 2017 Dec. 18.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide a screening platform for the combinatorial screening of phenotypic interactions, a combinatorial screening platform that targets chromatin regulators, targets identified using the described screening platform for use as therapeutic candidates, and a synthetic lethality and buffering gene methodology. In certain embodiments, the screening platform can be used to define a chromatin landscape, weaknesses, and/or vulnerabilities in a disease (e.g., cancer), thus, informing therapeutic approaches.

Combinatorial genetic screening using CRISPR-Cas9 is a useful approach to uncover redundant genes and to explore complex gene networks. However, current approaches suffer from interference between the single-guide RNAs (sgRNAs) and from limited gene targeting activity. Applicants developed an approach that relies on orthogonal Cas9 enzymes, from S. pyogenes and S. aureus (SpCas9 and SaCas9), to overcome practical limitations of previous approaches and to achieve dual-knockout efficiencies that enable robust screening.

Applicants used machine learning to establish S. aureus Cas9 sgRNA design rules and paired S. aureus Cas9 with S. pyogenes Cas9 to achieve dual targeting in a high fraction of cells. Applicants also developed a lentiviral vector and cloning strategy to generate high-complexity pooled dual-knockout libraries to identify synthetic lethal and buffering gene pairs across multiple cell types, including MAPK pathway genes and apoptotic genes. The orthologous approach enabled a screen combining gene knockouts with transcriptional activation, which revealed genetic interactions with TP53. The “Big Papi” (Paired aureus and pyogenes for interactions) approach described here is widely applicable for the study of combinatorial phenotypes.

This approach uncovered synthetic lethal and buffering relationships across multiple cell types with excellent correspondence between unique sgRNA pairs targeting the same gene pairs. As two sgRNAs independently program two different Cas9s, this approach can combine different activities in the same screen, such as knockout and overexpression (CRISPRa)¹⁵. The screening platform using paired aureus and pyogenes can be used for interaction screens to interrogate large combinatorial space at scale and has applications in many cellular models.

Genes that regulate chromatin are often mutated in cancer, and are commonly found in redundant pathways. Applicants have developed a chromatin regulator screening platform and have identified synthetic lethal gene combinations and buffering combinations. These combinations may be targeted pharmaceutically in disease.

### Screening Platform

In certain example embodiments, the present invention provides for a screening platform to allow for the perturbation of combinations of target sequences. The screening platform advantageously uses orthogonal CRISPR enzymes to perturb two target sequences in combination in a cell. The screening platform advantageously uses a library of pairwise perturbation target combinations to allow for a pooled screen in a population of cells.

The term “orthogonal CRISPR enzymes” refers to CRISPR enzymes (i) that do not cross-activate or interfere with each other; and (ii) do not interact with the sgRNAs of the other CRISPR enzyme. In certain embodiments, orthogonal CRISPR enzymes recognize different scaffold sequences and recognize different PAM sequences. In certain embodiments, orthogonal CRISPR enzymes can be naturally occurring CRISPR enzymes or engineered non-naturally occurring CRISPR enzymes. In certain embodiments, orthogonal CRISPR enzymes include, but are not limited to SaCas9 and SpCas9 (described further herein). In certain embodiments, the present invention includes any pair of orthogonal CRISPR enzymes having different PAM sequences and recognizing different scaffold sequences.

The terms “target nucleic acid,” “target site,” and “target sequence” may be used interchangeably throughout and refer to any nucleic acid sequence in a host cell that may be targeted by the CRISPR guide sequences described herein. The target nucleic acid is flanked downstream by a protospacer adjacent motif (PAM) that may interact with the endonuclease (e.g., orthogonal CRISPR enzymes) and be further involved in targeting the endonuclease activity to the target nucleic acid. It is generally thought that the PAM sequence flanking the target nucleic acid depends on the endonuclease and the source from which the endonuclease is derived. For example, for Cas9 endonucleases that are derived from Streptococcus pyogenes, the PAM sequence is NGG. For Cas9 endonucleases derived from Staphylococcus aureus, the PAM sequence is NNGRRT. For Cas9 endonucleases that are derived from Neisseria meningitidis, the PAM sequence is NNNNGATT. For Cas9 endonucleases derived from Streptococcus thermophilus, the PAM sequence is NNAGAA. For Cas9 endonuclease derived from Treponema denticola, the PAM sequence is NAAAAC. For a Cpf1 nuclease, the PAM sequence is TTN. As used herein, the term “targeting” of a selected DNA sequence means that a guide RNA is capable of hybridizing with a selected DNA sequence.

In any of the non-naturally-occurring CRISPR enzymes, the CRISPR enzyme may comprise a CRISPR enzyme from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium or Corynebacter.

In any of the non-naturally-occurring CRISPR enzymes, the CRISPR enzyme may comprise a chimeric Cas9 enzyme comprising a first fragment from a first Cas9 ortholog and a second fragment from a second Cas9 ortholog, and the first and second Cas9 orthologs are different. At least one of the first and second Cas9 orthologs may comprise a Cas9 from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium or Corynebacter.

In certain embodiments, the Cas9 endonuclease is derived from Streptococcus pyogenes, Staphylococcus aureus, Neisseria meningitidis, Streptococcus thermophilus, or Treponema denticola. In certain embodiments, the nucleotide sequence encoding the Cas9 endonuclease may be codon optimized for expression in a host cell or organism. In certain embodiments, the endonuclease is a Cas9 homology or ortholog.

In certain embodiments, the endonuclease is a Cpf1 nuclease (Cas12). In certain embodiments, the Cpf1 nuclease is derived from Pwvetella spp. or Francisella spp. In certain embodiments, the nucleotide sequence encoding the Cpf1 nuclease may be codon optimized for expression in a host cell or organism. Not being bound by a theory, expression of Cpf1 in a combinatorial screening approach as described herein may require that the Cpf1 nuclease is expressed at higher levels than an orthogonal CRISPR enzyme in order to account for lower gene editing efficiency.

In preferred embodiments, the orthogonal CRISPR enzymes are Cas9 endonucleases derived from Streptococcus pyogenes and Staphylococcus aureus.

In aspects of the invention the terms “guide sequence” “chimeric RNA”, “chimeric guide RNA”, “guide RNA”, “single guide RNA”, “sgRNA”, and “synthetic guide RNA” are used interchangeably and refer to the polynucleotide sequence comprising the guide sequence, preferably the tracr sequence and the tracr mate sequence. The term “guide sequence” refers to the about 20 bp sequence within the guide RNA that specifies the target site and may be used interchangeably with the terms “guide” or “spacer”. The term “tracr mate sequence” may also be used interchangeably with the term “direct repeat(s)”. In certain embodiments, the term “sgRNA sequence” may refer to a DNA sequence encoding for a sgRNA.

In a host cell, the DNA element comprising a CRISPR guide sequence and a scaffold sequence is transcribed and forms a CRISPR single guide RNA (sgRNA) that functions to recruit an endonuclease to a specific target nucleic acid in a host cell, which may result in site-specific CRISPR activity. As used herein, a “CRISPR guide sequence” refers to a nucleic acid sequence that is complementary to a target nucleic acid sequence in a host cell. The CRISPR guide sequence targets the sgRNA to a target nucleic acid sequence, also referred to as a target site. The CRISPR guide sequence that is complementary to the target nucleic acid may be between 15-25 nucleotides, 18-22 nucleotides, or 19-21 nucleotides in length. In certain embodiments, the CRISPR guide sequence that is complementary to the target nucleic acid is 15, 16, 17, 18, 19, 20, 21, 22, 23, 24, or 25 nucleotides in length. In certain embodiments, the CRISPR guide sequence that is complementary to the target nucleic acid is 20 nucleotides in length.

It will be appreciated that a CRISPR guide sequence is complementary to a target nucleic acid in a host cell if the CRISPR guide sequence is capable of hybridizing to the target nucleic acid. In certain embodiments, the CRISPR guide sequence is at least 50%, 55%, 60%, 65%, 70%, 75%, 80%, 85%, 90%, 95%, 96%, 97%, 98%, 99%, or at least 100% complementary to a target nucleic acid (see also U.S. Pat. No. 8,697,359, which is incorporated by reference for its teaching of complementarity of a CRISPR guide sequence with a target polynucleotide sequence). It has been demonstrated that mismatches between a CRISPR guide sequence and the target nucleic acid near the 3′ end of the target nucleic acid may abolish nuclease cleavage activity (Upadhyay, et al. Genes Genome Genetics (2013) 3(12):2233-2238). In certain embodiments, the CRISPR guide sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid). The CRISPR guide sequence may be obtained from any source known in the art. For example, the CRISPR guide sequence may be any nucleic acid sequence of the indicated length present in the nucleic acid of a host cell (e.g., genomic nucleic acid and/or extra-genomic nucleic acid). In certain embodiments, CRISPR guide sequences may be designed and synthesized to target desired nucleic acids, such as nucleic acids encoding transcription factors, signaling proteins, transporters, etc. In certain embodiments, the CRISPR guide sequences are designed and synthesized to target epigenetic genes.

In certain embodiments, the guide sequences are encoded for by a DNA construct comprising a nucleotide sequence. In certain embodiments, the DNA construct comprises a pair of orthologous guide sequences that are inverted in relation to each other. By inverted it is meant that the guide sequences are facing each other. In preferred embodiments, the nucleotide sequence encodes the pair of guide sequences such that the ends of the guide sequences are facing. In certain embodiments, the inverted configuration allows for construction of the orthologous screening platform, such that each construct encodes a guide sequence specific to each orthologous CRISPR enzyme.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

In certain embodiments, the screening platform constructs utilize two regulatory sequences that do not have 100% identity, such that each of the two guide sequences is operably linked to one of the regulatory sequences. In certain embodiments, the two regulatory sequences cannot recombine in the host cell because they do not have 100% identity. In certain embodiments, the regulatory sequences do not have 90, 80, 70, 60, 50, or less than 40% identity.

In certain embodiments, the regulatory sequences (e.g., promoters) operably linked to each guide sequence are convergent. In certain embodiments, the sequences encoding the guide sequences are inverted (e.g., the downstream sequence of each guide sequence face each other and each guide sequence is transcribed in the opposite direction). As used herein the term “convergent promoters” refers to promoters that are situated on either side of the inverted guide sequence cassette, such that the direction of transcription from each promoter is towards the center of the inverted guide sequences. In certain embodiments, this allows for a single construct comprising two inverted guide sequences to be inserted between the convergent regulatory sequences in a single cloning step.

Within an expression vector, “operably linked” is intended to mean that the nucleotide sequence of interest is linked to the regulatory sequence(s) in a manner which allows for expression of the nucleotide sequence (e.g., in an in vitro transcription/translation system or in a target cell when the vector is introduced into the target cell).

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In certain embodiments, a vector comprises one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1a promoter. The guide RNA(s), e.g., sgRNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) (e.g., a doxycycline inducible promoter) and/or tissue specific promoter(s). In certain embodiments, the invention can include inducible promoters and inducing expression. Also, encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

By “RNA polymerase III promoter” or “RNA pol III promoter” or “polymerase III promoter” or “pol III promoter” is meant any invertebrate, vertebrate, or mammalian promoter, e.g., human, murine, porcine, bovine, primate, simian, etc. that, in its native context in a cell, associates or interacts with RNA polymerase III to transcribe its operably linked gene, or any variant thereof, natural or engineered, that will interact in a selected host cell with an RNA polymerase III to transcribe an operably linked nucleic acid sequence. By U6 promoter (e.g., human U6, murine U6), H1 promoter, or 7SK promoter is meant any invertebrate, vertebrate, or mammalian promoter or polymorphic variant or mutant found in nature to interact with RNA polymerase III to transcribe its cognate RNA product, i.e., U6 RNA, H1 RNA, or 7SK RNA, respectively. Preferred in some applications are the Type III RNA pol III promoters including U6, H1, and 7SK which exist in the 5′ flanking region, include TATA boxes, and lack internal promoter sequences. Internal promoters occur for the pol III 5S rRNA, tRNA or VA RNA genes. The 7SLRNA pol III gene contains a weak internal promoter and a sequence in the 5′ flanking region of the gene necessary for transcription. RNA pol III promoters include any higher eukaryotic, including any vertebrate or mammalian, promoter containing any sequence variation or alteration, either natural or produced in the laboratory, which maintains or enhances but does not abolish the binding of RNA polymerase III to said promoter, and which is capable of transcribing a gene or nucleotide sequence, either natural or engineered, which is operably linked to said promoter sequence. Pol III promoters for utilization in an expression construct for a particular application, e.g., to express RNA effector molecules such as guide sequences, may advantageously be selected for optimal binding and transcription by the host cell RNA polymerase III, e.g., including murine pol III promoters and human or other mammalian pol III promoters in an expression construct designed to transcribe a plurality of guide sequences in human host cells.

In certain embodiments, the DNA construct further comprises a sequence encoding at least one selectable marker. The at least one selectable marker may be an antibiotic resistance gene. The at least one selectable marker may be a fluorescent gene.

Selectable markers are known in the art and enable screening for targeted integrations. Examples of selectable markers include, but are not limited to, antibiotic resistance genes, such as beta-lactamase, neo, FabI, URA3, cam, tet, blasticidin, hyg, puromycin and the like. A selectable marker useful in accordance with the invention may be any selectable marker appropriate for use in a eukaryotic cell, such as a mammalian cell, or more specifically a human cell. One of skill in the art will understand and be able to identify and use selectable markers in accordance with the invention.

In certain embodiments, the selectable marker is a fluorescent protein such as green fluorescent protein (GFP), enhanced green fluorescent protein (EGFP), red fluorescent protein (RFP), blue fluorescent protein (BFP), cyan fluorescent protein (CFP), yellow fluorescent protein (YFP), miRFP (e.g., miRFP670, see, Shcherbakova, et al., Nat Commun. 2016; 7: 12405), mCherry, tdTomato, DsRed-Monomer, DsRed-Express, DSRed-Express2, DsRed2, AsRed2, mStrawberry, mPlum, mRaspberry, HcRedl, E2-Crimson, mOrange, mOrange2, mBanana, ZsYellowl, TagBFP, mTagBFP2, Azurite, EBFP2, mKalamal, Sirius, Sapphire, T-Sapphire, ECFP, Cerulean, SCFP3A, mTurquoise, mTurquoise2, monomelic Midoriishi-Cyan, TagCFP, niTFP1, Emerald, Superfolder GFP, Monomeric Azami Green, TagGFP2, mUKG, mWasabi, Clover, mNeonGreen, Citrine, Venus, SYFP2, TagYFP, Monomeric Kusabira-Orange, mKOk, mK02, mTangerine, mApple, mRuby, mRuby2, HcRed-Tandem, mKate2, mNeptune, NiFP, mkeima Red, LSS-mKatel, LSS-mkate2, mBeRFP, PA-GFP, PAmCherryl, PATagRFP, TagRFP6457, IFP1.2, iRFP, Kaede (green), Kaede (red), KikGR1 (green), KikGR1 (red), PS-CFP2, mEos2 (green), mEos2 (red), mEos3.2 (green), mEos3.2 (red), PSmOrange, Dronpa, Dendra2, Timer, AmCyan1, or a combination thereof.

In certain embodiments, each guide sequence may further comprise a barcode sequence (e.g., to identify the guide sequence). The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA such that multiple species can be sequenced together. In certain embodiments, barcodes are designed using an error correcting scheme (T. K. Moon, Error Correction Coding: Mathematical Methods and Algorithms (Wiley, New York, ed. 1, 2005)).

In certain embodiments, the screening platform includes oligonucleotide constructs as described herein. The oligonucleotide construct may be present in a vector, such that the constructs can be delivered to a host cell. In certain embodiments, the screening platform includes a library of vectors wherein each vector of the library may comprise a different pairwise combination of guide sequences. As used herein, the term “vector” refers to a nucleic acid molecule capable of transporting another nucleic acid to which it has been linked. One type of vector is a “plasmid”, which refers to a circular double stranded DNA loop into which additional nucleic acid segments can be ligated. Another type of vector is a viral vector; wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors”, or more simply “expression vectors.” In general, expression vectors of utility in recombinant DNA techniques are often in the form of plasmids. In the present specification, “plasmid” and “vector” can be used interchangeably as the plasmid is the most commonly used form of vector. However, the methods and compositions described herein can include such other forms of expression vectors, such as viral vectors (e.g., replication defective retroviruses, lentiviruses, adenoviruses and adeno-associated viruses), which serve equivalent functions. In certain embodiments, the library of the present invention is introduced by a viral vector. The viral vector may be a lentivirus, adenovirus, or AAV. In preferred embodiments of the invention the viral vector is a lentivirus-derived vector. In certain embodiments, the vector is an Agrobacterium Ti or Ri plasmid for use in plants. In the case of screening for phenotypes in plant cells, plant specific guide sequences may be used.

In another aspect, the present invention provides for a method of combinatorial screening of phenotypic interactions between a set of target sequences in a population of cells comprising: introducing a library according to any embodiment herein to a population of cells, wherein two orthogonal CRISPR enzymes are expressed in said cells; selecting for cells comprising a vector of the library; selecting for cells having a desired phenotype; and determining in the cells having the desired phenotype the enrichment or depletion of combinations of guide sequences as compared to the representation in the library introduced. In certain embodiments, selecting for cells comprising a vector of the library comprises treating the population of cells with an antibiotic. In certain embodiments, the vector may further comprise a sequence encoding a CRISPR enzyme operably linked to a regulatory sequence. The CRISPR enzyme may be S. aureus Cas9.

As used herein, “expression” refers to the process by which a polynucleotide is transcribed from a DNA template (such as into and mRNA or other RNA transcript, such as guide sequence) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

In certain embodiments, the library is transduced at an MOI (multiplicity of infection) of about 1 or of about less than 1, about less than 0.75, about less than 0.5, about less than 0.4, about less than 0.3, about less than 0.2 or about less than 0.1. In a further embodiment, the cell is transduced with a multiplicity of infection (MOI) of 0.3-0.75, preferably, the MOI has a value close to 0.4, more preferably the MOI is 0.3 or 0.4. In certain embodiments, the MOI is about 0.3 or 0.4, thereby creating a panel of cells comprising about 1 CRISPR system sgRNA pair per cell, after appropriate selection for successfully transfected/transduced cells, thereby providing a panel of cells comprising a cellular library with pairwise knock outs of every gene in the set of genes. In certain embodiments, where a separate vector comprising a CRISPR enzyme is transduced, the MOI may be about 10, about 5, about 3, or about 1. A high MOI for the vector expressing a CRISPR enzyme provides an increased probability that every cell comprising a library vector will also express both orthogonal CRISPR enzymes.

In certain embodiments, following a combinatorial screen genomic DNA is extracted and the sgRNA readout is performed using PCR (e.g., guide sequence and/or barcode). In certain embodiments, the sgRNA readout is performed using two rounds of PCR (Shalem et al. 2014). In one embodiment, the first PCR step includes amplification of a region containing the paired sgRNA cassette in the lentiviral genomic integrant from extracted genomic DNA. In one further embodiment, the PCR products are used in a second PCR reaction to add on Illumina sequencing adaptors, barcodes and stagger sequences to prevent monotemplate sequencing issues.

In a preferred embodiment, the distribution of sgRNAs is determined before any selection pressure has been applied. In certain embodiments, the distribution of sgRNAs is determined at an early time point and compared to a later time point. This baseline sgRNA distribution is used to infer either depletion or enrichment of specific sgRNA species. For both positive and negative selection screens, hits are identified by comparing the distribution of sgRNAs after selection with the baseline sgRNA distribution. Paired sgRNA sequences are identified by searching for sgRNA pairs whose frequency has either significantly reduced or increased after selection for negative and positive screens respectively.

In certain embodiments, synthetic lethal combinations are determined using the fold change method as described herein. In certain embodiments, combinatorial data is generated using a Pi score method (see, e.g., Horn T, et al. Mapping of signaling networks through synthetic genetic interaction analysis by RNAi. Nature Methods. 2011; 8:341-346) and also a depletion score that measures the absolute decrease in a guide pair combination and averaged for all gene pairs tested. In certain embodiments, synthetic lethal pairs are identified using a Pi z-score to include more statistical confidence in the data as compared to fold change. Pi score takes single gene effects into account and looks for synergies.

Several methods of DNA extraction and analysis are encompassed in the methods of the invention. As used herein “deep sequencing” indicates that the depth of the process is many times larger than the length of the sequence under study. Deep sequencing is encompassed in next generation sequencing methods which include but are not limited to single molecule real-time sequencing (Pacific Bio), Ion semiconductor (Ion torrent sequencing), Pyrosequencing (454), Sequencing by synthesis (Illumina), Sequencing by ligations (SOLiD sequencing) and Chain termination (Sanger sequencing).

In certain embodiments, the present invention is used in a method of assaying combinatorial phenotypic interactions in a population of cells or host cells. In certain embodiments, a population of cells or host cells are derived or obtained from an organism or subject. In some methods of the invention the organism or subject is a eukaryote (including mammal including human) or a non-human eukaryote or a non-human animal or a non-human mammal. In some methods of the invention the organism or subject is a plant. In some methods of the invention the organism or subject is a mammal or a non-human mammal. In some methods of the invention the organism or subject is algae.

The library comprises guide sequences that target a genomic region of interest of an organism. In certain embodiments of the invention the organism or subject is a eukaryote (including mammal including human) or a non-human eukaryote or a non-human animal or a non-human mammal. In certain embodiments, the organism or subject is a non-human animal, and may be an arthropod, for example, an insect, or may be a nematode. In some methods of the invention the organism or subject is a plant. In some methods of the invention the organism or subject is a mammal or a non-human mammal. A non-human mammal may be for example a rodent (preferably a mouse or a rat), an ungulate, or a primate. In some methods of the invention the organism or subject is algae, including microalgae, or is a fungus.

In certain embodiments, a population of cells or host cells is transiently or non-transiently transfected or transduced with one or more vectors described herein to arrive at a tissue culture model. In certain embodiments, a cell is transfected or transduced in vivo in a subject (e.g., an animal model). In certain embodiments, the animal expresses one or more orthogonal CRISPR enzymes from one or more transgenes. In certain embodiments, cells from a transgenic animal are screened ex vivo (see, e.g., US20180255751A1). In certain embodiments, a cell that is transfected is taken from a subject. In certain embodiments, the cell is derived from cells taken from a subject, such as a cell line. A wide variety of cell lines for tissue culture models are known in the art. In certain embodiments, any disease specific cells may be used (e.g., cancer cell lines). In certain embodiments, any immune specific cells may be used (e.g., T cells). In certain embodiments, any pluripotent cell lines may be used (e.g., stem cells).

Examples of cell lines include, but are not limited to, C8161, CCRF-CEM, MOLT, mIMCD-3, NHDF, HeLa-S3, Huhl, Huh4, Huh7, HUVEC, HASMC, HEKn, HEKa, MiaPaCell, Pancl, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

Pluripotent cells may include any mammalian stem cell. As used herein, the term “stem cell” refers to a multipotent cell having the capacity to self-renew and to differentiate into multiple cell lineages. Mammalian stem cells may include, but are not limited to embryonic stem cells of various types, such as murine embryonic stem cells, e.g., as described by Evans & Kaufman 1981 (Nature 292: 154-6) and Martin 1981 (PNAS 78: 7634-8); rat pluripotent stem cells, e.g., as described by Iannaccone et al. 1994 (Dev Biol 163: 288-292); hamster embryonic stem cells, e.g., as described by Doetschman et al. 1988 (Dev Biol 127: 224-227); rabbit embryonic stem cells, e.g., as described by Graves et al. 1993 (Mol Reprod Dev 36: 424-433); porcine pluripotent stem cells, e.g., as described by Notarianni et al. 1991 (J Reprod Fertil Suppl 43: 255-60) and Wheeler 1994 (Reprod Fertil Dev 6: 563-8); sheep embryonic stem cells, e.g., as described by Notarianni et al. 1991 (supra); bovine embryonic stem cells, e.g., as described by Roach et al. 2006 (Methods Enzymol 418: 21-37); human embryonic stem (hES) cells, e.g., as described by Thomson et al. 1998 (Science 282: 1 145-1 147); human embryonic germ (hEG) cells, e.g., as described by Shamblott et al. 1998 (PNAS 95: 13726); embryonic stem cells from other primates such as Rhesus stem cells, e.g., as described by Thomson et al. 1995 (PNAS 92:7844-7848) or marmoset stem cells, e.g., as described by Thomson et al. 1996 (Biol Reprod 55: 254-259). In certain embodiments, the pluripotent cells may include, but are not limited to lymphoid stem cells, myeloid stem cells, neural stem cells, skeletal muscle satellite cells, epithelial stem cells, endodermal and neuroectodermal stem cells, germ cells, extraembryonic and embryonic stem cells, mesenchymal stem cells, intestinal stem cells, embryonic stem cells, and induced pluripotent stem cells (iPSCs).

As noted, prototype “human ES cells” are described by Thomson et al. 1998 (supra) and in U.S. Pat. No. 6,200,806. The scope of the term covers pluripotent stem cells that are derived from a human embryo at the blastocyst stage, or before substantial differentiation of the cells into the three germ layers. ES cells, in particular hES cells, are typically derived from the inner cell mass of blastocysts or from whole blastocysts. Derivation of hES cell lines from the morula stage has been documented and ES cells so obtained can also be used in the invention (Strelchenko et al. 2004. Reproductive BioMedicine Online 9: 623-629). As noted, prototype “human EG cells” are described by Shamblott et al. 1998 (supra). Such cells may be derived, e.g., from gonadal ridges and mesenteries containing primordial germ cells from fetuses. In humans, the fetuses may be typically 5-11 weeks post-fertilization.

Human embryonic stem cells may include, but are not limited to the HUES66, HUES64, HUES3, HUES8, HUES53, HUES28, HUES49, HUES9, HUES48, HUES45, HUES1, HUES44, HUES6, H1, HUES62, HUES65, H7, HUES13 and HUES63 cell lines.

General techniques useful in the practice of this invention in cell culture and media uses are known in the art (e.g., Large Scale Mammalian Cell Culture (Hu et al. 1997. Curr Opin Biotechnol 8: 148); Serum-free Media (K. Kitano. 1991. Biotechnology 17: 73); or Large Scale Mammalian Cell Culture (Curr Opin Biotechnol 2: 375, 1991). The terms “culturing” or “cell culture” are common in the art and broadly refer to maintenance of cells and potentially expansion (proliferation, propagation) of cells in vitro. Typically, animal cells, such as mammalian cells, such as human cells, are cultured by exposing them to (i.e., contacting them with) a suitable cell culture medium in a vessel or container adequate for the purpose (e.g., a 96-, 24-, or 6-well plate, a T-25, T-75, T-150 or T-225 flask, or a cell factory), at art-known conditions conducive to in vitro cell culture, such as temperature of 37° C., 5% v/v CO₂ and >95% humidity.

Methods related to culturing stem cells are also useful in the practice of this invention (see, e.g., “Teratocarcinomas and embryonic stem cells: A practical approach” (E. J. Robertson, ed., IRL Press Ltd. 1987); “Guide to Techniques in Mouse Development” (P. M. Wasserman et al. eds., Academic Press 1993); “Embryonic Stem Cells: Methods and Protocols” (Kursad Turksen, ed., Humana Press, Totowa N.J., 2001); “Embryonic Stem Cell Differentiation in vitro” (M. V. Wiles, Meth. Enzymol. 225: 900, 1993); “Properties and uses of Embryonic Stem Cells: Prospects for Application to Human Biology and Gene Therapy” (P. D. Rathjen et al., al., 1993). Differentiation of stem cells is reviewed, e.g., in Robertson. 1997. Meth Cell Biol 75: 173; Roach and McNeish. 2002. Methods Mol Biol 185: 1-16; and Pedersen. 1998. Reprod Fertil Dev 10: 31). For further elaboration of general techniques useful in the practice of this invention, the practitioner can refer to standard textbooks and reviews in cell biology, tissue culture, and embryology (see, e.g., Culture of Human Stem Cells (R. Ian Freshney, Glyn N. Stacey, Jonathan M. Auerbach—2007); Protocols for Neural Cell Culture (Laurie C. Doering—2009); Neural Stem Cell Assays (Navjot Kaur, Mohan C. Vemuri—2015); Working with Stem Cells (Henning Ulrich, Priscilla Davidson Negraes—2016); and Biomaterials as Stem Cell Niche (Krishnendu Roy—2010)).

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4+, CD8+, effector Th, memory Th, regulatory Th, CD4+/CD8+ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

As used throughout this specification, “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MHC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MHC class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response)), antigen presentation, and/or cytokine secretion.

In certain embodiments, the methods as described herein may comprise providing a Cas transgenic cell in which a vector encoding guide RNAs of the screening platform are provided. As used herein, the term “Cas transgenic cell” refers to a cell, such as a eukaryotic cell, in which a Cas gene or pair of orthologous Cas genes have been genomically integrated. The nature, type, or origin of the cell are not particularly limiting according to the present invention. Also, the way how the Cas transgene is introduced in the cell is may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus, such as for instance one or more oncogenic mutations, as for instance and without limitation described in Platt et al. (2014), Chen et al., (2014) or Kumar et al. (2009).

The current invention comprehends the use of the compositions of the current invention to establish and utilize conditional or inducible CRISPR transgenic cell/animals; see, e.g., Platt et al., “CRISPR-Cas9 Knockin Mice for Genome Editing and Cancer Modeling” Cell (2014), 159(2): 440-455, or PCT patent publications cited herein, such as WO 2014/093622 (PCT/US2013/074667). For example, cells or animals such as non-human animals, e.g., vertebrates or mammals, such as rodents, e.g., mice, rats, or other laboratory or field animals, e.g., cats, dogs, sheep, etc., may be ‘knock-in’ whereby the animal conditionally or inducibly expresses Cas9 akin to Platt et al. The target cell or animal thus comprises the CRISPR enzyme (e.g., Cas9) conditionally or inducibly (e.g., in the form of Cre dependent constructs), on expression of a vector introduced into the target cell, the vector expresses that which induces or gives rise to the condition of the CRISPR enzyme (e.g., Cas9) expression in the target cell. By applying the teaching and compositions as defined herein with the known method of creating a CRISPR complex, inducible genomic events are also an aspect of the current invention. Examples of such inducible events have been described herein elsewhere. In certain embodiments, the present invention may be used for determining combinatorial phenotypic interactions in immune cells by expressing orthogonal CRISPR enzymes. In certain embodiments, a transgenic animal may express two orthogonal CRISPR enzymes. In other embodiments, the transgenic animal expresses a single CRISPR enzyme and an orthogonal CRISPR enzyme is expressed from a vector comprising combinatorial sgRNA sequences. In certain embodiments, leukocytes are obtained from transgenic animals expressing a CRISPR enzyme (see, e.g., WO2016049251). The library according to the present invention may be introduced to the leukocytes and assayed for a phenotype.

In certain embodiments, a library for the combinatorial screening of phenotypic interactions between a set of target sequences is constructed. The first step in generating a library according to the present invention is synthesizing a set of oligonucleotides targeting all target sequences in a set of target sequences. The set of target sequences may include genes with known drugs, inhibitors, agonists, and/or antagonists. The set of target sequences may include regulatory sequences present in a genome of interest. The set of target sequences may be genome wide. The set of target sequences may include a set of genes that function in a specific pathway. The set of target genes may include genes expressed in specific cell types (e.g., diseased cells, cancer cells, immune cells, stem cells). In certain embodiments, the genes may represent a subset of the entire genome; for example, genes relating to a particular pathway (for example, an enzymatic pathway) or a particular disease or group of diseases or disorders may be selected. One or more of the genes may include a plurality of target sequences; that is, one gene may be targeted by a plurality of guide sequences (e.g., two or more guide sequences).

In certain embodiments, the present invention may be used to target non-coding DNA regions in addition to coding genes. In certain embodiments, guide RNAs may target microRNAs, microRNA clusters, long noncoding RNAs (LncRNA), long intergenic noncoding RNAs (LincRNA), regulatory regions, such as, but not limited to promoters, enhancers, insulators. In certain embodiments, CRISPRa/i/x, as described herein, is targeted to a regulatory region associated with a gene.

In certain embodiments of the invention the unique CRISPR-Cas system guide sequences are selected by an algorithm that predicts the efficacy of the guide sequences based on the primary nucleotide sequence of the guide sequence and/or by a heuristic that ranks the guide sequences based on off target scores. In certain embodiments, orthologous guide sequences are based on the rules described herein (see, examples).

Oligonucleotides can be synthesized at the same time or at separate times. Oligonucleotides can be synthesized by a commercial oligonucleotide service. Generating oligonucleotides may comprise synthesizing a first set of oligonucleotides, each oligonucleotide comprising a guide sequence specific for a target sequence in the set of target sequences and specific for a first orthogonal CRISPR enzyme, wherein the oligonucleotides comprise a first non-palindromic hybridization sequence at the 3′ end and a site for cloning into a vector at the 5′end and synthesizing a second set oligonucleotides, each oligonucleotide comprising a guide sequence specific for a target sequence in the set of target sequences and specific for a second orthogonal CRISPR enzyme, wherein the oligonucleotides comprise a second hybridization sequence at the 3′ end of the sequence that is complementary to the first hybridization sequence and a site for cloning into a vector at the 5′end. The oligonucleotides corresponding to the first and second set of oligonucleotides include a non-palindromic hybridization sequence so that the oligonucleotides from the first set only hybridize to oligonucleotides in the second set and not to each other.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

In certain embodiments, the next step is hybridizing the first and second set of oligonucleotides to obtain oligonucleotides that are partially double stranded at the hybridization sequence and include a sequence encoding one guide sequence from the first set and one guide sequence from the second set. DNA extension is performed on the partially double stranded oligonucleotide using the hybridization region as priming sequences to generate a pool of double stranded DNA oligonucleotides comprising pairs of inverted guide sequences, each specific for orthogonal CRISPR enzymes. Multiple copies of each guide sequence in the two sets are synthesize, such that all pairwise combinations of guide sequences from the first and second set of oligonucleotides is represented in the pool of oligonucleotides.

The double stranded oligonucleotides from the pool of dsDNA oligonucleotides can then be joined into a vector comprising two convergent regulatory sequences flanking a cloning site, wherein the two convergent regulatory sequences do not have 100% sequence identity to one another, and wherein the oligonucleotides are joined between the convergent regulatory sequences. In certain embodiments, the ends of the oligonucleotides comprise restriction enzyme sites and the vector comprises compatible restriction enzyme site(s) between the convergent regulatory sequences, whereby joining is by ligation of compatible restriction enzyme digested ends on the oligonucleotides and the vector. In certain embodiments, the ends of the oligonucleotides comprise homologous sequences configured for recombination and the vector comprises compatible homologous sequences between the convergent regulatory sequences, whereby joining is by recombination of the oligonucleotides into the vector. The ends of the oligonucleotides can be made compatible with either cloning method and can be designed such that each oligonucleotide synthesized for each set of oligonucleotides includes a sequence for restriction enzyme sites or homologous recombination.

The convergent regulatory sequences may be RNA polymerase III (RNAP III) promoters. In certain embodiments, one RNAP III promoter comprises the U6 promoter and one RNAP III promoter comprises the H1 promoter.

In certain embodiments, the nucleotide sequence encoding the Cas9 endonuclease is modified to alter the activity of the protein. In certain embodiments, the Cas9 endonuclease is a catalytically inactive Cas9. For example, dCas9 contains mutations of catalytically active residues (D10 and H840) and does not have nuclease activity. One skilled in the art may modify orthogonal Cas9 endonucleases to contain homologous mutations to generate catalytically inactive enzymes.

In certain embodiments, the CRISPR enzyme may comprise one or more heterologous functional domains. The CRISPR enzyme may be fused to a functional domain or may recruit a functional domain. In preferred embodiments, a CRISPR enzyme comprising a functional domain is a dCas9.

The one or more heterologous functional domains may have one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity.

The at least one or more heterologous functional domains may be at or near the amino-terminus of the enzyme and/or at or near the carboxy-terminus of the enzyme. The one or more heterologous functional domains may be fused to the CRISPR enzyme, or tethered to the CRISPR enzyme, or linked to the CRISPR enzyme by a linker moiety.

As used herein the term “CRISPR interference” (CRISPRi) refers to the use of a CRISPR system to interfere with the expression of a gene and “CRISPR activation” (CRISPRa) refers to the use of CRISPR system to activate expression of a gene. Both CRISPRa and CRISPRi do not result in cutting or cleavage of a target sequence. CRISPRi can sterically repress transcription in two ways—by blocking transcriptional initiation or elongation. This is accomplished by designing sgRNA complementary to the promoter or exonic sequences, respectively. The level of transcriptional repression for exonic sequences is strand-specific. sgRNA complementary to the non-template strand more strongly represses transcription compared to sgRNA complementary to the template strand. One hypothesis to explain this effect is from the activity of helicase, which unwinds the RNA:DNA heteroduplex ahead of RNA pol II when the sgRNA is complementary to exons of the template strand. In prokaryotes, this steric inhibition can repress transcription of the target gene by almost 99.9%. Whereas in human cells, up to 90% repression was observed (Qi, L. S., et al. (2013). “Repurposing CRISPR as an RNA-guided platform for sequence-specific control of gene expression”. Cell. 152 (5): 1173-83).

CRISPRi can also repress transcription via an effector domain. Fusing a repressor domain to dCas9 allows transcription to be further repressed by inducing heterochromatinization. For example, the well-studied Kruppel associated box (KRAB) domain can be fused to dCas9 to repress transcription of the target gene up to 99% in human cells (Gilbert, L. A., et al., (2013). “CRISPR-mediated modular RNA-guided regulation of transcription in eukaryotes”. Cell. 154 (2): 442-51). In preferred embodiments, the one or more heterologous functional domains comprises one or more transcriptional repression domains. A transcriptional repression domain may comprise a KRAB domain or a SID domain or concatemers of SID (e.g., SID4X).

CRISPRa can be used to activate transcription of the target gene by fusing a transcriptional activator to dCas9. For example, the transcriptional activator VP16 can increase gene expression by up to 25-fold in human cells on a Tet-ON reporter system (Gilbert, L. A., et al., (2013). “CRISPR-mediated modular RNA-guided regulation of transcription in eukaryotes”. Cell. 154 (2): 442-51). In preferred embodiments, the one or more heterologous functional domains comprises one or more transcriptional activation domains. A transcriptional activation domain may comprise VP64.

In certain embodiments, such dCas9 fusion proteins are used with the constructs described herein for combinatorial gene repression (e.g. CRISPR interference (CRISPRi)). In certain embodiments, such dCas9 fusion proteins are used with the constructs described herein for combinatorial gene activation (e.g. CRISPR activation (CRISPRa)).

In certain embodiments, dCas9 is fused to an epigenetic modulating domain, such as a histone demethylase domain, a histone acetyltransferase domain, DNA methyltransferase domain, or DNA demethylation domain (e.g., TET1, see Xu et al., Cell Discov. 2016 May 3; 2:16009; and Choudhury et al., Oncotarget. 2016 Jul. 19; 7(29):46545-46556). In certain embodiments, dCas9 is fused to a LSD1 or p300, or a portion thereof. In certain embodiments, the dCas9 fusion is used for CRISPR-based epigenetic modulation. In certain embodiments, dCas9 or Cas9 is fused to a Fokl nuclease domain. In preferred embodiments, Cas9 or dCas9 fused to a Fokl nuclease domain is used for combinatorial gene editing.

As used herein the term “CRISPR-X” refers to a strategy to repurpose the somatic hypermutation machinery for protein engineering in situ to specifically mutagenize endogenous targets with limited off-target damage (see, e.g., Komor et al., 2016, Programmable editing of a target base in genomic DNA without double-stranded DNA cleavage, Nature 533, 420-424; Nishida et al., 2016, Targeted nucleotide editing using hybrid prokaryotic and vertebrate adaptive immune systems, Science 353(6305); Yang et al., 2016, Engineering and optimising deaminase fusions for genome editing, Nat Commun. 7:13330; Hess et al., 2016, Directed evolution using dCas9-targeted somatic hypermutation in mammalian cells, Nature Methods 13, 1036-1042; and Ma et al., 2016, Targeted AID-mediated mutagenesis (TAM) enables efficient genomic diversification in mammalian cells, Nature Methods 13, 1029-1035). In certain embodiments, the Cas9 endonuclease is fused another protein or portion thereof to allow the introduction of somatic mutations. In certain embodiments, catalytically inactive dCas9 is used to recruit variants of cytidine deaminase (AID) with MS2-modified sgRNAs. In certain embodiments, dCas9-AID-P182X (AIDx) is used as the CRISPR enzyme. In certain embodiments, AID-P182X is recruited by the CRISPR enzyme. Not being bound by a theory, sgRNAs may be used to target sequences by the CRISPR enzyme to directly change cytidines or guanines to the other three bases independent of AID hotspot motifs. Unmethylated cytosines are converted to uracil and are repaired in a cell by uracil-DNA glycosylase. In certain embodiments, CRISPR-X is coupled with an uracil-DNA glycosylase inhibitor, such that dCas9-AIDx can convert targeted cytidines specifically to thymines, creating specific point mutations. In certain embodiments, AID is fused to any dCas9 orthologue. In certain embodiments, AID is fused to an adapter protein specific for binding an aptamer.

In certain embodiments, the RNA of the CRISPR-Cas system, e.g., the guide or sgRNA, can be modified; for instance, to include an aptamer or a functional domain (see e.g., WO2016049258A2). In certain embodiments, the aptamer may be incorporated during synthesis of the oligonucleotides used for generating the library of the present invention. Not being bound by a theory, modifying the sgRNA with an aptamer allows for the recruitment of a functional domain without generating orthogonal CRISPR fusion enzymes.

An aptamer is a synthetic oligonucleotide that binds to a specific target molecule; for instance, a nucleic acid molecule that has been engineered through repeated rounds of in vitro selection or SELEX (systematic evolution of ligands by exponential enrichment) to bind to various molecular targets such as small molecules, proteins, nucleic acids, and even cells, tissues and organisms. Aptamers are useful in that they offer molecular recognition properties that rival that of antibodies. In addition to their discriminate recognition, aptamers offer advantages over antibodies including that they elicit little or no immunogenicity in therapeutic applications. Accordingly, in the practice of the invention, either or both of the enzyme or the RNA can include a functional domain.

In certain embodiments, the invention provides for introduction of an RNA sequence into a transcript recruitment sequence that forms a loop secondary structure and binds to an adapter protein. In one embodiment, the invention provides a herein-discussed composition, wherein the insertion of distinct RNA sequence(s) that bind to one or more adaptor proteins is an aptamer sequence. In one embodiment, the invention provides a herein-discussed composition, wherein the aptamer sequence is two or more aptamer sequences specific to the same adaptor protein. In an aspect, the invention provides a herein-discussed composition, wherein the aptamer sequence is two or more aptamer sequences specific to a different adaptor protein. In one embodiment, the invention provides a herein-discussed composition, wherein the adaptor protein comprises MS2, PP7, Q13, F2, GA, fr, JP501, M12, R17, BZ13, JP34, JP500, KU1, M11, MX1, TW18, VK, SP, FI, ID2, NL95, TW19, AP205, 4Cb5, Kb8r, 4Cb12r, 4Cb23r, 7s, PRR1. In one embodiment, the invention provides a herein-discussed composition, wherein the cell is a eukaryotic cell. In one embodiment, the invention provides a herein-discussed composition, wherein the eukaryotic cell is a mammalian cell, optionally a mouse cell. one embodiment, the invention provides a herein-discussed composition, wherein the mammalian cell is a human cell. Aspects of the invention encompass embodiments relating to MS2 adaptor proteins described in Konermann et al. “Genome-scale transcriptional activation by an engineered CRISPR-Cas9 complex” Nature. 2014 Dec. 10. doi: 10.1038/nature14136, the contents of which are herein incorporated by reference in its entirety.

In certain embodiments, the adaptor protein domain is an RNA-binding protein domain. The RNA-binding protein domain recognizes corresponding distinct RNA sequences, which may be aptamers. For example, the MS2 RNA-binding protein recognizes and binds specifically to the MS2 aptamer (or visa versa).

Similarly, an MS2 variant adaptor domain may also be used, such as the N55 mutant, especially the N55K mutant. This is the N55K mutant of the MS2 bacteriophage coat protein (shown to have higher binding affinity than wild type MS2 in Lim, F., M. Spingola, and D. S. Peabody. “Altering the RNA binding specificity of a translational repressor.” Journal of Biological Chemistry 269.12 (1994): 9006-9010).

One guide with a first aptamer/RNA-binding protein pair can be linked or fused to an activator, whilst a second guide with a second aptamer/RNA-binding protein pair can be linked or fused to a repressor. The guides are for different targets (loci), so this allows one gene to be activated and one repressed. For example, the following schematic shows such an approach: Guide 1—MS2 aptamer-------MS2 RNA-binding protein-------VP64 activator; and Guide 2—PP7 aptamer-------PP7 RNA-binding protein-------SID4x repressor.

The present invention also relates to orthogonal PP7/MS2 gene targeting. In this example, sgRNA targeting different loci are modified with distinct RNA loops in order to recruit MS2-VP64 or PP7-SID4X, which activate and repress their target loci, respectively. PP7 is the RNA-binding coat protein of the bacteriophage Pseudomonas. Like MS2, it binds a specific RNA sequence and secondary structure. The PP7 RNA-recognition motif is distinct from that of MS2. Consequently, PP7 and MS2 can be multiplexed to mediate distinct effects at different genomic loci simultaneously. For example, an sgRNA targeting locus A can be modified with MS2 loops, recruiting MS2-VP64 activators, while another sgRNA targeting locus B can be modified with PP7 loops, recruiting PP7-SID4X repressor domains. In the same cell, dCas9 can thus mediate orthogonal, locus-specific modifications. This principle can be extended to incorporate other orthogonal RNA-binding proteins such as Q-beta.

An alternative option for orthogonal repression includes incorporating non-coding RNA loops with transactive repressive function into the guide (either at similar positions to the MS2/PP7 loops integrated into the guide or at the 3′ terminus of the guide). For instance, guides can be designed with non-coding (but known to be repressive) RNA loops (e.g. using the Alu repressor (in RNA) that interferes with RNA polymerase II in mammalian cells). The Alu RNA sequence can be located: in place of the MS2 RNA sequences as used herein (e.g. at tetraloop and/or stem loop 2); and/or at 3′ terminus of the guide. This gives possible combinations of MS2, PP7 or Alu at the tetraloop and/or stemloop 2 positions, as well as, optionally, addition of Alu at the 3′ end of the guide (with or without a linker).

The use of two different aptamers (distinct RNA) allows an activator-adaptor protein fusion and a repressor-adaptor protein fusion to be used, with different guides, to activate expression of one gene, whilst repressing another. In certain embodiments, the present invention allows for combinatorial phenotypic screening such that phenotypic interactions between a gene that is activated with a gene that is repressed can be determined. In certain embodiments, the population of cells used may express multiple adapter fusion proteins.

The adaptor protein may be associated (preferably linked or fused to) one or more activators or one or more repressors. For example, the adaptor protein may be associated with a first activator and a second activator. The first and second activators may be the same, but they are preferably different activators. For example, one might be VP64, whilst the other might be p65, although these are just examples and other transcriptional activators are envisaged. Three or more or even four or more activators (or repressors) may be used, but package size may limit the number being higher than 5 different functional domains. Linkers are preferably used, over a direct fusion to the adaptor protein, where two or more functional domains are associated with the adaptor protein. Suitable linkers might include the GlySer linker.

The fusion between the adaptor protein and the activator or repressor may include a linker. For example, GlySer linkers GGGS can be used. They can be used in repeats of 3 ((GGGGS)₃ (SEQ ID NO:45,516)) or 6 (SEQ ID NO:45,517), 9 (SEQ ID NO:45,518) or even 12 (SEQ ID NO:45,519) or more, to provide suitable lengths, as required. Linkers can be used between the RNA-binding protein and the functional domain (activator or repressor), or between the CRISPR Enzyme (Cas9) and the functional domain (activator or repressor). The linkers the user to engineer appropriate amounts of “mechanical flexibility”.

It is also envisaged that the enzyme-guide complex as a whole may be associated with two or more functional domains. For example, there may be two or more functional domains associated with the enzyme, or there may be two or more functional domains associated with the guide (via one or more adaptor proteins), or there may be one or more functional domains associated with the enzyme and one or more functional domains associated with the guide (via one or more adaptor proteins).

In general, the sgRNAs are modified in a manner that provides specific binding sites (e.g. aptamers) for adapter proteins comprising one or more functional domains (e.g. via fusion protein) to bind to. The modified sgRNA are modified such that once the sgRNA forms a CRISPR complex (i.e. CRISPR enzyme binding to sgRNA and target) the adapter proteins bind and, the functional domain on the adapter protein is positioned in a spatial orientation which is advantageous for the attributed function to be effective. For example, if the functional domain is a transcription activator (e.g. VP64 or p65), the transcription activator is placed in a spatial orientation which allows it to affect the transcription of the target. Likewise, a transcription repressor will be advantageously positioned to affect the transcription of the target and a nuclease (e.g. Fokl) will be advantageously positioned to cleave or partially cleave the target.

The skilled person will understand that modifications to the sgRNA which allow for binding of the adapter+functional domain but not proper positioning of the adapter+functional domain (e.g. due to steric hindrance within the three-dimensional structure of the CRISPR complex) are modifications which are not intended. The one or more modified sgRNA may be modified at the tetra loop, the stem loop 1, stem loop 2, or stem loop 3, as described herein, preferably at either the tetra loop or stem loop 2, and most preferably at both the tetra loop and stem loop 2. In exemplary embodiments, the MS2-binding loop ggccAACATGAGGATCACCCATGTCTGCAGggcc (SEQ ID NO:45,520) may replace nucleotides+13 to +16 and nucleotides+53 to +56 of the standard sgRNA backbone. The resulting structure is an sgRNA scaffold in which the tetraloop and stemloop 2 sequences have been replaced by an MS2 binding loop. Without being bound by theory, the tetraloop and stemloop 2 were selected for replacement based on information obtained from the Cas9/RNA/DNA crystal structure. Specifically, the tetraloop and stemloop 2 were found to protrude from the Cas9 protein in such a way which suggested that adding an MS2 binding loop would not interfere with any Cas9 residues. Additionally, the proximity of the tetraloop and stemloop 2 sites to the DNA suggested that localization to these locations would result in a high degree of interaction between the DNA and any recruited protein, such as a transcriptional activator. In short, a specific RNA sequence may be inserted into the exposed guide loop(s) and a corresponding RNA-binding protein may be used, whether that is fused to a functional domain, or a further element which in turn recognizes or binds specifically to a functional domain. The functional domain may be a transacting activator or a repressor.

Although single MS2 addition (i.e. to one or other of the tetraloop or stem loop 2) shows an improvement in terms of Gain of Function (gene upregulation) compared to a standard guide, the double addition (MS2 on both loops) shows even stronger upregulation. The use of two or more functional domains with the guide is therefore preferred.

As mentioned herein, having one activator, such as VP64, bound to Cas9 and a separate similar activator, again VP64, bound to the guide via MS2 shows the greatest improvement in terms of Gain of Function (gene upregulation). Other activators or repressors may be exchanged here for the activator mentioned. In certain embodiments, host cells for screening combinatorial interactions using CRISPRa may express orthogonal CRISPR enzyme activator fusion proteins and adapter activator fusion proteins.

### Epigenetic Targeting Platform

Epigenetic modifications play an important role in gene expression and regulation, and are involved in numerous cellular processes such as differentiation, development, and tumorigenesis. For example, the chromatin regulatory network provides for chromatin interaction, reinforcing genes, antagonistic genes, genes often found in multiprotein complexes, readers, writers and erasers. In certain embodiments, combinations of guide sequences targeting combinations of epigenetic or chromatin regulation genes are screened. Genes that regulate chromatin are able to be targeted pharmaceutically, i.e., druggable, are often mutated in cancer, and are commonly found in redundant pathways. Chromatin regulators include enzymatic proteins with functional domains that can be targeted. For example, enzymes include, but are not limited to EZH2, DOT1L, KDM and MT. Chromatin regulator genes with known inhibitors include, but are not limited to DOT1L, EZH2, EHMT1, EHMT2, SETD7, SMYD2, DNMT1, PRMT1, PRMT3, PRMT5, PRMT4, PRMT6, PRMT8, KDM1A, KDM6A, KDM6B, HDAC1, HDAC2, HDAC3, HDAC6, HDAC8, SIRT1, SIRT2, SIRT6, BAZ2A, BAZ2B, BRD4, BRD9/7, EP300, CECR2, SMARCA4, P300, CDK7, EED, SMYD3, BRPF1, KDM4A, KDM4B, KDM4C, KDM4D, KDM4E, KDM5A, KDM5B, KDM5C and KDM5D. In certain embodiments, chromatin regulators may include a gene in Table 1.

TABLE 1

| Gene Symbol | Gene ID |
|---|---|
| AADAT | 51166 |
| AASS | 10157 |
| ABAT | 18 |
| ABT1 | 29777 |
| ACD | 65057 |
| ACTB | 60 |
| ACTL6A | 86 |
| ACTL6B | 51412 |
| ACTR5 | 79913 |
| ACTR6 | 64431 |
| ACTR8 | 93973 |
| ADHFE1 | 137872 |
| ADI1 | 55256 |
| ADO | 84890 |
| ADRM1 | 11047 |
| AEBP2 | 121536 |
| AES | 166 |
| AFF1 | 4299 |
| AFF2 | 2334 |
| AFF3 | 3899 |
| AFF4 | 27125 |
| AGXT | 189 |
| AGXT2 | 64902 |
| AICDA | 57379 |
| AIFM1 | 9131 |
| AIM1 | 202 |
| AIP | 9049 |
| AIRE | 326 |
| AJUBA | 84962 |
| AKAP1 | 8165 |
| AKIRIN2 | 55122 |
| ALDHIB1 | 219 |
| ALDH2 | 217 |
| ALDH3A2 | 224 |
| ALDH7A1 | 501 |
| ALDH9A1 | 223 |
| ALG13 | 79868 |
| ALKBH1 | 8846 |
| ALKBH2 | 121642 |
| ALKBH3 | 221120 |
| ALKBH5 | 54890 |
| ALKBH6 | 84964 |
| ALYREF | 10189 |
| ANKRD1 | 27063 |
| ANP32B | 10541 |
| ANP32E | 81611 |
| APBB1 | 322 |
| APEX1 | 328 |
| APOBEC1 | 339 |
| APPL1 | 26060 |
| APPL2 | 55198 |
| ARHGAP35 | 2909 |
| ARID1A | 8289 |
| ARID1B | 57492 |
| ARID2 | 196528 |
| ARID3A | 1820 |
| ARID3B | 10620 |
| ARID3C | 138715 |
| ARID4A | 5926 |
| ARID4B | 51742 |
| ARID5A | 10865 |
| ARID5B | 84159 |
| ARL2BP | 23568 |
| ARRB1 | 408 |
| AS3MT | 57412 |
| ASF1A | 25842 |
| ASF1B | 55723 |
| ASH1L | 55870 |
| ASH2L | 9070 |
| ASPH | 444 |
| ASPHD1 | 253982 |
| ASPHD2 | 57168 |
| ASXL1 | 171023 |
| ASZ1 | 136991 |
| ATAD2 | 29028 |
| ATAD2B | 54454 |
| ATF2 | 1386 |
| ATF3 | 467 |
| ATF7IP | 55729 |
| ATF7IP2 | 80063 |
| ATN1 | 1822 |
| ATRX | 546 |
| ATXN7 | 6314 |
| ATXN7L3 | 56970 |
| AURKB | 9212 |
| AURKC | 6795 |
| AXIN2 | 8313 |
| BABAM1 | 29086 |
| BAG6 | 7917 |
| BAHD1 | 22893 |
| BANF1 | 8815 |
| BANP | 54971 |
| BAP1 | 8314 |
| BASP1 | 10409 |
| BAZ1A | 11177 |
| BAZ1B | 9031 |
| BAZ2A | 11176 |
| BAZ2B | 29994 |
| BBOX1 | 8424 |
| BCAT1 | 586 |
| BCAT2 | 587 |
| BCMO1 | 53630 |
| BCO2 | 83875 |
| BCOR | 54880 |
| BCORL1 | 63035 |
| BDP1 | 55814 |
| BIRC2 | 329 |
| BMI1 | 648 |
| BPTF | 2186 |
| BRCA1 | 672 |
| BRCA2 | 675 |
| BRCC3 | 79184 |
| BRD1 | 23774 |
| BRD2 | 6046 |
| BRD3 | 8019 |
| BRD4 | 23476 |
| BRD7 | 29117 |
| BRD8 | 10902 |
| BRD9 | 65980 |
| BRDT | 676 |
| BRE | 9577 |
| BRF1 | 2972 |
| BRPF1 | 7862 |
| BRPF3 | 27154 |
| BRWD1 | 54014 |
| BRWD3 | 254065 |
| BUB1 | 699 |
| C11orf30 | 56946 |
| C14orf169 | 79697 |
| C17orf49 | 124944 |
| C1orf85 | 112770 |
| C1QBP | 708 |
| C9orf64 | 84267 |
| CABIN1 | 23523 |
| CALCOCO1 | 57658 |
| CAMK2D | 817 |
| CARM1 | 10498 |
| CASC5 | 57082 |
| CASP8AP2 | 9994 |
| CBFA2T2 | 9139 |
| CBX1 | 10951 |
| CBX2 | 84733 |
| CBX3 | 11335 |
| CBX4 | 8535 |
| CBX5 | 23468 |
| CBX6 | 23466 |
| CBX7 | 23492 |
| CBX8 | 57332 |
| CCAR1 | 55749 |
| CCAR2 | 57805 |
| CCBL1 | 883 |
| CCBL2 | 56267 |
| CCDC101 | 112869 |
| CCNC | 892 |
| CCND1 | 595 |
| CCNE1 | 898 |
| CCNH | 902 |
| CCNT1 | 904 |
| CCNT2 | 905 |
| CD1D | 912 |
| CD3D | 915 |
| CD3EAP | 10849 |
| CDAN1 | 146059 |
| CDC40 | 51362 |
| CDC5L | 988 |
| CDC73 | 79577 |
| CDCA2 | 157313 |
| CDCA5 | 113130 |
| CDCA8 | 55143 |
| CDK12 | 51755 |
| CDK13 | 8621 |
| CDK19 | 23097 |
| CDK2 | 1017 |
| CDK2AP1 | 8099 |
| CDK7 | 1022 |
| CDK8 | 1024 |
| CDK9 | 1025 |
| CDKN2A | 1029 |
| CDO1 | 1036 |
| CDY1 | 9085 |
| CDY1B | 9085 |
| CDY2A | 9085 |
| CDY2B | 9426 |
| CDYL | 9425 |
| CDYL2 | 124359 |
| CECR2 | 27443 |
| CENPA | 1058 |
| CENPB | 1059 |
| CENPC | 1060 |
| CENPH | 64946 |
| CENPI | 2491 |
| CENPK | 64105 |
| CENPL | 91687 |
| CENPM | 79019 |
| CENPN | 55839 |
| CENPO | 79172 |
| CENPP | 401541 |
| CENPQ | 55166 |
| CENPU | 79682 |
| CENPV | 201161 |
| CENPW | 387103 |
| CHAF1A | 10036 |
| CHAF1B | 8208 |
| CHD1 | 1105 |
| CHD1L | 9557 |
| CHD2 | 1106 |
| CHD3 | 1107 |
| CHD4 | 1108 |
| CHD5 | 26038 |
| CHD6 | 84181 |
| CHD7 | 55636 |
| CHD8 | 57680 |
| CHD9 | 80205 |
| CHEK1 | 1111 |
| CHMP1A | 5119 |
| CHRAC1 | 54108 |
| CHUK | 1147 |
| CIITA | 4261 |
| CIR1 | 9541 |
| CITED1 | 4435 |
| CITED2 | 10370 |
| CITED4 | 163732 |
| CLP1 | 10978 |
| COPRS | 55352 |
| COPS5 | 10987 |
| COQ3 | 51805 |
| CORO2A | 7464 |
| CPA4 | 51200 |
| CPSF1 | 29894 |
| CPSF2 | 53981 |
| CPSF3 | 51692 |
| CPSF7 | 79869 |
| CREBBP | 1387 |
| CREG1 | 8804 |
| CRTC1 | 23373 |
| CRYM | 1428 |
| CSNK2A1 | 1457 |
| CSNK2A2 | 1459 |
| CSNK2B | 1460 |
| CSRP2BP | 57325 |
| CSTF1 | 1477 |
| CSTF2 | 1478 |
| CSTF3 | 1479 |
| CTBP1 | 1487 |
| CTBP2 | 1488 |
| CTCF | 10664 |
| CTCFL | 140690 |
| CTDP1 | 9150 |
| CTNNB1 | 1499 |
| CTR9 | 9646 |
| CTSL | 1514 |
| CUL4B | 8450 |
| CXXC1 | 30827 |
| DACH1 | 1602 |
| DAPK3 | 1613 |
| DAXX | 1616 |
| DCAF6 | 55827 |
| DDB1 | 1642 |
| DDB2 | 1643 |
| DDX1 | 1653 |
| DDX17 | 10521 |
| DDX5 | 1655 |
| DDX54 | 79039 |
| DEAF1 | 10522 |
| DEK | 7913 |
| DEPDC1 | 55635 |
| DHTKD1 | 55526 |
| DHX36 | 170506 |
| DHX38 | 9785 |
| DIDO1 | 11083 |
| DLC1 | 10395 |
| DLD | 1738 |
| DLST | 1743 |
| DLX5 | 1749 |
| DMAP1 | 55929 |
| DNAJC1 | 64215 |
| DNAJC2 | 27000 |
| DNMT1 | 1786 |
| DNMT3A | 1788 |
| DNMT3B | 1789 |
| DNMT3L | 29947 |
| DOT1L | 84444 |
| DPF1 | 8193 |
| DPF2 | 5977 |
| DPF3 | 8110 |
| DPPA3 | 359787 |
| DPY30 | 84661 |
| DR1 | 1810 |
| DTX1 | 1840 |
| DTX3L | 151636 |
| DYNLL1 | 8655 |
| DYRK1B | 9149 |
| E2F1 | 1869 |
| E2F6 | 1876 |
| E2F7 | 144455 |
| E2F8 | 79733 |
| E4F1 | 1877 |
| ECD | 11319 |
| EDF1 | 8721 |
| EED | 8726 |
| EGLN1 | 54583 |
| EGLN2 | 112398 |
| EGLN3 | 112399 |
| EHMT1 | 79813 |
| EHMT2 | 10919 |
| EID1 | 23741 |
| ELANE | 1991 |
| ELL | 8178 |
| ELL2 | 22936 |
| ELL3 | 80237 |
| ELP2 | 55250 |
| ELP3 | 55140 |
| ELP4 | 26610 |
| ENO1 | 2023 |
| ENY2 | 56943 |
| EP300 | 2033 |
| EP400 | 57634 |
| EPC1 | 80314 |
| EPC2 | 26122 |
| ERCC1 | 2067 |
| ERCC2 | 2068 |
| ERCC3 | 2071 |
| ERCC4 | 2072 |
| ERCC5 | 2073 |
| ESPL1 | 9700 |
| ETHE1 | 23474 |
| EYA1 | 2138 |
| EYA2 | 2139 |
| EYA3 | 2140 |
| EYA4 | 2070 |
| EZH1 | 2145 |
| EZH2 | 2146 |
| FAM175A | 84142 |
| FAM60A | 58516 |
| FAM64A | 54478 |
| FBL | 2091 |
| FBXL19 | 54620 |
| FBXO11 | 80204 |
| FGF2 | 2247 |
| FHL2 | 2274 |
| FKBP6 | 8468 |
| FLCN | 201163 |
| FLT1 | 2321 |
| FTO | 79068 |
| GATAD1 | 57798 |
| GATAD2A | 54815 |
| GATAD2B | 57459 |
| GFI1 | 2672 |
| GFI1B | 8328 |
| GFPT1 | 2673 |
| GFPT2 | 9945 |
| GLO1 | 2739 |
| GLOD4 | 51031 |
| GLOD5 | 392465 |
| GLS | 2744 |
| GLS2 | 27165 |
| GLUD1 | 2746 |
| GLUD2 | 2747 |
| GLYR1 | 84656 |
| GMEB1 | 10691 |
| GMEB2 | 26205 |
| GMNC | 647309 |
| GOT1 | 2805 |
| GOT2 | 2806 |
| GPS2 | 2874 |
| GPT | 2875 |
| GPT2 | 84706 |
| GRIP1 | 23426 |
| GSG2 | 83903 |
| GTF2A1 | 2957 |
| GTF2A1L | 11036 |
| GTF2A2 | 2958 |
| GTF2B | 2959 |
| GTF2E1 | 2960 |
| GTF2E2 | 2961 |
| GTF2F1 | 2962 |
| GTF2F2 | 2963 |
| GTF2H1 | 2965 |
| GTF2H2 | 2966 |
| GTF2H3 | 2967 |
| GTF2H4 | 2968 |
| GTF3C1 | 2975 |
| GTF3C2 | 2976 |
| GTF3C3 | 9330 |
| GTF3C4 | 9329 |
| GTF3C5 | 9328 |
| GTF3C6 | 112495 |
| H1F0 | 3005 |
| H1FOO | 132243 |
| H1FX | 8971 |
| H2AFB1 | 474382 |
| H2AFB2 | 83740 |
| H2AFX | 3014 |
| H2AFY | 9555 |
| H2AFY2 | 55506 |
| H2AFZ | 3015 |
| H2BFS | 54145 |
| H3F3A | 3020 |
| H3F3B | 3020 |
| H3F3C | 440093 |
| HAAO | 23498 |
| HAT1 | 8520 |
| HCFC1 | 3054 |
| HCFC2 | 29915 |
| HDAC1 | 3065 |
| HDAC10 | 83933 |
| HDAC11 | 79885 |
| HDAC2 | 3066 |
| HDAC3 | 8841 |
| HDAC4 | 9759 |
| HDAC5 | 10014 |
| HDAC6 | 10013 |
| HDAC7 | 51564 |
| HDAC8 | 55869 |
| HDAC9 | 9734 |
| HDGF | 3068 |
| HDGFRP2 | 84717 |
| HELLS | 3070 |
| HELZ2 | 85441 |
| HESS | 388585 |
| HEY2 | 23493 |
| HEYL | 26508 |
| HGD | 3081 |
| HIF1AN | 55662 |
| HIF3A | 64344 |
| HILS1 | 373861 |
| HINFP | 25988 |
| HINT1 | 3094 |
| HIPK2 | 28996 |
| HIRA | 7290 |
| HIST1H1A | 3024 |
| HIST1H1B | 3009 |
| HIST1H1C | 3006 |
| HIST1H1D | 3007 |
| HIST1H1E | 3008 |
| HIST1H2BA | 255626 |
| HIST1H2BB | 3018 |
| HIST1H2BC | 8339 |
| HIST1H2BD | 3017 |
| HIST1H2BE | 8339 |
| HIST1H2BF | 8339 |
| HIST1H2BG | 8339 |
| HIST1H2BH | 8345 |
| HIST1H2BI | 8339 |
| HIST1H2BJ | 8970 |
| HIST1H2BK | 85236 |
| HIST1H2BL | 8340 |
| HIST1H2BM | 8342 |
| HIST1H2BN | 8341 |
| HIST1H2BO | 8348 |
| HIST1H3A | 8350 |
| HIST1H4A | 8294 |
| HIST2H2BE | 8349 |
| HIST2H2BF | 440689 |
| HIST3H2A | 92815 |
| HIST3H2BB | 128312 |
| HJURP | 55355 |
| HLCS | 3141 |
| HLTF | 6596 |
| HMCES | 56941 |
| HMG20A | 10363 |
| HMG20B | 10362 |
| HMGA1 | 3159 |
| HMGA2 | 8091 |
| HMGB1 | 3146 |
| HMGB2 | 3148 |
| HMGB3 | 3149 |
| HMGB4 | 127540 |
| HMGN1 | 3150 |
| HMGN2 | 3151 |
| HMGN3 | 9324 |
| HMGN4 | 10473 |
| HMGN5 | 79366 |
| HMGXB3 | 22993 |
| HMGXB4 | 10042 |
| HNRNPA2B1 | 3181 |
| HNRNPC | 3183 |
| HNRNPK | 3190 |
| HP1BP3 | 50809 |
| HPD | 3242 |
| HPDL | 84842 |
| HR | 55806 |
| HSBP1 | 3281 |
| HSPBAP1 | 79663 |
| HTATIP2 | 10553 |
| HUWE1 | 10075 |
| ID3 | 3399 |
| ID4 | 3400 |
| IDH1 | 3417 |
| IDH2 | 3418 |
| IDH3A | 3419 |
| IDH3B | 3420 |
| IDH3G | 3421 |
| IDO1 | 3620 |
| IDO2 | 169355 |
| IGBP1 | 3476 |
| IKBKAP | 8518 |
| IL1B | 3553 |
| IL31RA | 133396 |
| INCENP | 3619 |
| ING1 | 3621 |
| ING2 | 3622 |
| ING3 | 54556 |
| ING4 | 51147 |
| ING5 | 84289 |
| INO80 | 54617 |
| INO80B | 83444 |
| INO80C | 125476 |
| INO80D | 54891 |
| INO80E | 283899 |
| INSM1 | 3642 |
| INTS12 | 57117 |
| IPO4 | 79711 |
| IPO9 | 55705 |
| IRF1 | 3659 |
| ITGB3BP | 23421 |
| IWS1 | 55677 |
| JADE1 | 79960 |
| JADE2 | 23338 |
| JADE3 | 9767 |
| JAK2 | 3717 |
| JARID2 | 3720 |
| JAZF1 | 221895 |
| JMJD1C | 221037 |
| JMJD4 | 65094 |
| JMJD6 | 23210 |
| JMJD7 | 100137047 |
| JMJD8 | 339123 |
| JMY | 133746 |
| JIB | 10899 |
| JUP | 3728 |
| KANSL1 | 284058 |
| KANSL2 | 54934 |
| KANSL3 | 55683 |
| KAT2A | 2648 |
| KAT2B | 8850 |
| KAT5 | 10524 |
| KAT6A | 7994 |
| KAT6B | 23522 |
| KAT7 | 11143 |
| KAT8 | 84148 |
| KCNIP3 | 30818 |
| KCTD1 | 284252 |
| KDM1A | 23028 |
| KDM1B | 221656 |
| KDM2A | 22992 |
| KDM2B | 84678 |
| KDM3A | 55818 |
| KDM3B | 51780 |
| KDM4A | 9682 |
| KDM4B | 23030 |
| KDM4C | 23081 |
| KDM4D | 55693 |
| KDM4E | 390245 |
| KDM5A | 5927 |
| KDM5B | 10765 |
| KDM5C | 8242 |
| KDM5D | 8284 |
| KDM6A | 7403 |
| KDM6B | 23135 |
| KDM7A | 80853 |
| KDM8 | 79831 |
| KIAA0101 | 9768 |
| KIAA2026 | 158358 |
| KLF1 | 10661 |
| KLF12 | 11278 |
| KLF7 | 8609 |
| KMT2A | 4297 |
| KMT2B | 9757 |
| KMT2C | 58508 |
| KMT2D | 8085 |
| KMT2E | 55904 |
| KTI12 | 112970 |
| L2HGDH | 79944 |
| L3MBTL1 | 26013 |
| L3MBTL2 | 83746 |
| L3MBTL3 | 84456 |
| L3MBTL4 | 91133 |
| LAS1L | 81887 |
| LDB1 | 8861 |
| LEF1 | 51176 |
| LEO1 | 123169 |
| LEPRE1 | 64175 |
| LEPREL1 | 55214 |
| LEPREL2 | 10536 |
| LIF | 3976 |
| LIMD1 | 8994 |
| LIN9 | 286826 |
| LMCD1 | 29995 |
| LOXL2 | 4017 |
| LPIN2 | 9663 |
| LRWD1 | 222229 |
| LSM10 | 84967 |
| LSM11 | 134353 |
| LZTS1 | 11178 |
| M1AP | 130951 |
| MAEL | 84944 |
| MAGED1 | 9500 |
| MAGOH | 4116 |
| MAK | 4117 |
| MAML1 | 9794 |
| MAML2 | 84441 |
| MAML3 | 55534 |
| MAP3K10 | 4294 |
| MAP3K12 | 7786 |
| MAP3K4 | 4216 |
| MAP3K7 | 6885 |
| MAPK11 | 5600 |
| MAPK14 | 1432 |
| MAPK3 | 5595 |
| MAPK8 | 5599 |
| MAPKAPK2 | 9261 |
| MAPKAPK3 | 7867 |
| MAZ | 4150 |
| MBD1 | 4152 |
| MBD2 | 8932 |
| MBD3 | 53615 |
| MBD3L1 | 85509 |
| MBD3L2 | 125997 |
| MBD4 | 8930 |
| MBD5 | 55777 |
| MBD6 | 114785 |
| MBIP | 51562 |
| MBTD1 | 54799 |
| MCEE | 84693 |
| MCMBP | 79892 |
| MCRS1 | 10445 |
| MDC1 | 9656 |
| MEAF6 | 64769 |
| MECOM | 2122 |
| MECP2 | 4204 |
| MED1 | 5469 |
| MED10 | 84246 |
| MED11 | 400569 |
| MED12 | 9968 |
| MED12L | 116931 |
| MED13 | 9969 |
| MED13L | 23389 |
| MED14 | 9282 |
| MED15 | 51586 |
| MED16 | 10025 |
| MED17 | 9440 |
| MED18 | 54797 |
| MED19 | 219541 |
| MED20 | 9477 |
| MED21 | 9412 |
| MED22 | 6837 |
| MED23 | 9439 |
| MED24 | 9862 |
| MED25 | 81857 |
| MED26 | 9441 |
| MED27 | 9442 |
| MED28 | 80306 |
| MED29 | 55588 |
| MED30 | 90390 |
| MED31 | 51003 |
| MED4 | 29079 |
| MED6 | 10001 |
| MED7 | 9443 |
| MED8 | 112950 |
| MED9 | 55090 |
| MEG3 | 55384 |
| MEIS1 | 4211 |
| MEMO1 | 51072 |
| MEN1 | 4221 |
| METTL13 | 51603 |
| MGA | 23269 |
| MGMT | 4255 |
| MIER1 | 57708 |
| MIER2 | 54531 |
| MIER3 | 166968 |
| MINA | 84864 |
| MIS18A | 54069 |
| MIS18BP1 | 55320 |
| MKL1 | 57591 |
| MKL2 | 57496 |
| MKRN1 | 23608 |
| MLLT1 | 4298 |
| MLLT10 | 8028 |
| MLLT3 | 4300 |
| MLLT6 | 4302 |
| MMS19 | 64210 |
| MNAT1 | 4331 |
| MNT | 4335 |
| MORF4L1 | 10933 |
| MORF4L2 | 9643 |
| MPHOSPH8 | 54737 |
| MRGBP | 55257 |
| MSC | 9242 |
| MSH6 | 2956 |
| MSL1 | 339287 |
| MSL2 | 55167 |
| MSL3 | 10943 |
| MSL3P1 | 151507 |
| MT3 | 4504 |
| MTA1 | 9112 |
| MTA2 | 9219 |
| MTA3 | 57504 |
| MTDH | 92140 |
| MTERF | 7978 |
| MTF1 | 4520 |
| MTF2 | 22823 |
| MTRR | 4552 |
| MUC1 | 4582 |
| MXD1 | 4084 |
| MXD4 | 10608 |
| MXI1 | 4601 |
| MYC | 4609 |
| MYCBP | 26292 |
| MYOCD | 93649 |
| MYOD1 | 4654 |
| MYSM1 | 114803 |
| N4BP2L2 | 10443 |
| NAA40 | 79829 |
| NAA50 | 80218 |
| NAA60 | 79903 |
| NAB2 | 4665 |
| NACA | 4666 |
| NACC2 | 138151 |
| NANOG | 79923 |
| NAP1L1 | 4673 |
| NAP1L2 | 4674 |
| NAP1L3 | 4675 |
| NAP1L4 | 4676 |
| NAP1L5 | 266812 |
| NASP | 4678 |
| NCAPD2 | 9918 |
| NCAPD3 | 23310 |
| NCAPG | 64151 |
| NCAPG2 | 54892 |
| NCAPH | 23397 |
| NCBP1 | 4686 |
| NCBP2 | 22916 |
| NCOA1 | 8648 |
| NCOA2 | 10499 |
| NCOA3 | 8202 |
| NCOA4 | 8031 |
| NCOA6 | 23054 |
| NCOA7 | 135112 |
| NCOR1 | 9611 |
| NCOR2 | 9612 |
| NCR1 | 9437 |
| NDUFAF5 | 79133 |
| NEK2 | 4751 |
| NELFA | 7469 |
| NELFB | 25920 |
| NELFCD | 51497 |
| NELFE | 7936 |
| NEUROG1 | 4762 |
| NEUROG3 | 50674 |
| NFATC3 | 4775 |
| NFATC4 | 4776 |
| NFE2 | 4778 |
| NFRKB | 4798 |
| NFX1 | 4799 |
| NFYA | 4800 |
| NFYB | 4801 |
| NFYC | 4802 |
| NIPBL | 25836 |
| NIT2 | 56954 |
| NKX2B | 4821 |
| NOC2L | 26155 |
| NOTCH2 | 4853 |
| NPAT | 4863 |
| NPM1 | 4869 |
| NPM2 | 10361 |
| NPM3 | 10360 |
| NRIP1 | 8204 |
| NSD1 | 64324 |
| NSL1 | 25936 |
| NSMCE2 | 286053 |
| NUDT21 | 11051 |
| OAT | 4942 |
| OGDH | 4967 |
| OGDHL | 55753 |
| OGFOD1 | 55239 |
| OGFOD2 | 79676 |
| OGFOD3 | 79701 |
| OGT | 8473 |
| OIP5 | 11339 |
| OTUB1 | 55611 |
| P4HA1 | 5033 |
| P4HA2 | 8974 |
| P4HA3 | 283208 |
| P4HB | 5034 |
| P4HTM | 54681 |
| PABPN1 | 8106 |
| PADI1 | 29943 |
| PADI2 | 11240 |
| PADI3 | 51702 |
| PADI4 | 23569 |
| PAF1 | 54623 |
| PAGR1 | 79447 |
| PAK1 | 5058 |
| PAPOLA | 10914 |
| PAPOLB | 56903 |
| PARP1 | 142 |
| PARP10 | 84875 |
| PARP12 | 64761 |
| PARP14 | 54625 |
| PARP15 | 165631 |
| PARP2 | 10038 |
| PARP3 | 10039 |
| PARP4 | 143 |
| PARP9 | 83666 |
| PAWR | 5074 |
| PAXIP1 | 22976 |
| PBRM1 | 55193 |
| PBXIP1 | 57326 |
| PCBD1 | 5092 |
| PCF11 | 51585 |
| PCGF1 | 84759 |
| PCGF2 | 7703 |
| PCGF3 | 10336 |
| PCGF5 | 84333 |
| PCGF6 | 84108 |
| PCNA | 5111 |
| PDS5A | 23244 |
| PDS5B | 23047 |
| PELP1 | 27043 |
| PER1 | 5187 |
| PER2 | 8864 |
| PEX14 | 5195 |
| PFDN5 | 5204 |
| PGR | 5241 |
| PHB | 5245 |
| PHC1 | 1911 |
| PHC2 | 1912 |
| PHC3 | 80012 |
| PHF1 | 5252 |
| PHF10 | 55274 |
| PHF11 | 51131 |
| PHF12 | 57649 |
| PHF13 | 148479 |
| PHF14 | 9678 |
| PHF19 | 26147 |
| PHF2 | 5253 |
| PHF20 | 51230 |
| PHF20L1 | 51105 |
| PHF21A | 51317 |
| PHF21B | 112885 |
| PHF23 | 79142 |
| PHF3 | 23469 |
| PHF5A | 84844 |
| PHF6 | 84295 |
| PHF7 | 51533 |
| PHF8 | 23133 |
| PHGDH | 26227 |
| PHIP | 55023 |
| PHRF1 | 57661 |
| PHYH | 5264 |
| PHYHD1 | 254295 |
| PIAS1 | 8554 |
| PIAS2 | 9063 |
| PICK1 | 9463 |
| PIF1 | 80119 |
| PIM1 | 5292 |
| PIWIL2 | 55124 |
| PIWIL4 | 143689 |
| PKN1 | 5585 |
| PLD6 | 201164 |
| PLK1 | 5347 |
| PLOD1 | 5351 |
| PLOD2 | 5352 |
| PLOD3 | 8985 |
| PLRG1 | 5356 |
| PMF1 | 11243 |
| PML | 5371 |
| POLE3 | 54107 |
| POLE4 | 56655 |
| POLR1A | 25885 |
| POLR1B | 84172 |
| POLR1C | 9533 |
| POLR1D | 51082 |
| POLR2A | 5430 |
| POLR2B | 5431 |
| POLR2C | 5432 |
| POLR2D | 5433 |
| POLR2E | 5434 |
| POLR2F | 5435 |
| POLR2G | 5436 |
| POLR2H | 5437 |
| POLR2I | 5438 |
| POLR2J | 5439 |
| POLR2K | 5440 |
| POLR2L | 5441 |
| POLR3A | 11128 |
| POLR3B | 55703 |
| POLR3C | 10623 |
| POLR3D | 661 |
| POLR3E | 55718 |
| POLR3F | 10621 |
| POLR3G | 10622 |
| POLR3GL | 84265 |
| POLR3H | 171568 |
| POLR3K | 51728 |
| POT1 | 25913 |
| POU5F1 | 5460 |
| PPARG | 5468 |
| PPARGC1A | 10891 |
| PPARGC1B | 133522 |
| PPP1CA | 5499 |
| PPP1CB | 5500 |
| PPP1CC | 5501 |
| PPP1R13L | 10848 |
| PPP4R2 | 151987 |
| PQBP1 | 10084 |
| PRDM10 | 56980 |
| PRDM11 | 56981 |
| PRDM12 | 59335 |
| PRDM13 | 59336 |
| PRDM15 | 63977 |
| PRDM16 | 63976 |
| PRDM2 | 7799 |
| PRDM4 | 11108 |
| PRDM5 | 11107 |
| PRDM6 | 93166 |
| PRDM7 | 11105 |
| PRDM8 | 56978 |
| PRDM9 | 56979 |
| PRKAA1 | 5562 |
| PRKAA2 | 5563 |
| PRKCA | 5578 |
| PRKCB | 5579 |
| PRKD1 | 5587 |
| PRKD2 | 25865 |
| PRMT1 | 3276 |
| PRMT2 | 3275 |
| PRMT3 | 10196 |
| PRMT5 | 10419 |
| PRMT6 | 55170 |
| PRMT7 | 54496 |
| PRMT8 | 56341 |
| PRPF31 | 26121 |
| PRPF6 | 24148 |
| PSAT1 | 29968 |
| PSIP1 | 11168 |
| PSMC3 | 5702 |
| PSMC3IP | 29893 |
| PSMD14 | 10213 |
| PSMD9 | 5715 |
| PSME4 | 23198 |
| PTGS1 | 5742 |
| PTGS2 | 5743 |
| PTPN2 | 5771 |
| PTRF | 284119 |
| PWWP2B | 170394 |
| PYGO1 | 26108 |
| PYGO2 | 90780 |
| RAD18 | 56852 |
| RAD21 | 5885 |
| RAD54L | 8438 |
| RAG1 | 5896 |
| RAG2 | 5897 |
| RAI1 | 10743 |
| RAN | 5901 |
| RAP2C | 57826 |
| RARA | 5914 |
| RB1 | 5925 |
| RBBP4 | 5928 |
| RBBP5 | 5929 |
| RBBP7 | 5931 |
| RBBP8 | 5932 |
| RBFOX2 | 23543 |
| RBL1 | 5933 |
| RBL2 | 5934 |
| RBM14 | 10432 |
| RBM8A | 9939 |
| RBP1 | 5947 |
| RBP2 | 5948 |
| RBPMS | 11030 |
| RCBTB1 | 55213 |
| RCC1 | 1104 |
| RCOR1 | 23186 |
| RCOR2 | 283248 |
| RCOR3 | 55758 |
| RERE | 473 |
| REST | 5978 |
| RFX5 | 5993 |
| RFXAP | 5994 |
| RING1 | 6015 |
| RIPK3 | 11035 |
| RLIM | 51132 |
| RNF14 | 9604 |
| RNF168 | 165918 |
| RNF169 | 254225 |
| RNF17 | 56163 |
| RNF2 | 6045 |
| RNF20 | 56254 |
| RNF4 | 6047 |
| RNF40 | 9810 |
| RNF8 | 9025 |
| RNMT | 8731 |
| RNPS1 | 10921 |
| RPE65 | 6121 |
| RPRD1B | 58490 |
| RPS6KA4 | 8986 |
| RPS6KA5 | 9252 |
| RRP8 | 23378 |
| RSF1 | 51773 |
| RUVBL1 | 8607 |
| RUVBL2 | 10856 |
| RYBP | 23429 |
| SALL1 | 6299 |
| SAP130 | 79595 |
| SAP18 | 10284 |
| SAP25 | 100316904 |
| SAP30 | 8819 |
| SAP30L | 79685 |
| SATB1 | 6304 |
| SATB2 | 23314 |
| SCAI | 286205 |
| SCAND1 | 51282 |
| SCG2 | 7857 |
| SCML2 | 10389 |
| SENP3 | 26168 |
| SENP6 | 26054 |
| SET | 6418 |
| SETBP1 | 26040 |
| SETD1A | 9739 |
| SETD1B | 23067 |
| SETD2 | 29072 |
| SETD3 | 84193 |
| SETD4 | 54093 |
| SETD5 | 55209 |
| SETD6 | 79918 |
| SETD7 | 80854 |
| SETD8 | 387893 |
| SETD9 | 133383 |
| SETDB1 | 9869 |
| SETDB2 | 83852 |
| SETMAR | 6419 |
| SF1 | 7536 |
| SFMBT1 | 51460 |
| SFMBT2 | 57713 |
| SFPQ | 6421 |
| SHMT1 | 6470 |
| SHPRH | 257218 |
| SIAH2 | 6478 |
| SIK1 | 150094 |
| SIN3A | 25942 |
| SIN3B | 23309 |
| SIRT1 | 23411 |
| SIRT2 | 22933 |
| SIRT3 | 23410 |
| SIRT4 | 23409 |
| SIRTS | 23408 |
| SIRT6 | 51548 |
| SIRT7 | 51547 |
| SKI | 6497 |
| SKIL | 6498 |
| SKOR2 | 652991 |
| SKP1 | 6500 |
| SLBP | 7884 |
| SLC22A11 | 55867 |
| SLC22A12 | 116085 |
| SLC22A13 | 9390 |
| SLC22A20 | 440044 |
| SLC22A25 | 387601 |
| SLC22A6 | 9356 |
| SLC22A7 | 10864 |
| SLC22A8 | 9376 |
| SMAD2 | 4087 |
| SMAD3 | 4088 |
| SMAD4 | 4089 |
| SMAP2 | 64744 |
| SMARCA1 | 6594 |
| SMARCA2 | 6595 |
| SMARCA4 | 6597 |
| SMARCA5 | 8467 |
| SMARCAD1 | 56916 |
| SMARCAL1 | 50485 |
| SMARCB1 | 6598 |
| SMARCC1 | 6599 |
| SMARCC2 | 6601 |
| SMARCD1 | 6602 |
| SMARCD2 | 6603 |
| SMARCD3 | 6604 |
| SMARCE1 | 6605 |
| SMC1A | 8243 |
| SMC1B | 27127 |
| SMC2 | 10592 |
| SMC3 | 9126 |
| SMC4 | 10051 |
| SMC5 | 23137 |
| SMNDC1 | 10285 |
| SMYD1 | 150572 |
| SMYD2 | 56950 |
| SMYD3 | 64754 |
| SMYD4 | 114826 |
| SMYD5 | 10322 |
| SNAI2 | 6591 |
| SNAPC4 | 6621 |
| SNCA | 6622 |
| SND1 | 27044 |
| SNRPB | 6628 |
| SNRPD3 | 6634 |
| SNRPE | 6635 |
| SNRPF | 6636 |
| SNRPG | 6637 |
| SNW1 | 22938 |
| SP100 | 6672 |
| SP110 | 3431 |
| SP140 | 11262 |
| SP140L | 93349 |
| SP4 | 6671 |
| SPEN | 23013 |
| SPI1 | 6688 |
| SPIN1 | 10927 |
| SPRTN | 83932 |
| SRA1 | 10011 |
| SRCAP | 10847 |
| SRRM1 | 10250 |
| SRSF1 | 6426 |
| SRSF11 | 9295 |
| SRSF2 | 6427 |
| SRSF3 | 6428 |
| SRSF4 | 6429 |
| SRSF5 | 6430 |
| SRSF6 | 6431 |
| SRSF7 | 6432 |
| SRSF9 | 8683 |
| SRY | 6736 |
| SS18 | 6760 |
| SS18L1 | 26039 |
| SSRP1 | 6749 |
| SSX1 | 6756 |
| STAT5B | 6777 |
| STK3 | 6788 |
| STK31 | 56164 |
| STK38 | 11329 |
| SUB1 | 10923 |
| SUDS3 | 64426 |
| SUFU | 51684 |
| SUPT16H | 11198 |
| SUPT20H | 55578 |
| SUPT3H | 8464 |
| SUPT4H1 | 6827 |
| SUPT5H | 6829 |
| SUPT6H | 6830 |
| SUPT7L | 9913 |
| SUV39H1 | 6839 |
| SUV39H2 | 79723 |
| SUV420H1 | 51111 |
| SUV420H2 | 84787 |
| SUZ12 | 23512 |
| SYCP3 | 50511 |
| TADA1 | 117143 |
| TADA2A | 6871 |
| TADA2B | 93624 |
| TADA3 | 10474 |
| TAF1 | 6872 |
| TAF10 | 6881 |
| TAF11 | 6882 |
| TAF12 | 6883 |
| TAF13 | 6884 |
| TAF1A | 9015 |
| TAF1B | 9014 |
| TAF1C | 9013 |
| TAF1D | 79101 |
| TAF1L | 138474 |
| TAF2 | 6873 |
| TAF3 | 83860 |
| TAF4 | 6874 |
| TAF4B | 6875 |
| TAF5 | 6877 |
| TAF5L | 27097 |
| TAF6 | 6878 |
| TAF6L | 10629 |
| TAF7 | 6879 |
| TAF7L | 54457 |
| TAF8 | 129685 |
| TAF9 | 6880 |
| TAF9B | 51616 |
| TAL1 | 6886 |
| TANC1 | 85461 |
| TANC2 | 26115 |
| TAT | 6898 |
| TBL1X | 6907 |
| TBL1XR1 | 79718 |
| TBP | 6908 |
| TBPL1 | 9519 |
| TBX18 | 9096 |
| TBX20 | 57057 |
| TCEA1 | 6917 |
| TCEA2 | 6919 |
| TCEAL1 | 9338 |
| TCEAL2 | 140597 |
| TCEAL3 | 85012 |
| TCEAL4 | 79921 |
| TCEAL5 | 340543 |
| TCEAL6 | 158931 |
| TCEAL7 | 56849 |
| TCEAL8 | 90843 |
| TCEB1 | 6921 |
| TCEB2 | 6923 |
| TCEB3 | 6924 |
| TCERG1 | 10915 |
| TDG | 6996 |
| TDO2 | 6999 |
| TDP2 | 51567 |
| TDRD1 | 56165 |
| TDRD12 | 91646 |
| TDRD3 | 81550 |
| TDRD5 | 163589 |
| TDRD6 | 221400 |
| TDRD7 | 23424 |
| TDRD9 | 122402 |
| TDRKH | 11022 |
| TERF1 | 7013 |
| TERF2 | 7014 |
| TERF2IP | 54386 |
| TERT | 7015 |
| TET1 | 80312 |
| TET2 | 54790 |
| TET3 | 200424 |
| TEX10 | 54881 |
| TFCP2L1 | 29842 |
| TFDP1 | 7027 |
| TFEC | 22797 |
| TFPT | 29844 |
| TGFB111 | 7041 |
| THRAP3 | 9967 |
| THRB | 7068 |
| THUMPD2 | 80745 |
| TICRR | 90381 |
| TINF2 | 26277 |
| TLE1 | 7088 |
| TLE2 | 7089 |
| TLK1 | 9874 |
| TLK2 | 11011 |
| TMLHE | 55217 |
| TNKS | 8658 |
| TNKS2 | 80351 |
| TNP1 | 7141 |
| TONSL | 4796 |
| TOP1 | 7150 |
| TOP1MT | 116447 |
| TOP2B | 7155 |
| TP53BP1 | 7158 |
| TPR | 7175 |
| TRAF7 | 84231 |
| TRDMT1 | 1787 |
| TRERF1 | 55809 |
| TRIB3 | 57761 |
| TRIM16 | 10626 |
| TRIM22 | 10346 |
| TRIM24 | 8805 |
| TRIM28 | 10155 |
| TRIM32 | 22954 |
| TRIM33 | 51592 |
| TRIM66 | 9866 |
| TRIP11 | 9321 |
| TRIP12 | 9320 |
| TRIP4 | 9325 |
| TRRAP | 8295 |
| TSG101 | 7251 |
| TSHZ3 | 57616 |
| TSPYL2 | 64061 |
| TTF1 | 7270 |
| TTF1 | 7080 |
| TTF2 | 8458 |
| TWIST1 | 7291 |
| TYW5 | 129450 |
| U2AF1 | 7307 |
| U2AF2 | 11338 |
| UBAP2L | 9898 |
| UBE2A | 7319 |
| UBE2B | 7320 |
| UBE2E1 | 7324 |
| UBE2I | 7329 |
| UBE2L3 | 7332 |
| UBE2N | 7334 |
| UBE2NL | 389898 |
| UBE2V1 | 7335 |
| UBN1 | 29855 |
| UBP1 | 7342 |
| UBR2 | 23304 |
| UBR5 | 51366 |
| UBTF | 7343 |
| UCHL5 | 51377 |
| UHRF1 | 29128 |
| UHRF2 | 115426 |
| UIMC1 | 51720 |
| UPF1 | 5976 |
| UPF3B | 65109 |
| URI1 | 8725 |
| USP11 | 8237 |
| USP16 | 10600 |
| USP17L2 | 377630 |
| USP21 | 27005 |
| USP22 | 23326 |
| USP3 | 9960 |
| USP49 | 25862 |
| USP7 | 7874 |
| UTF1 | 8433 |
| UTP3 | 57050 |
| UTY | 7404 |
| UXT | 8409 |
| VEGFA | 7422 |
| VGLL1 | 51442 |
| VPRBP | 9730 |
| VPS72 | 6944 |
| VRK1 | 7443 |
| WAC | 51322 |
| WAPAL | 23063 |
| WBP2 | 23558 |
| WBSCR22 | 114049 |
| WDHD1 | 11169 |
| WDR11 | 55717 |
| WDR5 | 11091 |
| WDR61 | 80349 |
| WDR77 | 79084 |
| WDR82 | 80335 |
| WDR92 | 116143 |
| WHSC1 | 7468 |
| WHSC1L1 | 54904 |
| WNT4 | 54361 |
| WWC1 | 23286 |
| WWTR1 | 25937 |
| XIAP | 331 |
| YAF2 | 10138 |
| YAP1 | 10413 |
| YBX2 | 51087 |
| YBX3 | 8531 |
| YEATS2 | 55689 |
| YEATS4 | 8089 |
| YY1 | 7528 |
| ZBTB16 | 7704 |
| ZBTB32 | 27033 |
| ZBTB33 | 10009 |
| ZCCHC12 | 170261 |
| ZCWPW1 | 55063 |
| ZEB1 | 6935 |
| ZFP57 | 346171 |
| ZFPM1 | 161882 |
| ZFPM2 | 23414 |
| ZHX1 | 11244 |
| ZMIZ2 | 83637 |
| ZMYND11 | 10771 |
| ZMYND8 | 23613 |
| ZNF136 | 7695 |
| ZNF217 | 7764 |
| ZNF224 | 7767 |
| ZNF274 | 10782 |
| ZNF281 | 23528 |
| ZNF335 | 63925 |
| ZNF350 | 59348 |
| ZNF366 | 167465 |
| ZNF473 | 25888 |
| ZNF593 | 51042 |
| ZNF85 | 7639 |
| ZNRD1 | 30834 |
| ZSCAN1 | 284312 |
| ZZZ3 | 26009 |

In one aspect, the present invention provides for a screening platform that enables screening of at least 274 chromatin regulators (“300K library screen”). In certain embodiments, 2 or more guide sequences target each gene for each Cas9 ortholog. In certain embodiments, the screening platform includes non-target control guide sequences for each ortholog. In certain embodiments, the platform includes more than 2, 4, 10, or 20 non-target guide sequences. In certain embodiments, the platform includes one or more essential positive control genes (e.g., 2 or more guide sequences for each ortholog). In certain embodiments, the library screen includes 552 (S. aureus)×552 (S. pyogenes) guide sequences=304,704 combinatorial perturbations.

In certain embodiments, each S. aureus guide sequence is included in an oligonucleotide having a framework that allows construction of the orthogonal combinatorial library as described herein. In certain embodiments, the S. aureus guide sequence used for the 300K library are SEQ ID NOS: 1-552. In certain embodiments, the frame work includes from 5′ to 3′ a restriction enzyme site for cloning into the vector comprising convergent regulatory sequences, a 20-21 nucleotide S. aureus guide sequence, a S. aureus tracr sequence, optionally, a barcode identifying the guide sequence, and an overlap sequence for hybridization to a complementary overlap sequence on the S. pyogenes oligonucleotide framework. In certain embodiments, the 300K library is designed such that the S. aureus guide sequences are inserted into the vector such that they are operably linked to the H1 cassette. In certain embodiments, the S. aureus oligonucleotide framework is a 140 nucleotide oligonucleotide that includes a BsmBI cassette (e.g., GCCGTCTCGTCCCG) (SEQ ID NO:45,521), the 21 nucleotide S. aureus guide sequence described above, the S. aureus tracr sequence (e.g., GTTTAAGTACTCTGGAAACAGAATCTACTTAAACAAGGCAAAATGCCGTGTTTAT CTCGTCAACTTGTTGGCGAGATTTTTT (SEQ ID NO:45,522)), a 6 nucleotide barcode sequence, and the overlap sequence (e.g., GTGCACGAGATCATCCG (SEQ ID NO:45,523)). In certain embodiment, the S. aureus oligonucleotide is GCCGTCTCGTCCCG—(SEQ ID NO:45,524) 21-nucleotide guide sequence—GTTTAAGTACTCTGGAAACAGAATCTACTTAAACAAGGCAAAATGCCGTGTTTAT CTCGTCAACTTGTTGGCGAGATTTTTT (SEQ ID NO:45,525)—6 nucleotide barcode—GTGCACGAGATCATCCG (SEQ ID NO:45,526).

In certain embodiments, each S. pyogenes guide sequence is included in an oligonucleotide having a framework that allows construction of the orthogonal combinatorial library as described herein. In certain embodiments, the S. pyogenes guide sequence used for the 300K library are SEQ ID NOS: 553-1104. In certain embodiments, the frame work includes from 5′ to 3′ a restriction enzyme site for cloning into the vector comprising convergent regulatory sequences, a 20-21 nucleotide S. pyogenes guide sequence, a S. pyogenes tracr sequence, optionally, a barcode identifying the guide sequence, and an overlap sequence for hybridization to a complementary overlap sequence on the S. aureus oligonucleotide framework. In certain embodiments, the 300K library is designed such that the S. pyogenes guide sequences are inserted into the vector such that they are operably linked to the U6 cassette. In certain embodiments, the S. pyogenes oligonucleotide framework is a 139 nucleotide oligonucleotide that includes a BsmBI cassette (e.g., GCCGTCTCGCACCG (SEQ ID NO:45,527)), the 20 nucleotide S. pyogenes guide sequence described above, the S. pyogenes tracr sequence (e.g., GTTTGAGAGCTAGAAATAGCAAGTTCAAATAAGGCTAGTCCGTTATCAACTTGA AAAAGTGGCACCGAGTCGGTGCTTTTTT (SEQ ID NO:45,528)), a 6 nucleotide barcode sequence, and the overlap sequence (e.g., ACGGATGATCTCGTGCA (SEQ ID NO:45,529)). In certain embodiment, the S. pyogenes oligonucleotide is GCCGTCTCGCACCG (SEQ ID NO:45,530) —20-nucleotide guide sequence-GTTTGAGAGCTAGAAATAGCAAGTTCAAATAAGGCTAGTCCGTTATCAACTTGA AAAAGTGGCACCGAGTCGGTGCTTTTTT (SEQ ID NO:45,531) —6 nucleotide barcode-ACGGATGATCTCGTGCA (SEQ ID NO:45,532).

In certain embodiments, the screen can include guide sequences targeting pfam domains in any chromatin regulator. In certain embodiments, the guide sequences are included in the framework described above. In certain embodiments, the S. aureus pfam domain targeting guide sequences are selected from SEQ ID NOS: 1105-23903. In certain embodiments, the S. pyogenes pfam domain targeting guide sequences are selected from SEQ ID NOS: 23904-45515.

Screening for Combinations of Targets that Confer Specific Phenotypes

In certain embodiments, the combinatorial screening platform can be used to identify combinations of targets that confer specific phenotypes. A “selected phenotype” refers to any phenotype, e.g., any observable characteristic or functional effect that can be measured in an assay such as changes in cell growth, proliferation, morphology, enzyme function, signal transduction, expression patterns, downstream expression patterns, reporter gene activation, hormone release, growth factor release, neurotransmitter release, ligand binding, apoptosis, and product formation. In certain embodiments, a positive or negative screen can be performed. In a negative screen guide sequences pairs that are depleted are identified (e.g., screening for viability, sensitivity to a drug). In certain embodiments, a phenotype can include viability, differentiation, or changes in cell state. In a positive screen guide sequence pairs that are enriched in a population of cells having a specific phenotype are identified (e.g., expression of a cell marker).

Exemplary assay embodiments include, e.g., transformation assays, e.g., changes in proliferation, anchorage dependence, growth factor dependence, foci formation, growth in soft agar, tumor proliferation in nude mice, and tumor vascularization in nude mice; apoptosis assays, e.g., DNA laddering and cell death, expression of genes involved in apoptosis; signal transduction assays, e.g., changes in intracellular calcium, cAMP, cGMP, inositol trisphosphate (IP3), changes in hormone and neurotransmittor release; receptor assays, e.g., estrogen receptor and cell growth; growth factor assays, e.g., EPO, hypoxia and erythrocyte colony forming units assays; enzyme product assays, e.g., FAD-2 induced oil desaturation; transcription assays, e.g., reporter gene assays; and protein production assays, e.g., VEGF. A candidate gene or genetic element is “associated with” a selected phenotype if modulation of gene expression of the candidate gene or modulation of the genetic element causes a change in the selected phenotype.

In certain embodiments, cells are assayed for changes in cell state (e.g., immune state, proliferation, senescence). For example, chronic viral infections and cancer often lead to the emergence of dysfunctional or ‘exhausted’ CD8+ T cells, and the restoration of their functions is currently the focus of therapeutic interventions (see, e.g., Wang, Singer and Anderson, 2017, Molecular Dissection of CD8+ T-Cell Dysfunction. Trends in Immunology, volume 38, issue 8, p567-576). In certain embodiments, CD8+ T cells are assayed for markers of dysfunction (e.g., coinhibitory receptors, such as PD-1, TIM-3, CTLA4, LAG3). Determining combinations of chromatin regulators that when targeted restore cell function may advantageously be used in adoptive cell transfer strategies described further herein (e.g., CAR T cells). In certain embodiments, an immune response is screened using the present invention.

Chromatin regulation and epigenetics is involved in stem cell differentiation and development (see, e.g., Atlasi and Stunnenberg, 2017, The interplay of epigenetic marks during stem cell differentiation and development. Nature Reviews Genetics volume 18, pages 643-658). In certain embodiments, cells are assayed for differentiation markers or markers present on undifferentiated or differentiated cell types (neurons, immune cells, tissue subtype cells).

In certain embodiments, the present invention may be used with transgenic mice expressing one or more orthologous CRISPR enzymes. In certain embodiments, the transgenic mice are mouse models of disease or are treated with an agent to model a disease. Not being bound by a theory, the present invention may be used for screening combinatorial synthetic lethality phenotypes in the background of a disease model.

In certain embodiments, the present invention may be used to screen cells in an animal model ex vivo or in vivo. In one aspect, the present invention provides for a method of combinatorial screening of phenotypic interactions between a set of target sequences in a population of cells comprising: introducing a library according to any embodiment described herein to a transgenic animal expressing at least one CRISPR enzyme from a transgene, wherein the cells of the transgenic animal express two orthogonal CRISPR enzymes; obtaining dissected tissue from said transgenic animal; and determining the enrichment or depletion of combinations of sgRNA sequences in said tissue compared to the representation in the library introduced. In one embodiment, the library is introduced to the brain of a transgenic mouse and synthetic lethality of neurons is determined. In another aspect, the library of the present invention is introduced to cells ex vivo and the cells transferred to an animal model. In certain embodiments, the library is introduced to tumor cells. Cells may be selected that express a vector of the library. The tumor cells may be transferred to a mouse model and the representation of sgRNA combinations may be detected in tumor cells grown in the animal model. Not being bound by a theory, combinations of sgRNAs that are lethal in vivo will be reduced as compared to the library input and combinations of sgRNAs that allow proliferation will be enriched as compared to the library input.

Methods and compositions described herein are broadly applicable to any study that could benefit from the targeting of combinatorial sets of genetic elements. For example, this approach could lead to identification of novel drug targets elucidated by network perturbation, which could define subtler enzymatic pathways leading to disease, or enable drug discovery of novel chemical or biological mediators (including combinations of chemical and/or biological mediators) for treating disease. Additionally, technologies described herein could be applied to the discovery of combinations of existing drug targets for disease treatment and/or prevention, and could lead to novel combination treatments using FDA-approved therapeutics.

The present invention advantageously allows for assaying changes in phenotypes caused by combinatorial targeting of genetic elements. In certain embodiments, the CRISPR single guide sequence combinations associated with a phenotype may be identified by sequencing the CRISPR single guide sequences or associated barcodes. Several non-limiting examples of phenotypes of interest that may be screened or selected for according to aspects of the invention include, in mammalian cells: gene expression, cell proliferation, synthetic lethality, reduction of disease state, production of disease state, complex multifactorial diseases, aging and age-related diseases, neurodegeneration, drug resistance or sensitivity, chemotherapy resistance, pathway modulation (e.g., stress response, apoptosis, immune cell dysfunction or activation), resistance to infection, stem cell differentiation, cell type transdifferentiation and potentiation of FDA-approved drugs.

In certain embodiments, methods are provided for identifying combinations of genetic elements that when inhibited or activated reduce or prevent proliferation of a cell or population of cells. In certain embodiments, cell proliferation may be assayed by culturing cells comprising a vector of the present invention for at least two periods of time and identifying combinations of sgRNAs. The methods may involve contacting two populations of cells with a combinatorial library of the present invention. The two populations of cells may be cultured for different durations of time. For example, one population of cells may be cultured for 3-15 days and the other population of cells is cultured for 20-30 days. The identification of the combinations of CRISPR guide sequences are determined for each population of cells, e.g. by sequencing methods. The abundance of each combination of CRISPR single guide sequences in the population of cells that was cultured for a longer duration of time is compared to the abundance of each combination of CRISPR guide sequences in the population of cells that was cultured for the shorter duration of time. Not being bound by a theory, combinations of CRISPR guide sequences that reduced proliferation of the cells will be less abundant in the population of cells that was cultured for the longer duration of time compared to the abundance of the CRISPR guide sequence in the population of cells that was cultured for the shorter duration of time. Such combinations are identified as combinations that reduce cell proliferation.

The application similarly provides methods of screening for genomic sites associated with resistance to a chemical compound whereby the cells are contacted with the chemical compound and screened based on the phenotypic reaction to said compound. More particularly such methods may comprise introducing the library of CRISPR/Cas system guide RNAs envisaged herein into a population of cells (that are either adapted to contain a Cas protein or whereby the Cas protein is simultaneously introduced), treating the population of cells with the chemical compound; and determining the representation of guide RNAs after treatment with the chemical compound at a later time point as compared to an early time point. In these methods, the genomic sites associated with resistance to the chemical compound are determined by enrichment of guide RNAs.

Aspects of the invention relate to modulation of gene expression in response to combinatorial CRISPR targeting and modulation can be assayed by determining any parameter that is indirectly or directly affected by the expression of a target gene. Such parameters include, e.g., changes in RNA or protein levels, changes in protein activity, changes in product levels, changes in downstream gene expression, changes in reporter gene transcription (luciferase, CAT, beta-galactosidase, beta-glucuronidase, GFP or any fluorescent protein (see, e.g., Mistili & Spector, Nature Biotechnology 15:961-964 (1997)); changes in signal transduction, phosphorylation and dephosphorylation, receptor-ligand interactions, second messenger concentrations (e.g., cGMP, cAMP, IP3, and Ca²⁺), cell growth, and neovascularization, etc., as described herein. These assays can be in vitro, in vivo, and ex vivo. Such functional effects can be measured by any means known to those skilled in the art, e.g., measurement of RNA or protein levels, measurement of RNA stability, identification of downstream or reporter gene expression, e.g., via chemiluminescence, fluorescence, calorimetric reactions, antibody binding, inducible markers, ligand binding assays; changes in intracellular second messengers such as cGMP and inositol triphosphate (IP3); changes in intracellular calcium levels; cytokine release, and the like, as described herein.

Aspects of the invention comprehend many types of screens and selection mechanisms that can also be used with CRISPR screening. Screens for resistance to viral or bacterial pathogens may be used to identify genes that prevent infection or pathogen replication. As in drug resistance screens, survival after pathogen exposure provides strong selection. In cancer, negative selection CRISPR screens may identify “oncogene addictions” in specific cancer subtypes that can provide the foundation for molecular targeted therapies. For developmental studies, screening in human and mouse pluripotent cells may pinpoint genes required for pluripotency or for differentiation into distinct cell types. To distinguish cell types, fluorescent or cell surface marker reporters of gene expression may be used and cells may be sorted into groups based on expression level. Gene-based reporters of physiological states, such as activity-dependent transcription during repetitive neural firing or from antigen-based immune cell activation, may also be used. Any phenotype that is compatible with rapid sorting or separation may be harnessed for pooled screening. CRISPR screening may also be used as a diagnostic tool: With patient-derived iPS cells, genome-wide libraries may be used to examine multi-gene interactions (similar to synthetic lethal screens) or how different loss-of-functions mutations accumulated through aging or disease can interact with particular drug treatments.

Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). In an aspect of the invention, a reporter gene which includes but is not limited to glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In a further embodiment of the invention, the DNA molecule encoding the gene product may be introduced into the cell via a vector. In a preferred embodiment of the invention the gene product is luciferase. In a further embodiment of the invention the expression of the gene product is decreased.

### Screening for Phenotypes in Plants

With recent advances in crop genomics, the ability to use CRISPR-Cas systems to perform efficient and cost effective gene editing and manipulation will allow the rapid selection and comparison of single and multiplexed genetic manipulations to transform such genomes for improved production and enhanced traits. In this regard reference is made to US patents and publications: U.S. Pat. No. 6,603,061—Agrobacterium-Mediated Plant Transformation Method; U.S. Pat. No. 7,868,149—Plant Genome Sequences and Uses Thereof and US 2009/0100536—Transgenic Plants with Enhanced Agronomic Traits, all the contents and disclosure of each of which are herein incorporated by reference in their entirety. In the practice of the invention, the contents and disclosure of Morrell et al “Crop genomics: advances and applications” (Nat Rev Genet. 2011 Dec. 29; 13(2):85-96) are also herein incorporated by reference in their entirety. In some methods of the invention the vector is an Agrobacterium Ti or Ri plasmid for use in plants. In exemplary embodiments, the DNA constructs according to the present invention may be used in a vector configured for use in plants and plant cells. In certain embodiments, a library of the present invention is transformed into protoplasts. Plants may be regenerated from the protoplasts and plants having desired characteristics may be selected. The sgRNA combinations may then be identified. Not being bound by a theory, the present invention may allow for pairwise combinations of perturbations to be screened in plants in an unbiased manner.

In plants, pathogens are often host-specific. For example, Fusarium oxysporum f. sp. lycopersici causes tomato wilt but attacks only tomato, and F. oxysporum f. dianthii Puccinia graminis f sp. tritici attacks only wheat. Plants have existing and induced defenses to resist most pathogens. Mutations and recombination events across plant generations lead to genetic variability that gives rise to susceptibility or reduced susceptibility or resistance, especially as pathogens reproduce with more frequency than plants. In plants, there can be non-host resistance, e.g., the host and pathogen are incompatible. There can also be Horizontal Resistance, e.g., partial resistance against all races of a pathogen, typically controlled by many genes and Vertical Resistance, e.g., complete resistance to some races of a pathogen but not to other races, typically controlled by a few genes. In a Gene-for-Gene level, plants and pathogens evolve together, and the genetic changes in one balance changes in other. Accordingly, using Natural Variability, breeders combine most useful genes for Yield, Quality, Uniformity, Hardiness, Resistance. The sources of resistance genes include native or foreign Varieties, Heirloom Varieties, Wild Plant Relatives, and Induced Mutations, e.g., treating plant material with mutagenic agents. Using the present invention, plant breeders are provided with a new tool to assay combinatorial mutations.

### Diseases

Epigenetic and chromatin regulation is important for the pathogenicity of various diseases, and may play a crucial role in disease prevention and treatment (e.g., hypertension, coronary heart disease, type II diabetes, osteoporosis, tumors, HIV infection, autoimmune disease, inflammatory diseases and metabolic diseases) (see, e.g., Esteller, Epigenetic drugs: More than meets the eye. Epigenetics. 2017; 12(5): 307; Banerjee, et al., (2012). “BET bromodomain inhibition as a novel strategy for reactivation of HIV-1”. Journal of Leukocyte Biology. 92 (6): 1147-1154; Anand, et al., (2013). “BET Bromodomains Mediate Transcriptional Pause Release in Heart Failure”. Cell. 154 (3): 569; and Mumby et al., Bromodomain and extra-terminal protein mimic JQ1 decreases inflammation in human vascular endothelial cells: Implications for pulmonary arterial hypertension. Respirology. 2017 January; 22(1): 157-164). In certain embodiments, agents targeting combinations of chromatin regulators are used to treat such diseases in a subject in need thereof (e.g., BRD4 and WDR77). The methods involve administering to a subject a combination of two or more inhibitors of epigenetic genes in an effective amount.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

In certain embodiments, the invention relates to methods and compositions for treating cancer in a subject. Cancer is a disease characterized by uncontrolled or aberrantly controlled cell proliferation and other malignant cellular properties. As used herein, the term “cancer” refers to any type of cancer known in the art, including without limitation, liquid tumors such as leukemia (e.g., acute myeloid leukemia (AML), acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases. In preferred embodiments, the cancer is AML. Most AMLs do not have rearrangements. Certain AMLs have rearrangements in the TEL gene. Certain AMLs have rearrangements in the MLL gene (see, e.g., Ayton and Cleary, Molecular mechanisms of leukemogenesis mediated by MLL fusion proteins, Oncogene. 2001 Sep. 10; 20(40):5695-707). The present invention can be used to target combinations of genes to treat these AMLs.

The cancer cell may be a cancer cell in vivo (i.e., in an organism), ex vivo (i.e., removed from an organism and maintained in vitro), or in vitro.

In certain embodiments, the subject is a subject having, suspected of having, or at risk of developing cancer. In certain embodiments, the subject is a mammalian subject, including but not limited to a dog, cat, horse, cow, pig, sheep, goat, chicken, rodent, or primate. In certain embodiments, the subject is a human subject, such as a patient. The human subject may be a pediatric or adult subject. Whether a subject is deemed “at risk” of having a cancer may be determined by a skilled practitioner.

In certain embodiments, the cancer treated has a mutation in the MAPK pathway. As used herein the “MAPK pathway” may be used interchangeably with “MAPK/ERK pathway” and “Ras-Raf-MEK-ERK pathway.” The MAPK/ERK pathway is a chain of proteins in the cell that communicates a signal from a receptor on the surface of the cell to the DNA in the nucleus of the cell (see, e.g., Orton R J, et al., (2005). “Computational modelling of the receptor-tyrosine-kinase-activated MAPK pathway” The Biochemical Journal. 392 (Pt 2): 249-61). The signal starts when a signaling molecule binds to the receptor on the cell surface and ends when the DNA in the nucleus expresses a protein and produces some change in the cell, such as cell division. The pathway includes many proteins, including MAPK (mitogen-activated protein kinases, originally called ERK, extracellular signal-regulated kinases), which communicate by adding phosphate groups to a neighboring protein, which acts as an “on” or “off” switch. When one of the proteins in the pathway is mutated, it can become stuck in the “on” or “off” position, which is a necessary step in the development of many cancers. In preferred embodiments, the cancer has a mutation in BRAF, KRAS or NRAS. In specific embodiments, the mutations are BRAF V600E, KRAS G12S or NRAS Q61L. BRAF mutations are most common in melanoma. Currently, it is estimated that eight percent of all cancers have mutations in the BRAF gene, and they are present in a wide range of malignant tumors including ˜50% of melanomas, ˜40% of papillary thyroid cancer (PTC), ˜30% of serous ovarian cancer, ˜10% of colorectal cancers (CRC), and ˜2%-3% of lung cancers (Obaid et al., Strategies for Overcoming Resistance in Tumours Harboring BRAF Mutations. Int J Mol Sci. 2017 Mar. 8; 18(3)). Somatic KRAS mutations are found at high rates in leukemias, colorectal cancer, pancreatic cancer and lung cancer (Chiosea S I, et al., (2011) Modern Pathology. 24 (12): 1571-7; Hartman D J, et al., (2012) International Journal of Cancer. 131 (8): 1810-7; and Krasinskas A M, et al., (2013) Modern Pathology. 26 (10): 1346-54). NRAS mutations arise in 15-20% of all melanomas (Johnson and Puzanov, (2015) Curr Treat Options Oncol. 16(4):15) and also occur in colorectal cancer (De Roock W, et al. Lancet Oncol 2010; 11: 753-762).

In certain embodiments, the cancer has a mutation in PIK3CA. As used herein PIK3CA may refer to the gene or protein according accession number NM_006218.3 and may also include associated fragments and splicing variants, proteins with conservative substitutions and proteins having at least 90% sequence identity. Mutations in PIK3CA occur in colorectal cancer, cervical cancers and breast cancers (De Roock W, et al. Lancet Oncol 2010; 11: 753-762; Samuels, et al., (2010) in Human Cancers. Current Topics in Microbiology and Immunology. Springer Berlin Heidelberg. pp. 21-41; Ma Y Y, et al., (2000) Oncogene. 19 (23): 2739-44; and Zardavas, et al., (2014) Breast Cancer Research. 16 (1)).

Diseases that may be treated by the foregoing include, without limitation, infection, inflammation, immune-related disorders or aberrant immune responses.

Diseases with an abberant or pathologic immune response include, for example, Acquired Immunodeficiency Syndrome (AIDS, which is a viral disease with an autoimmune component), Crohn's disease, systemic lupus erythematosus, ulcerative colitis, multiple sclerosis (MS), inflammatory bowel disease and chronic and acute inflammatory disorders. Examples of inflammatory disorders include asthma, atopic allergy, allergy, eczema, glomerulonephritis, graft vs. host disease. In certain embodiments, latent HIV is reactivated by a combination therapy. Reactivation of latent HIV can also be screened to identify additional combination of targets using the screening platform.

In certain embodiments, the pathological condition may be an infection, inflammation, proliferative disease, autoimmune disease, or allergy.

The term “infection” as used herein refers to presence of an infective agent, such as a pathogen, e.g., a microorganism, in or on a subject, which, if its presence or growth were inhibited, would result in a benefit to the subject. Hence, the term refers to the state produced by the establishment, more particularly invasion and multiplication, of an infective agent, such as a pathogen, e.g., a microorganism, in or on a suitable host. An infection may produce tissue injury and progress to overt disease through a variety of cellular and toxic mechanisms.

The term “inflammation” generally refers to a response in vasculated tissues to cellular or tissue injury usually caused by physical, chemical and/or biological agents, that is marked in the acute form by the classical sequences of pain, heat, redness, swelling, and loss of function, and serves as a mechanism initiating the elimination, dilution or walling-off of noxious agents and/or of damaged tissue. Inflammation histologically involves a complex series of events, including dilation of the arterioles, capillaries, and venules with increased permeability and blood flow, exudation of fluids including plasma proteins, and leukocyte migration into the inflammatory focus.

Further, the term encompasses inflammation caused by extraneous physical or chemical injury or by biological agents, e.g., viruses, bacteria, fungi, protozoan or metazoan parasite infections, as well as inflammation which is seemingly unprovoked, e.g., which occurs in the absence of demonstrable injury or infection, inflammation responses to self-antigens (auto-immune inflammation), inflammation responses to engrafted xenogeneic or allogeneic cells, tissues or organs, inflammation responses to allergens, etc. The term covers both acute inflammation and chronic inflammation. Also, the term includes both local or localised inflammation, as well as systemic inflammation, i.e., where one or more inflammatory processes are not confined to a particular tissue but occur generally in the endothelium and/or other organ systems.

Systemic inflammatory conditions may particularly encompass systemic inflammatory response syndrome (SIRS) or sepsis. “SIRS” is a systemic inflammatory response syndrome with no signs of infection. It can be characterised by the presence of at least two of the four following clinical criteria: fever or hypothermia (temperature of 38.0° C.) or more, or temperature of 36.0° C. or less); tachycardia (at least 90 beats per minute); tachypnea (at least 20 breaths per minute or PaCO₂ less than 4.3 kPa (32.0 mm Hg) or the need for mechanical ventilation); and an altered white blood cell (WBC) count of 12×10⁶ cells/mL or more, or an altered WBC count of 4×10⁶ cells/mL or less, or the presence of more than 10% band forms. “Sepsis” can generally be defined as SIRS with a documented infection, such as for example a bacterial infection. Infection can be diagnosed by standard textbook criteria or, in case of uncertainty, by an infectious disease specialist. Bacteraemia is defined as sepsis where bacteria can be cultured from blood. Sepsis may be characterised or staged as mild sepsis, severe sepsis (sepsis with acute organ dysfunction), septic shock (sepsis with refractory arterial hypotension), organ failure, multiple organ dysfunction syndrome and death.

As used throughout the present specification, the terms “autoimmune disease” or “autoimmune disorder” used interchangeably refer to a diseases or disorders caused by an immune response against a self-tissue or tissue component (self-antigen) and include a self-antibody response and/or cell-mediated response. The terms encompass organ-specific autoimmune diseases, in which an autoimmune response is directed against a single tissue, as well as non-organ specific autoimmune diseases, in which an autoimmune response is directed against a component present in two or more, several or many organs throughout the body.

Non-limiting examples of autoimmune diseases include but are not limited to acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behçet's disease; coeliac disease; dermatomyositis; diabetes mellitus type I; Goodpasture's syndrome; Graves' disease; Guillain-Barré syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjögren's syndrome; systemic lupus erythematosus; Takayasu's arteritis; temporal arteritis; vitiligo; warm autoimmune hemolytic anemia; or Wegener's granulomatosis.

### Therapeutic Agents

In certain embodiments, the present invention provides for one or more therapeutic agents against combinations of targets identified. Targeting the identified combinations may provide for enhanced or otherwise previously unknown activity in the treatment of disease. In certain embodiments, an agent against one of the targets in a combination may already be known or used clinically. In certain embodiments, targeting the combination may require less of the agent as compared to the current standard of care and provide for less toxicity and improved treatment. In certain embodiments, the agents are used to modulate cell types. For example, the agents may be used to modulate cells for adoptive cell transfer (e.g., BRD4 inhibitors in combination with another agent, such as WDR77). In certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

For example, in methods for treating cancer in a subject, an effective amount of a combination of inhibitors targeting epigenetic genes is any amount that provides an anticancer effect, such as reduces or prevents proliferation of a cancer cell or is cytotoxic towards a cancer cell. In certain embodiments, the effective amount of an inhibitor targeting an epigenetic gene is reduced when an inhibitor is administered concomitantly or in combination with one or more additional inhibitors targeting epigenetic genes as compared to the effective amount of the inhibitor when administered in the absence of one or more additional inhibitors targeting epigenetic genes. In certain embodiments, the inhibitor targeting an epigenetic gene does not reduce or prevent proliferation of a cancer cell when administered in the absence of one or more additional inhibitors targeting epigenetic genes.

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). Specific small molecule degraders targeting bromodomain and extra-terminal (BET) family proteins, consisting of BRD2, BRD3, BRD4, and testis-specific BRDT members (e.g., BETd-260/ZBC260) are specifically applicable for targeting the identified synthetic lethal combinations comprising BRD4 (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481).

As described herein, small molecules targeting epigenetic proteins are currently being developed and/or used in the clinic to treat disease (see, e.g., Qi et al., HEDD: the human epigenetic drug database. Database, 2016, 1-10; and Ackloo et al., Chemical probes targeting epigenetic proteins: Applications beyond oncology. Epigenetics 2017, VOL. 12, NO. 5, 378-400). In certain embodiments, the one or more agents comprise a histone acetylation inhibitor, histone deacetylase (HDAC) inhibitor, histone lysine methylation inhibitor, histone lysine demethylation inhibitor, DNA methyltransferase (DNMT) inhibitor, inhibitor of acetylated histone binding proteins, inhibitor of methylated histone binding proteins, sirtuin inhibitor, protein arginine methyltransferase inhibitor or kinase inhibitor. In certain embodiments, any small molecule exhibiting the functional activity described above may be used in the present invention. In certain embodiments, the DNA methyltransferase (DNMT) inhibitor is selected from the group consisting of azacitidine (5-azacytidine), decitabine (5-aza-2′-deoxycytidine), EGCG (epigallocatechin-3-gallate), zebularine, hydralazine, and procainamide. In certain embodiments, the histone acetylation inhibitor is C646. In certain embodiments, the histone deacetylase (HDAC) inhibitor is selected from the group consisting of vorinostat, givinostat, panobinostat, belinostat, entinostat, CG-1521, romidepsin, ITF-A, ITF-B, valproic acid, OSU-HDAC-44, HC-toxin, magnesium valproate, plitidepsin, tasquinimod, sodium butyrate, mocetinostat, carbamazepine, SB939, CHR-2845, CHR-3996, JNJ-26481585, sodium phenylbutyrate, pivanex, abexinostat, resminostat, dacinostat, droxinostat, and trichostatin A (TSA). In certain embodiments, the histone lysine demethylation inhibitor is selected from the group consisting of pargyline, clorgyline, bizine, GSK2879552, GSK-J4, KDM5-C70, JIB-04, and tranylcypromine. In certain embodiments, the histone lysine methylation inhibitor is selected from the group consisting of EPZ-6438, GSK126, CPI-360, CPI-1205, CPI-0209, DZNep, GSK343, EI1, BIX-01294, UNC0638, EPZ004777, GSK343, UNC1999 and UNC0224. In certain embodiments, the inhibitor of acetylated histone binding proteins is selected from the group consisting of AZD5153 (see e.g., Rhyasen et al., AZD5153: A Novel Bivalent BET Bromodomain Inhibitor Highly Active against Hematologic Malignancies, Mol Cancer Ther. 2016 November; 15(11):2563-2574. Epub 2016 Aug. 29), PFI-1, CPI-203, CPI-0610, RVX-208, OTX015, I-BET151, I-BET762, I-BET-726, dBET1, ARV-771, ARV-825, BETd-260/ZBC260 and MZ1. In certain embodiments, the inhibitor of methylated histone binding proteins is selected from the group consisting of UNC669 and UNC1215. In certain embodiments, the sirtuin inhibitor comprises nicotinamide.

### Genetic Modifying Agents

In certain embodiments, the one or more modulating agents may be a genetic modifying agent. The genetic modifying agent may comprise a CRISPR system, a zinc finger nuclease system, a TALEN, a meganuclease or RNAi system. In certain embodiments, the orthogonal CRISPR enzymes may be any CRISPR enzyme described herein. The following description of CRISPR can be applied for therapeutic purposes as well as in the screening methods described herein.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx. doi. org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

### Guide Molecules

The methods described herein may be used to screen inhibition of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoide cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′ phosphorothioate (MS), S-constrained ethyl(cEt), or 2′-O-methyl 3′ thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (melΨ), 5-methoxyuridine(5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of th guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas proten (Chen et al. Cell. (2013); 155(7): 1479-1491). In particular embodiments the stem of the stemloop is extended by at least 1, 2, 3, 4, 5 or more complementary basepairs (i.e. corresponding to the addition of 2,4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the compelementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928). Aptamers may also be constructed that function as molecular switches, responding to a que by changing properties, such as RNA aptamers that bind fluorophores to mimic the activity of green flourescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans;4/164/rs2), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (4OHT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100.mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

### CRiSPR RNA-Targeting Effector Proteins

In one example embodiment, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a). It will be understood that the term “C2c2” herein is used interchangeably with “Cas13a”. “C2c2” is now referred to as “Cas13a”, and the terms are used interchangeably herein unless indicated otherwise. As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023., which is incorporated herein in its entirety by reference.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprise one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In certain other example embodiments, the CRISPR system effector protein is a C2c2 nuclease (also referred to as Cas13a). The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

In certain embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, or the C2c2 effector protein is an organism selected from the group consisting of: Leptotrichia shahii, Leptotrichia. wadei, Listeria seeligeri, Clostridium aminophilum, Carnobacterium gallinarum, Paludibacter propionicigenes, Listeria weihenstephanensis, or the C2c2 effector protein is a L. wadei F0279 or L. wadei F0279 (Lw2) C2C2 effector protein. In another embodiment, the one or more guide RNAs are designed to detect a single nucleotide polymorphism, splice variant of a transcript, or a frameshift mutation in a target RNA or DNA.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum.

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

In other example embodiments, the Type VI RNA-targeting Cas enzyme is Cas13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

### Cas13 RNA Editing

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytindine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g, Cox et al., Science. 2017 Nov. 24; 358(6366):1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the invention relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present invention may also use a Cas12 CRISPR enzyme. Cas12 enzymes include Cas12a (Cpf1), Cas12b (C2c1), and Cas12c (C2c3), described further herein. The Cas12 may be an ultraCas12. IDT developed a “Alt-R Cas12a” reagent that has 3 main components: a) optimized crRNA; b) A.s. Cas12a; and (c) an electroporation enhancer (for better transfection). The variant is an improved version of IDT's Alt-R Cas12a and is named “Alt-R Cas12a Ultra.”

A further aspect relates to an isolated cell obtained or obtainable from the methods described herein comprising the composition described herein or progeny of said modified cell, preferably wherein said cell comprises a hypoxanthine or a guanine in replace of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp PA. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et al, “Engineered Cpf1 Enzymes with Altered PAM Specificities,” bioRxiv 091611; doi: http://dx.doi.org/10.1101/091611 (Dec. 4, 2016).
Cox et al., “RNA editing with CRISPR-Cas13,” Science. 2017 Nov. 24; 358(6366):1019-1027. doi: 10.1126/science.aaq0180. Epub 2017 Oct. 25.
Gaudelli et al. “Programmable base editing of A-T to G-C in genomic DNA without DNA cleavage” Nature 464(551); 464-471 (2017).
Strecker et al., “Engineering of CRISPR-Cas12b for human genome editing,” Nature Communications volume 10, Article number: 212 (2019).

each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors we developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.
Cox et al., (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cast. In further embodiments, the CRISPR array is used as a seed to identify new effector proteins.

Also, “Dimeric CRISPR RNA-guided Fokl nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

Also, Harrington et al. “Programmed DNA destruction by miniature CRISPR-Cas14 enzymes” Science 2018 doi:10/1126/science.aav4293, relates to Cas14.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 15, PROTECTED GUIDE RNAS (PGRNAS); U.S. application 62/091,455, filed, 12 Dec. 14, PROTECTED GUIDE RNAS (PGRNAS); U.S. application 62/096,708, 24 Dec. 14, PROTECTED GUIDE RNAS (PGRNAS); U.S. applications 62/091,462, 12 Dec. 14, 62/096,324, 23 Dec. 14, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 14 and 62/180,692, 17 Jun. 2015, ESCORTED AND FUNCTIONALIZED GUIDES FOR CRISPR-CAS SYSTEMS; U.S. application 62/091,461, 12 Dec. 14, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 14, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 14, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 14, 62/181,641, 18 Jun. 2015, and 62/181,667, 18 Jun. 2015, RNA-TARGETING SYSTEM; U.S. application 62/096,656, 24 Dec. 14 and 62/181,151, 17 Jun. 2015, CRISPR HAVING OR ASSOCIATED WITH DESTABILIZATION DOMAINS; U.S. application 62/096,697, 24 Dec. 14, CRISPR HAVING OR ASSOCIATED WITH AAV; U.S. application 62/098,158, 30 Dec. 14, ENGINEERED CRISPR COMPLEX INSERTIONAL TARGETING SYSTEMS; U.S. application 62/151,052, 22 Apr. 15, CELLULAR TARGETING FOR EXTRACELLULAR EXOSOMAL REPORTING; U.S. application 62/054,490, 24 Sep. 14, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12-F

EB-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 14, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 14, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 14 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 14, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 14, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 14, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 14 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 14, FUNCTIONAL SCREENING WITH OPTIMIZED FUNCTIONAL CRISPR-CAS SYSTEMS; U.S. application 62/087,546, 4 Dec. 14 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 14, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153(4):910-8).

In particular embodiments, the ribonucleoprotein is delivered by way of a polypeptide-based shuttle agent as described in WO2016161516. WO2016161516 describes efficient transduction of polypeptide cargos using synthetic peptides comprising an endosome leakage domain (ELD) operably linked to a cell penetrating domain (CPD), to a histidine-rich domain and a CPD. Similarly these polypeptides can be used for the delivery of CRISPR-effector based RNPs in eukaryotic cells.

### Tale Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

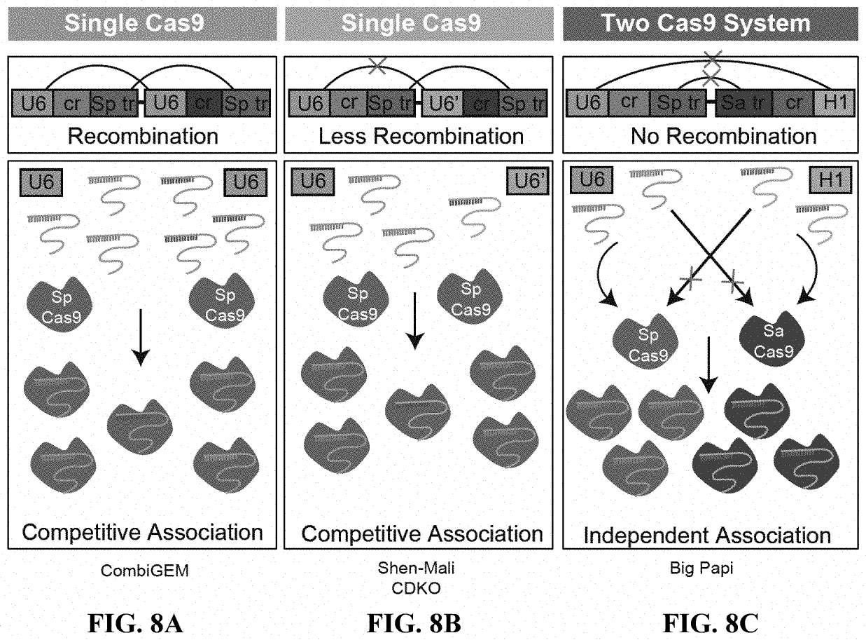

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 45,533)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ ID NO: 45,534)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### ZN-Finger Nucleases

Other preferred tools for genome editing for use in the context of this invention include zinc finger systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme Fokl. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Antibodies

In certain embodiments, the one or more agents is an antibody. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by (3 pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}—Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Aptamers

In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colo.). In certain embodiments, the present invention may utilize any aptamer containing any modification as described herein.

### Adoptive Cell Transfer

In certain embodiments, one or more agents targeting one or more combinations of targets identified by the screening platform described herein are used to modulate cells used for adoptive cell transfer. In certain embodiments, the one or more agents comprises BRD4. In certain embodiments, the one or more agents target the expression, activity, substrate or products of WDR77 and BRD4. As described herein, inhibitors of BRD4 are useful in enhancing T cell persistence and function in immunotherapy models (Kagoya et al., BET bromodomain inhibition enhances T cell persistence and function in adoptive immunotherapy models. J Clin Invest. 2016; 126(9):3479-3494). Applicants have identified for the first time that WDR77 and BRD4 interact genetically and thus the targeting of the combination may provide enhanced T cell persistence and function in adoptive cell transfer.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostate; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGSS; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDCl27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190 KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR α and 13 chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3 or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/0X40/4-1BB-CD3; see U.S. Pat. Nos. 8,911,993; 8,916,381; 8,975,071; 9,101,584; 9,102,760; 9,102,761). Third-generation CARs include a combination of costimulatory endodomains, such a CD3-chain, CD97, GDI 1a-CD18, CD2, ICOS, CD27, CD154, CDS, 0X40, 4-1BB, CD2, CD7, LIGHT, LFA-1, NKG2C, B7-H3, CD30, CD40, PD-1, or CD28 signaling domains (for example scFv-CD28-4-1BB-CD3 or scFv-CD28-0X40-CD3; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3 or FcRγ. In certain embodiments, the one or more costimulatory signaling domains comprise a functional signaling domain of a protein selected, each independently, from the group consisting of: CD27, CD28, 4-1BB (CD137), 0X40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3 chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS) (SEQ ID NO:45,535)). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3 chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO:45,536) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor a-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3 chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY and continuing all the way to the carboxy-terminus of the protein. The sequence is reproduced herein: IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS (SEQ ID NO:45,537). Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MI-IC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3 and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with y-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+Th1 cells and CD8+CTLs to induce a synergistic antitumour response (see, e.g., Li et al., Adoptive cell therapy with CD4+T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and 13, which assemble to form a heterodimer and associates with the CD3-transducing subunits to form the T cell receptor complex present on the cell surface. Each a and 13 chain of the TCR consists of an immunoglobulin-like N-terminal variable (V) and constant (C) region, a hydrophobic transmembrane domain, and a short cytoplasmic region. As for immunoglobulin molecules, the variable region of the a and 13 chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor a-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or MR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGITNstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells, bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3X28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of ¹²⁵I labeled β2-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™ FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

In certain embodiments, the combination therapies described herein are used in further combination with cancer therapies according to the standard of care for the particular cancer.

### Perturb-Seq

In certain embodiments, the combination screens described herein are compatible within single cell transcriptome studies. In certain embodiments, barcodes associated with combinations of guide sequences as described herein are transcribed into poly A tailed transcripts. In certain embodiments, single cell transcriptomes and transcripts comprising the guide sequence barcodes are labeled with cell of origin barcodes, thus allowing the combination perturbations to be associated with single cell gene expression. Methods and tools for genome-scale screening of perturbations in single cells using CRISPR-Cas9 have been described, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; Feldman et al., Lentiviral co-packaging mitigates the effects of intermolecular recombination and multiple integrations in pooled genetic screens, bioRxiv 262121, doi: doi.org/10.1101/262121; Datlinger, et al., 2017, Pooled CRISPR screening with single-cell transcriptome readout. Nature Methods. Vol. 14 No. 3 DOI: 10.1038/nmeth.4177; Hill et al., On the design of CRISPR-based single cell molecular screens, Nat Methods. 2018 April; 15(4): 271-274; and International publication serial number WO/2017/075294). The present invention is compatible with perturb-seq, such that combinations of genes may be perturbed and the perturbation may be identified and assigned to the proteomic and gene expression readouts of single cells. In certain embodiments, signature genes may be perturbed in single cells and gene expression analyzed. Not being bound by a theory, networks of genes that are disrupted due to perturbation of a signature gene may be determined. Understanding the network of genes effected by a perturbation may allow for a gene to be linked to a specific pathway that may be targeted to modulate the signature and treat a cancer. Thus, in certain embodiments, perturb-seq is used to discover novel drug targets to allow treatment of specific cancer patients having the gene signature of the present invention. In certain embodiments, perturbation barcodes are transcribed from a RNA polymerase II promoter to produce a transcript that can be captured using single cell RNA-seq techniques, such as in CROP-seq (see, e.g., Datlinger, et al., 2017).

The perturbation methods and tools allow reconstructing of a cellular network or circuit. In one embodiment, the method comprises (1) introducing single-order or combinatorial perturbations to a population of cells, (2) measuring genomic, genetic, proteomic, epigenetic and/or phenotypic differences in single cells and (3) assigning a perturbation(s) to the single cells. Not being bound by a theory, a perturbation may be linked to a phenotypic change, preferably changes in gene or protein expression. In preferred embodiments, measured differences that are relevant to the perturbations are determined by applying a model accounting for co-variates to the measured differences. The model may include the capture rate of measured signals, whether the perturbation actually perturbed the cell (phenotypic impact), the presence of subpopulations of either different cells or cell states, and/or analysis of matched cells without any perturbation. In certain embodiments, the measuring of phenotypic differences and assigning a perturbation to a single cell is determined by performing single cell RNA sequencing (RNA-seq). In preferred embodiments, the single cell RNA-seq is performed by any method as described herein (e.g., Drop-seq, InDrop, 10× genomics). In certain embodiments, unique barcodes are used to perform Perturb-seq. In certain embodiments, a guide RNA is detected by RNA-seq using a transcript expressed from a vector encoding the guide RNA. The transcript may include a unique barcode specific to the guide RNA. Not being bound by a theory, a guide RNA and guide RNA barcode is expressed from the same vector and the barcode may be detected by RNA-seq. Not being bound by a theory, detection of a guide RNA barcode is more reliable than detecting a guide RNA sequence, reduces the chance of false guide RNA assignment and reduces the sequencing cost associated with executing these screens. Thus, a perturbation may be assigned to a single cell by detection of a guide RNA barcode in the cell. In certain embodiments, a cell barcode is added to the RNA in single cells, such that the RNA may be assigned to a single cell. Generating cell barcodes is described herein for single cell sequencing methods. In certain embodiments, a Unique Molecular Identifier (UMI) is added to each individual transcript and protein capture oligonucleotide. Not being bound by a theory, the UMI allows for determining the capture rate of measured signals, or preferably the binding events or the number of transcripts captured. Not being bound by a theory, the data is more significant if the signal observed is derived from more than one protein binding event or transcript. In preferred embodiments, Perturb-seq is performed using a guide RNA barcode expressed as a polyadenylated transcript, a cell barcode, and a UMI.

A CRISPR system may be delivered to primary mouse T-cells. Over 80% transduction efficiency may be achieved with Lenti-CRISPR constructs in CD4 and CD8 T-cells. Despite success with lentiviral delivery, recent work by Hendel et al, (Nature Biotechnology 33, 985-989 (2015) doi:10.1038/nbt.3290) showed the efficiency of editing human T-cells with chemically modified RNA, and direct RNA delivery to T-cells via electroporation. In certain embodiments, perturbation in mouse primary T-cells may use these methods.

In certain embodiments, whole genome screens can be used for understanding the phenotypic readout of perturbing potential target genes. In preferred embodiments, perturbations target expressed genes as defined by a gene signature using a focused sgRNA library. Libraries may be focused on expressed genes in specific networks or pathways. In other preferred embodiments, regulatory drivers are perturbed. In certain embodiments, Applicants perform systematic perturbation of key genes that regulate T-cell function in a high-throughput fashion. In certain embodiments, Applicants perform systematic perturbation of key genes that regulate cancer cell function in a high-throughput fashion (e.g., immune resistance or immunotherapy resistance). Applicants can use gene expression profiling data to define the target of interest and perform follow-up single-cell and population RNA-seq analysis. Not being bound by a theory, this approach will accelerate the development of therapeutics for human disorders, in particular cancer. Not being bound by a theory, this approach will enhance the understanding of the biology of T-cells and tumor immunity, and accelerate the development of therapeutics for human disorders, in particular cancer, as described herein.

Not being bound by a theory, perturbation studies targeting the genes and gene signatures described herein could (1) generate new insights regarding regulation and interaction of molecules within the system that contribute to suppression of an immune response, such as in the case within the tumor microenvironment, and (2) establish potential therapeutic targets or pathways that could be translated into clinical application.

In certain embodiments, after determining Perturb-seq effects in cancer cells and/or primary T-cells, the cells are infused back to the tumor xenograft models (melanoma, such as B16F10 and colon cancer, such as CT26) to observe the phenotypic effects of genome editing. Not being bound by a theory, detailed characterization can be performed based on (1) the phenotypes related to tumor progression, tumor growth, immune response, etc. (2) the TILs that have been genetically perturbed by CRISPR-Cas9 can be isolated from tumor samples, subject to cytokine profiling, qPCR/RNA-seq, and single-cell analysis to understand the biological effects of perturbing the key driver genes within the tumor-immune cell contexts. Not being bound by a theory, this will lead to validation of TILs biology as well as lead to therapeutic targets.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

The present invention advantageously provides for screening platforms that can provide for diagnostic tools. The screening platform can be scaled up to be genome wide. The present invention can be used for chemical genomics by pairing the knockout with drug treatment dose dependence for combinations identified. The screening method can be used to knockout oncogenes and activate tumor suppressors in the same cell. The methods of the present invention can be used to identify drug resistance routes. For example, drug resistant clones can be screened for second mutations that can be used to treat the clones. Although the present invention and its advantages have been described in detail, it should be understood that various changes, substitutions and alterations can be made herein without departing from the spirit and scope of the invention as defined in the appended claims.

The invention is further described in the following examples, which do not limit the scope of the invention described in the claims.

### EXAMPLES

### Example 1—Generating Double Knockouts Using a Dual Cas9 System

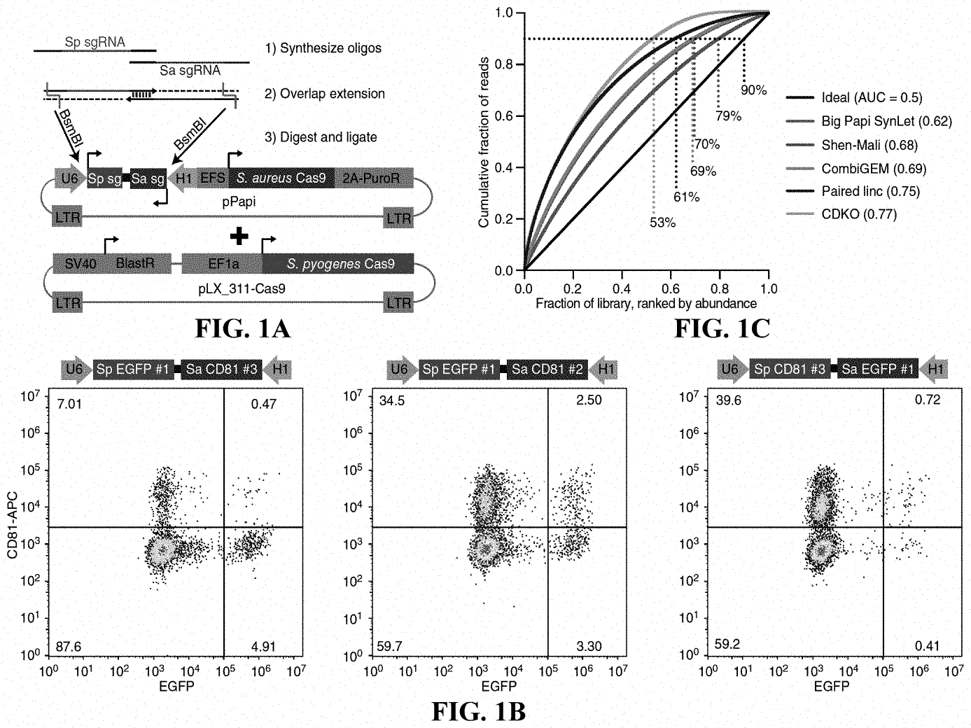

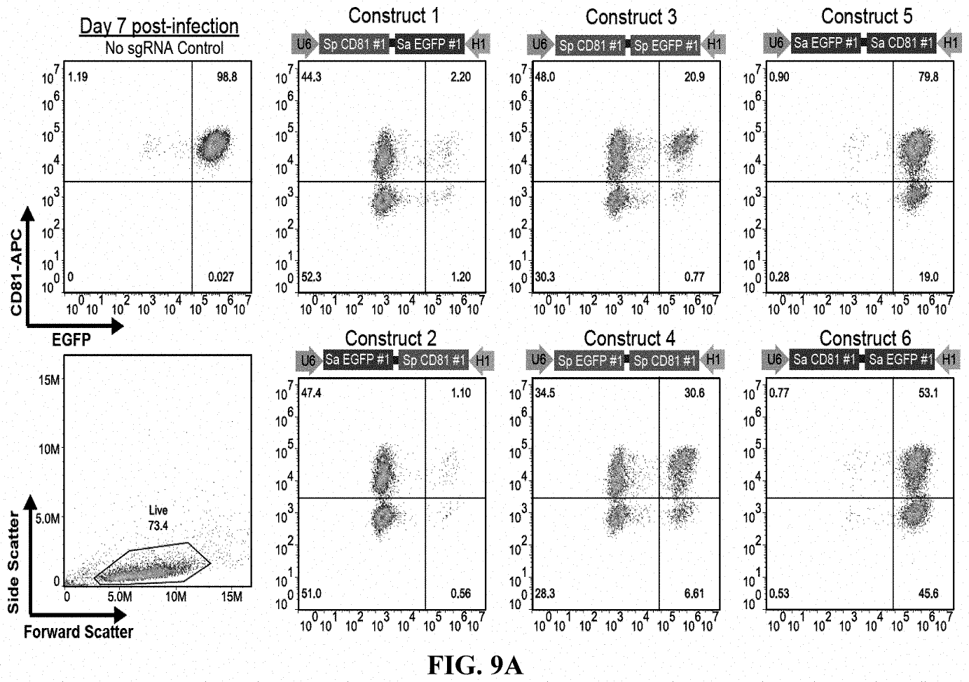

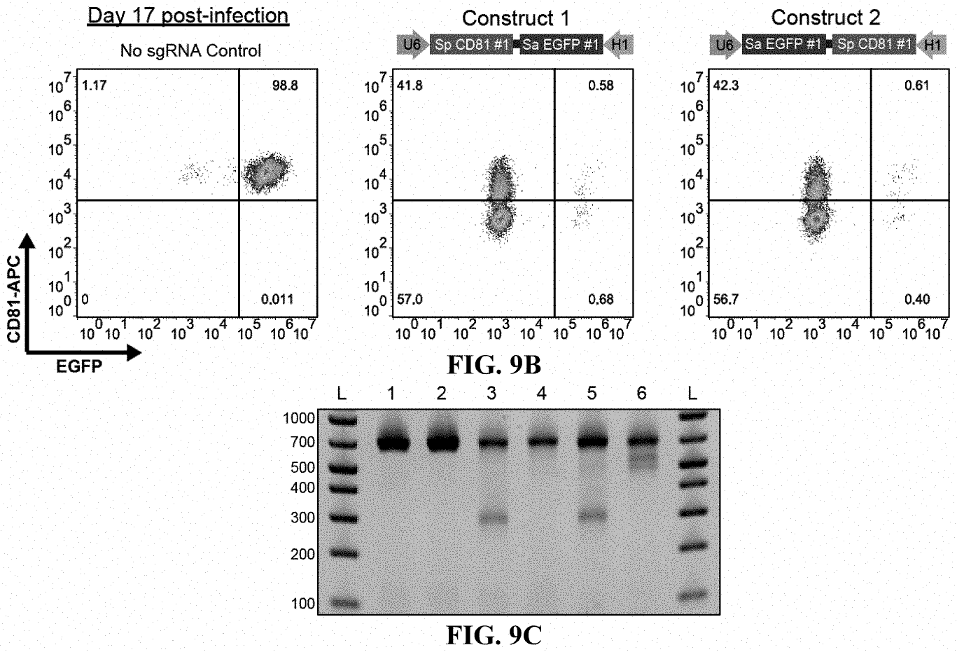

Applicants aimed to develop a system with maximal on-target efficiency at two independent genomic sites, postulating that using two independent Cas9 enzymes would mitigate several sources of inefficiency (FIG. 8). Applicants designed a lentiviral construct, pPapi, to express SaCas9 and two sgRNAs from the U6 and H1 promoters (FIG. 1a). A flow cytometry assay assessed dual targeting of EGFP and endogenous CD81 in A375 cells engineered to stably express SpCas9 and EGFP, and Applicants measured the effect of varying the promoter and Cas9 ortholog employed by each sgRNA. Partnering SaCas9 and SpCas9 sgRNAs achieved dual knockout in 50-87% of cells with 4 different combinations of sgRNAs (FIG. 1b, FIG. 9), indicating a potential for high efficiency.

To enable efficient construction of pooled, multiplex libraries, Applicants developed a cloning scheme with synthesized oligonucleotides (˜140 nts), overlap extension, and a single transformation step into E. coli (FIG. 1a). A pool consisting of M SpCas9 sgRNAs and N SaCas9 sgRNAs, a total of M+N oligos, generates a pool comprising M×N pairwise combinations. Applicants generated a Synthetic Lethal (SynLet) library, described below, with 96 unique sgRNAs cloned into each position, totaling 962=9,216 dual-sgRNA elements. The proximity of the sgRNAs permits them to be amplified and sequenced together in a single NextGen sequencing read. The cumulative distribution function for the pool had an area-under-the-curve (AUC) of 0.62 (FIG. 1c), comparable to the previously-described Brunello genome-wide library (AUC of 0.64; AUC=0.5 for a perfectly uniform distribution)¹⁶. Applicants compared this SynLet library to four other published libraries^{6-8, 17}, which all rely on two transformation steps into E. coli; AUCs for these libraries ranged from 0.68-0.77. Likewise, for the SynLet library, 79% of sgRNA pairs were found in the top 90% of reads, whereas the other four libraries showed more attrition, capturing 53%-70% of elements at this threshold (FIG. 1c).

### Example 2—Optimizing sgRNA Design for SaCas9

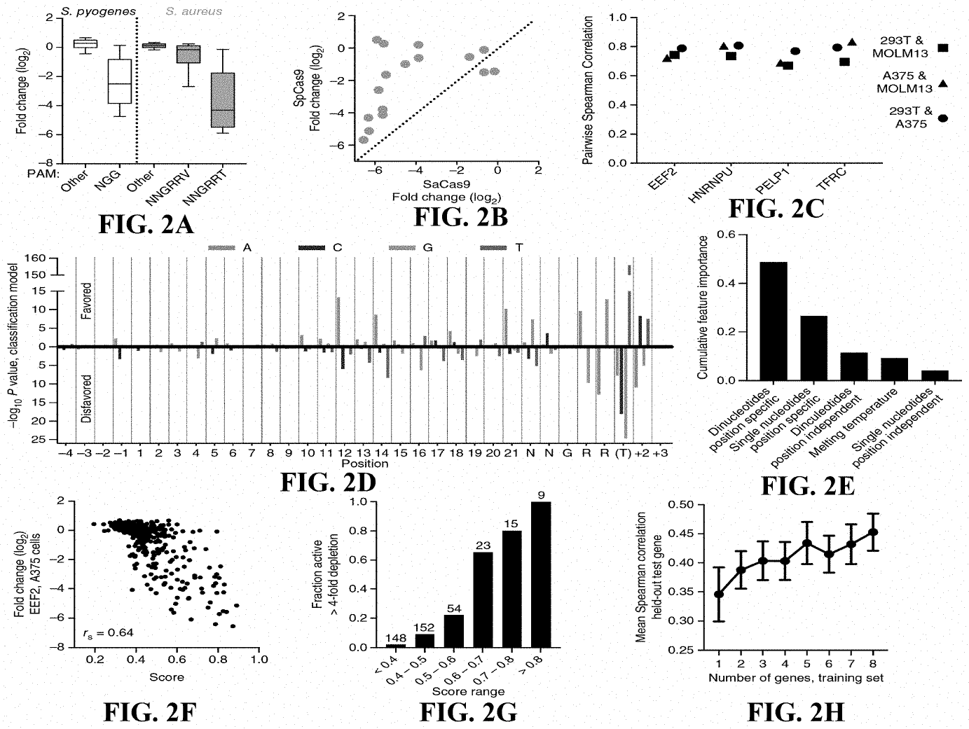

Previously, Applicants determined rules to predict high-performing SpCas9 sgRNAs by coupling experimentation with machine learning^{16,18.} Applicants took a similar approach to optimize the design of SaCas9 sgRNAs. Applicants developed a SaCas9-version of lentiCRISPR-v2, replacing the SpCas9 and tracrRNA scaffold with their S. aureus counterparts. Applicants designed a pooled tiling library to compare SpCas9 and SaCas9 by targeting EEF2, a common essential gene, with all possible sgRNA sequences regardless of protospacer adjacent motif (PAM), and assayed activity by a viability screen in A375 cells. As expected for SpCas9, the set of sgRNAs utilizing an NGG PAM (n=449) were depleted compared to those using all other PAMs (n=4,087), with a median log₂-fold-change of ˜2.5 relative to the plasmid DNA (FIG. 2a). For SaCas9, some sgRNAs with an NNGRRV PAM (n=349; R=A or G; V=A, C, or G) were active, but as expected^{19, 20}, NNGRRT (n=47) was most active, with a median log₂-fold-change of ˜4.3. Applicants compared sgRNAs sharing a common cut site between SaCas9 and SpCas9, and observed that SaCas9 typically had higher activity (FIG. 2b).

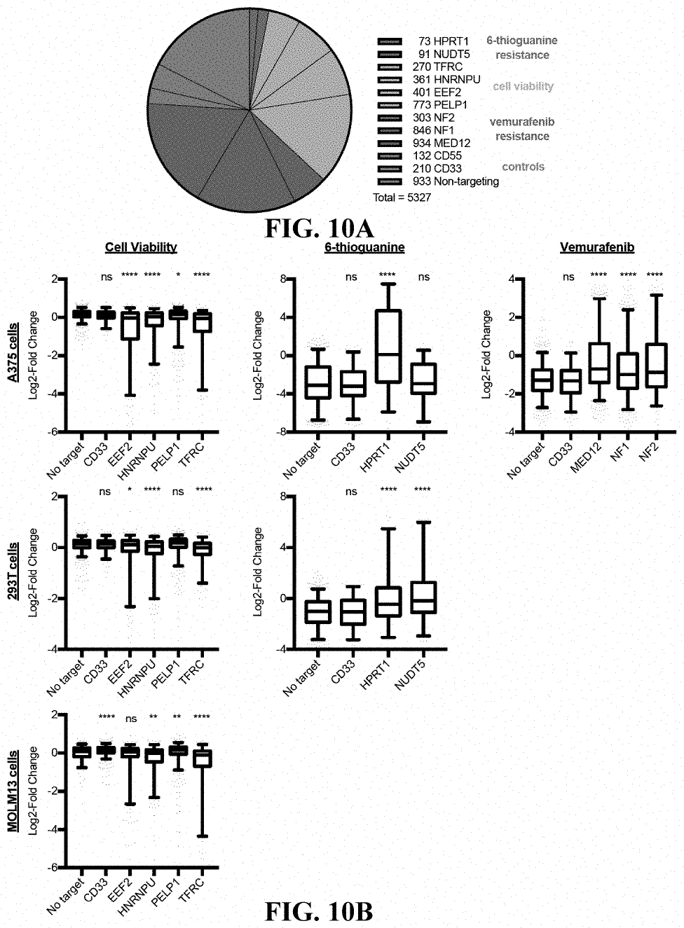

Applicants designed a second tiling library with all sgRNAs with an NNGRR PAM that targeted 9 genes with known phenotypes in viability and drug resistance assays, a total of 5,327 sgRNAs, including controls (FIG. 10a). Applicants performed viability screens in three cell lines (A375, 293T, MOLM13), and screened relevant cell lines for 6-thioguanine (A375, 293T) and vemurafenib resistance (A375). Applicants observed the expected activities for sgRNAs targeting these nine genes (FIG. 10b, Najm et al., 2017 Supplementary Table 1), with consistent performance across the 3 cell lines (FIG. 2c), suggesting that predictive sequence features are likely to generalize across cell types.

Applicants first used a classification model to determine sequence features correlated with high activity, examining all single and dinucleotides¹⁶. The feature most predictive of high activity was thymine immediately 3′ of the core PAM sequence, NNGRR (FIG. 2d). However, thymine is neither necessary nor sufficient for high activity: of 1,805 sgRNAs targeting viability genes in A375 cells, a non-thymine nucleotide was present in 58% of the top quintile of most-active sgRNAs, whereas 14% of sgRNAs with thymine scored in the bottom half of activity. In the RR portion of the PAM, Applicants observed that AG is favored over other combinations of purines.

To improve predictions, Applicants used gradient boosted regression trees on the rank-transformed activity values¹⁶. Features included position-specific and position-independent single and dinucleotides, and thermodynamic properties; position-specific dinucleotides proved the most important for predicting activity (FIG. 2e). To illustrate performance, Applicants used a version of the model in which EEF2 sgRNAs were held out of the training set, and compared the predicted scores to the measured activities in A375 cells, observing a Spearman correlation of 0.64 (FIG. 2f). Whereas high-scoring sgRNAs (score >0.6) represent only 12% of all EEF2 sgRNAs, they are quite likely to be active, with 77% resulting in >4-fold decrease in viability (FIG. 2g). Downsampling the number of genes used for training indicated diminishing returns for model parameter estimation with 9 genes (FIG. 2h). The model developed here for SaCas9 sgRNA design, available online (portals.broadinstitute.org/gpp/publicianalysis-tools/sgrna-design) will enable more effective application of CRISPR technology.

### Example 3—Combinatorial Gene Targeting Using a Dual Cas9 System

Applicants first tested the Big Papi approach by screening for synthetic lethal gene combinations. As few such relationships have been validated across many cell lines, Applicants assembled an ad hoc list of target genes (Najm et al., 2017 Supplementary Table 2). BRCA and PARP genes have a clinically-appreciated synthetic lethal relationship^{21,22}. Likewise, for anti-apoptotic genes, the ability of expression of one to rescue inhibition of another is well-documented, necessitating combinatorial targeting²³. Applicants also selected gene families with known or potential redundancy in their function, including MAPKs, AKTs, and ubiquitins²⁴⁻²⁶. Finally, Applicants included several genes computationally predicted to engage in multiple synthetic lethal interactions²⁷. Applicants designed 3 sgRNAs against these 25 genes for both SaCas9 and SpCas9 (Najm et al., 2017 Supplementary Table 2). Each gene pair is assessed with 18 unique sgRNA combinations (2 Cas9s×3 gene A sgRNAs×3 gene B sgRNAs); an ineffective individual sgRNA affects 3 of the combinations, emphasizing the importance of effective design. Applicants targeted two control genes: EEF2 (3 sgRNAs), a core essential gene, and CD81 (10 sgRNAs), a cell surface marker with no known viability effect in most cells. Applicants added two sets of negative controls, sgRNAs that target introns of HPRT1 (5 sgRNAs), and 3 expression cassettes that terminate transcription due to a run of 6 thymidines (6T). The resulting 96×96=9,216 member SynLet library was packaged into lentivirus for use in six diverse tumor cell lines engineered to express SpCas9: A375 (skin); Meljuso (skin); HT29 (colon); A549 (lung); 7860 (kidney); and OVCAR8 (ovary).

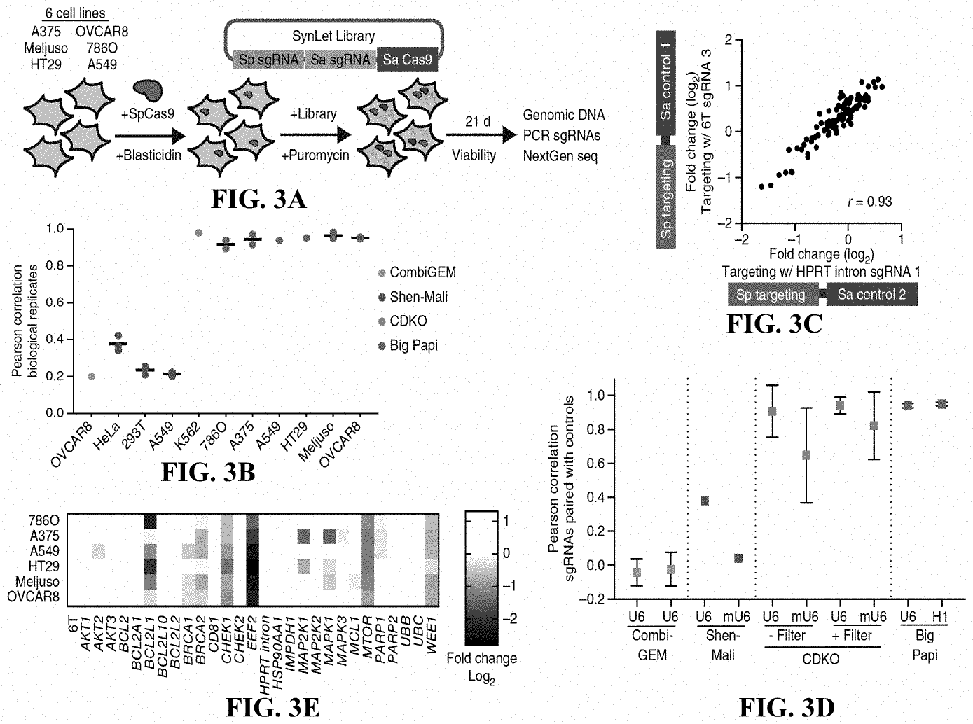

Cells were transduced at low MOI (˜0.5) in biological duplicate, selected with puromycin, and cultured for 21 days; for some, an earlier time point was also collected (FIG. 3a). Applicants prepared genomic DNA, PCR-amplified the dual-sgRNA cassette, and quantitated library distribution by sequencing (Najm et al., 2017 Supplementary Table 3). Applicants compared abundance at day 21 to the starting abundance (plasmid DNA) to determine the effect of each sgRNA pair on viability. Biological replicates were well correlated for all six cell lines (Pearson correlations of 0.89-0.98, FIG. 3b). Applicants performed this same analysis for three other dual-knockout combinatorial studies, and found that replicate reproducibility was high for the CDKO screen (0.98) low for the CombiGem (0.2) and Shen-Mali (0.21-0.42) screens (FIG. 3b).

The orthologous Cas9 approach seeks to diminish competition between two sgRNAs, which may arise from differences in transcription, RNA stability, or binding affinity to Cas9 (FIG. 8). Applicants compared performance of individual targeting sgRNAs in one position when partnered with varying control sgRNAs in the second position (FIG. 3c). For targeting sgRNAs utilizing either Cas9, the average log 2-fold-changes were well-correlated regardless of the control sgRNA (FIG. 3d). In contrast, the effects of individual sgRNAs paired with different controls in the CombiGEM and Shen-Mali libraries were not well-correlated (FIG. 3d). The CDKO library, after removing 31% of sgRNA combinations (read counts below 50), showed much better correlation but the decreased consistency for sgRNAs driven from the mouse U6 promoter, evident in the unfiltered data, remained apparent in the filtered data (FIG. 3d). The Shen-Mali data showed the same trend, suggesting that lower expression from the mouse U6 promoter results in unequal competition for Cas9, an issue avoided by the dual-Cas9 approach.

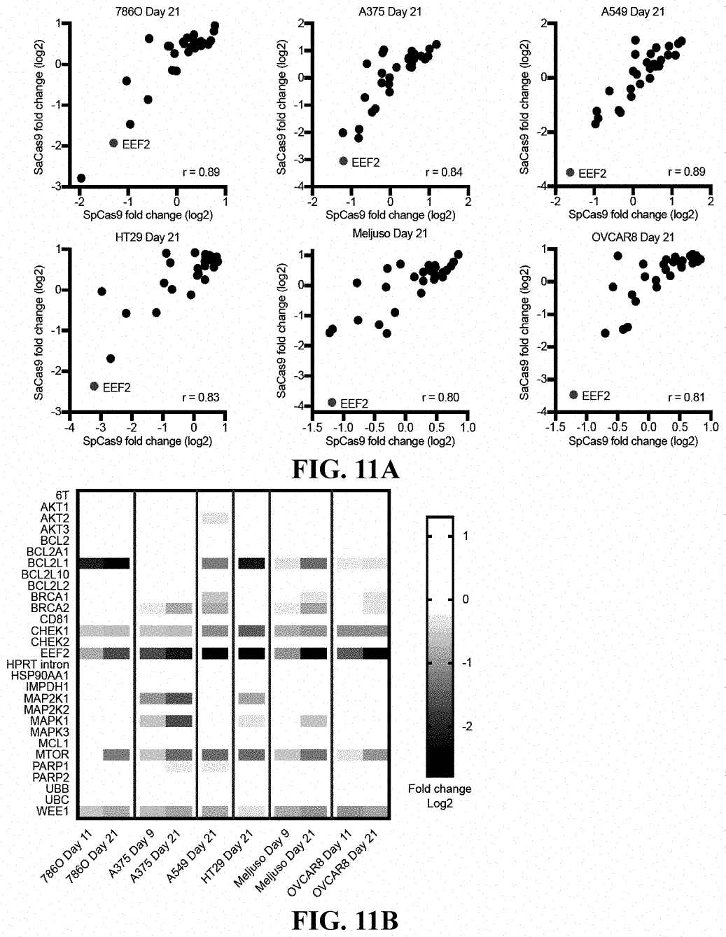

Applicants next examined phenotypic consistency at the gene level between the two Cas9s and observed good agreement, with Pearson correlations of 0.80-0.89 across the 6 cell lines (FIG. 11a). Combining measurements from both Cas9s, single knockouts of EEF2, CHEK1, MTOR, and WEE1 consistently exhibited viability effects, with stronger depletion at day 21, consistent with their classification as fitness genes28; other genes showed cell line specific viability effects (FIG. 3e, FIG. 11b). Thus, SaCas9 and SpCas9 produced mutually consistent knockout phenotypes across cell lines.

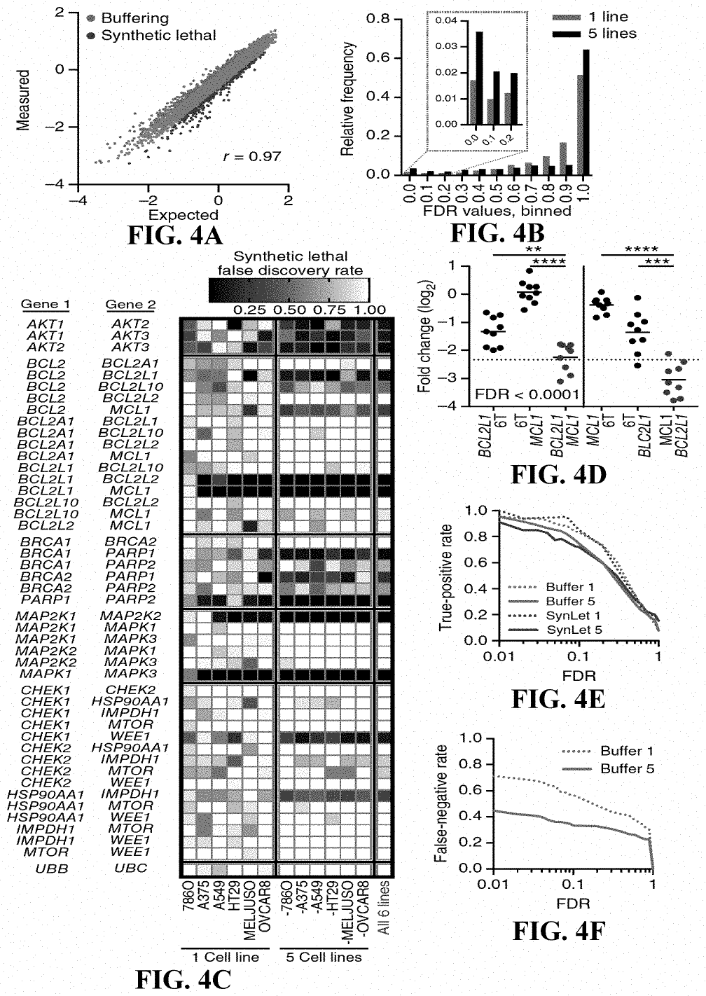

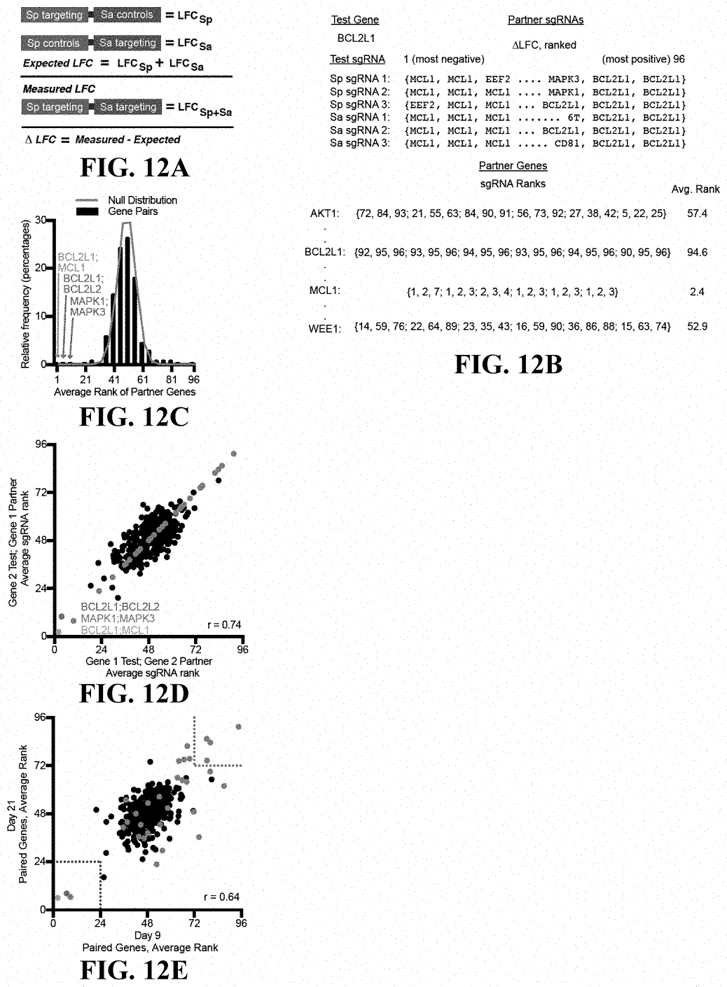

Applicants next assessed synthetic lethal and buffering relationships. Applicants modeled the expected log 2-fold-change from sgRNA pairs as the sum of the log 2-fold-change (LFC) for each individual sgRNA when partnered with controls, and then calculated the difference (ΔLFC) by comparing this expectation to the measured value (FIG. 12a). A positive ΔLFC represents a buffering relationship and a negative ΔLFC represents synthetic lethality. The measured data matched the expectation of this model well (Pearson correlation=0.97), suggesting this is an effective metric for gene interaction (FIG. 4a). Applicants combined information for multiple sgRNA pairs targeting the same gene pairs, and performed the same calculations with randomized input data to generate a null distribution, allowing the calculation of a false discovery rate (FDR, FIG. 12b, FIG. 12c). Using this framework, Applicants analyzed all 6 cell lines harvested at the day 21 time point (FIG. 4b, Najm et al., 2017 Supplementary Table 4).

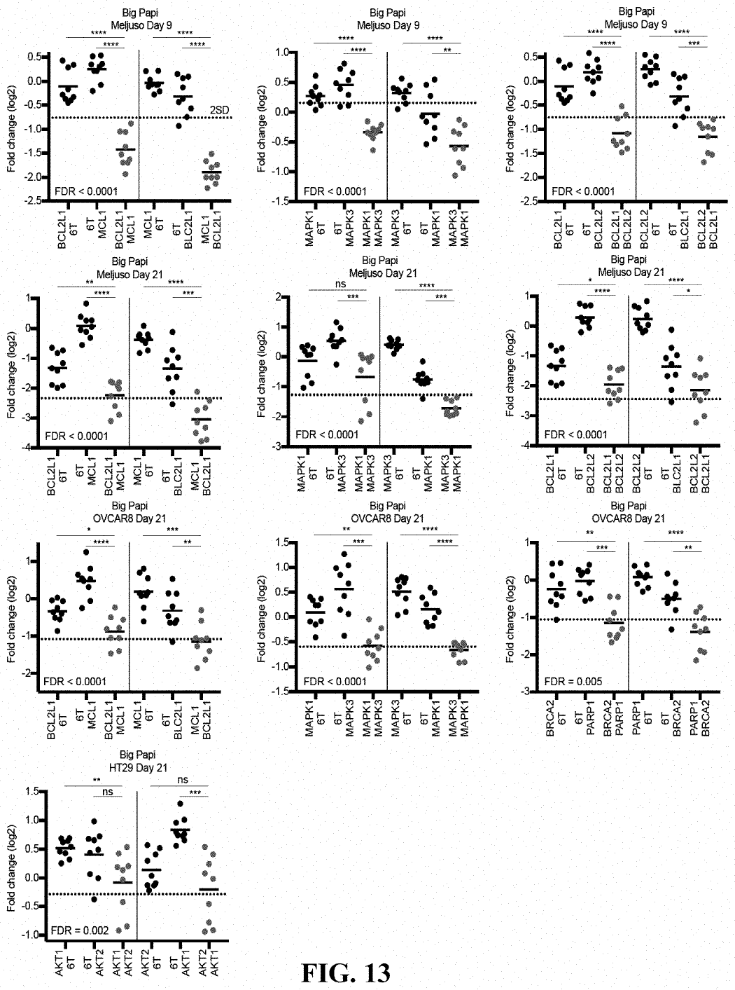

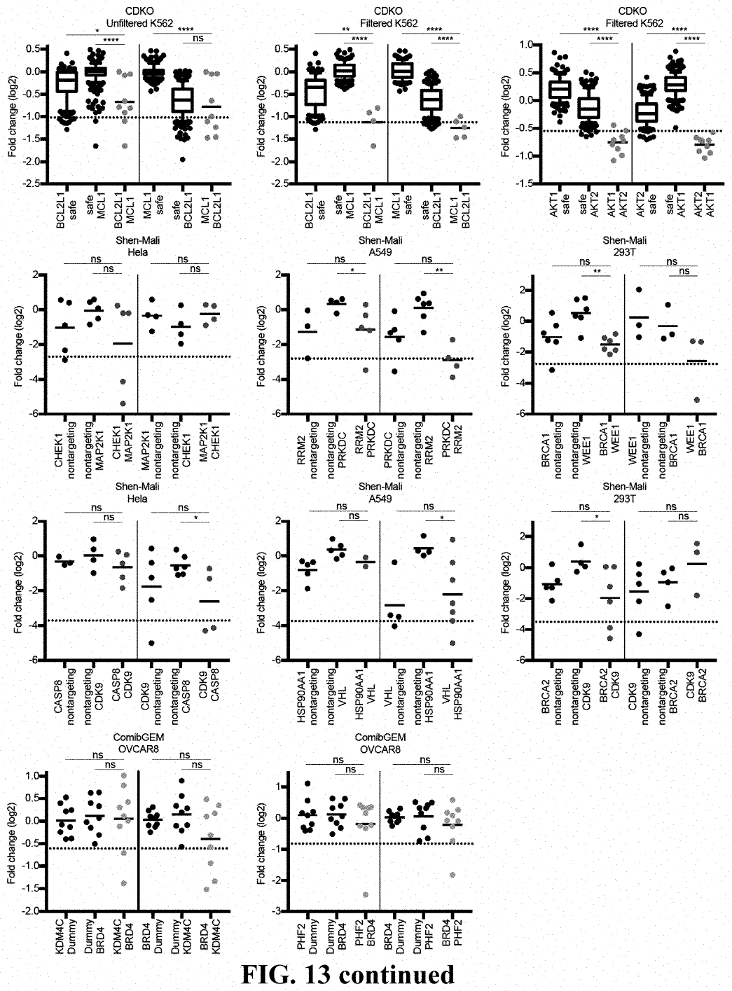

Examining interactions within the pre-defined gene groups, several expected synthetic lethal relationships emerge (FIG. 4c). For example, the anti-apoptotic genes BCL2L1 (Bcl-xL) and MCL1 scored strongly (FDR<0.01) in 5 of 6 cell lines, with 12 of the 18 sgRNA combinations depleted more than two standard deviations from the log 2-fold-change of the individual sgRNAs when paired with controls in Meljuso cells (FIG. 4d). The CDKO approach also found this interaction with strong statistical significance in the filtered data (FIG. 13)⁸. In the CombiGEM and Shen-Mali screens, few sgRNA pairs exceeded two standard deviations versus control pairings, and sets of all sgRNA pairings for the top hit gene pairs showed modest statistical significance across several examples (FIG. 13). From this analysis, Applicants conclude that the Big Papi approach can identify hits consistently across sgRNA pairs.

Although some gene pairs, such as MAPK1-MAPK3 and BCL2L1-MCL1, showed strong effects in most cell lines, other interactions scored strongly in one cell line but were modest or absent in others (FIG. 4c). Applicants hypothesized that combining information across cell lines could improve detection of weaker but generalizable interactions, minimizing technical and cell-line-specific sources of variation; this proved an effective strategy (Najm et al., 2017 Supplementary Table 4). For example, in OVCAR8 cells, BRCA1-PARP1 scored with an FDR of 0.18 and the other 5 individual lines ranged from 0.46-0.94, whereas combining those 5 lines gave an FDR of 0.22, and all 6 cell lines gave an FDR of 0.06. The three AKT isoforms had a similar pattern. Conversely, some interactions with modest FDRs in one cell line are not supported in other lines, such as BCL2A1 and BCL2L10, which has an FDR of 0.48 in A375 cells and 1.0 in the combination of the other 5 lines; such examples may be truly cell line specific or could represent false positives. Overall, conducting primary screens across multiple cell lines is an effective strategy for discovering generalizable interactions.

By making some conservative assumptions about the correctness of particular subsets of synthetic lethal or buffering interactions, Applicants were able to estimate in two independent ways true positive rates for the SynLet screens (Methods). Using these models, Applicants calculated the true positive rate at different FDR thresholds for data from both individual cell lines as well as all leave-one-out iterations and obtained similar estimates whether based on synthetic lethal effects or on buffering interactions, suggesting the independent assumptions made for each were reasonable (FIG. 4e). At an FDR threshold of 0.1, the empirically-determined true positive rate ranged from 72-85%, not far from the theoretical value of 90% (i.e. 10% false discoveries).

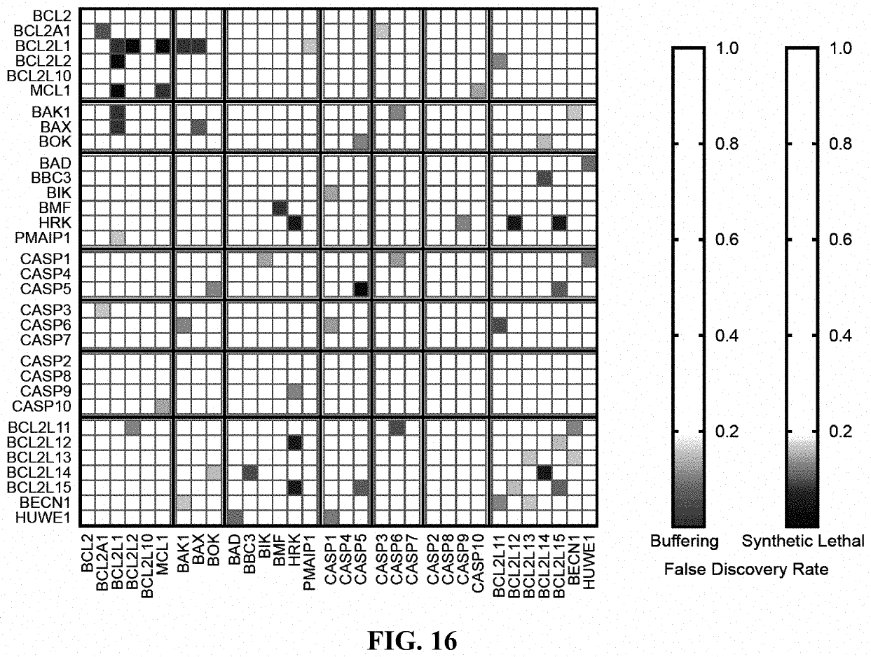

Similarly, Applicants modeled false negative rates based on conservative assumptions about same-gene buffering interactions (Methods). Applicants observed a lower false negative rate when combining information from multiple cell lines (FIG. 4f); for example, at an FDR of 0.1, Applicants determine a false negative rate of 57% when using individual cell lines, whereas combining 5 lines gives a false negative rate of 33%. Overall, the empirically determined true positive and false negative rates suggest that Big Papi is an efficient screening approach (FIG. 16), especially when assayed across multiple cell lines.

### Example 4—Genetic Interactions

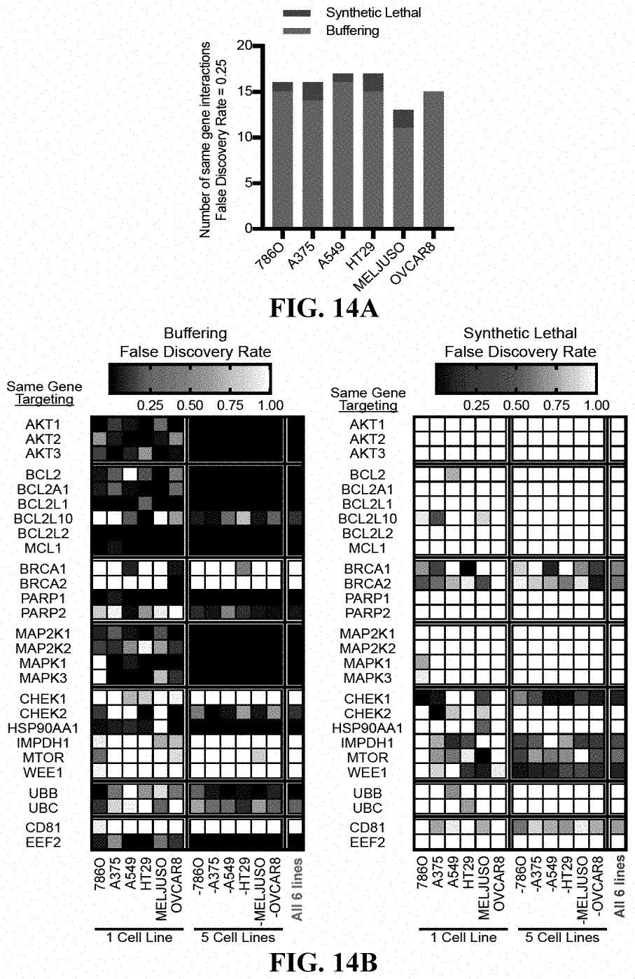

Applicants examined synthetic lethal interactions within the pre-defined groups across the 6 cell lines (FIG. 4c). Applicants did not observe a relationship between the putatively-redundant genes UBB and UBC, despite analysis of buffering interactions indicating that the sgRNAs are active (FIG. 14b). Among the set of genes computationally predicted to engage in synthetic lethal interactions Applicants did not observe strong interactions²⁷. Applicants note that these genes generally performed poorly in the analysis of buffering interactions (FIG. 14b) and thus may represent false negative findings. Combining information from all cell lines, however, identified an interaction between CHEK1 and WEE1 (FDR=0.10), which has also been seen with small molecule inhibitors²⁹. The other 4 pre-defined groups revealed many interactions for further analysis and study.

### Anti-Apoptotic Genes

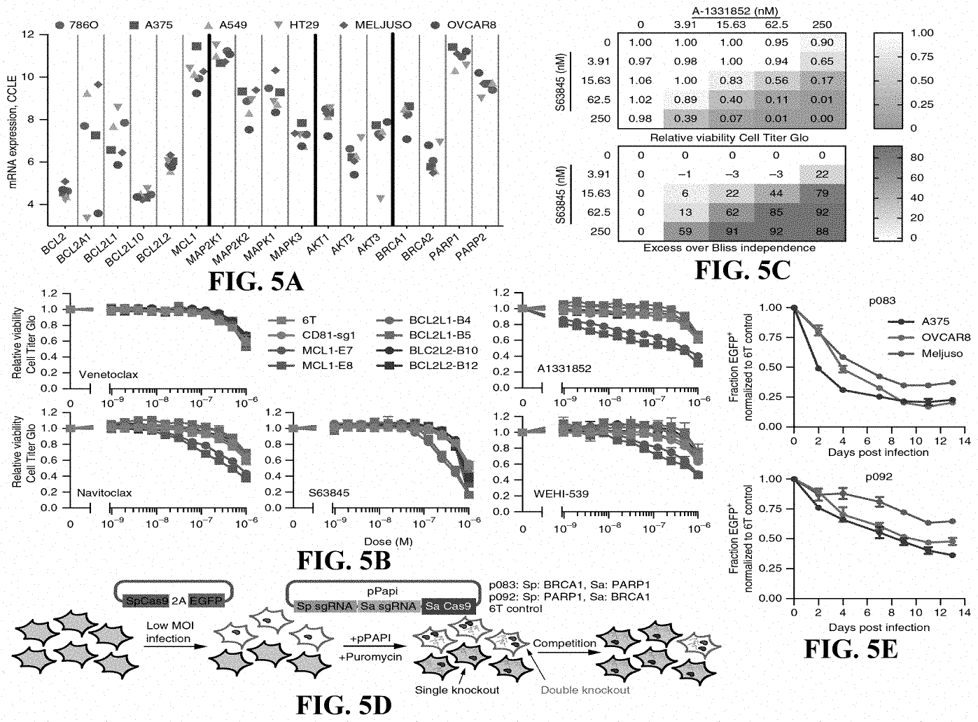

In addition to the interaction between BCL2L1 and MCL1, synthetic lethality between BCL2L1 and BCL2L2 (Bcl-w) was detected in Meljuso, OVCAR8, and A375 at an FDR<0.01, HT29 (0.03) and A549 (0.31). To the best of Applicants knowledge, this interaction has not previously been observed. BCL2L2 is less studied than BCL2L1, with ˜20-fold fewer publications indexed in PubMed. BCL2 is poorly expressed in these cells, but in Meljuso, with the highest expression, BCL2 interacted with BCL2L1 (FDR<0.01) and MCL1 (0.20) (FIG. 5a). Applicants did not observe any strong interactions involving the anti-apoptotic proteins BCL2L10 and BCL2A1, despite high expression of the latter in some lines (FIG. 5a).

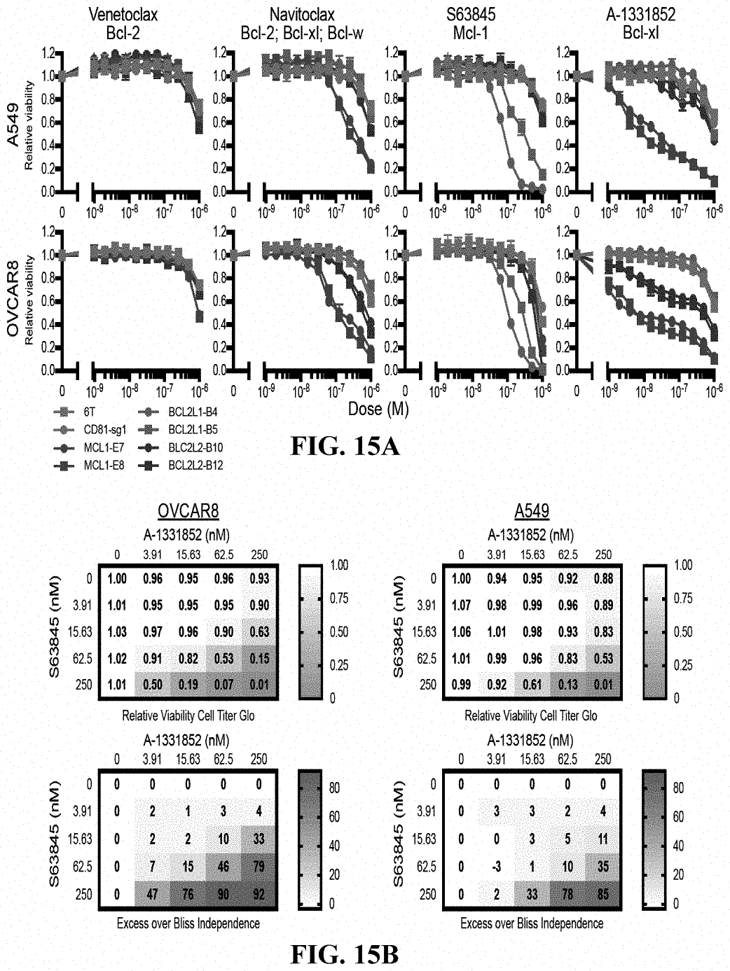

Applicants confirmed these interactions with small molecule inhibitors. Meljuso, OVCAR8, and A549 cells were transduced with single SaCas9 sgRNAs targeting MCL1, BCL2L1, or BCL2L2, or controls. Cells were treated with various inhibitors of anti-apoptotic proteins: venetoclax, an FDA-approved BCL2 inhibitor³⁰; navitoclax, an extensively-characterized inhibitor of BCL2, BCL2L1, and BCL2L231, A-1331852 and WEHI-539, tool compounds described as BCL2L1 inhibitors³²′³³; and 563845, an MCL1 inhibitor in clinical development³⁴. Cells were dosed from 1 nM to 1 μM, and cell viability assessed (FIG. 5b, FIG. 15a). Both sgRNAs targeting MCL1 strongly synergized with navitoclax, A-1331852, and WEHI-539; conversely, sgRNAs targeting BCL2L1 synergized specifically with S63845. Dual small molecule treatment with A-1331852 and S63845 likewise synergized, with excess over Bliss independence scores of 85 or greater at combinations with 250 nM (FIG. 5c, FIG. 15b). Thus, small molecules confirmed the synthetic lethal interaction between MCL1 and BCL2L1.

### MAPK Genes

Applicants detected a strong interaction between MAPK1 (ERK2) and MAPK3 (ERK1) in A375 (FDR=0.04), A549 (<0.01), HT29 (<0.01), Meljuso (<0.01), and OVCAR8 (<0.01) (FIG. 4c), all lines with activating mutations in the MAPK pathway (BRAF V600E; KRAS G12S; BRAF V600E; NRAS Q61L and HRASG13D; and KRAS P121H, respectively). 7860 cells, with no known mutations in the MAPK pathway, showed a weaker interaction (FDR=0.57). MAP2K1 (MEK1) and MAP2K2 (MEK2) synergized in 4 of the 5 MAPK pathway mutant cell lines: HT29 (FDR=0.01), OVCAR8 (0.05), Meljuso (0.06), and A549 (0.11). The exception, A375 (FDR=1.0), was sensitive to loss of MAP2K1 individually (FIG. 3e).

### AKT Genes

Applicants saw a strong interaction for AKT1-AKT2 in HT29 cells (FDR<0.01), the only line with a known PIK3CA mutation (P449T); the CDKO library also detected this interaction8. In contrast to the other 5 lines, HT29 cells express low levels of AKT3, potentially explaining the strong interaction (FIG. 5a). Likewise, AKT1-AKT3 scored strongly in OVCAR8 cells (FDR=0.13), which express the lowest levels of AKT2. However, the interaction between AKT2-AKT3 in Meljuso cells (FDR=0.09) is not predicted based on AKT1 expression. Finally, Applicants observe relationships across all three AKT proteins in 7860 cells, with moderate FDRs ranging from 0.35-0.46. No AKT isoforms show low expression in this line; thus, expression of one may partially compensate for loss of the other two. The relationships between AKT proteins are well-studied and complex, and they have both redundant and unique activities dependent on cellular context^{35,36}. Despite these differences across cell lines, combining information across all 6 lines gave FDRs of 0.04-0.12 (FIG. 4c).

### BRCA & PARP Genes

Applicants observed a relationship between PARP1-PARP2 in four cell lines: OVCAR8 (FDR=0.06), A549 (0.07), A375 (0.12), and Meljuso (0.13), and across all cell lines (<0.01). Only OVCAR8 showed a strong interaction between BRCA2-PARP1 (FDR<0.01); BRCA1 expression is lowest in these cells (FIG. 5a). That the interactions across these genes was most pronounced in the ovarian line may have been anticipated, as PARP inhibitors have shown clinical efficacy in BRCA-deficient ovarian cancers²², although the dissimilar strength across cell lines for BRCA1-PARP1 may not have been expected. To further investigate, Applicants performed a competition assay in three cell types: one that originally scored strongly (OVCAR8, FDR=0.18), weakly (A375, 0.68), or was essentially null (Meljuso, 0.94). Here, EGFP+ cells have dual knockout, whereas EGFP-cells are single knockouts of the gene targeted by the SaCas9 sgRNA; the relative viability of these populations can be monitored over time with flow cytometry (FIG. 5d). In OVCAR8 and A375, double knockout cells were strongly depleted relative to single knockouts, however the viability effect on double knockout cells was notably weaker in Meljuso (FIG. 5e). This result validates the interaction originally detected with weaker significance in A375 and demonstrates that Meljuso are indeed less sensitive to combinatorial BRCA1-PARP1 loss. Consistent with this, Shen-Mali classified BRCA1-PARP1 as a “private” synthetic lethal interaction in 293T cells but not A549 or Hela cells7, and BRCA mutant cells show varying sensitivity to PARP inhibitors both in cell culture and clinical settings^{37,38}.

### Example 5—Apoptosis Screen

Interaction networks for pro- and anti-apoptotic genes have been assembled by biochemical approaches, and although some interactions are consistently detected, others show less consistent results³⁹. Because pro-apoptotic genes were robust and reproducible hits in the initial screen, Applicants investigated the apoptotic network further with a Big Papi screen. Applicants selected 32 genes implicated in apoptosis and targeted them each with 4 sgRNAs, for a total of 20,736 perturbations including controls (Najm et al., 2017 Supplementary Table 5); sequencing of plasmid DNA gave an AUC of 0.67, with 73% of combinations present in the top 90% of reads.

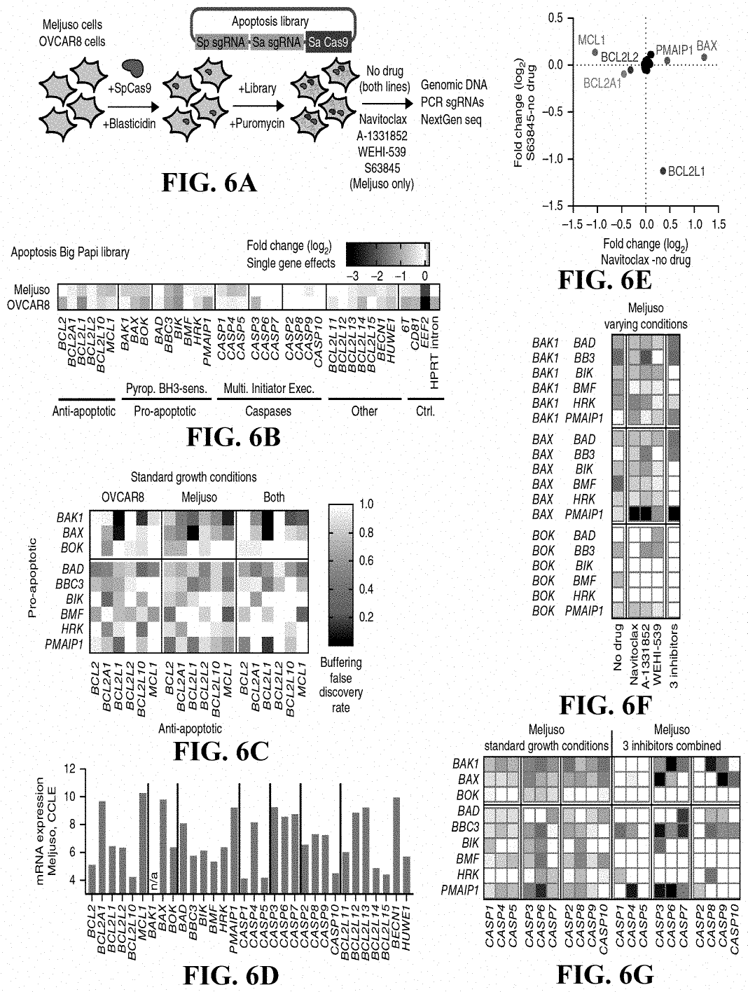

Applicants screened this library in Meljuso and OVCAR8 in duplicate for 21 days in standard growth conditions; further, in Meljuso Applicants challenged the population with various inhibitors of anti-apoptotic proteins (FIG. 6a, Najm et al., 2017 Supplementary Table 6). Knockout of some anti-apoptotic genes had minor growth effects, whereas knockout of pro-apoptotic genes did not decrease cell viability (FIG. 6b). Applicants analyzed these screens for synthetic lethal and buffering interactions, and confirmed a strong synthetic lethal interaction between BCL2L1 and both MCL1 and BCL2L2 (FIG. 16, Najm et al., 2017 Supplementary Table 7; this interaction was also observed above FIG. 4). In both cell lines, Applicants observed buffering interactions between pro- and anti-apoptotic genes (FIG. 6c). The strongest interactions were detected between BCL2L1 and both BAK1 and BAX (FDR<0.01 for combined data), multi-BH-domain proteins that direct mitochondrial outer membrane permeabilization (MOMP); interactions between these proteins have been detected biochemically³⁹. BOK did not engage in strong interactions, potentially expected based on its low expression (FIG. 6d).

Applicants next analyzed Meljuso cells screened with inhibitors, first examining single gene effects. As expected, navitoclax and 563845 synergized with MCL1 and BCL2L1 knockout, respectively (FIG. 6e). Knockout of BCL2A1, which did not show strong interactions when screened in standard growth conditions (FIG. 4c, FIG. 16), sensitized the cells to navitoclax. Conversely, knockout of BAX and PMAIP1 (Noxa) led to navitoclax resistance (FIG. 6d). Thus, these screening conditions identified both sensitization and resistance phenotypes.

To examine combinatorial phenotypes, Applicants combined data across the three BCL2L1 inhibitors, to minimize effects due to molecule-specific mechanism of action. Whereas BAX-PMAIP1 knockout showed a minimal buffering interaction in standard growth conditions (FDR=0.89), they synergized strongly to protect cells from death when treated with anti-apoptotic inhibitors (FDR<0.01) (FIG. 6f). Similarly, caspase—pro-apoptotic knockouts produced modest buffering interactions in standard growth conditions; only PMAIP1-CASP6 scored strongly (FDR=0.12) (FIG. 6g). However, inhibitors led to clearer detection of specific interactions. For example, the strongest initiator and effector caspases to interact with BAK1 were CASP8 (FDR=0.10) and CASP6 (FDR=0.01), respectively. These two caspases directly interact with each other⁴⁰. Although caspase interactions are complex, CASP8 is generally associated with the extrinsic cell death pathway⁴¹. Conversely, BAX interacted strongly with CASP9 (FDR<0.01) and CASP3 (FDR=0.04), caspases with a well-established relationship⁴², with CASP9 associated most strongly with the mitochondrial cell death pathway. Although BAK1 and BAX are generally considered functional redundant, differences in localization and binding partners have been documented^{43,44}; to Applicants knowledge this is the first report of differences in genetic interactions with downstream caspases in human cells.

### Example 6—Orthogonal Activities

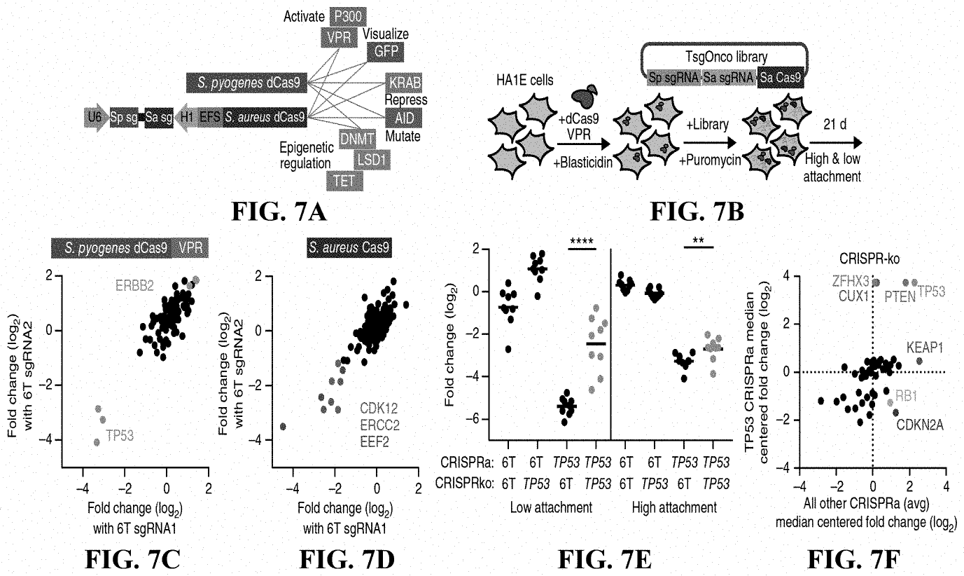

The Big Papi approach is readily applied to concomitant screening of orthogonal modalities (FIG. 7a), for example repressing one gene while activating another. To test the ability to combine distinct gene-targeting activities, Applicants designed a Big Papi library to overexpress 38 annotated oncogenes with CRISPRa technology with 3 sgRNAs each, using a nuclease-dead SpCa9 (dCas9) fused to the “VPR” domain comprised of three transcriptional activators⁴⁵. Applicants employed SaCas9 to knockout 45 tumor suppressor genes, also with 3 sgRNAs each (Najm et al., 2017 Supplementary Table 8). With controls, the TsgOnco library totaled 19,250 constructs; pDNA sequencing gave an AUC of 0.63, and 77% of constructs were detected in the top 90% of reads. Applicants screened HAlE cells, a kidney line immortalized by large T antigen, which inactivates TP53. After infection, cells were grown in standard conditions and on low attachment culture plates (FIG. 7b); the latter are a surrogate for soft agar and select for transformation phenotypes⁴⁶. Applicants first examined performance of targeting sgRNAs paired with control sgRNAs, and observed good consistency, with overexpression of TP53 dramatically reducing viability with all three sgRNAs (FIG. 7c, Najm et al., 2017 Supplementary Table 9). Likewise, SaCas9-mediated knockout of EEF2, CDK12, and ERCC2 decreased cell viability with all three sgRNAs for each gene (FIG. 7d).

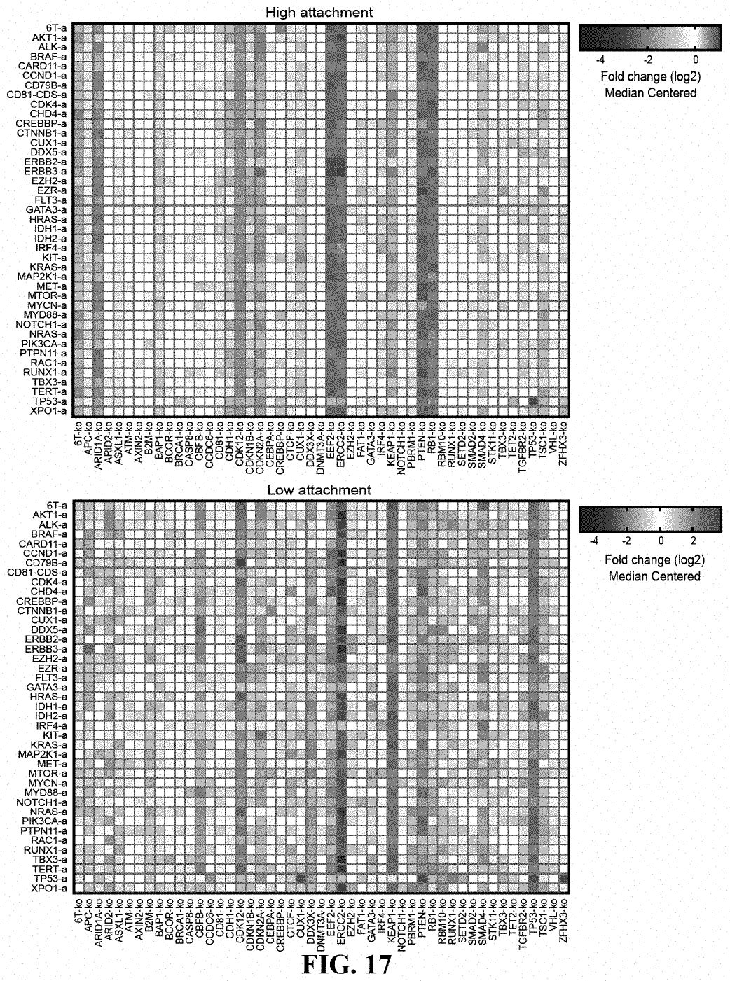

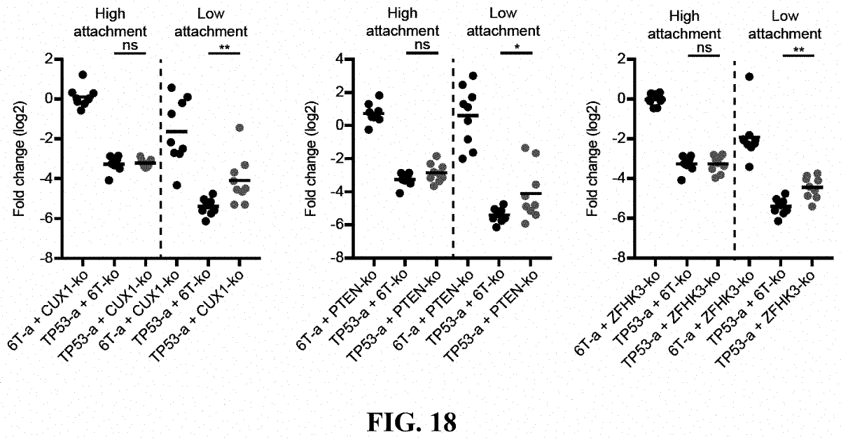

Applicants next examined the data for genetic interactions (FIG. 17). A strong interaction was observed between dSpCas9-VPR sgRNAs targeting TP53 for overexpression, which is lethal, and SaCas9 sgRNAs targeting TP53 for knockout, which buffered this lethality. This effect was stronger in low attachment conditions, and serves as technical validation that overexpression and knockout are co-active in cells (FIG. 7e). Several other interactions with TP53 overexpression were likewise more apparent in the stringent, low attachment conditions. Knockouts of both ZFHX3 (ATBF1) and CUX1, which had minimal effects on viability on their own, partially rescued the lethality caused by TP53 overexpression (FIG. 7f, FIG. 18). ZFHX3 directly interacts with TP53 to activate the CDKN1A promoter (p21^{Cip1}) leading to cell cycle arrest, and thus ZFHX3 loss buffers this TP53 activity47. Likewise, CUX1 deficiency activates PI3K signaling⁴⁸; consistent with this observation, knockout of PTEN increased proliferation, an effect that persisted in cells overexpressing TP53. Conversely, although KEAP1 knockout generally led to increased cell viability, this effect was muted upon TP53 overexpression (FIG. 7f), which is consistent with the opposing actions of KEAP1 and TP53 on the transcription factor NFE2L2 (Nrf2). Normally, KEAP1 degrades NFE2L2, so KEAP1 loss leads to NFE2L2 stabilization; TP53 suppresses the metabolic target genes of NFE2L2, thereby nullifying the effect of KEAP1 knockout⁴⁹. Notably, knockout of both CDKN2A (p16) and RB1 gave increased viability in the absence of TP53, but overexpression of TP53 reversed this phenotype. That these two genes are immediately upstream and downstream, respectively, of the cell cycle kinases CDK4 and CDK6 suggests this counterintuitive observation merits further exploration. These results serve as proof-of-principle that the Big Papi approach can combine multiple Cas9 activities in a single screen to reveal genetic interactions.

### Example 7—Discussion

Applicants developed a dual-Cas9 system to identify genetic interactions. This system is efficient, cost effective, and supports pooled library generation and screening. Synthetic lethal screens using the present system identified interactions within several groups of functionally related genes, including the MAPK pathway, AKT signaling, DNA damage repair, and apoptosis, with high statistical confidence. Applicants also applied this system to map buffering interactions between genes involved in apoptosis, both in standard growth conditions and in the presence of small molecules, which revealed additional genetic interactions. Finally, Applicants combined CRISPR-mediated knockout and overexpression to uncover interactions with TP53.

SaCas9 has been utilized previously for in vivo gene editing^{19,20} and in an orthologous, chemically induced CRISPRa and CRISPRi system, although it was noted to have lower efficiency than SpCas9 in that study, most likely due to suboptimal sgRNA selection⁵⁰. To increase SaCas9 utility, Applicants assessed the activity of thousands of sgRNAs to define rules enabling selection of highly-active sgRNAs. GUIDE-Seq results have shown that SaCas9 has fewer off-target effects than SpCas9, based on the modest sampling of sgRNAs assessed thus far by this technique⁵¹. These performance properties and the design rules provided here, coupled with its smaller size (˜1 kilobase shorter than SpCas9), highlight SaCas9 as an attractive genome editing tool.

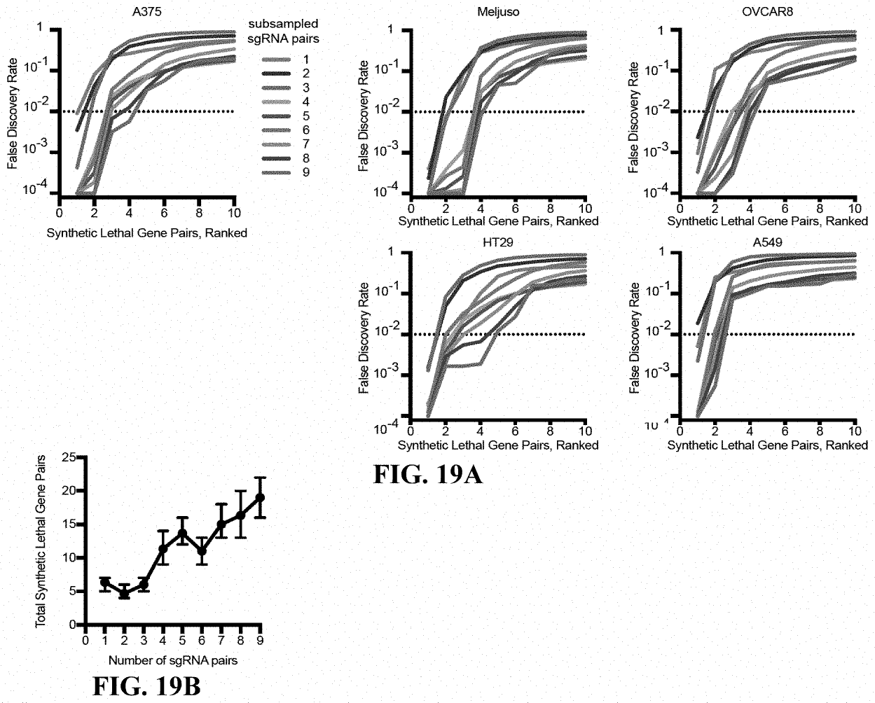

The number of genes that can be screened is typically limited by the scale of cell culture, which dictates the size of the library; generally, genome-wide single-gene sgRNA libraries contain ˜100,000 perturbations and require 1,000 cells per perturbation. The Big Papi approach achieves reasonable performance with only 2 sgRNAs per gene (FIG. 19), and thus a screen to examine pairwise combinations of 158 genes with 2 sgRNAs per gene would have a similar number of perturbations: (158×2) x (158×2)=99,856.

The results highlight the importance of cell context in detecting interactions. With the SynLet library, no gene pair scored strongly (FDR<0.01) in all 6 cell lines, and some showed strong interactions in only one line. One outlier was 7860, a renal clear cell carcinoma line with VHL deletion, in which Applicants identified no synthetic lethal gene pairs at an FDR<0.01. Biological replicates of the 7860 screen were well-correlated, and Applicants also detected buffering interactions when targeting the same gene with both Cas9s, suggesting that the screen was well-executed and the reagents were active. Heterogeneity of small molecules on different cell lines is well-documented and it is reasonable to expect the same heterogeneity across cell lines for genetic interactions. Although mutation status and mRNA expression could be used post facto to rationalize why some interactions were detected more strongly in some lines compared to others, combining information across cell lines proved a useful strategy for detecting generalizable interactions with increased confidence.

In summary, the Big Papi approach described here for dual-gene perturbation screens represents a powerful means to map genetic interactions in mammalian cells that can be applied across many biological questions and model systems.

### Example 8—Methods

Vectors. Plasmids were cloned by synthesis and assembly (Genscript) and are available to the academic research community through Addgene:
pPapi (also known as pXPR_207): U6 and H1 promoters express two sgRNAs; short EF1a promoter (EFS) expresses SaCas9-2A-PuromycinR (Addgene 96921).
pXPR 034: U6 promoter expresses SaCas9 sgRNAs; EFS expresses SaCas9-2A-PuromycinR. An updated version of this plasmid with more convenient restriction sites, pXPR 206, has been deposited in Addgene (96920).
pLX_311-Cas9: SV40 promoter expresses blasticidin resistance; EFla promoter expresses SpCas9 (generated by Sefi Rosenbluh, Hahn lab, Addgene 96924). pXPR 120: EF1a promoter expresses dSpCas9-VPR-2A-BlasticidinR (Addgene 96917).

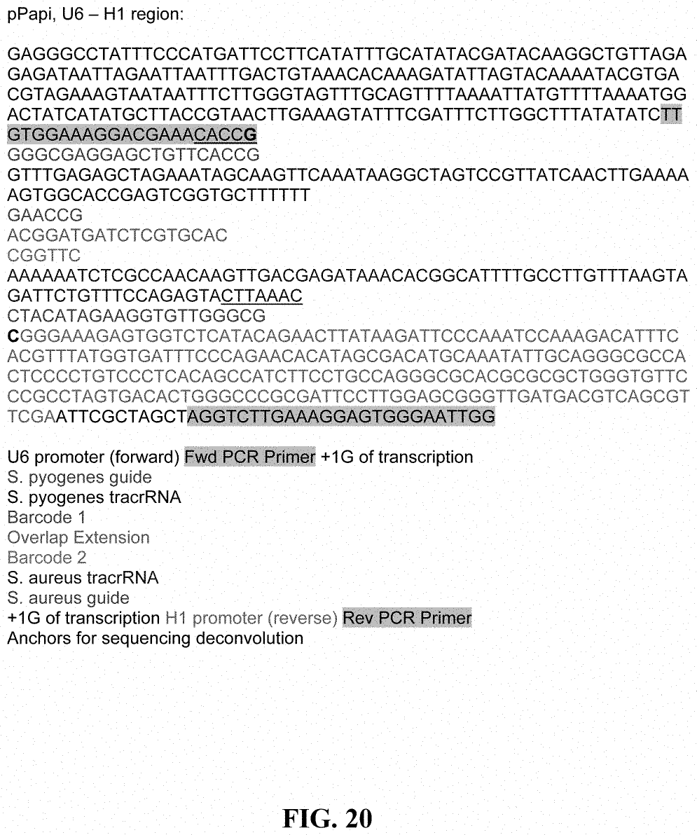

Library production. Pooled libraries for expression of single sgRNAs were made as previously described, with oligonucleotide pools obtained from CustomArray²⁷. For cloning of Big Papi pools, oligonucleotide inserts (Ultramers, IDT) were designed with 5′ BsmBI sites followed by 20 or 21 nt crRNA, 82 nt tracrRNA, 6 nt barcode, and 17 nt complementary sequence (FIG. 1a, FIG. 20). The oligonucleotides for SpCas9 sgRNAs and SaCas9 sgRNAs were separately mixed together at a concentration of 5 μM each. 10 μL, of each pool of oligonucleotides was then combined in a 100 μL, reaction and extended using NEBNext (New England Biolabs) with an annealing temperature of 48° C. The resulting dsDNA was purified by spin-column then ligated into the BsmBI-digested pPapi vector using 100 cycles of Golden Gate assembly with 100 ng insert and 500 ng vector using Esp3I and T7 ligase, as Applicants have done previously for single sgRNA pools²⁷. The DNA was isopropanol precipitated and electroporated into STBL4 cells. A zero-generation (G0) plasmid DNA pool was then amplified by a second electroporation into STBL4 cells to create the G1 plasmid DNA pool, which was then used for virus production. Applicants note that individual constructs to express two sgRNAs can be constructed either by the overlap-extension of individual oligonucleotides or by the use of gBlocks (IDT), which may be a more cost-effective option.

Virus production. For individual virus production: 24 hours before transfection, HEK293T cells were seeded in 6-well dishes at a density of 1.5×10⁶ cells per well in 2 mL of DMEM+10% FBS. Transfection was performed using TransIT-LT1 (Mirus) transfection reagent according to the manufacturer's protocol. In brief, one solution of Opti-MEM (Corning, 66.25 μL) and LT1 (8.75 μL) was combined with a DNA mixture of the packaging plasmid pCMV VSVG (Addgene 8454, 1250 ng), psPAX2 (Addgene 12260, 1250 ng), and the sgRNA-containing vector (e.g. pPapi, 1250 ng). The two solutions were incubated at room temperature for 20-30 minutes, during which time the HEK293T cells were replenished with fresh media. After this incubation, the transfection mixture was added dropwise to the surface of the HEK293T cells, and the plates were centrifuged at 1000×g for 30 minutes. Following centrifugation, plates were transferred to a 37° C. incubator for 6-8 hours, then the media was removed and replaced with media supplemented with 1% BSA. A larger-scale procedure was used for production of the sgRNA library; 24 hours before transfection, 18×10⁶ HEK293T cells were seeded in a 175 cm² tissue culture flask, with transfection performed as described above using 6 mL of Opti-MEM and 300 μL of LT1. Flasks were transferred to a 37° C. incubator for 6-8 hours, then media aspirated and replaced with BSA-supplemented media. Virus was harvested 36 hours after this media change.

Cell culture. A375, HT29, OVCAR8, 7860, A549, and Meljuso cells were obtained from the Cancer Cell Line Encyclopedia; HAlE cells were obtained from the Connectivity Map; HEK293T cells were obtained from ATCC (CRL-3216). All cell lines were routinely tested for mycoplasma contamination and maintained in a 37° C. humidity-controlled incubator with 5.0% CO₂ Cells were maintained in exponential phase growth by passaging every 2 or 3 days. Cell lines were maintained without antibiotics and supplemented with 1% penicillin/streptomycin during screens. Cas9 derivatives were made by transducing with the lentiviral vector pLX_311-Cas9, which expresses blasticidin resistance from the SV40 promoter and Cas9 from the EF1a promoter, as described previously²⁹. The following list includes, respectively, cell line, media, and concentration of puromycin, blasticidin, and polybrene:

A375; RPMI+10% FBS; 1 μg/ml; 5 μg/ml; 1 μg/ml.

HEK293T; DMEM+10% FBS; 1 μg/ml; 5 μg/ml; 1 μg/ml.

HT29; DMEM+10% FBS; 2 μg/ml; 5 μg/ml; 1 μg/ml.

MOLM13; RPMI+10% FBS; 2 μg/ml; 5 μg/ml; 4 μg/ml.

Meljuso; RPMI+10% FBS; 1 μg/mL; 2 μg/mL; 4 μg/mL

A549; DMEM+10% FBS; 1.5 μg/mL; 5 μg/mL; 1 μg/mL

OVCAR8; RPMI+10% FBS; 2 μg/mL; 3 μg/mL; 4 μg/mL

7860; RPMI+10% FBS; 1 μg/mL; 2 μg/mL; 4 μg/mL

HA1E; MEM-alpha+10% FBS; 1 μg/mL; 8 μg/mL; 4 μg/mL

Flow Cytometry. For experiments carried out in FIG. 1 and FIG. 9, A375 cells stably expressing SpCas9 and GFP were transduced at an MOI of −1 in 12-well plates. Two days after transduction, cells were selected with puromycin (1 μg/mL) for five days. Cells were stained with APC-conjugated CD81 antibody (Biolegend 349510) diluted 1:100 in flow buffer (PBS, 2% FBS, 5 μM EDTA) for 30 minutes on ice. Residual antibody was removed with two flow buffer washes, and cells were re-suspended in flow buffer. Flow cytometry was performed on the BDAccuri C6 Sampler system or Live cell populations were gated using forward and side scatter to exclude debris. CD81+ and EGFP+gates were set using non-transduced A375-SpCas9-EGFP cells.

SaCas9 activity rules. Computational modeling for SaCas9 activity was done as previously for SpCas9²⁷. In contrast to the previous work, Applicants did not use the NGGX interaction feature (which is SpCas9 PAM-specific). Also, previously Applicants generated two models for SpCas9, one which used gene positional features (nucleotide cut, percent peptide), and one that omitted them. Applicants have since found the latter to be used more frequently, as it does not assume the target DNA encodes a protein, and thus Applicants did not use gene positional features for the derivation of the SaCas9 activity model.

SynLet library screening. To determine lentiviral titer, cell lines were transduced in 12-well plates with 150, 300, 500, and 800 μL virus with 3.0×10⁶ cells per well in the presence of polybrene. The plates were centrifuged at 640×g for 2 hours then transferred to a 37° C. incubator for 4-6 hours. Each well was then trypsinized, and an equal number of cells seeded into each of two wells of a 6-well dish. Two days post-transduction, puromycin was added to one well out of the pair. After 5 days, both wells were counted for viability by trypan exclusion. A viral dose resulting in 30-50% transduction efficiency, corresponding to an MOI of ˜0.35-0.70, was used for subsequent library screening. Prior to screening-scale transduction, Cas9-expressing cell lines were selected with blasticidin then transduced in two or three biological replicates; puromycin selection began two days post-transduction. Transductions were performed with enough cells to achieve a representation of at least 500 cells per sgRNA per replicate, taking into account a 30-50% transduction efficiency. Puromycin selection was maintained for 5-7 days. Throughout the screen, cells were split at a density to maintain a representation of at least 500 cells per sgRNA. Cell counts were taken at each passage to monitor growth. After this screen, cells were pelleted by centrifugation, resuspended in PBS, and frozen promptly for genomic DNA isolation.

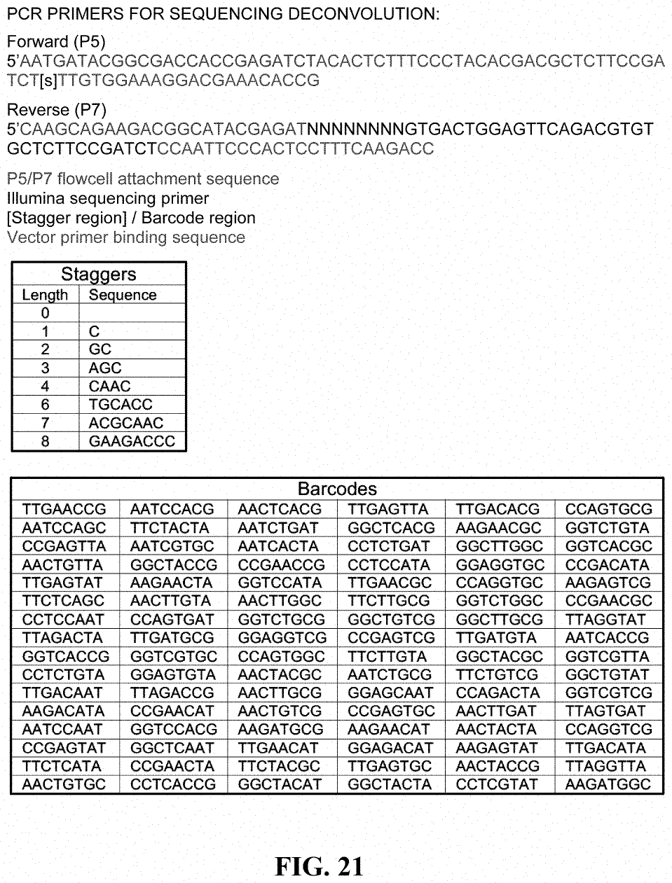

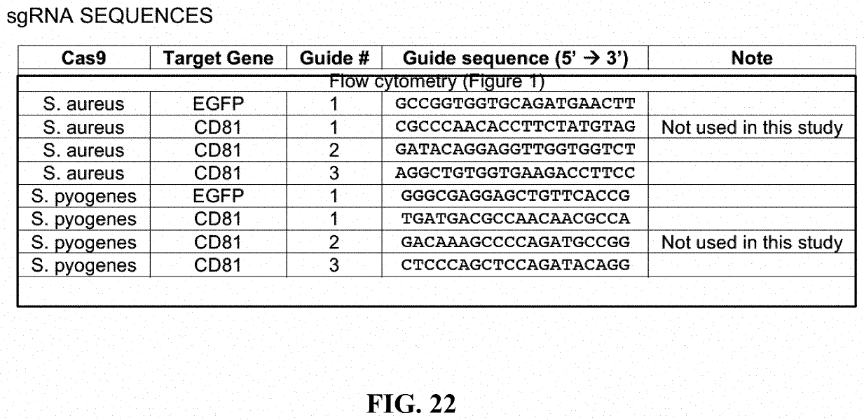

Genomic DNA preparation and sequencing. Genomic DNA (gDNA) was isolated using the QIAamp DNA Blood Midi Kit (Qiagen) as per the manufacturer's instructions. The concentration of these preparations was determined by UV spectroscopy (Nanodrop). PCR of single sgRNA expressing vectors was as described²⁷. For the pPapi vector, dual sgRNA cassettes and plasmid DNA were PCR-amplified and barcoded with sequencing adaptors using ExTaq DNA Polymerase (Clontech), following the same procedure. Primer sequences (IDT) can be found in FIG. 21. Amplified samples were then purified with Agencourt AMPure XP SPRI beads (Beckman Coulter, A63880) according to manufacturer's instructions and sequenced on a NextSeq sequencer (Illumina) with 300 nt single-end reads, with a 10% spike-in of PhiX DNA. Deconvolution of single sgRNA expressing vectors was as described²⁷. For the pPapi vector, reads of the first sgRNA were counted by first searching in the sequencing read for CACCG, the part of the vector sequence that immediately precedes the 20-nucleotide U6 promoter-driven SpCas9 sgRNA. The sgRNA sequence following this search string was mapped to a reference file with all sgRNAs in the library. To find the H1 promoter-driven SaCas9 sgRNA, two 21-nucleotide sequences were compared: the sequence beginning 194 nucleotides after the SpCas9 sgRNA and the sequence following the S. aureus tracr sequence (CTTAAAC). If the sequences matched, the 21 nt sequence was then mapped to the reference file with all SaCas9 sgRNA. For some sequencing lanes with poorer quality, the reference file with the SaCas9 sgRNAs sequences was shortened, such that fewer than 21 nts were needed to match in order to determine the identity of the sgRNA in that position. See also FIG. 21. Reads were then assigned to the appropriate experimental condition based on the 8-nucleotide P7-appended barcode. The resulting matrix of read counts was normalized to reads per million (rpm) within each condition by the following formula: reads per sgRNA/total reads per condition x 10⁶. A pseudocount of 1 was added, and the rpm was then log₂-transformed.

Validation of Hits in the Apoptosis Pathway. Gene pairs associated with a synthetic lethal phenotype in the library screen were validated using combinatorial viability screening of sgRNA perturbations with 5 small molecule inhibitors: navitoclax (ABT-263, Active Biochem A-1001), A1331852 (Active Biochem A-6048); venetoclax (ABT-199, Active Biochem A-1231); WEHI539 (MedChem Express, HY-15607A); and the MCL1 inhibitor S63845 (a gift from Guo Wei, Golub lab). Meljuso cells were transduced in 12-well plates, as described above, with lentivirus containing a single sgRNA targeting one of the anti-apoptotic genes (BCL2L1, BCL2L2, MCL1) or a control sgRNA either targeting CD81 or containing a run of 6 thymidines. Two days after transduction, cells were selected using puromycin at 1 μg/mL for five days. After puromycin selection, 3,000 cells were seeded into 96-well plates. Across each row of the 96-well plate, a different small molecule was added at 11 log 2-dilutions ranging from 1 μM to approximately 1 nM in duplicate for each of the cell lines. The last well in the row did not receive small molecule. After 3 days in the presence of the small molecule, viability of the cell population was assayed by CellTiterGlo (Promega) according to the manufacturer's instructions.

BRCA1/PARP1 competition assay. A375, OVCAR8, and Meljuso cells were transduced with in a 24-well plate with 10 μA Cas9-2A-EGFP virus (Dharmacon, VCAS11862), with 2.0×10⁵ cells per well with 1 μg/mL of polybrene. The plates were centrifuged at 2250 rpm for 2 hours and then transferred to a 37° C. incubator for 4 hours before changing media. The day after transduction, each well was trypsinized and passaged into a T75 flask. The population was confirmed to be a mixture of EGFP+ and EGFP-cells (˜30% EGFP+ for each cell line) and then transduced with the pPapi BRCA1/PARP1 constructs. The vector p083 contains SpCas9 BRCA1 sgRNA B07 and SaCas9 PARP1 sgRNA F01; p092 contains SpCas9 PARP1 sgRNA F06 and SaCas9 BRCA1 sgRNA C02; sgRNAs sequences are listed in Supplementary Table 2. The plates were centrifuged at 2250 rpms for 2 hours and then transferred to a 37° C. incubator for 4-6 hours. Two days post-transduction, puromycin was added to wells for the duration of the assay. Cells were passaged and flow cytometry measurements were taken on the BDAccuri C6 Sampler system at days 0, 2, 4, 7, 9, 11, and 13 post-infection with the pPapi vector.

Apoptosis library screen. Infections were conducted as described above for the SynLet library. OVCAR8 cells were passaged in standard growth conditions for 21 days post-infection. In Meljuso cells, each of three biological replicates was split into five arms 7 days post-infection: Navitoclax, A-1331852, 563845, WEHI-539 and no drug (standard growth conditions). All small molecules were screened at 250 nM with an on/off dosing schedule, in which cells were treated with small-molecule for 4 days and then grown in standard growth conditions for 3 days, and then this cycle was repeated for an additional week. All arms were collected at 21 days post-infection. For Meljuso cells, all three replicates were prepared and sequenced separately. In OVCAR8 cells, one replicate was lost during genomic DNA preparation, and the remaining two replicates were combined prior to sequencing.

CRISPRa/CRISPRko Tsg/Onco screen. Oncogenes and tumor suppressors were selected for screening based on their high frequency of mutation in patient tumor samples⁶ and their annotation in the COSMIC database⁶⁶. HAlE cells were infected with pXPR 120 and selected with blasticidin. For the pooled screen, cells were seeded into 7 T175 flasks at 30% confluence and infected with the TSG/Onco library in biological replicate. After 48 hours, puromycin was added, and cells were maintained under puromycin for 5 days. Cells were then split into two conditions. For the High-Attachment conditions, cells were seeded into standard tissue culture treated T225 flasks; for the Low-Attachment conditions, cells were seeded into a 1-layer untreated low-attachment cell stacker (Costar 3303). The High-Attachment conditions was passaged and maintained, and the cells were harvested on Day 14. The Low-Attachment conditions received media changes until cells that adhered reached confluence, and the cells were harvested on Day 19.

### Example 9—Estimating False Positive and False Negative Rates

To estimate the specificity of the screening system and analytical approach, Applicants assumed that true positive synthetic lethal interactions occur only within the pre-defined gene groups, whereas interactions across pre-defined groups are false positives. Likewise, for buffering interactions, Applicants assumed that all true-positive interactions occur in the special case where both Cas9s are targeted to the same gene, which is expected from the model of independent gene action and has been observed previously in combinatorial screens^{16,20}. Both of these assumptions are conservative, in that true (but currently uncharacterized) synthetic lethal interactions across the pre-defined groups or buffering interactions between genes will be counted as false positives. Applicants calculated the true positive rate at different FDR thresholds for data from both individual cell lines as well as all leave-one-out iterations (FIG. 4e). Applicants see similar estimates for the true positive rate for both synthetic lethal and buffering interactions, suggesting the independent assumptions made for each were reasonable. At an FDR threshold of 0.1, the empirically-determined true positive rate ranged from 72-85%, not far from the theoretical value of 90% (i.e. 10% false discoveries), suggesting that the analysis approach is well-calibrated.

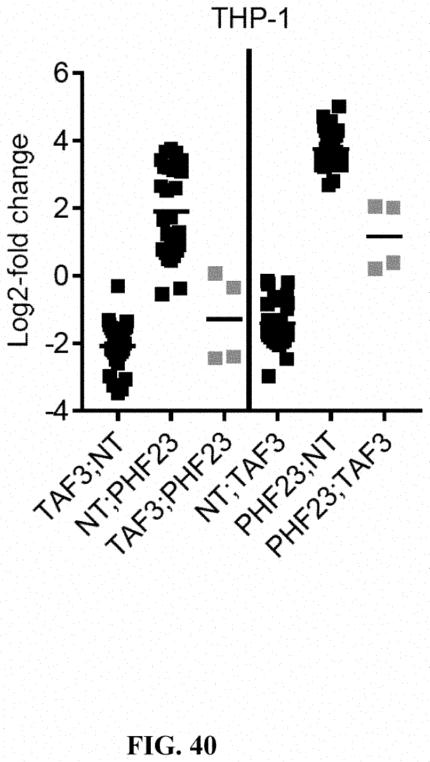

The false negative rate of a genetic screen is notoriously difficult to determine empirically, because for the majority of screens, there are not well-validated sets of true positive genes. For synthetic lethal interactions, there is no reference set of interactions validated to occur in all cell lines. False negatives arise when the reagents targeting the gene are ineffective; for genetic screens that target single genes, there is no data-driven way to determine which genes failed to score because of ineffective targeting purely on the basis of screening results. In these data, however, Applicants can use buffering interactions where both Cas9s are targeted to the same gene to validate the effectiveness of the sgRNAs. Buffering in this special case indicates that both the SaCas9 and SpCas9 sgRNAs must have effectively targeted the gene. Failure to detect a buffering interaction for an individual gene is evidence of failure to effectively target the gene with either or both Cas9s, and thus Applicants can empirically determine a false negative rate. This is a conservative assumption, as it assumes that a gene has a measurable viability effect in a cell, which will not always be true. Buffering interactions were detected with approximately equal prevalence across all cell lines, including 7860 cells, which were bereft of strong synthetic lethal interactions (FIG. 14a). Applicants observed a lower false negative rate when information from multiple cell lines was combined (FIG. 40. For example, at an FDR of 0.1, Applicants determine a false negative rate of 57% when using individual cell lines, whereas combining information from 5 lines gives a false negative rate of 33%. The empirically determined true positive and false negative rates of the Big Papi screening system suggest that this is an efficient screening approach, especially when assayed across multiple cell lines.

### Example 10—Combinatorial Screening of Chromatin Regulators in Cancer

Applicants sought to determine if the screening platform could be used to robustly identify synthetic lethal combinations of chromatin regulators and combinations that are therapeutically actionable. Synthetic lethal interactions in cancer have been previously found for chromatin regulators (see, e.g., Zhao et al., Synthetic essentiality of chromatin remodelling factor CHD1 in PTEN-deficient cancer, Nature. 2017 Feb. 23; 542(7642):484-488; and Helming et al., ARID1B is a specific vulnerability in ARID1A-mutant cancers. Nat Med. 2014 March; 20(3):251-4). Applicants used the chromatin regulator screening platform described herein to identify synthetic lethal interactions in cancer cell lines (300K platform). Indeed, using the chromatin 300K screening platform, Applicants identified ARID1B and ARID1A synthetic lethality in REH cells. The synthetic lethal pairs identified in REH cells included ASF1B and ASF1A, ARID1B and ARID1A, SMARCAL1 and ATRX, ING5 and ING4, HDAC2 and HDAC1, WDR77 and HDAC6, KAT6B and CHD8, WDR77 and KAT6B, KDM3B and ARID1A, KDM3B and CHD3, SETD2 and NSD1, ING2 and ING1, MTA1 and DOT1L, KDM3B and BRD1, KDM4A and KAT6A, INO80 and CBX1, HDAC6 and EZH2, SMARCAL1 and HDAC8, KAT5 and CHAF1B, SUV39H1 and HDAC6, KDM3B and BRD4, KMT2B and BRD8, PRMT5 and KAT5, SIRT4 and CBX1, KAT6A and CHD6, WDR77 and DOT1L, KAT2B and EHMT1, KMT2E and KAT6A, KDM3B and DOT1L, KDM3B and KDM3A, CHD8 and BRD1, HIRA and ATRX, KDM5C and KDM3B, PRDM6 and KDM3B, KAT6B and KAT6A, SMARCB1 and KDM6A, MECP2 and KDM4B, KAT2A and HDAC5, SETD2 and KDM3B, RFWD2 and CHD6, SMARCB1 and ARID3C, SETMAR and BRD1, HDAC2 and DIDO1, HDAC2 and DNMT3B, KDM4D and BRD1, PRDM1 and HDAC8, SMARCA5 and KAT6A, and KMT2D and ARID1A.

Applicants also identified 2STD genes in REH cells, including: SRCAP, WDR77, CHAF1B, TAF5, CSTF1, WDHD1, BRD4, DNMT1, WDR61, GTF3C2, PRMT5, RBBP5, HDAC3, TRIM24, CHD7, HIRA and SMC1A. Applicants also identified border 2STD genes in REH cells, including: SMC2, SMC3, TAF1, WDR92, KDM2B and HUWE1. Applicants used the 300K screening platform to screen for synthetic lethality and buffering in THP-1 and REH cell lines (Table 2). Comparison to lethality of single genes using the Achilles screening platform is also shown (see, e.g., portals.broadinstitute.org/achilles/about; and Cheung et al., Systematic investigation of genetic vulnerabilities across cancer cell lines reveals lineage-specific dependencies in ovarian cancer. Proc Natl Acad Sci USA. 2011 Jul. 26; 108(30): 12372-12377). “Synlet score” refers to the number of synthetic lethal combinations observed with that gene. Buffering is the same. “Amplified” refers to KOs that induced the cells to expand.

TABLE 2

| | THP-1 | THP-1 | | THP-1 | THP-1 | | Reh | | Reh | Reh |
|---|---|---|---|---|---|---|---|---|---|---|
| | ACHILLES | Lethal@ | THP-1 | Synlet | Buffer | Inhibitor | Lethal@ | Reh | Synlet | Buffer |
| Gene | lethal | 2STD | Amplified | Score | Score | available | 2STD | Amplified | Score | Score |
| BRD2 | 0 | 0 | 0 | 10 | 3 | 0 | 0 | 0 | 0 | 0 |
| WDR77 | 0 | 0 | 0 | 7 | 0 | 0 | 1 | 0 | 3 | 0 |
| KDM3B | 0 | 0 | 0 | 4 | 1 | 0 | 0 | 0 | 9 | 2 |
| KAT6A | 0 | 0 | 0 | 4 | 0 | 0 | 0 | 0 | 5 | 1 |
| CHD8 | 0 | 0 | 0 | 4 | 1 | 0 | 0 | 0 | 2 | 0 |
| ING1 | 0 | 0 | 1 | 4 | 1 | 0 | 0 | 0 | 1 | 0 |
| CHD6 | 0 | 0 | 0 | 3 | 0 | 0 | 0 | 0 | 2 | 1 |
| INO80 | 0 | 0 | 0 | 3 | 0 | 0 | 0 | 0 | 1 | 0 |
| HDAC2 | 0 | 0 | 0 | 2 | 0 | 1 | 0 | 0 | 3 | 0 |
| KAT6B | 0 | 0 | 0 | 2 | 0 | 0 | 0 | 0 | 3 | 0 |
| SMARCB1 | 1 | 0 | 0 | 2 | 0 | 0 | 0 | 0 | 2 | 0 |
| KAT5 | 1 | 0 | 0 | 2 | 1 | 0 | 0 | 0 | 2 | 0 |
| BRD4 | 1 | 1 | 0 | 2 | 0 | 1 | 1 | 0 | 1 | 1 |
| ING2 | 0 | 0 | 1 | 2 | 0 | 0 | 0 | 0 | 1 | 0 |
| HDAC3 | 1 | 1 | 0 | 2 | 2 | 1 | 1 | 0 | 0 | 2 |
| PHF23 | 0 | 0 | 1 | 2 | 0 | 0 | 0 | 0 | 0 | 0 |
| BRD3 | 0 | 0 | 0 | 2 | 0 | 0 | 0 | 0 | 0 | 5 |
| TAF1 | 0 | 0 | 0 | 2 | 0 | 0 | 0.5 | 0 | 0 | 0 |
| SUV39H2 | 0 | 0 | 0 | 2 | 1 | 0 | 0 | 0 | 0 | 3 |
| CHD7 | 0 | 0 | 0 | 2 | 4 | 0 | 1 | 0 | 0 | 0 |
| BRD1 | 0 | 0 | 0 | 1 | 0 | 0 | 0 | 0 | 4 | 0 |
| DOT1L | 0 | 0 | 0 | 1 | 0 | 1 | 0 | 0 | 3 | 0 |
| ARID1A | 0 | 0 | 0 | 1 | 0 | 0 | 0 | 0 | 3 | 0 |
| ATRX | 0 | 0 | 0 | 1 | 1 | 0 | 0 | 0 | 2 | 0 |
| SMARCAL1 | 0 | 0 | 0 | 1 | 2 | 0 | 0 | 0 | 2 | 2 |
| CHAF1B | 1 | 1 | 0 | 1 | 0 | 0 | 1 | 0 | 1 | 0 |
| HIRA | 1 | 1 | 0 | 1 | 2 | 0 | 1 | 0 | 1 | 0 |
| PRMT5 | 1 | 0.5 | 0 | 1 | 5 | 1 | 1 | 0 | 1 | 2 |
| EHMT1 | 0 | 0 | 0 | 1 | 0 | 1 | 0 | 0 | 1 | 1 |
| HDAC1 | 0 | 0 | 0 | 1 | 0 | 1 | 0 | 0 | 1 | 1 |
| KDM4A | 0 | 0 | 0 | 1 | 2 | 1 | 0 | 0 | 1 | 0 |
| ASF1A | 0 | 0 | 0 | 1 | 0 | 0 | 0 | 0 | 1 | 0 |
| ASF1B | 0 | 0 | 0 | 1 | 0 | 0 | 0 | 0 | 1 | 0 |
| CHD3 | 0 | 0 | 0 | 1 | 0 | 0 | 0 | 0 | 1 | 0 |
| DNMT3B | 0 | 0 | 0 | 1 | 0 | 0 | 0 | 0 | 1 | 0 |
| KAT2B | 0 | 0 | 0 | 1 | 0 | 0 | 0 | 0 | 1 | 0 |
| SETMAR | 0 | 0 | 0 | 1 | 0 | 0 | 0 | 1 | 1 | 0 |
| MTA1 | 0 | 0 | 0 | 1 | 1 | 0 | 0 | 0 | 1 | 0 |
| NSD1 | 0 | 0 | 0 | 1 | 1 | 0 | 0 | 0 | 1 | 2 |
| KMT2B | 0 | 0 | 0 | 1 | 2 | 0 | 0 | 0 | 1 | 1 |
| DNMT1 | 0 | 0.5 | 0 | 1 | 3 | 1 | 1 | 0 | 0 | 5 |
| KMT2A | 1 | 0.5 | 0 | 1 | 0 | 0 | 0 | 0 | 0 | 0 |
| MORF4L1 | 0 | 0 | 1 | 1 | 0 | 0 | 0 | 0 | 0 | 0 |
| KDM4E | 0 | 0 | 0 | 1 | 0 | 1 | 0 | 0 | 0 | 0 |
| SIRT6 | 0 | 0 | 0 | 1 | 0 | 1 | 0 | 0 | 0 | 0 |
| ARID2 | 0 | 0 | 0 | 1 | 0 | 0 | 0 | 0 | 0 | 1 |
| ARID4B | 0 | 0 | 0 | 1 | 0 | 0 | 0 | 0 | 0 | 0 |
| ASH1L | 0 | 0 | 0 | 1 | 0 | 0 | 0 | 0 | 0 | 0 |
| BPTF | 0 | 0 | 0 | 1 | 0 | 0 | 0 | 0 | 0 | 0 |
| CHD2 | 0 | 0 | 0 | 1 | 0 | 0 | 0 | 0 | 0 | 0 |
| CHD4 | 1 | 0 | 0 | 1 | 0 | 0 | 0 | 0 | 0 | 2 |
| CORO2A | 0 | 0 | 0 | 1 | 0 | 0 | 0 | 0 | 0 | 0 |
| INTS12 | 0 | 0 | 0 | 1 | 0 | 0 | 0 | 0 | 0 | 0 |
| PHF12 | 0 | 0 | 0 | 1 | 0 | 0 | 0 | 0 | 0 | 0 |
| SETD1A | 1 | 0 | 0 | 1 | 0 | 0 | 0 | 0 | 0 | 1 |
| SETD3 | 0 | 0 | 0 | 1 | 0 | 0 | 0 | 0 | 0 | 0 |
| SETD6 | 0 | 0 | 0 | 1 | 0 | 0 | 0 | 0 | 0 | 0 |
| SETDB1 | 1 | 0 | 0 | 1 | 0 | 0 | 0 | 0 | 0 | 0 |
| BRD9 | 0 | 0 | 0 | 1 | 1 | 0 | 0 | 0 | 0 | 0 |
| HDAC6 | 0 | 0 | 0 | 0 | 0 | 1 | 0 | 0 | 3 | 0 |
| HDAC8 | 0 | 0 | 1 | 0 | 0 | 1 | 0 | 1 | 2 | 0 |
| CBX1 | 0 | 0 | 0 | 0 | 0 | 0 | 0 | 0 | 2 | 1 |
| SETD2 | 0 | 0 | 0 | 0 | 0 | 0 | 0 | 0 | 2 | 1 |
| KMT2E | 0 | 0 | 1 | 0 | 0 | 0 | 0 | 0 | 1 | 0 |
| KDM4B | 0 | 0 | 0 | 0 | 0 | 1 | 0 | 0 | 1 | 0 |
| KDM4D | 0 | 0 | 0 | 0 | 0 | 1 | 0 | 0 | 1 | 0 |
| KDM5C | 0 | 0 | 0 | 0 | 0 | 1 | 0 | 1 | 1 | 0 |
| KDM6A | 0 | 0 | 0 | 0 | 0 | 1 | 0 | 1 | 1 | 0 |
| EZH2 | 0 | 0 | 0 | 0 | 2 | 1 | 0 | 0 | 1 | 2 |
| ARID3C | 0 | 0 | 0 | 0 | 0 | 0 | 0 | 0 | 1 | 0 |
| BRD8 | 0 | 0 | 0 | 0 | 0 | 0 | 0 | 0 | 1 | 0 |
| DIDO1 | 1 | 0 | 0 | 0 | 0 | 0 | 0 | 0 | 1 | 0 |
| HDAC5 | 0 | 0 | 0 | 0 | 0 | 0 | 0 | 0 | 1 | 2 |
| ING4 | 0 | 0 | 0 | 0 | 0 | 0 | 0 | 0 | 1 | 0 |
| ING5 | 0 | 0 | 0 | 0 | 0 | 0 | 0 | 0 | 1 | 0 |
| KDM3A | 0 | 0 | 0 | 0 | 0 | 0 | 0 | 0 | 1 | 0 |
| KMT2D | 0 | 0 | 0 | 0 | 0 | 0 | 0 | 0 | 1 | 0 |
| MECP2 | 0 | 0 | 0 | 0 | 0 | 0 | 0 | 0 | 1 | 0 |
| PRDM1 | 0 | 0 | 0 | 0 | 0 | 0 | 0 | 0 | 1 | 0 |
| PRDM6 | 0 | 0 | 0 | 0 | 0 | 0 | 0 | 0 | 1 | 1 |
| SIRT4 | 0 | 0 | 0 | 0 | 0 | 0 | 0 | 0 | 1 | 0 |
| SMARCA5 | 1 | 0 | 0 | 0 | 0 | 0 | 0 | 0 | 1 | 0 |
| SUV39H | 0 | 0 | 0 | 0 | 0 | 0 | 0 | 0 | 1 | 0 |
| ARID1B | 0 | 0 | 0 | 0 | 1 | 0 | 0 | 0 | 1 | 0 |
| RFWD2 | 0 | 0 | 0 | 0 | 3 | 0 | 0 | 0 | 1 | 0 |
| KAT2A | 0 | 0 | 0 | 0 | 9 | 0 | 0 | 1 | 1 | 5 |
| CREBBP | 0 | 1 | 0 | 0 | 0 | 0 | 0 | 0 | 0 | 0 |
| TAF5 | 1 | 1 | 0 | 0 | 0 | 0 | 1 | 0 | 0 | 1 |
| CHD1 | 0 | 1 | 0 | 0 | 1 | 0 | 0 | 0 | 0 | 0 |
| TAF5L | 0 | 1 | 0 | 0 | 1 | 0 | 0 | 0 | 0 | 0 |
| TRIM24 | 1 | 1 | 0 | 0 | 1 | 0 | 1 | 0 | 0 | 1 |
| CBX3 | 0 | 0.5 | 0 | 0 | 0 | 0 | 0 | 0 | 0 | 0 |
| CSTF1 | 1 | 0.5 | 0 | 0 | 0 | 0 | 1 | 0 | 0 | 0 |
| SMC1A | 0 | 0.5 | 0 | 0 | 0 | 0 | 1 | 0 | 0 | 0 |
| SMC2 | 1 | 0.5 | 0 | 0 | 0 | 0 | 0.5 | 0 | 0 | 0 |
| SRCAP | 1 | 0.5 | 0 | 0 | 0 | 0 | 1 | 0 | 0 | 0 |
| KDM2B | 0 | 0.5 | 0 | 0 | 1 | 0 | 0.5 | 0 | 0 | 0 |
| RBBP5 | 1 | 0.5 | 0 | 0 | 1 | 0 | 1 | 0 | 0 | 1 |
| WDHD1 | 0 | 0.5 | 0 | 0 | 1 | 0 | 1 | 0 | 0 | 1 |
| TAF3 | 0 | 0.5 | 0 | 0 | 3 | 0 | 0 | 0 | 0 | 0 |
| BRDT | 0 | 0 | 1 | 0 | 0 | 0 | 0 | 0 | 0 | 0 |
| JADE2 | 0 | 0 | 1 | 0 | 0 | 0 | 0 | 0 | 0 | 0 |
| RBBP7 | 0 | 0 | 1 | 0 | 0 | 0 | 0 | 0 | 0 | 0 |
| WHSC1 | 0 | 0 | 1 | 0 | 0 | 0 | 0 | 0 | 0 | 0 |
| BAZ2A | 0 | 0 | 0 | 0 | 0 | 1 | 0 | 0 | 0 | 0 |
| BAZ2B | 0 | 0 | 0 | 0 | 0 | 1 | 0 | 0 | 0 | 0 |
| BRPF1 | 1 | 0 | 0 | 0 | 0 | 1 | 0 | 0 | 0 | 0 |
| CECR2 | 0 | 0 | 0 | 0 | 0 | 1 | 0 | 0 | 0 | 0 |
| EED | 0 | 0 | 0 | 0 | 0 | 1 | 0 | 0 | 0 | 0 |
| KDM4C | 0 | 0 | 0 | 0 | 0 | 1 | 0 | 0 | 0 | 1 |
| KDM5A | 0 | 0 | 0 | 0 | 0 | 1 | 0 | 0 | 0 | 2 |
| KDM5B | 0 | 0 | 0 | 0 | 0 | 1 | 0 | 1 | 0 | 1 |
| KDM6B | 0 | 0 | 0 | 0 | 0 | 1 | 0 | 0 | 0 | 0 |
| PRMT1 | 1 | 0 | 0 | 0 | 0 | 1 | 0 | 0 | 0 | 2 |
| SETD7 | 0 | 0 | 0 | 0 | 0 | 1 | 0 | 0 | 0 | 1 |
| SIRT1 | 0 | 0 | 0 | 0 | 0 | 1 | 0 | 0 | 0 | 0 |
| SIRT2 | 0 | 0 | 0 | 0 | 0 | 1 | 0 | 0 | 0 | 0 |
| SMYD2 | 0 | 0 | 0 | 0 | 0 | 1 | 0 | 0 | 0 | 0 |
| SMYD3 | 0 | 0 | 0 | 0 | 0 | 1 | 0 | 0 | 0 | 0 |
| EHMT2 | 0 | 0 | 0 | 0 | 1 | 1 | 0 | 0 | 0 | 1 |
| EP300 | 1 | 0 | 0 | 0 | 1 | 1 | 0 | 0 | 0 | 0 |
| KDM5D | 0 | 0 | 0 | 0 | 1 | 1 | 0 | 0 | 0 | 1 |
| SMARCA4 | 0 | 0 | 0 | 0 | 2 | 1 | 0 | 0 | 0 | 2 |
| KDM1A | 0 | 0 | 0 | 0 | 10 | 1 | 0 | 0 | 0 | 5 |
| ARID3A | 0 | 0 | 0 | 0 | 0 | 0 | 0 | 0 | 0 | 0 |
| ARID3B | 0 | 0 | 0 | 0 | 0 | 0 | 0 | 0 | 0 | 2 |
| ARID4A | 0 | 0 | 0 | 0 | 0 | 0 | 0 | 0 | 0 | 1 |
| ARID5A | 0 | 0 | 0 | 0 | 0 | 0 | 0 | 0 | 0 | 0 |
| ARID5B | 0 | 0 | 0 | 0 | 0 | 0 | 0 | 0 | 0 | 1 |
| ATAD2 | 0 | 0 | 0 | 0 | 0 | 0 | 0 | 0 | 0 | 0 |
| ATAD2B | 0 | 0 | 0 | 0 | 0 | 0 | 0 | 0 | 0 | 0 |
| BAZ1A | 0 | 0 | 0 | 0 | 0 | 0 | 0 | 0 | 0 | 0 |
| BAZ1B | 0 | 0 | 0 | 0 | 0 | 0 | 0 | 0 | 0 | 0 |
| BRPF3 | 0 | 0 | 0 | 0 | 0 | 0 | 0 | 0 | 0 | 0 |
| BRVVD1 | 0 | 0 | 0 | 0 | 0 | 0 | 0 | 0 | 0 | 1 |
| BRWD3 | 0 | 0 | 0 | 0 | 0 | 0 | 0 | 0 | 0 | 0 |
| CBX2 | 0 | 0 | 0 | 0 | 0 | 0 | 0 | 0 | 0 | 0 |
| CBX4 | 0 | 0 | 0 | 0 | 0 | 0 | 0 | 0 | 0 | 0 |
| CBX5 | 0 | 0 | 0 | 0 | 0 | 0 | 0 | 0 | 0 | 0 |
| CBX6 | 0 | 0 | 0 | 0 | 0 | 0 | 0 | 0 | 0 | 0 |
| CBX7 | 0 | 0 | 0 | 0 | 0 | 0 | 0 | 0 | 0 | 0 |
| CBX8 | 0 | 0 | 0 | 0 | 0 | 0 | 0 | 0 | 0 | 0 |
| CDYL | 0 | 0 | 0 | 0 | 0 | 0 | 0 | 0 | 0 | 0 |
| CDYL2 | 0 | 0 | 0 | 0 | 0 | 0 | 0 | 0 | 0 | 0 |
| CHAF1A | 1 | 0 | 0 | 0 | 0 | 0 | 0 | 0 | 0 | 0 |
| CHD1L | 0 | 0 | 0 | 0 | 0 | 0 | 0 | 0 | 0 | 2 |
| CHD5 | 0 | 0 | 0 | 0 | 0 | 0 | 0 | 0 | 0 | 0 |
| CHD9 | 0 | 0 | 0 | 0 | 0 | 0 | 0 | 0 | 0 | 0 |
| DDB2 | 0 | 0 | 0 | 0 | 0 | 0 | 0 | 0 | 0 | 0 |
| DNMT3A | 0 | 0 | 0 | 0 | 0 | 0 | 0 | 0 | 0 | 1 |
| DPF1 | 0 | 0 | 0 | 0 | 0 | 0 | 0 | 0 | 0 | 1 |
| DPF2 | 0 | 0 | 0 | 0 | 0 | 0 | 0 | 0 | 0 | 0 |
| DPF3 | 0 | 0 | 0 | 0 | 0 | 0 | 0 | 0 | 0 | 0 |
| ELP2 | 0 | 0 | 0 | 0 | 0 | 0 | 0 | 0 | 0 | 0 |
| ELP3 | 0 | 0 | 0 | 0 | 0 | 0 | 0 | 0 | 0 | 0 |
| EPC1 | 0 | 0 | 0 | 0 | 0 | 0 | 0 | 0 | 0 | 0 |
| EPC2 | 0 | 0 | 0 | 0 | 0 | 0 | 0 | 0 | 0 | 0 |
| EZH1 | 0 | 0 | 0 | 0 | 0 | 0 | 0 | 1 | 0 | 1 |
| FBXL19 | 0 | 0 | 0 | 0 | 0 | 0 | 0 | 0 | 0 | 0 |
| FBXW9 | 0 | 0 | 0 | 0 | 0 | 0 | 0 | 0 | 0 | 0 |
| GTF3C2 | 1 | 0 | 0 | 0 | 0 | 0 | 1 | 0 | 0 | 0 |
| HAT1 | 0 | 0 | 0 | 0 | 0 | 0 | 0 | 0 | 0 | 0 |
| HDAC11 | 0 | 0 | 0 | 0 | 0 | 0 | 0 | 0 | 0 | 0 |
| HDAC4 | 0 | 0 | 0 | 0 | 0 | 0 | 0 | 0 | 0 | 0 |
| HDAC7 | 0 | 0 | 0 | 0 | 0 | 0 | 0 | 0 | 0 | 1 |
| HDAC9 | 0 | 0 | 0 | 0 | 0 | 0 | 0 | 0 | 0 | 0 |
| HR | 0 | 0 | 0 | 0 | 0 | 0 | 0 | 0 | 0 | 0 |
| IL4I1 | 0 | 0 | 0 | 0 | 0 | 0 | 0 | 0 | 0 | 0 |
| ING3 | 0 | 0 | 0 | 0 | 0 | 0 | 0 | 0 | 0 | 0 |
| JADE1 | 0 | 0 | 0 | 0 | 0 | 0 | 0 | 0 | 0 | 0 |
| JADE3 | 0 | 0 | 0 | 0 | 0 | 0 | 0 | 0 | 0 | 0 |
| JARID2 | 0 | 0 | 0 | 0 | 0 | 0 | 0 | 0 | 0 | 0 |
| JMJD1C | 0 | 0 | 0 | 0 | 0 | 0 | 0 | 0 | 0 | 1 |
| JMJD4 | 0 | 0 | 0 | 0 | 0 | 0 | 0 | 0 | 0 | 0 |
| JMJD6 | 0 | 0 | 0 | 0 | 0 | 0 | 0 | 0 | 0 | 0 |
| KAT8 | 1 | 0 | 0 | 0 | 0 | 0 | 0 | 0 | 0 | 0 |
| KDM1B | 0 | 0 | 0 | 0 | 0 | 0 | 0 | 0 | 0 | 2 |
| KDM7A | 0 | 0 | 0 | 0 | 0 | 0 | 0 | 0 | 0 | 0 |
| KMT2C | 0 | 0 | 0 | 0 | 0 | 0 | 0 | 0 | 0 | 3 |
| MBD1 | 0 | 0 | 0 | 0 | 0 | 0 | 0 | 1 | 0 | 0 |
| MBD3L1 | 0 | 0 | 0 | 0 | 0 | 0 | 0 | 0 | 0 | 0 |
| MBD4 | 0 | 0 | 0 | 0 | 0 | 0 | 0 | 0 | 0 | 0 |
| METTL13 | 0 | 0 | 0 | 0 | 0 | 0 | 0 | 0 | 0 | 0 |
| MGMT | 0 | 0 | 0 | 0 | 0 | 0 | 0 | 0 | 0 | 0 |
| MSL3 | 0 | 0 | 0 | 0 | 0 | 0 | 0 | 0 | 0 | 0 |
| MTA3 | 0 | 0 | 0 | 0 | 0 | 0 | 0 | 0 | 0 | 0 |
| MTF2 | 0 | 0 | 0 | 0 | 0 | 0 | 0 | 0 | 0 | 0 |
| PHF1 | 0 | 0 | 0 | 0 | 0 | 0 | 0 | 0 | 0 | 0 |
| PHF10 | 0 | 0 | 0 | 0 | 0 | 0 | 0 | 0 | 0 | 0 |
| PHF13 | 0 | 0 | 0 | 0 | 0 | 0 | 0 | 0 | 0 | 0 |
| PHF14 | 0 | 0 | 0 | 0 | 0 | 0 | 0 | 0 | 0 | 0 |
| PHF19 | 0 | 0 | 0 | 0 | 0 | 0 | 0 | 0 | 0 | 0 |
| PHF2 | 0 | 0 | 0 | 0 | 0 | 0 | 0 | 0 | 0 | 0 |
| PHF21A | 0 | 0 | 0 | 0 | 0 | 0 | 0 | 0 | 0 | 0 |
| PHF21B | 0 | 0 | 0 | 0 | 0 | 0 | 0 | 0 | 0 | 1 |
| PHF3 | 0 | 0 | 0 | 0 | 0 | 0 | 0 | 0 | 0 | 0 |
| PHF8 | 0 | 0 | 0 | 0 | 0 | 0 | 0 | 0 | 0 | 0 |
| PHRF1 | 0 | 0 | 0 | 0 | 0 | 0 | 0 | 0 | 0 | 1 |
| PRPM11 | 0 | 0 | 0 | 0 | 0 | 0 | 0 | 0 | 0 | 1 |
| PRDM14 | 0 | 0 | 0 | 0 | 0 | 0 | 0 | 0 | 0 | 0 |
| PRDM2 | 0 | 0 | 0 | 0 | 0 | 0 | 0 | 0 | 0 | 1 |
| PRDM9 | 0 | 0 | 0 | 0 | 0 | 0 | 0 | 0 | 0 | 0 |
| PRMT2 | 0 | 0 | 0 | 0 | 0 | 0 | 0 | 0 | 0 | 0 |
| PYGO1 | 0 | 0 | 0 | 0 | 0 | 0 | 0 | 0 | 0 | 0 |
| PYGO2 | 0 | 0 | 0 | 0 | 0 | 0 | 0 | 0 | 0 | 1 |
| RAD54L | 0 | 0 | 0 | 0 | 0 | 0 | 0 | 0 | 0 | 0 |
| RAD54L2 | 0 | 0 | 0 | 0 | 0 | 0 | 0 | 0 | 0 | 0 |
| RBBP4 | 1 | 0 | 0 | 0 | 0 | 0 | 0 | 0 | 0 | 0 |
| RSF1 | 0 | 0 | 0 | 0 | 0 | 0 | 0 | 0 | 0 | 0 |
| SAP30 | 0 | 0 | 0 | 0 | 0 | 0 | 0 | 0 | 0 | 0 |
| SAP30L | 0 | 0 | 0 | 0 | 0 | 0 | 0 | 0 | 0 | 0 |
| SET | 0 | 0 | 0 | 0 | 0 | 0 | 0 | 0 | 0 | 0 |
| SETD4 | 0 | 0 | 0 | 0 | 0 | 0 | 0 | 0 | 0 | 2 |
| SETD9 | 0 | 0 | 0 | 0 | 0 | 0 | 0 | 0 | 0 | 0 |
| SETDB2 | 0 | 0 | 0 | 0 | 0 | 0 | 0 | 0 | 0 | 0 |
| SHPRH | 0 | 0 | 0 | 0 | 0 | 0 | 0 | 0 | 0 | 0 |
| SIRT3 | 0 | 0 | 0 | 0 | 0 | 0 | 0 | 0 | 0 | 0 |
| SIRT5 | 0 | 0 | 0 | 0 | 0 | 0 | 0 | 0 | 0 | 0 |
| SLBP | 0 | 0 | 0 | 0 | 0 | 0 | 0 | 0 | 0 | 0 |
| SMARCA1 | 0 | 0 | 0 | 0 | 0 | 0 | 0 | 0 | 0 | 0 |
| SMARCA2 | 0 | 0 | 0 | 0 | 0 | 0 | 0 | 0 | 0 | 0 |
| SMARCAD1 | 0 | 0 | 0 | 0 | 0 | 0 | 0 | 0 | 0 | 0 |
| SMC1B | 0 | 0 | 0 | 0 | 0 | 0 | 0 | 0 | 0 | 0 |
| SMC3 | 1 | 0 | 0 | 0 | 0 | 0 | 0.5 | 0 | 0 | 0 |
| SMC4 | 1 | 0 | 0 | 0 | 0 | 0 | 0 | 0 | 0 | 0 |
| SMYD1 | 0 | 0 | 0 | 0 | 0 | 0 | 0 | 0 | 0 | 0 |
| SMYD4 | 0 | 0 | 0 | 0 | 0 | 0 | 0 | 0 | 0 | 0 |
| SMYD5 | 0 | 0 | 0 | 0 | 0 | 0 | 0 | 0 | 0 | 0 |
| SP100 | 0 | 0 | 0 | 0 | 0 | 0 | 0 | 0 | 0 | 0 |
| SP140 | 0 | 0 | 0 | 0 | 0 | 0 | 0 | 0 | 0 | 0 |
| SP140L | 0 | 0 | 0 | 0 | 0 | 0 | 0 | 0 | 0 | 0 |
| TAF1L | 0 | 0 | 0 | 0 | 0 | 0 | 0 | 1 | 0 | 0 |
| TET1 | 0 | 0 | 0 | 0 | 0 | 0 | 0 | 0 | 0 | 0 |
| TET2 | 0 | 0 | 0 | 0 | 0 | 0 | 0 | 0 | 0 | 0 |
| TET3 | 0 | 0 | 0 | 0 | 0 | 0 | 0 | 0 | 0 | 0 |
| TRIM28 | 0 | 0 | 0 | 0 | 0 | 0 | 0 | 0 | 0 | 0 |
| TRIM33 | 0 | 0 | 0 | 0 | 0 | 0 | 0 | 0 | 0 | 0 |
| TRIM66 | 0 | 0 | 0 | 0 | 0 | 0 | 0 | 0 | 0 | 0 |
| TSPYL2 | 0 | 0 | 0 | 0 | 0 | 0 | 0 | 0 | 0 | 0 |
| UHRF1 | 0 | 0 | 0 | 0 | 0 | 0 | 0 | 0 | 0 | 0 |
| UHRF2 | 0 | 0 | 0 | 0 | 0 | 0 | 0 | 0 | 0 | 0 |
| UTY | 0 | 0 | 0 | 0 | 0 | 0 | 0 | 0 | 0 | 0 |
| WDR48 | 0 | 0 | 0 | 0 | 0 | 0 | 0 | 0 | 0 | 0 |
| WDR5 | 0 | 0 | 0 | 0 | 0 | 0 | 0 | 0 | 0 | 0 |
| WDR61 | 0 | 0 | 0 | 0 | 0 | 0 | 1 | 0 | 0 | 1 |
| WDR82 | 0 | 0 | 0 | 0 | 0 | 0 | 0 | 0 | 0 | 0 |
| WDR92 | 0 | 0 | 0 | 0 | 0 | 0 | 0.5 | 0 | 0 | 0 |
| WHSC1L1 | 0 | 0 | 0 | 0 | 0 | 0 | 0 | 0 | 0 | 0 |
| ZMYND11 | 0 | 0 | 0 | 0 | 0 | 0 | 0 | 1 | 0 | 0 |
| AIRE | 0 | 0 | 0 | 0 | 1 | 0 | 0 | 0 | 0 | 1 |
| CARM1 | 0 | 0 | 0 | 0 | 1 | 0 | 0 | 0 | 0 | 0 |
| CXXC1 | 0 | 0 | 0 | 0 | 1 | 0 | 0 | 0 | 0 | 0 |
| EP400 | 1 | 0 | 0 | 0 | 1 | 0 | 0 | 0 | 0 | 0 |
| HDAC10 | 0 | 0 | 0 | 0 | 1 | 0 | 0 | 0 | 0 | 2 |
| HUWE1 | 0 | 0 | 0 | 0 | 1 | 0 | 0.5 | 0 | 0 | 0 |
| KDM2A | 0 | 0 | 0 | 0 | 1 | 0 | 0 | 0 | 0 | 3 |
| MBD2 | 0 | 0 | 0 | 0 | 1 | 0 | 0 | 0 | 0 | 0 |
| MEAF6 | 0 | 0 | 0 | 0 | 1 | 0 | 0 | 0 | 0 | 0 |
| PBRM1 | 0 | 0 | 0 | 0 | 1 | 0 | 0 | 0 | 0 | 0 |
| PHIP | 0 | 0 | 0 | 0 | 1 | 0 | 0 | 0 | 0 | 0 |
| SIRT7 | 0 | 0 | 0 | 0 | 1 | 0 | 0 | 0 | 0 | 0 |
| ZMYND8 | 0 | 0 | 0 | 0 | 1 | 0 | 0 | 0 | 0 | 0 |
| KAT7 | 0 | 0 | 0 | 0 | 2 | 0 | 0 | 0 | 0 | 2 |
| MBD3 | 0 | 0 | 0 | 0 | 2 | 0 | 0 | 0 | 0 | 1 |
| MTA2 | 0 | 0 | 0 | 0 | 2 | 0 | 0 | 0 | 0 | 0 |
| SETD5 | 1 | 0 | 0 | 0 | 2 | 0 | 0 | 0 | 0 | 0 |
| WBSCR22 | 0 | 0 | 0 | 0 | 2 | 0 | 0 | 0 | 0 | 0 |
| SETD1B | 0 | 0 | 0 | 0 | 6 | 0 | 0 | 0 | 0 | 1 |

The screening platform also allows for identifying synthetic combinations that are more lethal in comparison to the others. As described herein, the 300K screen utilizes the platform described in FIG. 1. The 300K Library Screen targets 274 chromatin regulator genes (DNMT1, KDM5A, KDM5B, KDM5C, KDM5D, SETDB1, SETDB2, BAZ2A, BAZ2B, ASH1L, KMT2A, KMT2B, SUV39H1, SUV39H2, JARID2, KAT2A, KAT2B, CHD3, CHD4, CHD5, CHAF1A, ZMYND8, BRPF1, BRPF3, BRD1, MBD2, MBD3, MBD1, HDAC4, HDAC5, HDAC9, BRWD1, BRWD3, KDM2A, PHIP, PBRM1, CXXC1, SETMAR, EHMT1, EHMT2, ATAD2, ATAD2B, KMT2C, KMT2D, KMT2E, MGMT, WBSCR22, CARM1, KDM4A, KDM4B, KDM4C, KDM4D, KDM4E, ARID4A, ARID4B, PHF2, PHF8, SP140L, BPTF, BAZ1A, BAZ1B, KDM7A, TRIM24, TRIM33, TRIM66, KAT5, KAT6A, KAT6B, KATE, CHD1, CHD2, CHD6, CHD7, CHD8, CHD9, SMARCA2, SMARCA4, SMARCA1, SMARCA5, EPC1, EPC2, KDM1A, KDM1B, DNMT3A, DNMT3B, WHSC1, WHSC1L1, NSD1, ZMYND11, SHPRH, MBD4, MBD3L1, MBD3L2, MECP2, ASF1A, ASF1B, ELP3, ING1, ING2, ING3, ING4, ING5, SLBP, SAP30L, SAP30, HAT1, HDAC1, HDAC10, HDAC11, HDAC2, HDAC3, HDAC6, HDAC7, HDAC8, DOT1L, MEAF6, FBXW9, FBXL19, TAF5L, TAF5, WDHD1, WDR48, WDR5, WDR61, WDR77, WDR82, WDR92, CHAF1B, CSTF1, CORO2A, DDB2, ELP2, EED, GTF3C2, HIRA, KDM2B, MTA2, MTA3, MTA1, RBBP4, RBBP5, RBBP7, RFWD2, TET1, TET3, CBX1, CBX2, CBX3, CBX4, CBX5, CBX6, CBX7, CBX8, CDYL2, CDYL, CDY1, CDY1B, CDY2A, CDY2B, SIRT1, SIRT2, SIRT3, SIRT4, SIRT5, SIRT6, SIRT7, SMC1A, SMC1B, SMC2, SMC3, SMC4, PRDM1, PRDM11, PRDM14, PRDM16, PRDM2, PRDM6, PRDM9, SMYD1, SMYD2, SMYD3, SMYD4, SETD1A, SETD1B, SETD2, SETD3, SETD4, SETD5, SETD6, SETD9, SETD7, SMYD5, EZH1, EZH2, ARID1A, ARID1B, ARID2, ARID3A, ARID3B, ARID3C, ARID5A, ARID5B, CREBBP, EP300, SP100, SP140, TAF1L, TAF1, BRD2, BRD3, BRD4, BRD7, BRD8, BRD9, BRDT, CECR2, HR, JMJD1C, JMJD4, JMJD6, KDM3A, KDM3B, KDM6A, KDM6B, UTY, PHRF1, PHF1, PHF10, PHF12, PHF13, PHF14, PHF19, PHF21A, PHF21B, PHF23, PHF3, TAF3, AIRE, DIDO1, DPF1, DPF2, DPF3, INTS12, KAT7, MSL3, MTF2, METTL13, MORF4L1, PRMT1, PRMT2, PRMT5, PYGO1, PYGO2, RSF1, TRIM28, UHRF1, UHRF2, EP400, INO80, RAD54L, RAD54L2, SET, SMARCAL1, SMARCB1, SMARCAD1, SRCAP, TBP, TSPYL2, ATRX, CHD1L, IL4I1, JADE1, JADE2 and JADE3). The screen includes 2 sgRNAs per gene for each CRISPR enzyme ortholog (Sa: SEQ ID NOS: 9-348 and 353-548; Sp: SEQ ID NOS: 561-900 and 905-1100). The screen includes 14 non-targeting sgRNAs for each ortholog (Sa: SEQ ID NOS: 2-8, 350-352 and 549-552; Sp: SEQ ID NOS: 554-560, 902-904 and 1101-1104), 2 EEF2 sgRNAs for each ortholog (Sa: SEQ ID NOS: 1 and 349; Sp: SEQ ID NOS: 553 and 901). The screen thus provides for 552 (S. aureus)×552 (S. pyogenes) sgRNAs=304,704 perturbations.

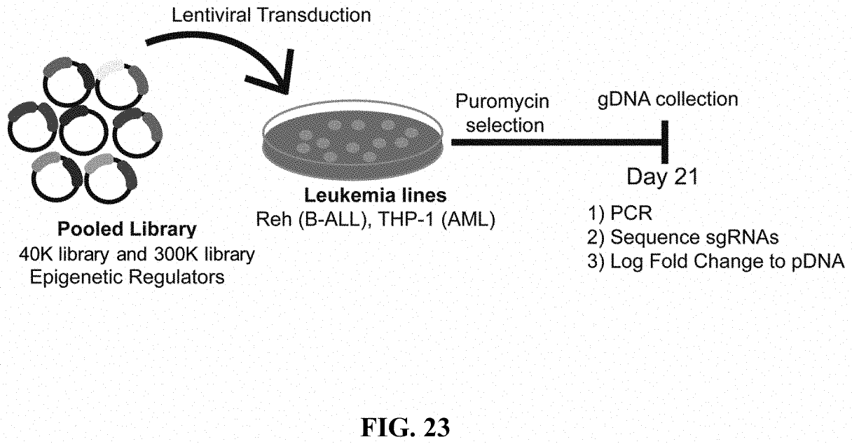

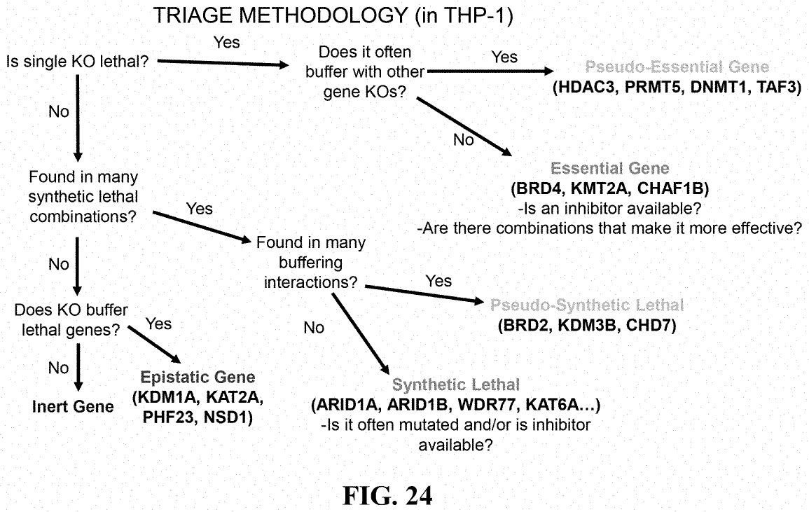

FIG. 23 shows a schematic of disease relevant screening in leukemia. Libraries targeting different epigenetic regulators (e.g., 40K library or 300K library) is transduced into a population of cells, such as REH or THP-1, cells transduced with the vectors are selected for with puromycin, genomic DNA is collected after day 21, the guide sequence cassettes are amplified by PCR, the sgRNAs sequenced, and the fold change as compared to the pool DNA is determined. FIG. 24 shows a flow diagram for selecting synthetic lethal genes and the top therapeutic actionable targets. This methodology can be performed using an algorithm to analyze data from large screens.

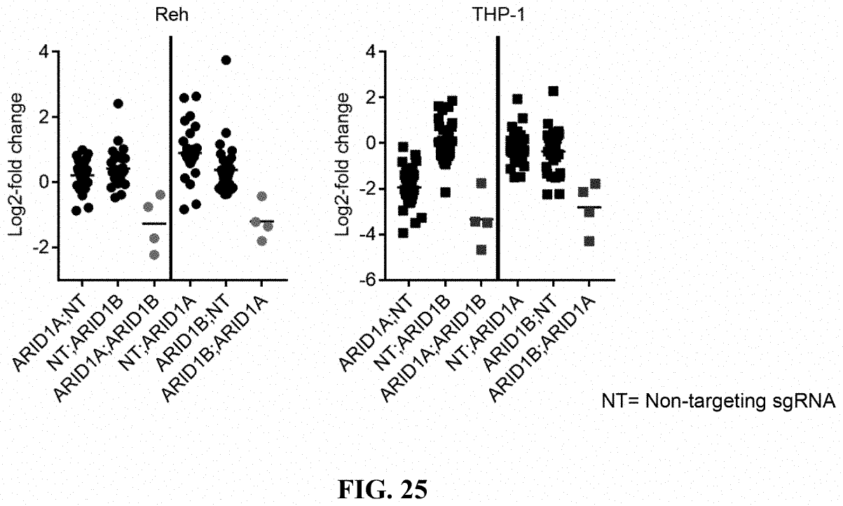

FIG. 25 shows results of the screening methodology for synthetic lethal combinations identified (ARID1A;ARID1B). Shown are each gene paired with a non-targeting sgRNA in both orthologous CRISPR enzyme orientations, as well as the combination of genes in both orthologous CRISPR enzyme orientations. Thus, the targeting by either orthologous CRISPR enzymes does not make a significant difference. The screening was performed in REH and THP-1 cell lines. In all combinations and in both cell lines there is a decrease in the sgRNA combinations as compared to the pooled library. Other examples of synthetic combinations identified were ASF1A;ASF1B, SMARCAL1;ATRX, ING4;ING5, and HDAC1;HDAC2.

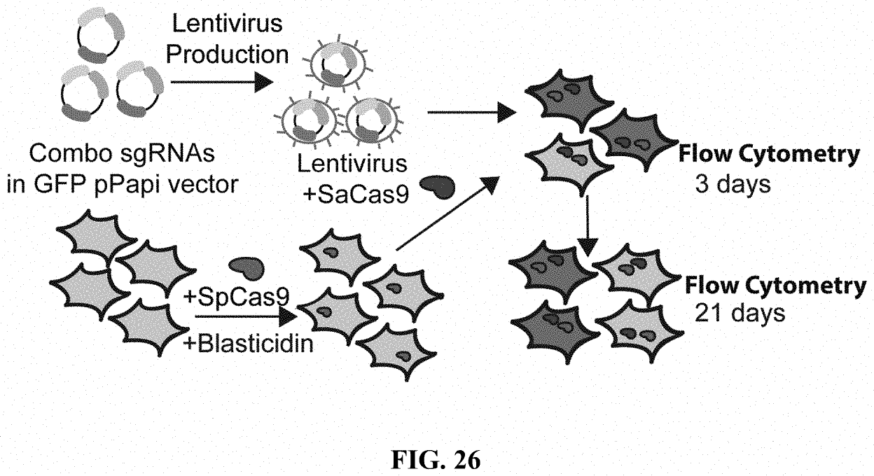

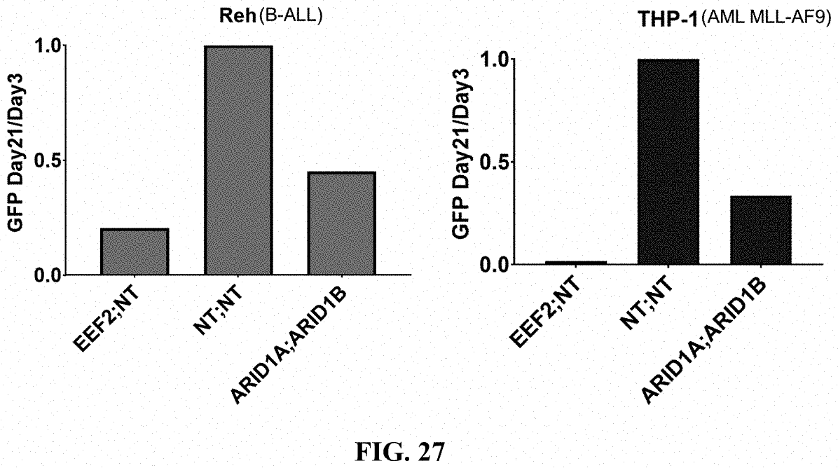

FIG. 26 shows a flow diagram for follow-up validation of identified combinations of guide sequences. The vector for combinatorial screening further includes a sequence encoding for GFP. Each combination of guide sequences can be validated individually. The vector can also include a sequence encoding for SaCas9. A population of cells are transduced or provided with SpCas9. Cells expressing SpCas9 can be selected for (e.g., Blasticidin). The selected cells are transduced with the vector encoding the combination for validation. GFP positive cells are quantitated at specific time points of interest (e.g., 3 days, and 21 days). FIG. 27 shows validation experiments for the ARID1A;ARID1B combination in REH and THP-1 cells (EEF2 is an essential gene control).

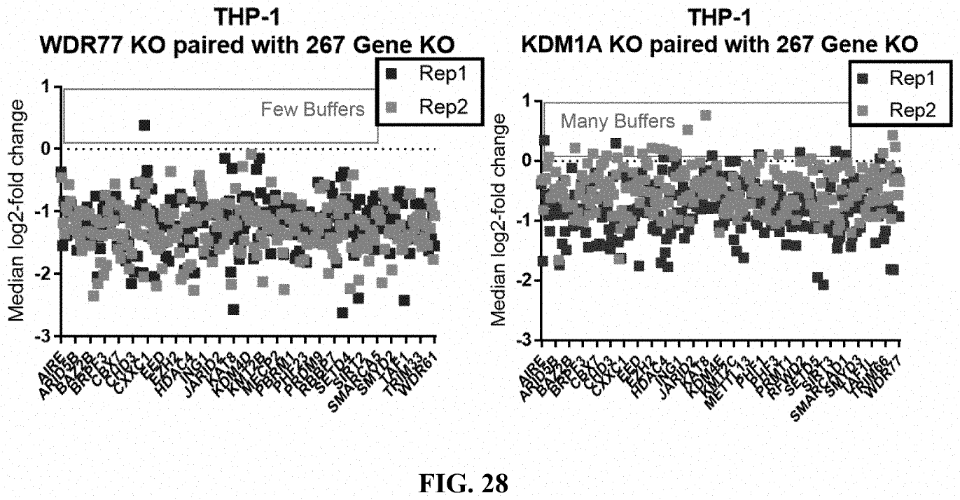

FIG. 28 shows an example of a good synthetic lethal gene and an epistatic gene paired with 267 gene knock outs in THP-1 cells (selected genes are indicated). Synthetic lethal genes rarely buffer, while, epistatic genes have many buffers. This approach may be used to further screen genes identified as synthetic lethal genes for the amount of buffering when the single knockout is paired with a large number of other gene knockouts.

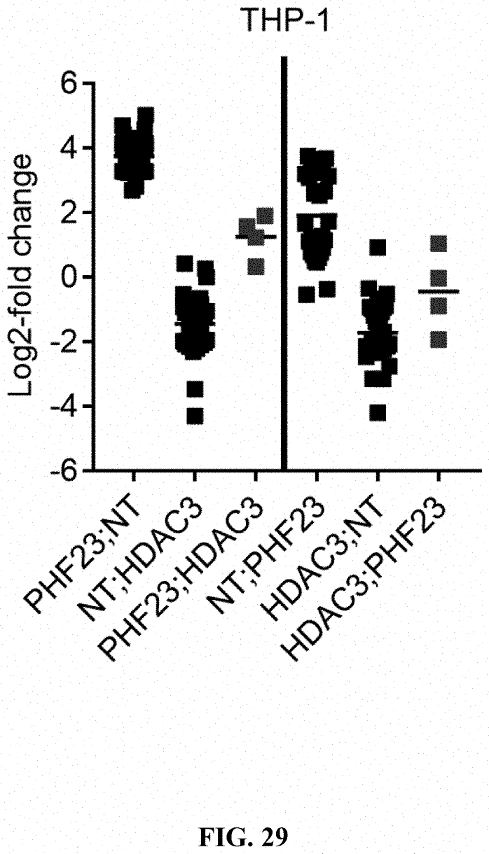

FIG. 29 shows an example of a pseudo-essential gene and buffering in THP-1 cells. HDAC3 is essential in certain backgrounds, but is not essential when knocked out in combination with the epistatic gene PHF23. Other examples showing buffering include BRD2;CHD1, BRD2;MTA2, and HDAC3;NSD1.

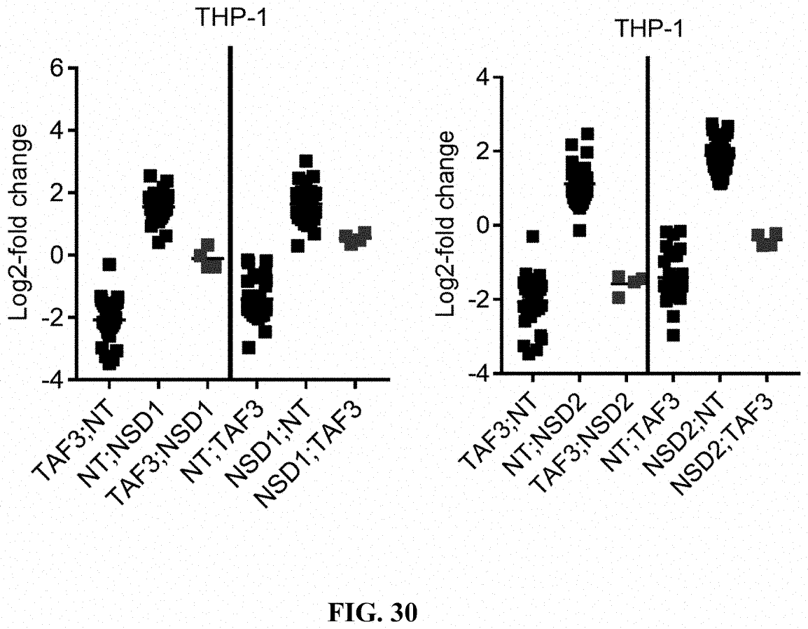

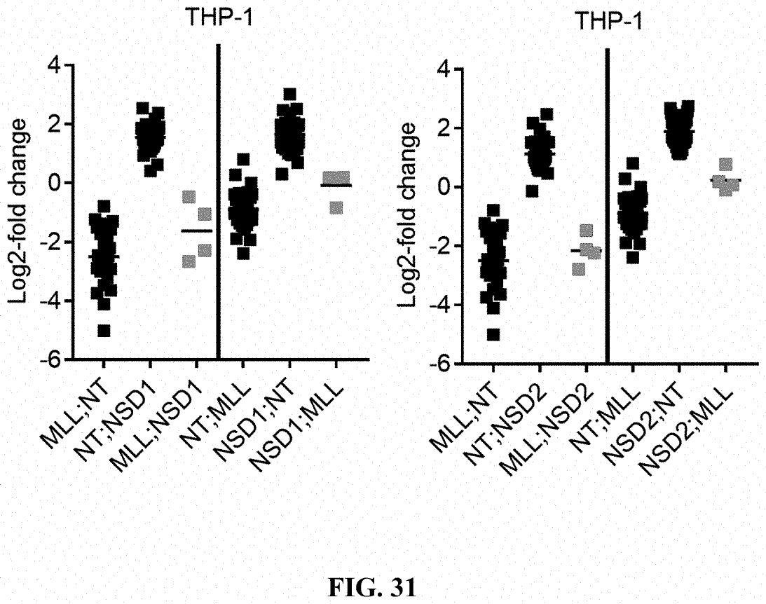

FIG. 30 shows examples of pseudo-essential genes and buffering in THP-1 cells. TAF3 is essential in certain backgrounds, but is not essential when knocked out in combination with NSD1/2. NSD1/2 are methyltransferases that generate H3K36mel/2 (monomethyl and demethylation) and recruit repressive complexes. FIG. 31 illustrates buffering as MLL knockout is partially rescued by NSD1/2.

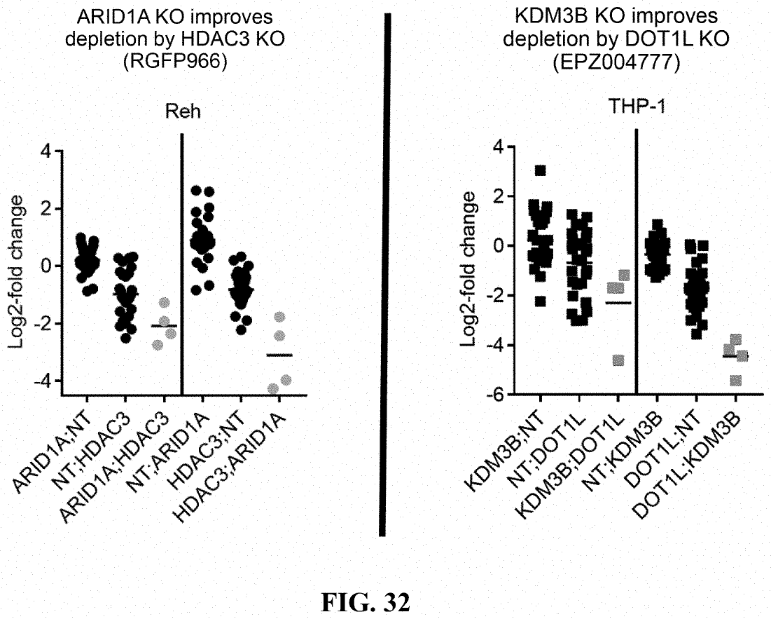

FIG. 32 shows that candidate therapeutic targets were identified that can be used in a combination therapy to improve and/or predict response to existing drugs. For example, ARID1A knock out improves depletion by HDAC3 knockout. HDAC3 inhibitors are known in the art (e.g., RGFP966). KDM3B knock out improves depletion by DOT1L knockout. DOT1L inhibitors are known in the art (e.g., EPZ004777).

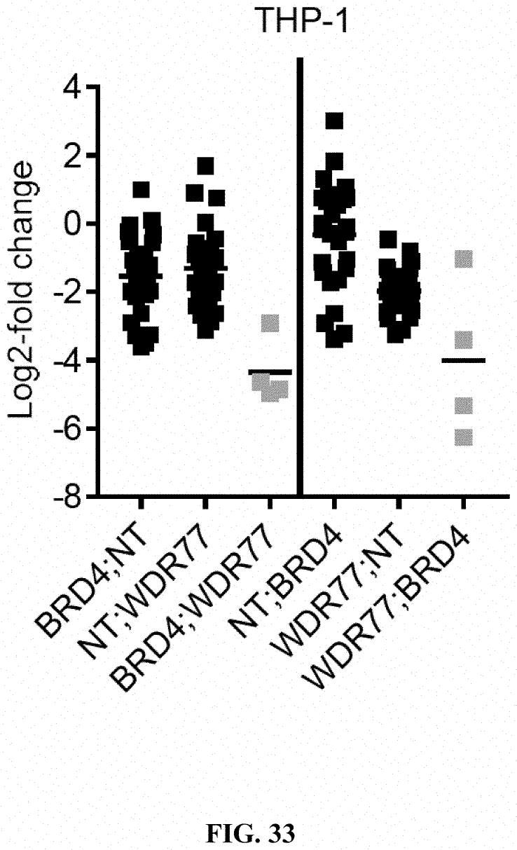

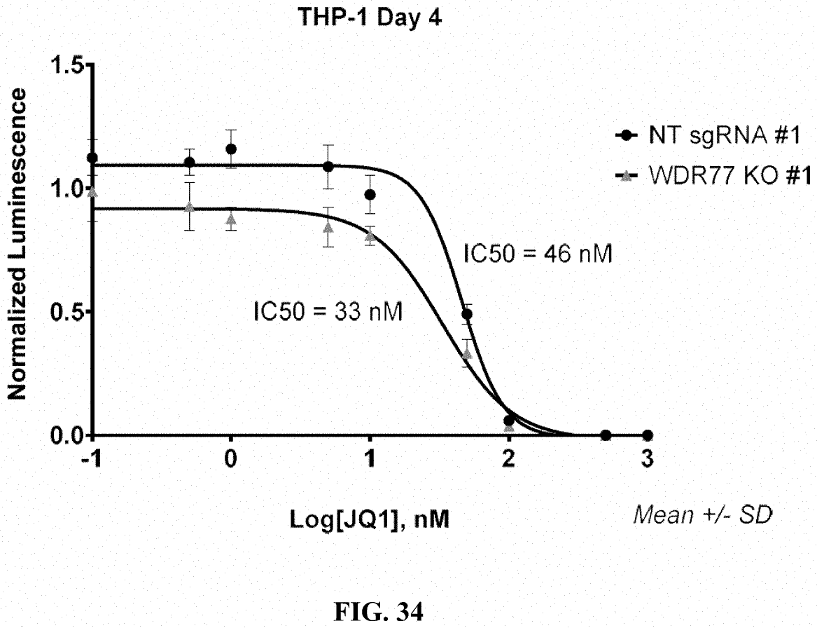

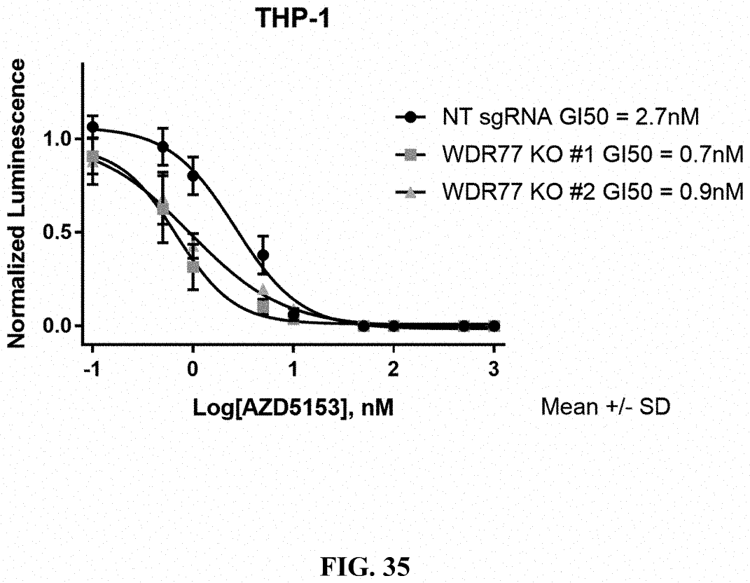

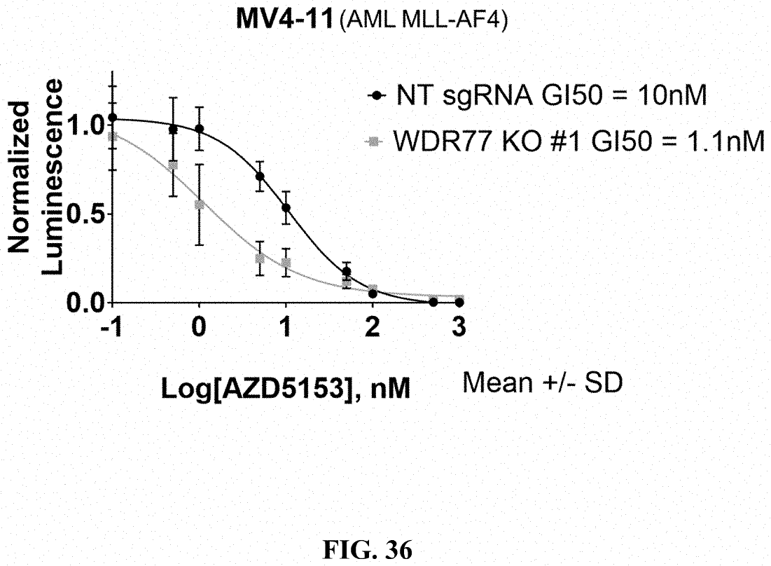

FIG. 33 shows that candidate therapeutic targets were identified that can be used in a combination therapy to improve and/or predict response to existing drugs. For example, WDR77 knock out improves depletion by BRD4 knockout. WDR77 is involved in repressive chromatin complexes (see, e.g., Migliori et al., Nat Struct Mol Biol. 2012 Jan. 8; 19(2):136-44). BRD4 inhibitors are known in the art (e.g., AZD5153, JQ1). FIG. 34 shows JQ1 dose response curves in THP-1 cells that are +/−WDR77 knock-out. The experiments were performed in octuplicate wells and repeated with two sgRNAs. FIGS. 35 and 36 show AZD5153 dose response curves in THP-1 and MV4-11 cells that are +/−WDR77 knock-out.

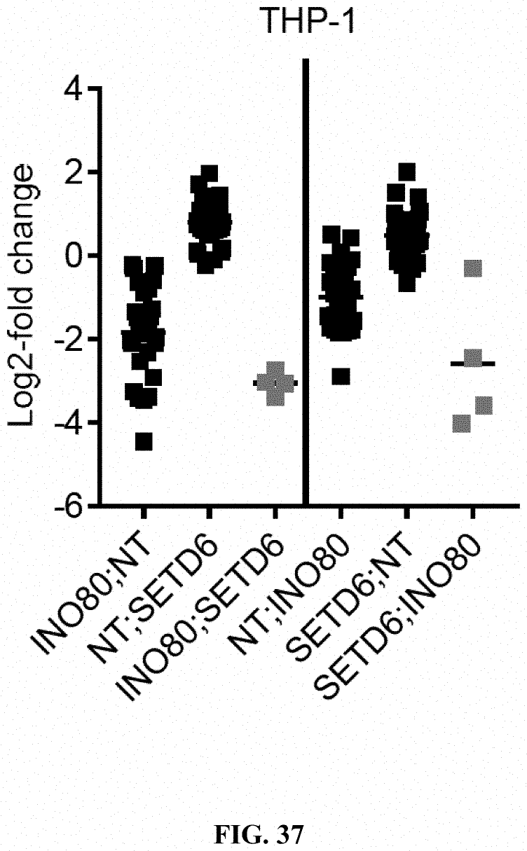

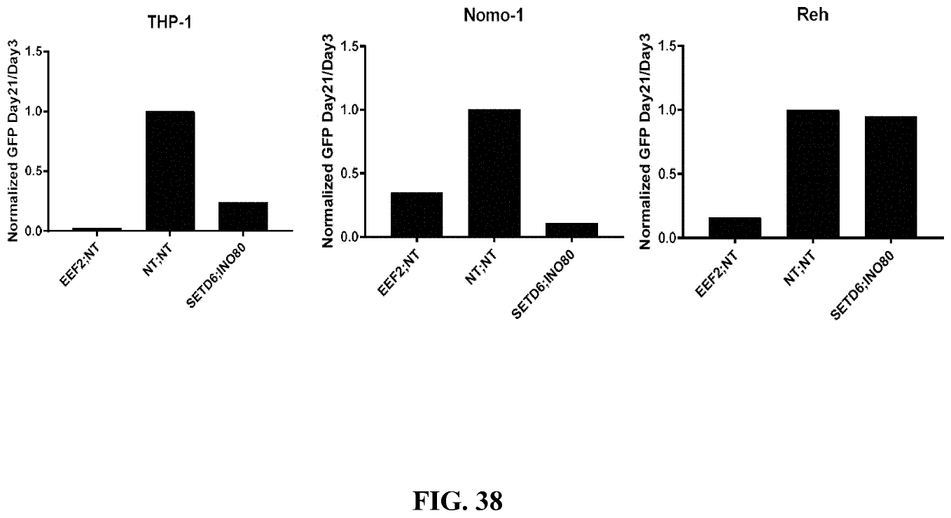

FIG. 37 shows that candidate therapeutic targets were identified that can be used in a combination therapy to improve and/or predict response to existing drugs. For example, SETD6 knock out improves depletion by INO80 knockout. FIG. 38 shows the results of follow-up experiments using the cell lines THP-1 and Nomo-1 (MLL-AF9 fusion AML) and REH (no MLL fusion). Cells were transduced with combo CRISPR GFP lentivirus and fluorescence analyzed at two time points (NT=non-targeting guide; EEF2 is an essential gene). SETD6 and INO80 may be suppressing transcription. Histone H2A.Z is found at active and poised promoters and are antagonized by both SETD6 and INO80, thus requiring both for the lethality observed (see, e.g., Surface et al., 2016, Cell Reports 14, 1142-1155; Subramanian, Fields, Boyer, F1000Prime Rep2015, 7:01; Brahma et al., 2017, Nature Communications volume 8, Article number: 15616; and Binda et al., Epigenetics. 2013 Feb. 1; 8(2): 177-183). Without the suppressors, the MLL fusion protein may bind to many sites in the genome resulting in death or differentiation.

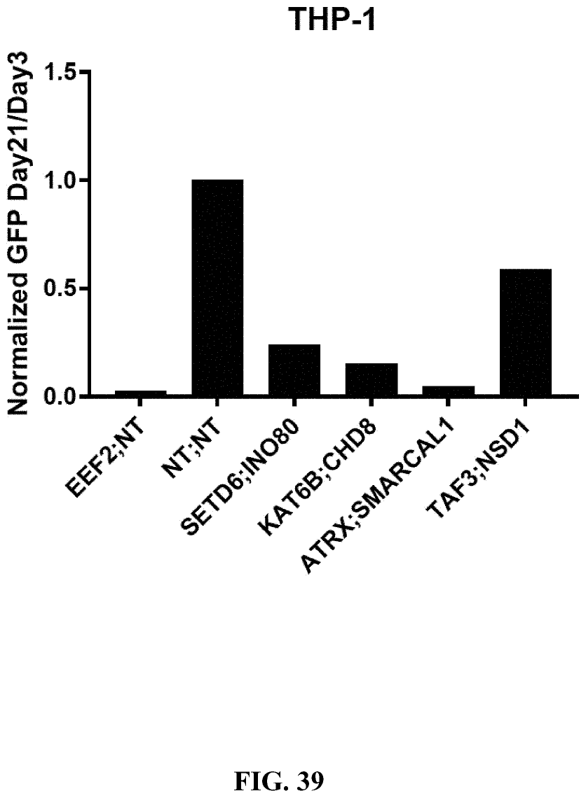

FIG. 39 shows follow-up validation experiments where cells were transduced with combo CRISPR GFP lentivirus for the indicated combinations and fluorescence analyzed at two time points. The experiment also shows the synthetic lethality of the combinations of SETD6;INO80, KAT6B;CHD8, and ATRX;SMARCAL1.

FIG. 40 shows that PHF23 knockout buffers TAF3 essentiality. PHF23 and NSD1 each contain PHD domains that target highly expressed regions in the genome and suppress them. This suppression may induce TAF3 essentiality.

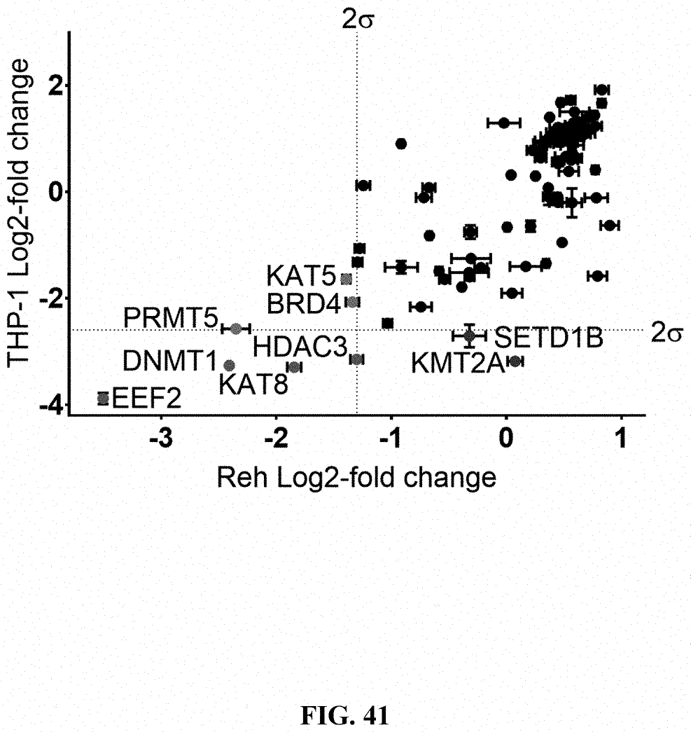

FIG. 41 shows essential genes in REH and THP-1 cells using a singleton gene knockout data library screen.

In summary, Applicants have shown that the screening platforms disclosed are a powerful tool for determining complex genetic interactions (e.g., epigenetic interactions). Applicants have validated a number of known synthetic lethal combinations including ARID1A;ARID1B and have discovered novel synthetic lethal interactions. Applicants also discovered reversible interactions (e.g., NSD1/2). Finally, Applicants findings inform small molecule treatments (e.g., BRD4 inhibitors; WDR77) and this was confirmed in multiple cell lines.

### Example 11—Combinatorial Screening of Chromatin Regulators in Cancer Using a Pi z-Score

Applicants have further used the combinatorial screening methods in additional cancer cell lines and have identified synthetic lethal interactions using a statistical analysis approach. Applicants have identified additional interactions that are applicable for therapeutic use in cancer subjects. The methods identified high confidence interactions.

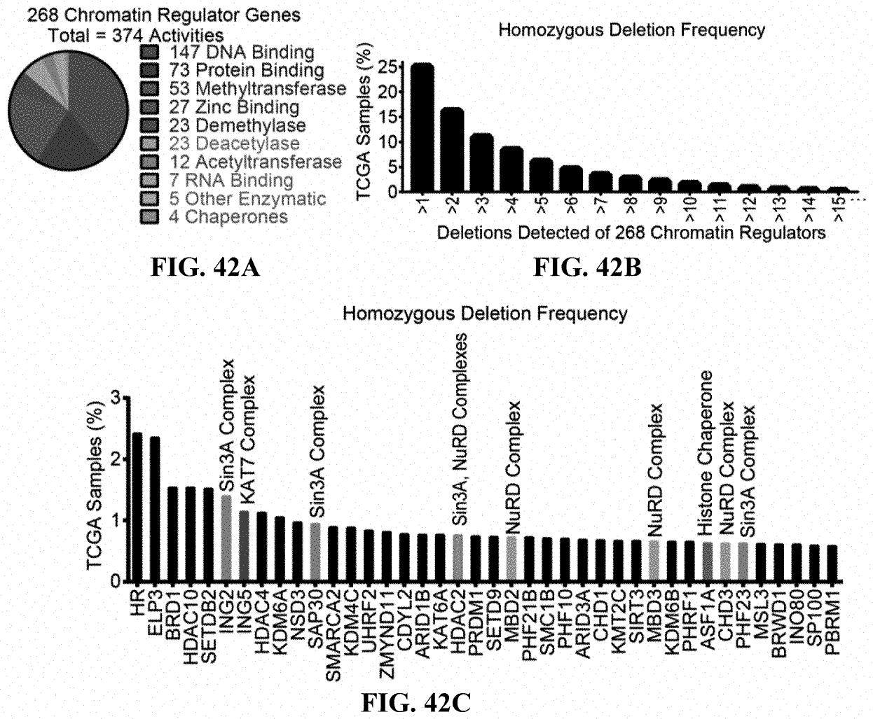

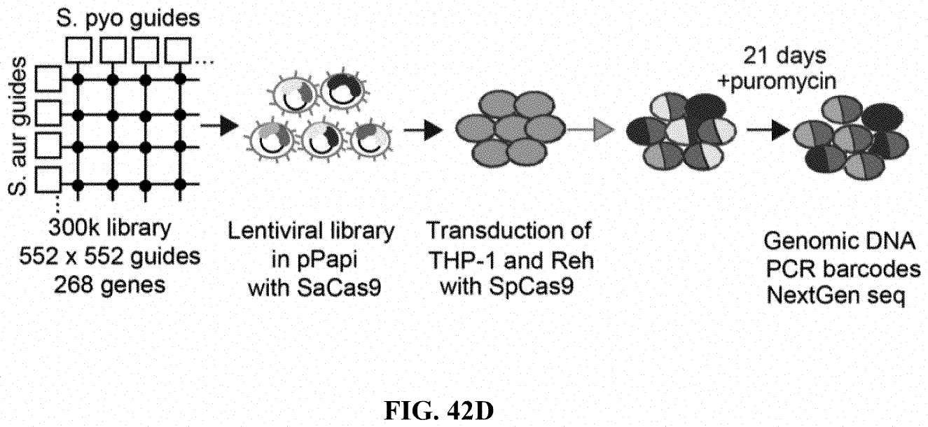

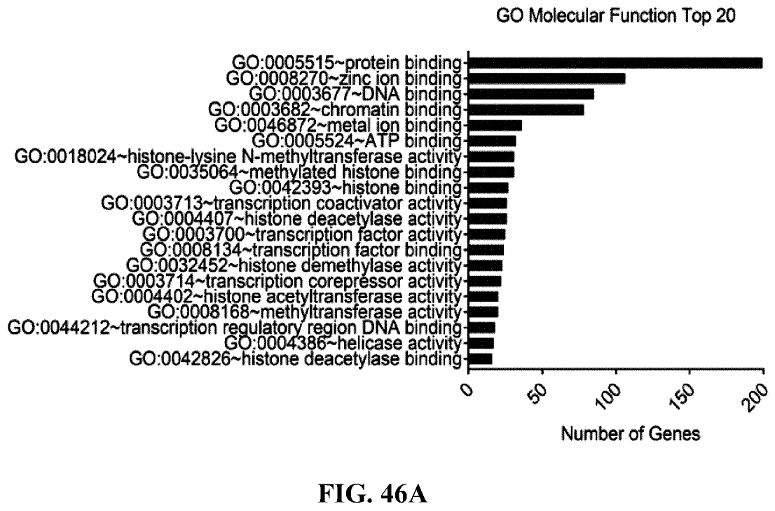

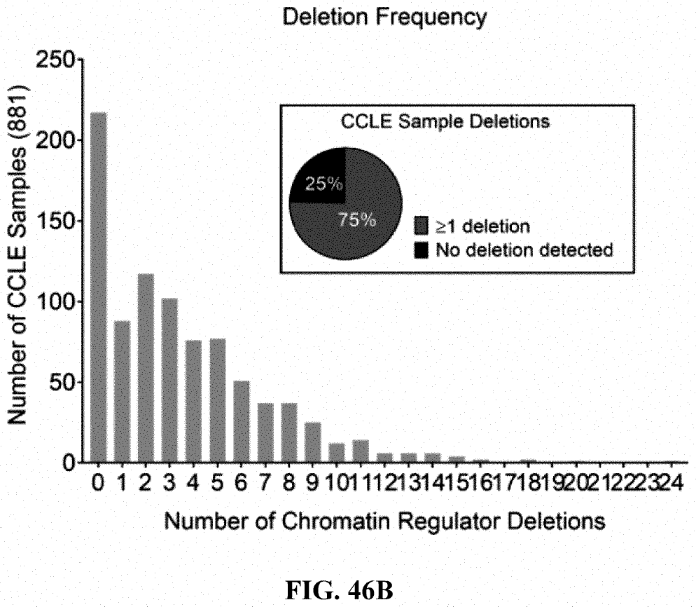

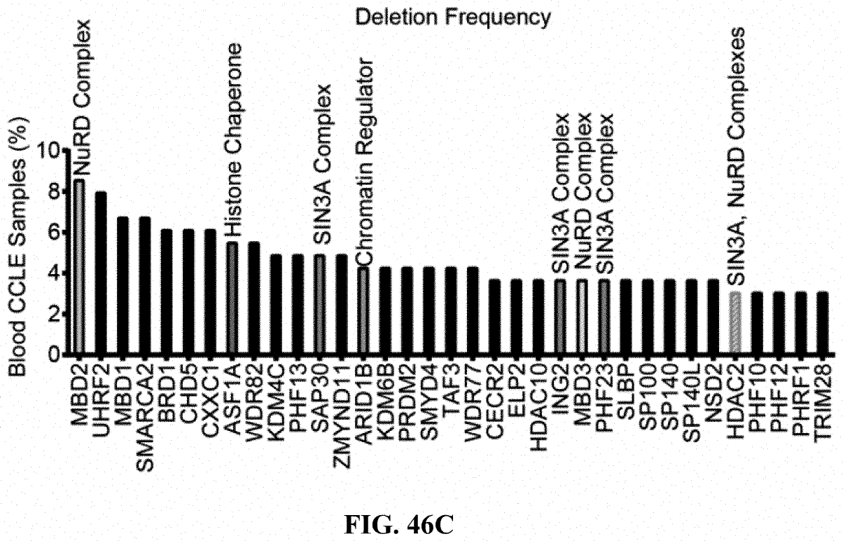

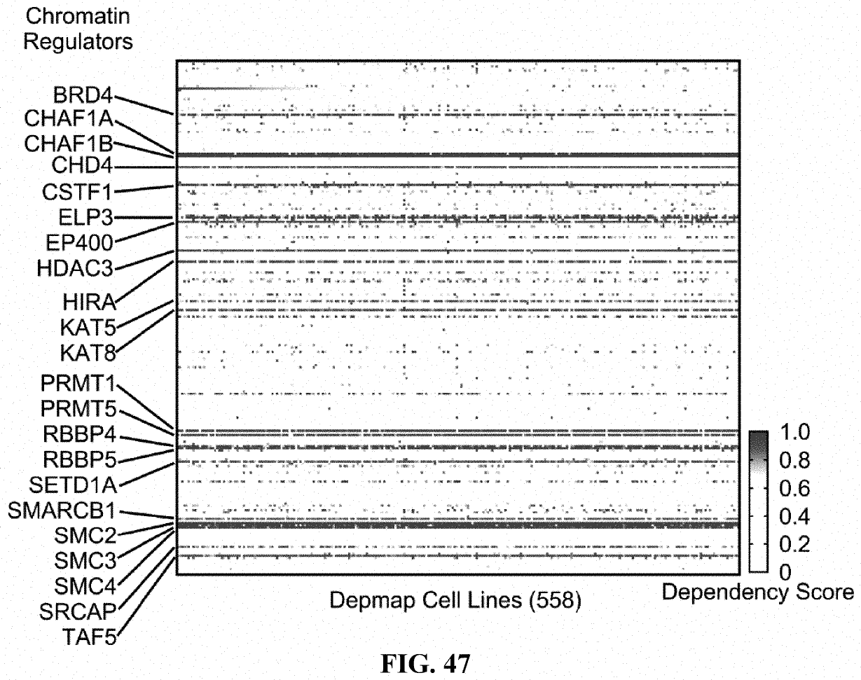

Applicants selected 268 chromatin regulator genes for further screening (FIG. 42a). 50 PFAM domains were selected and used to filter chromatin regulator genes. The deletion frequency of the 268 genes was determined in 10,967 TCGA samples (The Cancer Genome Atlas). Around 25% of 10,967 TCGA samples have 1 or more mutations in these 268 genes (FIG. 42b). The relatively high rate of deletions in TCGA samples suggests opportunities for cancer specific synthetic lethal combinations where only a single gene would need to be targeted by a therapeutic agent. There is a broad representation of these 268 gene deletions across TCGA samples. Individual chromatin regulator genes deleted in TCGA samples were identified and chromatin regulation protein complex members are indicated (FIG. 42c). Applicants also used Gene Ontology and CCLE mutation data to characterize these genes (see, e.g., Barretina, J. et al., 2012. The Cancer Cell Line Encyclopedia enables predictive modelling of anticancer drug sensitivity. Nature 483, 603-607) (FIGS. 46 and 47).

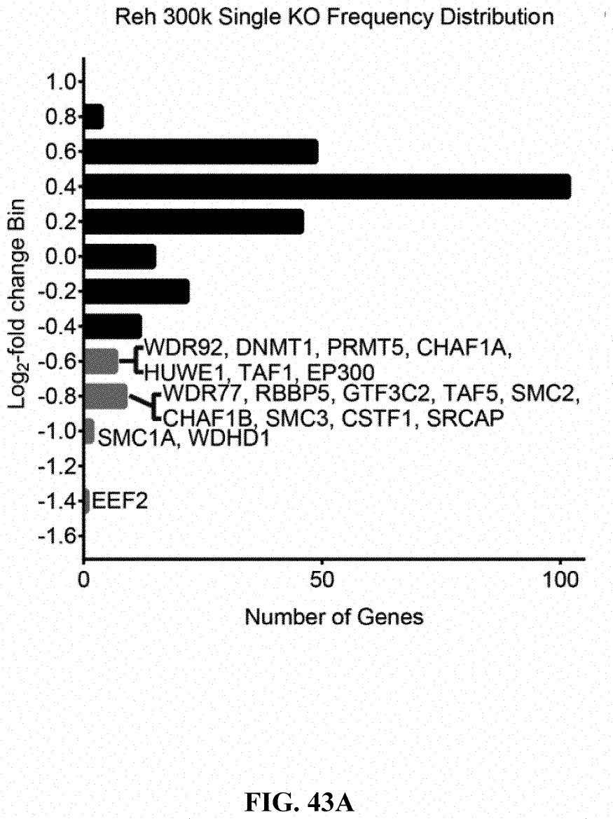

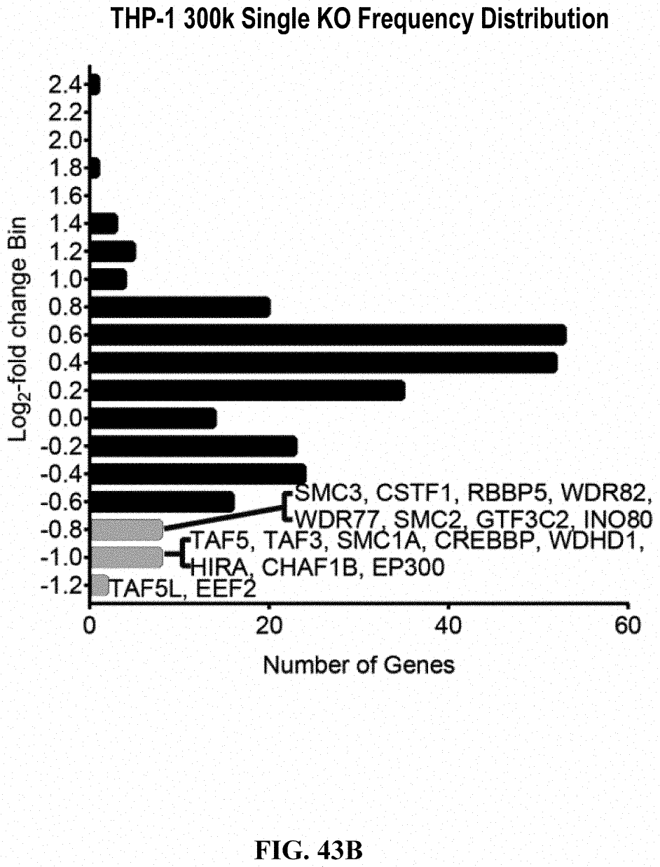

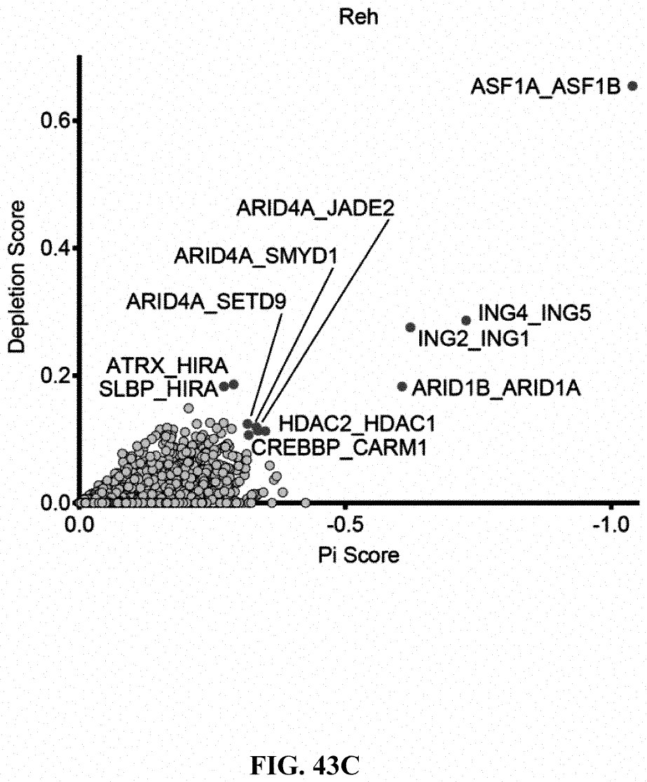

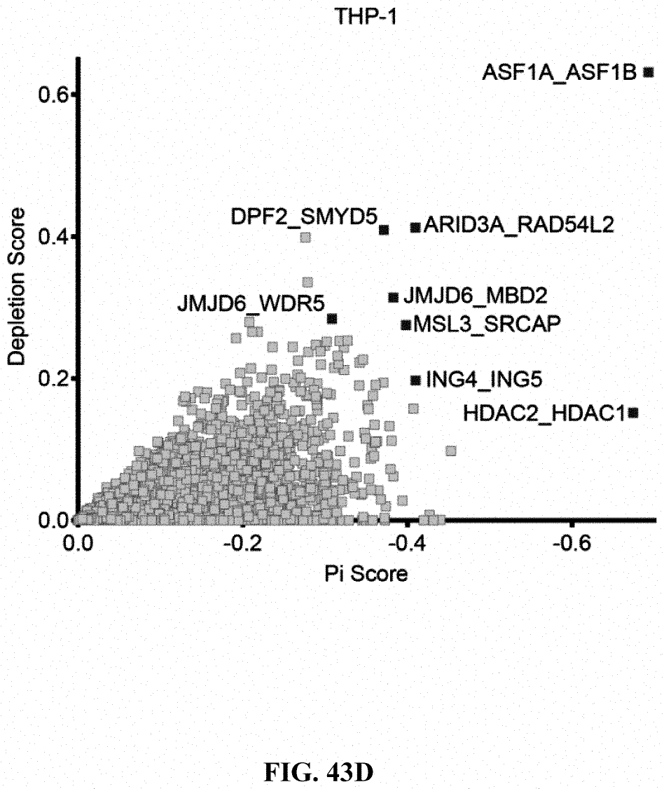

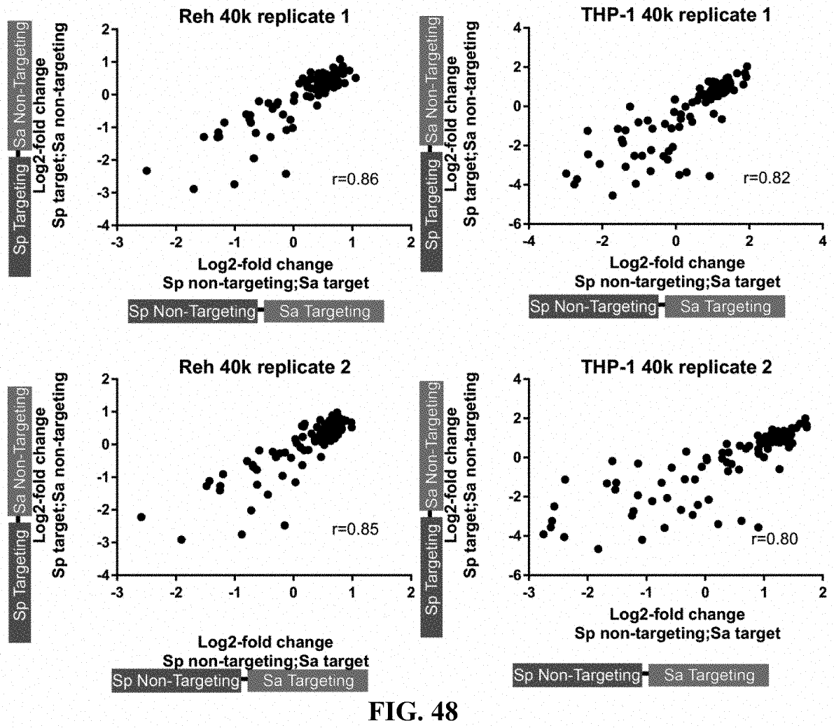

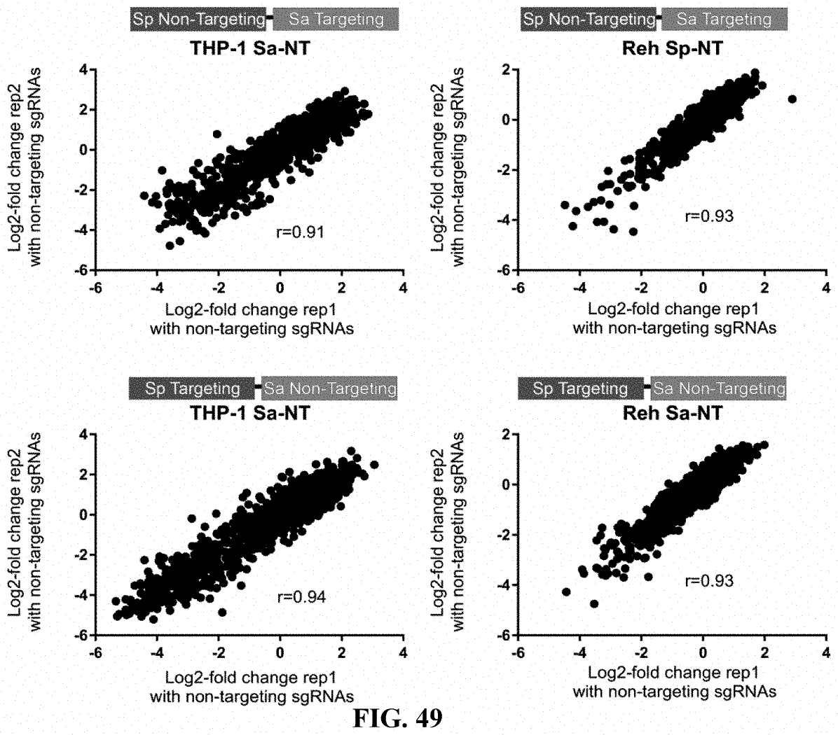

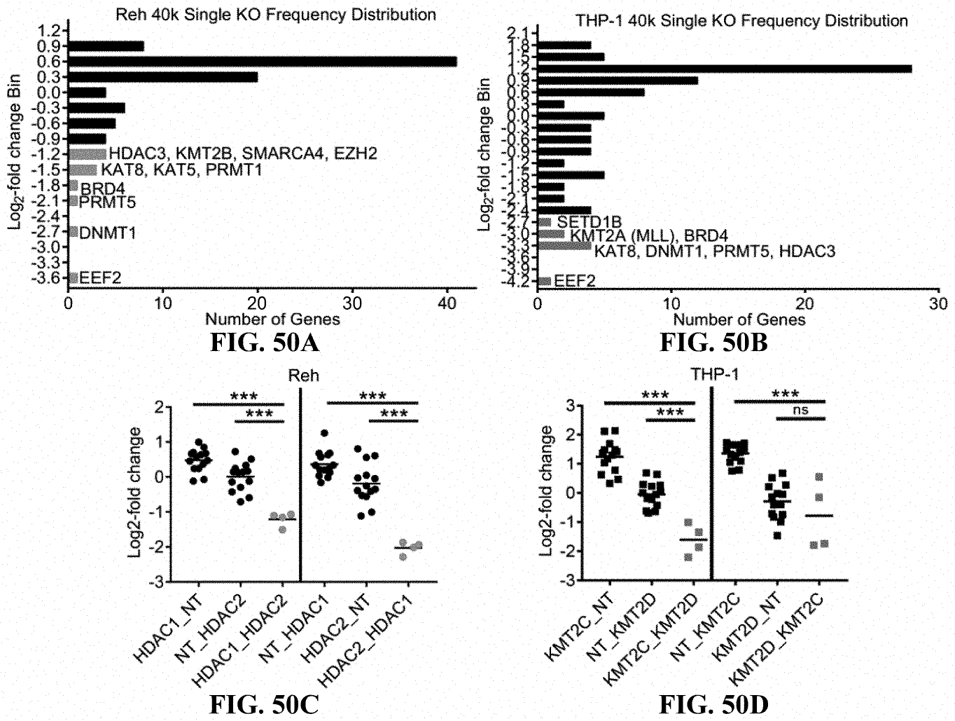

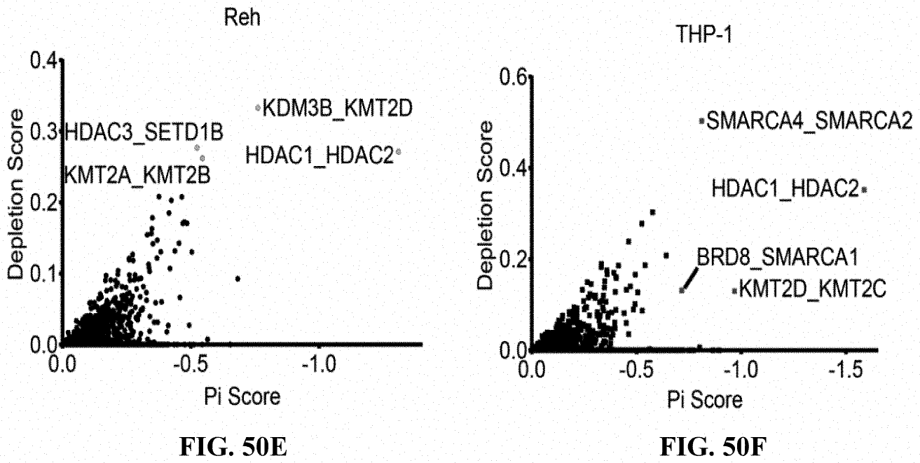

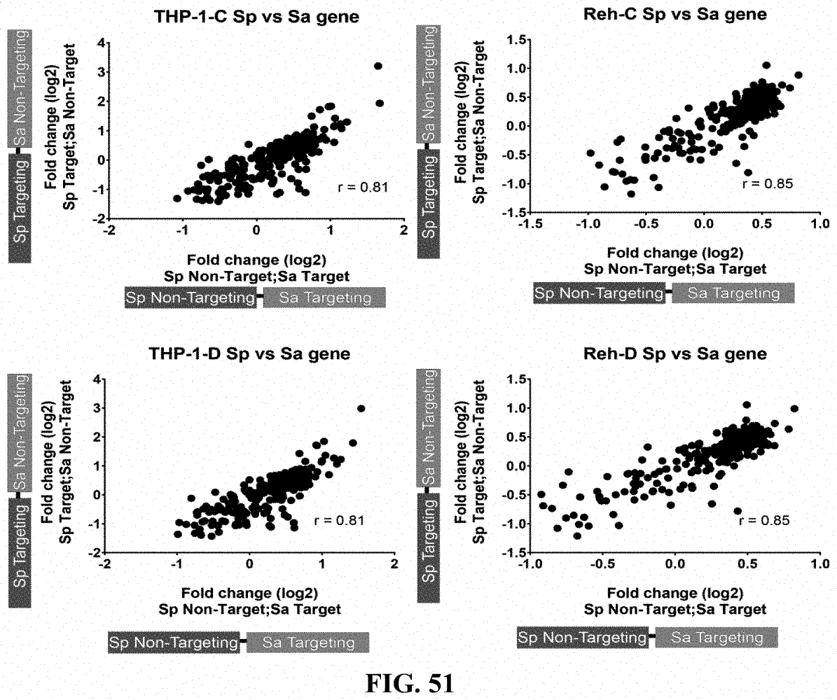

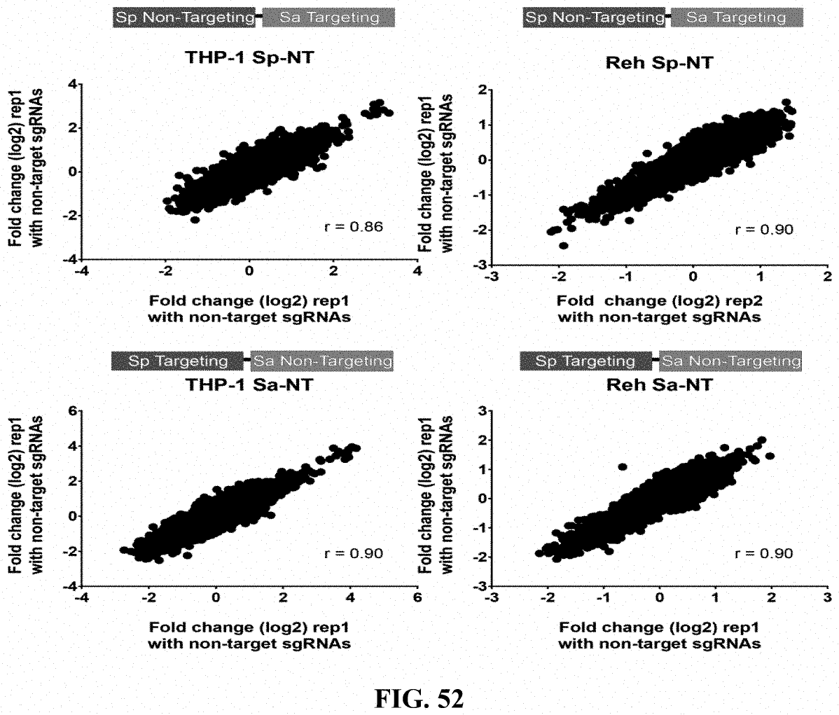

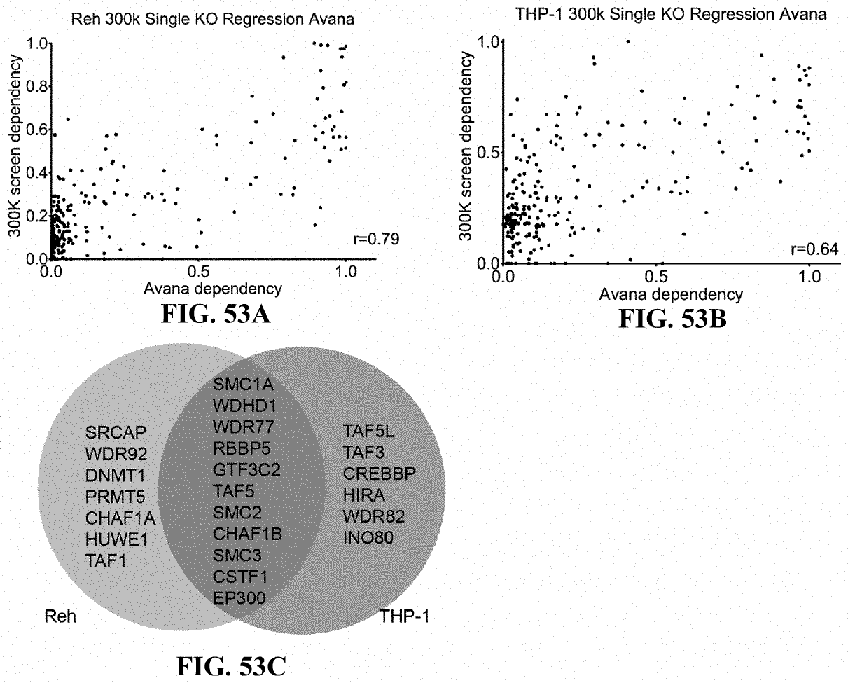

A schematic representation of the experimental flow-through shows that guides were paired all by all (FIG. 42d). A 300 k library of 552×552=304,704 perturbations was generated and screened in two cell lines (FIG. 42d). Applicants also screened specific chromatin regulator combinations. A 40 k library, with a selection of 98 of these genes, was screened (FIGS. 48-50). Cas9 ortholog performance and experimental replicates were as expected for the 300 k library screen, as the results were consistent with targeting for SpCas9 and SaCas9 (FIGS. 51-52). Singleton knockout data from the 300 k library screen correlated with the Avana library (see, e.g., Doench et al., 2016. Optimized sgRNA design to maximize activity and minimize off-target effects of CRISPR-Cas9. Nat Biotechnol; 34(2):184-191), with Reh at 0.79 and THP-1 at 0.65 (FIG. 53). Thus, single knockouts with the 300 k screen guide sequences were consistent with another library screen. Singleton screening produced a distribution of knockout frequencies and essential genes were identified in both cell lines, showing the efficacy of the 300 k library (FIG. 43a,b).

Combinatorial data were generated using a Pi score method (see, e.g., Horn T, et al. Mapping of signaling networks through synthetic genetic interaction analysis by RNAi. Nature Methods. 2011; 8:341-346) and also a depletion score that measures the absolute decrease in a guide pair combination and averaged for all gene pairs tested. Presumed synthetic lethal pairs were identified (FIG. 43c,d). Pi score takes single gene effects into account and looks for synergies. Applicants used a Pi z-score to include more statistical confidence in the data as compared to fold change.

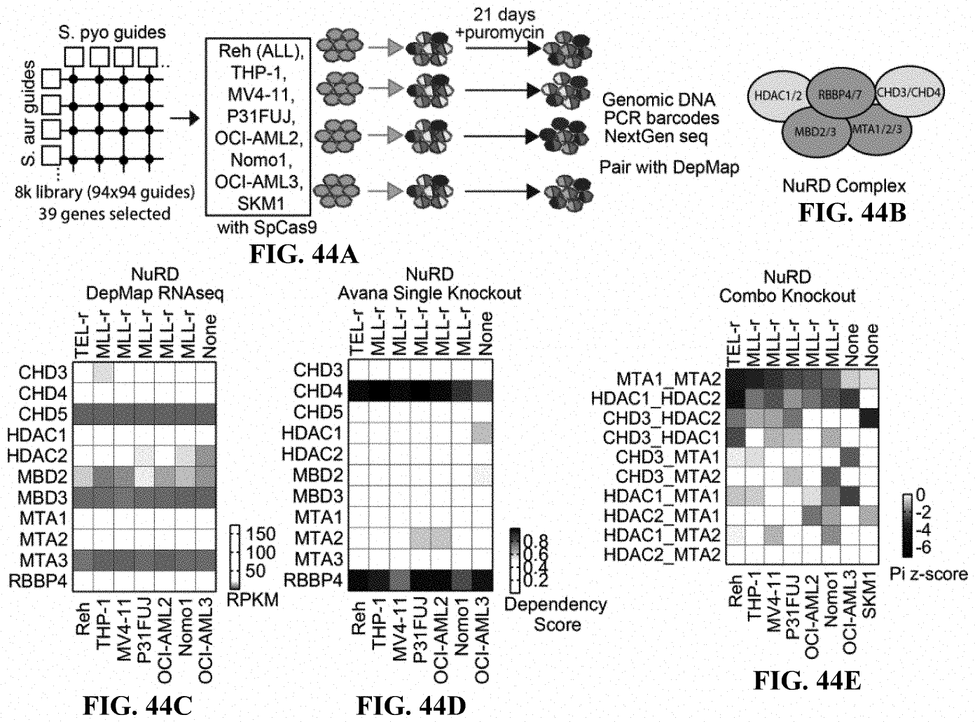

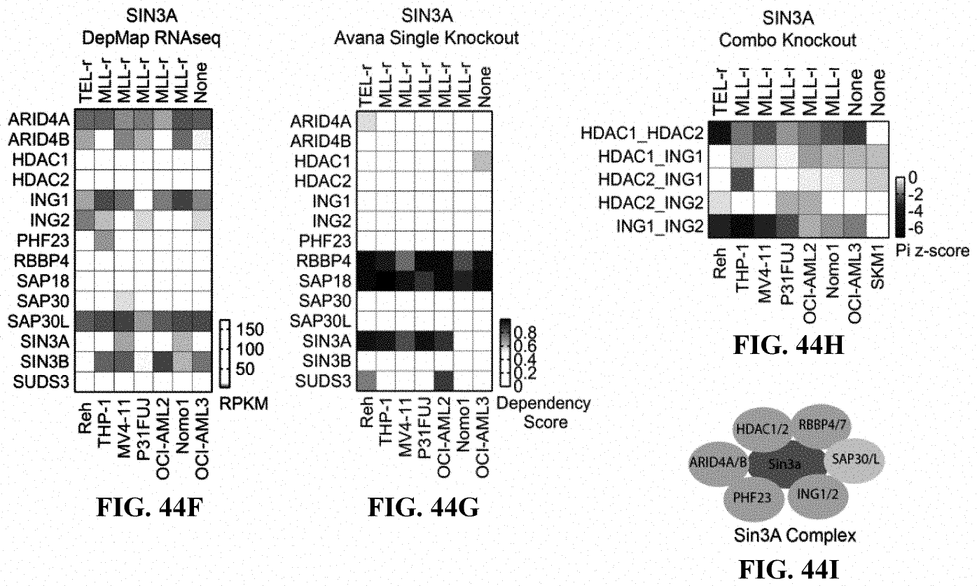

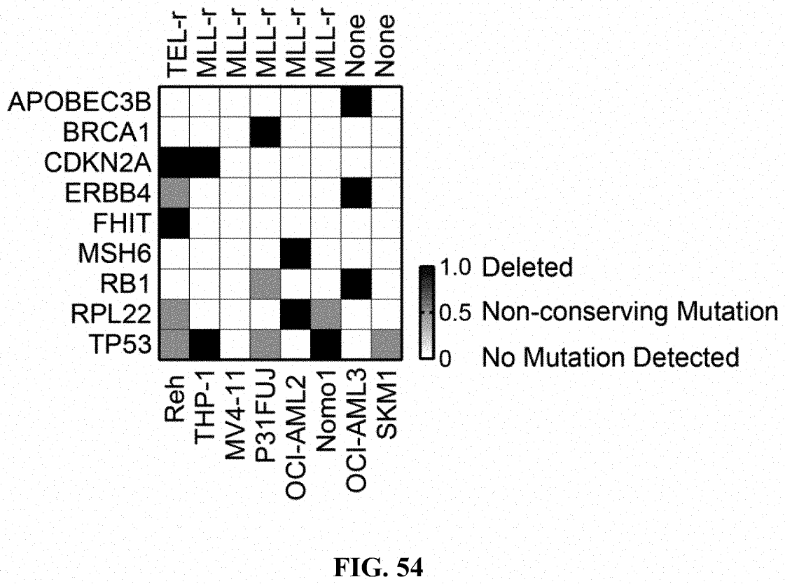

Validation screening identified vulnerabilities on repressive complexes. Thirty-nine combinatorial hits were selected to generate a new library (8K library). The 8 k library was used for screening in 8 leukemia cell lines (THP-1 (MLL-AF9), Reh (TEL-AML), MV4-11 (MLL-AF4), P31FUJ (MLLT10-PICALM), OCI-AML2 (MLL-AF6), Nomol (MLL-AF9), OCI-AML3, and SKM1) (FIG. 44a). No RNAseq or Avana data were available for the SKM1 cell line. Applicants also analyzed the tumor suppressor mutations in these lines (FIG. 54).

Applicants analyzed DepMap data (Cancer Dependency Map) with the combinatorial screening data (see, e.g., depmap.org; and Cancer Cell Line Encyclopedia Consortium, and Genomics of Drug Sensitivity in Cancer Consortium. 2015. Pharmacogenomic Agreement between Two Cancer Cell Line Data Sets. Nature 528 (7580):84-87). DepMap data complements the findings in the combinatorial screen. CHD4 and RBBP4 are strongly essential. CHD4 is well known in this context and RBBP4 is found in many complexes. In the combinatorial data Applicants see that TEL- and MLL-rearrangements (TEL-r, MLL-r) display a dependency on combinations of genes in the complex that alone are not essential. MTA1;MTA2 and HDAC1;HDAC2 appear to be targetable. Further, SKM1, a cell line without this rearrangement is susceptible to NuRD targeting, however through an alternate gene pair combination, CHD3;HDAC2. (FIG. 44b-e). Note that Pi score takes single gene effects into account and looks for synergies. Therefore, CHD4 paired with other genes does not score because it is lethal alone. SIN3A complex was targetable mostly through paralogs. Though ING1 is a lowly expressed gene, it is required with its paralog ING2. HDAC1 and HDAC2 appear to be synthetic lethal. Dependencies of these paralogs also seem to coincide with SIN3A dependency (FIG. 44f-i).

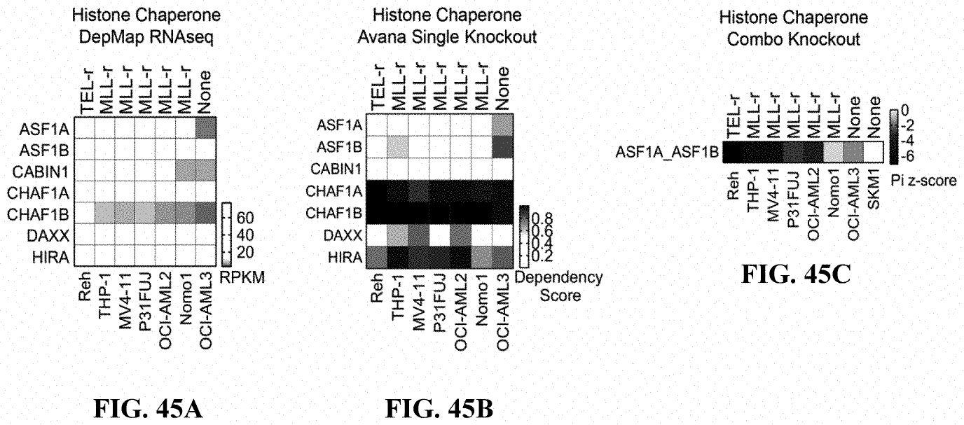

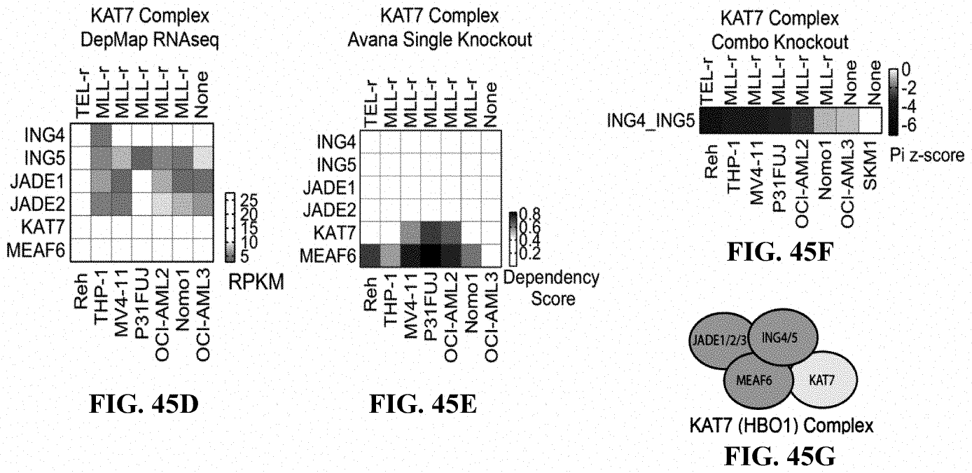

Validation screening also identified other dependent paralogs. ASF1A;ASF1B were the strongest pair in the screens. Typically many histone chaperones and related members are essential, though in this case it appears that ASF1A;ASF1B are quite redundant. It is interesting that in OCI-AML3 ASF1A is depleted and ASF1B is a dependency in the Avana library. Thus, the screening described herein provides for previously unknown therapeutic opportunities (FIG. 45a-c).

KAT7 complex screening identified that ING proteins may not be expressed highly, but result in strong effects. Furthermore, KAT7, which is the enzymatic member of the complex, is not as essential as either MEAF6 or the ING4;ING5 combination. Vulnerabilities related to MEAF6 have not been previously studied. (FIG. 45d-g).

Applicants further identified a trend that paralogs are more likely to be essential across cell lines, while gene pairs that appear to be unrelated or part of the same complex score in specific cell lines, or specific circumstances.

### REFERENCES

1. Roguev A, et al. Quantitative genetic-interaction mapping in mammalian cells. Nature Methods. 2013; 10:432-437.
2. Horn T, et al. Mapping of signaling networks through synthetic genetic interaction analysis by RNAi. Nature Methods. 2011; 8:341-346.
3. Tong A H Y, et al. Global mapping of the yeast genetic interaction network. Science. 2004; 303:808-813.
4. Costanzo M, et al. A global genetic interaction network maps a wiring diagram of cellular function. Science. 2016; 353:aaf1420-aaf1420.
5. Bassik M C, et al. A systematic mammalian genetic interaction map reveals pathways underlying ricin susceptibility. Cell. 2013; 152:909-922.
6. Wong A S L, et al. Multiplexed barcoded CRISPR-Cas9 screening enabled by CombiGEM. Proceedings of the National Academy of Sciences. 2016; 113:201517883-6.
7. Shen J P, et al. Combinatorial CRISPR-Cas9 screens for de novo mapping of genetic interactions. Nature Methods. 2017; 17:10-9.
8. Han K, et al. Synergistic drug combinations for cancer identified in a CRISPR screen for pairwise genetic interactions. Nat Biotechnol. 2017; 6:2781-15.
9. terBrake O, et al. Lentiviral vector design for multiple shRNA expression and durable HIV-1 inhibition. Mol Ther. 2008; 16:557-564.
10. Vidigal J A, Ventura A. Rapid and efficient one-step generation of paired gRNA CRISPR-Cas9 libraries. Nature Communications. 2015; 6:8083.
11. Adamson B, et al. A Multiplexed Single-Cell CRISPR Screening Platform Enables Systematic Dissection of the Unfolded Protein Response. Cell. 2016; 167:1867-1882.e21.
12. Zetsche B, et al. Multiplex gene editing by CRISPR-Cpf1 using a single crRNA array. Nat Biotechnol. 2016; 35:31-34.
13. McIntyre G J, Arndt A J, Gillespie K M, Mak W M, Fanning G C. A comparison of multiple shRNA expression methods for combinatorial RNAi. Genet Vaccines Ther. 2011; 9:9.
14. Stockman V B, et al. A High-Throughput Strategy for Dissecting Mammalian Genetic Interactions. PLoS ONE. 2016; 11:e0167617-13.
15. Gilbert L A, et al. Genome-Scale CRISPR-Mediated Control of Gene Repression and Activation. Cell. 2014; 159:647-661.
16. Doench J G, et al. Optimized sgRNA design to maximize activity and minimize off-target effects of CRISPR-Cas9. Nat Biotechnol. 2016; 34:184-191.
17. Zhu S, et al. Genome-scale deletion screening of human long non-coding RNAs using a paired-guide RNA CRISPR-Cas9 library. Nat Biotechnol. 2016; 34:1279-1286.
18. Doench J G, et al. Rational design of highly active sgRNAs for CRISPR-Cas9-mediated gene inactivation. Nat Biotechnol. 2014; 32:1262-1267.
19. Ran F A, et al. In vivo genome editing using Staphylococcus aureus Cas9. Nature. 2015; 520:186-191.
20. Friedland A E, et al. Characterization of Staphylococcus aureus Cas9: a smaller Cas9 for all-in-one adeno-associated virus delivery and paired nickase applications. Genome Biol. 2015; 16:1-10.
21. Hanzlikova H, Gittens W, Krejcikova K, Zeng Z, Caldecott KW. Overlapping roles for PARP1 and PARP2 in the recruitment of endogenous XRCC1 and PNKP into oxidized chromatin. Nucleic Acids Research. 2017; 45:2546-2557.
22. Farmer H, et al. Targeting the DNA repair defect in BRCA mutant cells as a therapeutic strategy. Nature. 2005; 434:917-921.
23. van Delft M F, et al. The BH3 mimetic ABT-737 targets selective Bcl-2 proteins and efficiently induces apoptosis via Bak/Bax if Mcl-1 is neutralized. Cancer Cell. 2006; 10:389-399.
24. Sun C, Bernards R. Feedback and redundancy in receptor tyrosine kinase signaling: relevance to cancer therapies. Trends Biochem Sci. 2014; 39:465-474.
25. Buscà R, Pouysségur J, Lenormand P. ERK1 and ERK2 Map Kinases: Specific Roles or Functional Redundancy? Front Cell Dev Biol. 2016; 4:53.
26. Uzgare A R, Isaacs J T. Enhanced redundancy in Akt and mitogen-activated protein kinase-induced survival of malignant versus normal prostate epithelial cells. Cancer Research. 2004; 64:6190-6199.
27. Srivas R, et al. A Network of Conserved Synthetic Lethal Interactions for Exploration of Precision Cancer Therapy. Mol. Cell. 2016; 63:1-13.
28. Hart T, et al. High-Resolution CRISPR Screens Reveal Fitness Genes and Genotype-Specific Cancer Liabilities. Cell. 2015; 163:1515-1526.
29. Chaudhuri L, et al. CHK1 and WEE1 inhibition combine synergistically to enhance therapeutic efficacy in acute myeloid leukemia ex vivo. Haematologica. 2014; 99:688-696.
30. Souers A J, et al. ABT-199, a potent and selective BCL-2 inhibitor, achieves antitumor activity while sparing platelets. Nat Med. 2013; 19:202-208.
31. Oltersdorf T, et al. An inhibitor of Bcl-2 family proteins induces regression of solid tumours. Nature. 2005; 435:677-681.
32. Leverson J D, et al. Exploiting selective BCL-2 family inhibitors to dissect cell survival dependencies and define improved strategies for cancer therapy. Sci Transl Med. 2015; 7: 279ra40-279ra40.
33. Lessene G, et al. Structure-guided design of a selective BCL-X(L) inhibitor. Nat Chem Biol. 2013; 9:390-397.
34. Kotschy A, et al. The MCL1 inhibitor 563845 is tolerable and effective in diverse cancer models. Nature. 2016; 538:477-482.
35. Dummler B, et al. Life with a Single Isoform of Akt: Mice Lacking Akt2 and Akt3 Are Viable but Display Impaired Glucose Homeostasis and Growth Deficiencies. Molecular and Cellular Biology. 2006; 26:8042-8051.
36. Nitulescu G M, et al. Akt inhibitors in cancer treatment: The long journey from drug discovery to clinical use (Review) Int J Oncol. 2016; 48:869-885.
37. Stordal B, et al. BRCA1/2 mutation analysis in 41 ovarian cell lines reveals only one functionally deleterious BRCA1 mutation. Mol Oncol. 2013; 7:567-579.
38. Helleday T. The underlying mechanism for the PARP and BRCA synthetic lethality: clearing up the misunderstandings. Mol Oncol. 2011; 5:387-393.
39. Rooswinkel R W, et al. Antiapoptotic potency of Bcl-2 proteins primarily relies on their stability, not binding selectivity. Blood. 2014; 123:2806-2815.
40. Cowling V, Downward J. Caspase-6 is the direct activator of caspase-8 in the cytochrome c-induced apoptosis pathway: absolute requirement for removal of caspase-6 prodomain. Cell Death Differ. 2002; 9:1046-1056.
41. Kroemer G, Galluzzi L, Brenner C. Mitochondrial membrane permeabilization in cell death. Physiol Rev. 2007; 87:99-163.
42. Li P, et al. Cytochrome c and dATP-dependent formation of Apaf-1/caspase-9 complex initiates an apoptotic protease cascade. Cell. 1997; 91:479-489.
43. Ma S B, et al. Bax targets mitochondria by distinct mechanisms before or during apoptotic cell death: a requirement for VDAC2 or Bak for efficient Bax apoptotic function. Cell Death Differ. 2014; 21:1925-1935.
44. Sarosiek K A, et al. BID preferentially activates BAK while BIM preferentially activates BAX, affecting chemotherapy response. Mol. Cell. 2013; 51:751-765.
45. Chavez A, et al. Highly efficient Cas9-mediated transcriptional programming. Nature Methods. 2015; 12:326-328.
46. Rotem A, et al. Alternative to the soft-agar assay that permits high-throughput drug and genetic screens for cellular transformation. Proceedings of the National Academy of Sciences. 2015; 112:5708-5713.
47. Miura Y, et al. Susceptibility to killer T cells of gastric cancer cells enhanced by Mitomycin-C involves induction of ATBF1 and activation of p21 (Waf1/Cip1) promoter. Microbiol Immunol. 2004; 48:137-145.
48. Wong C C, et al. Inactivating CUX1 mutations promote tumorigenesis. Nat Genet. 2014; 46:33-38.
49. Faraonio R, et al. p53 suppresses the Nrf2-dependent transcription of antioxidant response genes. Journal of Biological Chemistry. 2006; 281:39776-39784.
50. Gao Y, et al. Complex transcriptional modulation with orthogonal and inducible dCas9 regulators. Nature Methods. 2016; 13:1043-1049.
51. Kleinstiver B P, et al. Broadening the targeting range of Staphylococcus aureus CRISPR-Cas9 by modifying PAM recognition. Nat Biotechnol. 2015; 33:1293-1298.

### METHODS-ONLY REFERENCES

52. Lawrence M S, et al. Discovery and saturation analysis of cancer genes across 21 tumour types. Nature. 2014; 505:495-501.
53. Forbes S A, et al. COSMIC: somatic cancer genetics at high-resolution. Nucleic Acids Research. 2017; 45:D777-D783.

The invention is further described by the following numbered paragraphs:

1. A method for treating cancer in a subject in need thereof comprising administering to the subject a therapeutically effective amount of a combination therapy comprising one or more agents targeting the expression, activity, substrate or products of WDR77 and BRD4.

2. The method of paragraph 1, wherein the cancer is Acute myeloid leukemia (AML) NUT (nuclear protein in testis) midline carcinoma, or multiple myeloma.

3. A method for treating inflammation in a subject in need thereof comprising administering to the subject a therapeutically effective amount of a combination therapy comprising one or more agents targeting the expression, activity, substrate or products of WDR77 and BRD4.

4. The method of paragraph 3, wherein the inflammation is caused by an autoimmune disease.

5. The method of paragraph 3, wherein the inflammation is caused by a pathogen.

6. A method for reactivation of HIV in a subject in need thereof comprising administering to the subject a therapeutically effective amount of a combination therapy comprising one or more agents targeting the expression, activity, substrate or products of WDR77 and BRD4.

7. The method of any of paragraphs 1 to 6, wherein the one or more agents targeting BRD4 is selected from the group consisting of AZD5153, PFI-1, CPI-203, CPI-0610, RVX-208, OTX015, I-BET151, I-BET762, I-BET-726, dBET1, ARV-771, ARV-825, BETd-260/ZBC260 and MZ1.

8. A CD8+ T cell for use in adoptive cell transfer comprising a CD8+ T cell treated with a combination of one or more agents targeting the expression, activity, substrate or products of WDR77 and BRD4.

9. The CD8+ T cell of paragraph 8, wherein the CD8+ T cell is a CART cell.

10. The CD8+ T cell of paragraph 9 or 10, wherein the one or more agents targeting BRD4 is selected from the group consisting of AZD5153, JQ1, PFI-1, CPI-203, CPI-0610, RVX-208, OTX015, I-BET151, I-BET762, I-BET-726, dBET1, ARV-771, ARV-825, BETd-260/ZBC260 and MZ1.

11. A method for treating cancer in a subject in need thereof comprising administering to the subject a therapeutically effective amount of a combination therapy comprising one or more agents targeting the expression, activity, substrate or products of SETD6 and INO80.

12. The method of paragraph 11, wherein the cancer comprises an MLL fusion, such as Acute myeloid leukemia (AML).

13. A method for treating cancer in a subject in need thereof comprising administering to the subject a therapeutically effective amount of a combination therapy comprising one or more agents targeting the expression, activity, substrate or products of KAT6B and CHD8.

14. The method of paragraph 13, wherein the cancer is Acute myeloid leukemia (AML).

15. A method for treating cancer in a subject in need thereof comprising administering to the subject a therapeutically effective amount of a combination therapy comprising one or more agents targeting the expression, activity, substrate or products of ATRX and SMARCAL1.

16. The method of paragraph 15, wherein the cancer is Acute myeloid leukemia (AML).

17. A method for treating cancer in a subject in need thereof comprising administering to the subject a therapeutically effective amount of one or more agents targeting a first gene and one or more agents targeting a second gene, wherein said first and second genes are selected from the group consisting of ASF1B and ASF1A, ARID1B and ARID1A, ING5 and ING4, HDAC2 and HDAC1, WDR77 and HDAC6, WDR77 and KAT6B, KDM3B and ARID1A, KDM3B and CHD3, SETD2 and NSD1, ING2 and ING1, MTA1 and DOT1L, KDM3B and BRD1, KDM4A and KAT6A, INO80 and CBX1, HDAC6 and EZH2, SMARCAL1 and HDAC8, KAT5 and CHAF1B, SUV39H1 and HDAC6, KDM3B and BRD4, KMT2B and BRD8, PRMT5 and KAT5, SIRT4 and CBX1, KAT6A and CHD6, WDR77 and DOT1L, KAT2B and EHMT1, KMT2E and KAT6A, KDM3B and DOT1L, KDM3B and KDM3A, CHD8 and BRD1, HIRA and ATRX, KDM5C and KDM3B, PRDM6 and KDM3B, KAT6B and KAT6A, SMARCB1 and KDM6A, MECP2 and KDM4B, KAT2A and HDAC5, SETD2 and KDM3B, RFWD2 and CHD6, SMARCB1 and ARID3C, SETMAR and BRD1, HDAC2 and DIDO1, HDAC2 and DNMT3B, KDM4D and BRD1, PRDM1 and HDAC8, SMARCA5 and KAT6A, and KMT2D and ARID1A, and

wherein the one or more agents target the expression, activity, substrate or products of said first and second genes.

18. A method for treating cancer in a subject in need thereof comprising administering to the subject a therapeutically effective amount of one or more agents targeting a gene selected from the group consisting of:

a) SRCAP, WDR77, CHAF1B, TAF5, CSTF1, WDHD1, BRD4, DNMT1, WDR61, GTF3C2, PRMT5, RBBP5, HDAC3, TRIM24, CHD7, HIRA and SMC1A; or

b) HDAC3, PRMT5, DNMT1 and TAF3; or

c) BRD4, KMT2A and CHD7; or

d) SMC2, SMC3, TAF1, WDR92, KDM2B and HUWE1,

wherein the one or more agents target the expression, activity, substrate or products of said gene.

19. The method of paragraph any of paragraphs 1 to 18, wherein the one or more agents comprise a small molecule inhibitor, small molecule degrader, genetic modifying agent, antibody, antibody fragment, antibody-like protein scaffold, aptamer, protein, or any combination thereof.

20. The method of paragraph 19, wherein the one or more agents comprise a histone acetylation inhibitor, histone deacetylase (HDAC) inhibitor, histone lysine methylation inhibitor, histone lysine demethylation inhibitor, DNA methyltransferase (DNMT) inhibitor, inhibitor of acetylated histone binding proteins, inhibitor of methylated histone binding proteins, sirtuin inhibitor, protein arginine methyltransferase inhibitor or kinase inhibitor.

21. The method of paragraph 20, wherein the DNA methyltransferase (DNMT) inhibitor is selected from the group consisting of azacitidine (5-azacytidine), decitabine (5-aza-2′-deoxycytidine), EGCG (epigallocatechin-3-gallate), zebularine, hydralazine, and procainamide.

22. The method of paragraph 20, wherein the histone acetylation inhibitor is C646.

23. The method of paragraph 20, wherein the histone deacetylase (HDAC) inhibitor is selected from the group consisting of vorinostat, givinostat, panobinostat, belinostat, entinostat, CG-1521, romidepsin, ITF-A, ITF-B, valproic acid, OSU-HDAC-44, HC-toxin, magnesium valproate, plitidepsin, tasquinimod, sodium butyrate, mocetinostat, carbamazepine, SB939, CHR-2845, CHR-3996, JNJ-26481585, sodium phenylbutyrate, pivanex, abexinostat, resminostat, dacinostat, droxinostat, RGFP966, and trichostatin A (TSA).

24. The method of paragraph 20, wherein the histone lysine demethylation inhibitor is selected from the group consisting of pargyline, clorgyline, bizine, GSK2879552, GSK-J4, KDM5-C70, JIB-04, and tranylcypromine.

25. The method of paragraph 20, wherein the histone lysine methylation inhibitor is selected from the group consisting of EPZ004777, EPZ-6438, GSK126, CPI-360, CPI-1205, CPI-0209, DZNep, GSK343, EI1, BIX-01294, UNC0638, GSK343, UNC1999 and UNC0224.

26. The method of paragraph 20, wherein the inhibitor of acetylated histone binding proteins is selected from the group consisting of AZD5153, PFI-1, CPI-203, CPI-0610, RVX-208, OTX015, I-BET151, I-BET762, I-BET-726, dBET1, ARV-771, ARV-825, BETd-260/ZBC260 and MZ1.

27. The method of paragraph 20, wherein the inhibitor of methylated histone binding proteins is selected from the group consisting of UNC669 and UNC1215.

28. The method of paragraph 20, wherein the sirtuin inhibitor comprises nicotinamide.

29. The method of paragraph 19, wherein the genetic modifying agent comprises a CRISPR system, shRNA, a zinc finger nuclease system, a TALEN, or a meganuclease.

30. The method of paragraph 29, wherein the CRISPR system comprises a Cas13 system.

31. The method of paragraph 30, wherein the Cas13 system comprises Cas13-ADAR.

32. The method of paragraph 19, wherein the one or more agents target an active site.

33. The method of paragraph 17 or 18, wherein the cancer is Acute lymphoblastic leukemia (ALL) or Acute myeloid leukemia (AML).

34. The method of any of paragraphs 1 to 33, wherein the agents are administered concurrently or sequentially.

35. The method of any of paragraphs 1 to 34, wherein an additional cancer therapy is administered.

36. A DNA construct comprising a sequence encoding two CRISPR guide sequences positioned in an inverted orientation to each other and flanked by convergent regulatory sequences, wherein each guide sequence is operably linked to the regulatory sequence flanking the guide sequence, wherein each guide sequences is specific for an orthogonal CRISPR enzyme, and wherein the regulatory sequences do not have 100% sequence identity to one another.

37. The DNA construct according to paragraph 36, wherein each regulatory sequence is a RNA polymerase III (RNAP III) promoter.

38. The DNA construct according to paragraph 37, wherein one RNAP III promoter comprises the U6 promoter and one RNAP III promoter comprises the H1 promoter.

39. The DNA construct according to any of paragraphs 36 to 38, wherein the orthogonal CRISPR enzymes comprise S. aureus Cas9 and S. pyogenes Cas9.

40. The DNA construct according to any of paragraphs 36 to 39, further comprising a sequence encoding a CRISPR enzyme operably linked to a separate regulatory sequence.

41. The DNA construct according to paragraph 40, wherein the CRISPR enzyme is S. aureus Cas9.

42. The DNA construct according to any of paragraphs 36 to 41, further comprising a sequence encoding at least one selectable marker.

43. The DNA construct according to paragraph 42, wherein the at least one selectable marker is an antibiotic resistance gene.

44. The DNA construct according to paragraph 42, wherein the at least one selectable marker is a fluorescent gene.

45. The DNA construct according to any of paragraphs 36 to 44, wherein each guide sequence further comprises a barcode sequence.

46. The DNA construct according to any of paragraphs 36 to 45, wherein one or more of the regulatory sequences are inducible.

47. The DNA construct according to any of paragraphs 36 to 46, wherein one or both of the guide sequences comprise an aptamer sequence.

48. The DNA construct according to paragraph 47, wherein the aptamer sequence comprises an MS2 aptamer.

49. The DNA construct according to any of paragraphs 36 to 48, further comprising primer binding sequences flanking the guide sequences.

50. A vector comprising a DNA construct according to any of paragraphs 36 to 49.

51. The vector according to paragraph 50, wherein the vector is a viral vector.

52. The vector according to paragraph 51, wherein the viral vector is a lentivirus, adeno associated virus (AAV) or adenovirus vector.

53. A library for the combinatorial screening of phenotypic interactions between a set of target sequences comprising a plurality of vectors according to any of paragraphs 50 to 52, wherein the library comprises vectors comprising all possible pairwise combinations of guide sequences specific for the set of target sequences.

54. The library according to paragraph 53, wherein the set of target sequences comprises sequences targeting expression of at least two protein coding genes.

55. The library according to paragraph 54, wherein at least one protein coding gene is selected from the group consisting of:

a) genes in Table 1; or

b) DNMT1, KDM5A, KDM5B, KDM5C, KDM5D, SETDB1, SETDB2, BAZ2A, BAZ2B, ASH1L, KMT2A, KMT2B, SUV39H1, SUV39H2, JARID2, KAT2A, KAT2B, CHD3, CHD4, CHD5, CHAF1A, ZMYND8, BRPF1, BRPF3, BRD1, MBD2, MBD3, MBD1, HDAC4, HDAC5, HDAC9, BRWD1, BRWD3, KDM2A, PHIP, PBRM1, CXXC1, SETMAR, EHMT1, EHMT2, ATAD2, ATAD2B, KMT2C, KMT2D, KMT2E, MGMT, WBSCR22, CARM1, KDM4A, KDM4B, KDM4C, KDM4D, KDM4E, ARID4A, ARID4B, PHF2, PHF8, SP140L, BPTF, BAZ1A, BAZ1B, KDM7A, TRIM24, TRIM33, TRIM66, KAT5, KAT6A, KAT6B, KATE, CHD1, CHD2, CHD6, CHD7, CHD8, CHD9, SMARCA2, SMARCA4, SMARCA1, SMARCA5, EPC1, EPC2, KDM1A, KDM1B, DNMT3A, DNMT3B, WHSC1, WHSC1L1, NSD1, ZMYND11, SHPRH, MBD4, MBD3L1, MBD3L2, MECP2, ASF1A, ASF1B, ELP3, ING1, ING2, ING3, ING4, ING5, SLBP, SAP30L, SAP30, HAT1, HDAC1, HDAC10, HDAC11, HDAC2, HDAC3, HDAC6, HDAC7, HDAC8, DOT1L, MEAF6, FBXW9, FBXL19, TAF5L, TAF5, WDHD1, WDR48, WDR5, WDR61, WDR77, WDR82, WDR92, CHAF1B, CSTF1, CORO2A, DDB2, ELP2, EED, GTF3C2, HIRA, KDM2B, MTA2, MTA3, MTA1, RBBP4, RBBP5, RBBP7, RFWD2, TET1, TET3, CBX1, CBX2, CBX3, CBX4, CBX5, CBX6, CBX7, CBX8, CDYL2, CDYL, CDY1, CDY1B, CDY2A, CDY2B, SIRT1, SIRT2, SIRT3, SIRT4, SIRT5, SIRT6, SIRT7, SMC1A, SMC1B, SMC2, SMC3, SMC4, PRDM1, PRDM11, PRDM14, PRDM16, PRDM2, PRDM6, PRDM9, SMYD1, SMYD2, SMYD3, SMYD4, SETD1A, SETD1B, SETD2, SETD3, SETD4, SETD5, SETD6, SETD9, SETD7, SMYD5, EZH1, EZH2, ARID1A, ARID1B, ARID2, ARID3A, ARID3B, ARID3C, ARID5A, ARID5B, CREBBP, EP300, SP100, SP140, TAF1L, TAF1, BRD2, BRD3, BRD4, BRD7, BRD8, BRD9, BRDT, CECR2, HR, JMJD1C, JMJD4, JMJD6, KDM3A, KDM3B, KDM6A, KDM6B, UTY, PHRF1, PHF1, PHF10, PHF12, PHF13, PHF14, PHF19, PHF21A, PHF21B, PHF23, PHF3, TAF3, AIRE, DIDO1, DPF1, DPF2, DPF3, INTS12, KAT7, MSL3, MTF2, METTL13, MORF4L1, PRMT1, PRMT2, PRMT5, PYGO1, PYGO2, RSF1, TRIM28, UHRF1, UHRF2, EP400, INO80, RAD54L, RAD54L2, SET, SMARCAL1, SMARCB1, SMARCAD1, SRCAP, TBP, TSPYL2, ATRX, CHD1L, IL4I1, JADE1, JADE2 and JADE3; or

c) DOT1L, EZH2, EHMT1, EHMT2, SETD7, SMYD2, DNMT1, PRMT1, PRMT3, PRMT5, PRMT4, PRMT6, PRMT8, KDM1A, KDM6A, KDM6B, HDAC1, HDAC2, HDAC3, HDAC6, HDAC8, SIRT1, SIRT2, SIRT6, BAZ2A, BAZ2B, BRD4, BRD9/7, EP300, CECR2, SMARCA4, P300, CDK7, EED, SMYD3, BRPF1, KDM4A, KDM4B, KDM4C, KDM4D, KDM4E, KDM5A, KDM5B, KDM5C and KDM5D (Genes with inhibitors).

56. The library according to paragraph 54, wherein at least one protein coding gene comprises a protein domain selected from the group consisting of PF00439:Bromodomain, PF00145:C-5 cytosine-specific DNA methylase, PF02373:JmjC domain, hydroxylase, PF00385:Chromo (CHRromatin Organisation MOdifier) domain, PF00850:Histone deacetylase domain, PF01388:ARID/BRIGHT DNA binding domain, PF02375:jmjN domain, PF00856:SET domain, PF13508:Acetyltransferase (GNAT) domain, PF06466:PCAF (P300/CBP-associated factor)N-terminal domain, PF01853:MOZ/SAS family, PF11717:RNA binding activity-knot of a chromodomain, PF08241:Methyltransferase domain, PF13847:Methyltransferase domain, PF05185:PRMT5 arginine-N-methyltransferase, PF12047:Cytosine specific DNA methyltransferase replication foci domain, PF11531:Coactivator-associated arginine methyltransferase 1 N terminal, PF12589:Methyltransferase involved in Williams-Beuren syndrome, PF01035:6-O-methylguanine DNA methyltransferase, DNA binding domain, PF02870:6-O-methylguanine DNA methyltransferase, ribonuclease-like domain, PF00628:PHD-finger, PF05033:Pre-SET motif, PF00004:ATPase family associated with various cellular activities (AAA), PF02463:RecF/RecN/SMC N terminal domain, PF02146:Sir2 family, PF01426:BAH domain, PF02008:CXXC zinc finger domain, PF06464:DMAP1-binding Domain, PF00400:WD domain, G-beta repeat, PF08123:Histone methylation protein DOT1, PF09340:Histone acetyltransferase subunit NuA4, PF10394:Histone acetyl transferase HAT1 N-terminus, PF13867:Sin3 binding region of histone deacetylase complex subunit SAP30, PF12203:Glutamine rich N terminal domain of histone deacetylase 4, PF04729:ASF1 like histone chaperone, PF12998:Inhibitor of growth proteins N-terminal histone-binding, PF15247:Histone RNA hairpin-binding protein RNA-binding domain, PF00583:Acetyltransferase (GNAT) family, PF01429:Methyl-CpG binding domain, PF14048:C-terminal domain of methyl-CpG binding protein 2 and 3, PF00956:Nucleosome assembly protein (NAP), PF01593:Flavin containing amine oxidoreductase, PF06752:Enhancer of Polycomb C-terminus, PF10513:Enhancer of polycomb-like, PF12253:Chromatin assembly factor 1 subunit A, PF15539:CAF1 complex subunit p150, region binding to CAF1-p60 at C-term, PF15557:CAF1 complex subunit p150, region

57. The library according to paragraph 54, wherein each pairwise combination of guide sequences comprises a guide sequence selected from SEQ ID NOS: 1-552 (300K_oligos_All H1 Sa) and a guide sequence selected from SEQ ID NOS: 553-1104 (300K_oligos_All U6 Sp).

58. The library according to paragraph 54, wherein each pairwise combination of guide sequences comprises a guide sequence selected from the group consisting of SEQ ID NOS: 1105-23903 (Bernstein_pfam_saureus_guides_20160722_flagged_v2) and a guide sequence selected from the group consisting of SEQ ID NOS: 23904-45515 (Bernstein_pfam_spyo_guides_20160722_flagged).

59. A method of combinatorial screening of phenotypic interactions between a set of target sequences in a population of cells comprising:

a) introducing a library according to any of paragraphs 53 to 58 to a population of cells, wherein two orthogonal CRISPR enzymes are expressed in said cells;

b) selecting for cells comprising a vector of the library;

c) selecting for cells having a desired phenotype; and

d) determining in the cells having the desired phenotype the enrichment or depletion of combinations of guide sequences as compared to the representation in the library introduced.

60. The method according to paragraph 59, wherein the phenotypic interaction is lethality, wherein combinations of guide sequences depleted in viable cells indicate lethal combinations.

61. The method according to paragraph 59, further comprising treating the population of cells with a drug, wherein the phenotypic interaction is sensitivity or resistance to the drug.

62. The method according to paragraph 59, wherein the phenotypic interaction is differentiation, wherein combinations of guide sequences are detected in cells expressing a differentiation marker.

63. The method according to paragraph 59, wherein the phenotypic interaction is modulation of a cell state, wherein combinations of guide sequences are detected in cells expressing a marker of the cell state.

64. The method according to any of paragraphs 59 to 63, wherein selecting for cells comprising a vector of the library comprises treating the population of cells with an antibiotic.

65. The method according to any of paragraphs 59 to 64, wherein the population of cells is a population of cancer cells.

66. The method according to any of paragraphs 59 to 64, wherein the population of cells is a population of stem cells.

67. The method according to any of paragraphs 59 to 64, wherein the population of cells is a population of immune cells.

68. The method according to paragraph 67, wherein the method comprises screening for combinations of targets capable of altering the cell state in the immune cells.

69. The method according to paragraph 68, wherein the cell state is an effector or suppressive cell state.

70. The method according to paragraph 68, wherein the combinations of targets identified are used to treat autoimmunity.

71. The method according to paragraph 68, wherein the combinations of targets are used to treat cancer.

72. The method according to paragraph 68, wherein the combinations of targets are used to modulate cells for adoptive cell transfer (ACT).

73. The method according to paragraph 60, further comprising prioritizing candidate drug targets comprising determining epistatic genes, pseudo-essential genes, essential genes, pseudo-synthetic lethal genes and synthetic lethal genes, wherein candidate drug targets comprise synthetic lethal gene pairs.

74. The method according to paragraph 73, wherein determining epistatic genes, pseudo-essential genes, essential genes, pseudo-synthetic lethal genes and synthetic lethal genes comprises applying an algorithm to the pair wise combinations identified.

75. A method for generating a library for the combinatorial screening of phenotypic interactions between a set of target sequences comprising:

a) synthesizing a first set of oligonucleotides, each oligonucleotide comprising a guide sequence specific for a target sequence in the set of target sequences and specific for a first orthogonal CRISPR enzyme, wherein the oligonucleotides comprise a first non-palindromic hybridization sequence at the 3′ end and a site for cloning into a vector at the 5′end;

b) synthesizing a second set oligonucleotides, each oligonucleotide comprising a guide sequence specific for a target sequence in the set of target sequences and specific for a second orthogonal CRISPR enzyme, wherein the oligonucleotides comprise a second hybridization sequence at the 3′ end of the sequence that is complementary to the first hybridization sequence and a site for cloning into a vector at the 5′end;

c) hybridizing the first and second set of oligonucleotides;

d) performing DNA extension using the hybridization region as priming sequences to generate a pool of dsDNA oligonucleotides comprising pairs of inverted guide sequences specific for orthogonal CRISPR enzymes, wherein all pairwise combinations of guide sequences from the first and second set of oligonucleotides is represented in the pool;

e) joining the oligonucleotides from the pool of dsDNA oligonucleotides into a vector comprising two convergent regulatory sequences flanking a cloning site, wherein the two convergent regulatory sequences do not have 100% sequence identity to one another, and wherein the oligonucleotides are joined between the convergent regulatory sequences.

76. The method according to paragraph 75, wherein the ends of the oligonucleotides comprise restriction enzyme sites and the vector comprises compatible restriction enzyme site(s) between the convergent regulatory sequences, whereby joining is by ligation of compatible restriction enzyme digested ends on the oligonucleotides and the vector.

77. The method according to paragraph 75, wherein the ends of the oligonucleotides comprise homologous sequences configured for recombination and the vector comprises compatible homologous sequences between the convergent regulatory sequences, whereby joining is by recombination of the oligonucleotides into the vector.

78. The method according to any of paragraphs 75 to 77, wherein the convergent regulatory sequences are RNA polymerase III (RNAP III) promoters.

79. The method according to paragraph 78, wherein one RNAP III promoter comprises the U6 promoter and one RNAP III promoter comprises the H1 promoter.

80. The method according to any of paragraphs 75 to 79, wherein the orthogonal CRISPR enzymes comprise S. aureus Cas9 and S. pyogenes Cas9.

81. The method according to any of paragraphs 75 to 80, wherein the vector further comprises a sequence encoding a CRISPR enzyme operably linked to a regulatory sequence.

82. The method according to paragraph 81, wherein the CRISPR enzyme is S. aureus Cas9.

83. A method for treating cancer comprising a mutation in the MAPK pathway in a subject in need thereof, said method comprising administering to the subject a pharmaceutical composition capable of inhibiting the expression or activity of MAPK1 and MAPK3.

84. A method for treating cancer comprising a mutation in the MAPK pathway in a subject in need thereof, said method comprising administering to the subject a pharmaceutical composition capable of inhibiting the expression or activity of ERK1 and ERK2.

85. The method according to paragraph 83 or 84, wherein the mutation in the MAPK pathway comprises BRAF V600E, KRAS G12S or NRAS Q61L.

86. A method for treating cancer comprising a mutation in PIK3CA in a subject in need thereof, said method comprising administering to the subject a pharmaceutical composition capable of inhibiting the expression or activity of AKT1 and AKT2.

87. A kit comprising vectors according to any of paragraphs 50 to 52 or a library according to any of paragraphs 53 to 58 and instructions for use.

88. A system for generating a library for combinatorial screening, comprising a vector comprising convergent RNA polymerase III (RNAP III) promoters flanking a cloning site configured for accepting an oligonucleotide comprising inverted CRISPR guide sequences, optionally, a restriction enzyme and buffers specific to the cloning site.

89. A combination of one or more agents targeting a first gene and one or more agents targeting a second gene for use as a medicament, wherein said first and second genes are selected from the group consisting of WDR77 and BRD4, SETD6 and INO80, SMARCAL1 and ATRX, KAT6B and CHD8, ASF1B and ASF1A, ARID1B and ARID1A, ING5 and ING4, HDAC2 and HDAC1, WDR77 and HDAC6, WDR77 and KAT6B, KDM3B and ARID1A, KDM3B and CHD3, SETD2 and NSD1, ING2 and ING1, MTA1 and DOT1L, KDM3B and BRD1, KDM4A and KAT6A, INO80 and CBX1, HDAC6 and EZH2, SMARCAL1 and HDAC8, KAT5 and CHAF1B, SUV39H1 and HDAC6, KDM3B and BRD4, KMT2B and BRD8, PRMT5 and KAT5, SIRT4 and CBX1, KAT6A and CHD6, WDR77 and DOT1L, KAT2B and EHMT1, KMT2E and KAT6A, KDM3B and DOT1L, KDM3B and KDM3A, CHD8 and BRD1, HIRA and ATRX, KDM5C and KDM3B, PRDM6 and KDM3B, KAT6B and KAT6A, SMARCB1 and KDM6A, MECP2 and KDM4B, KAT2A and HDAC5, SETD2 and KDM3B, RFWD2 and CHD6, SMARCB1 and ARID3C, SETMAR and BRD1, HDAC2 and DIDO1, HDAC2 and DNMT3B, KDM4D and BRD1, PRDM1 and HDAC8, SMARCA5 and KAT6A, and KMT2D and ARID1A.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method for treating cancer in a subject in need thereof comprising administering to the subject a therapeutically effective amount of a combination therapy comprising one or more agents targeting the expression, activity, substrate or products of WDR77 and BRD4.

2. The method of claim 1, wherein the cancer is Acute myeloid leukemia (AML), NUT (nuclear protein in testis) midline carcinoma, or multiple myeloma.

3. A method for treating inflammation in a subject in need thereof comprising administering to the subject a therapeutically effective amount of a combination therapy comprising one or more agents targeting the expression, activity, substrate or products of WDR77 and BRD4.

4. The method of claim 3, wherein the inflammation is caused by an autoimmune disease.

5. The method of claim 3, wherein the inflammation is caused by a pathogen.

6. A method for reactivation of HIV in a subject in need thereof comprising administering to the subject a therapeutically effective amount of a combination therapy comprising one or more agents targeting the expression, activity, substrate or products of WDR77 and BRD4.

7. The method of any of claims 1 to 6, wherein the one or more agents targeting BRD4 is selected from the group consisting of AZD5153, PFI-1, CPI-203, CPI-0610, RVX-208, OTX015, I-BET151, I-BET762, I-BET-726, dBET1, ARV-771, ARV-825, BETd-260/ZBC260 and MZ1.

8. A CD8+ T cell for use in adoptive cell transfer comprising a CD8+ T cell treated with a combination of one or more agents targeting the expression, activity, substrate or products of WDR77 and BRD4.

9. The CD8+ T cell of claim 8, wherein the CD8+ T cell is a CAR T cell.

10. The CD8+ T cell of claim 9 or 10, wherein the one or more agents targeting BRD4 is selected from the group consisting of AZD5153, JQ1, PFI-1, CPI-203, CPI-0610, RVX-208, OTX015, I-BET151, I-BET762, I-BET-726, dBET1, ARV-771, ARV-825, BETd-260/ZBC260 and MZ1.

11. A method for treating cancer in a subject in need thereof comprising administering to the subject a therapeutically effective amount of a combination therapy comprising one or more agents targeting the expression, activity, substrate or products of SETD6 and INO80.

12. The method of claim 11, wherein the cancer comprises an MLL fusion, such as Acute myeloid leukemia (AML).

13. A method for treating cancer in a subject in need thereof comprising administering to the subject a therapeutically effective amount of a combination therapy comprising one or more agents targeting the expression, activity, substrate or products of KAT6B and CHD8.

14. The method of claim 13, wherein the cancer is Acute myeloid leukemia (AML).

15. A method for treating cancer in a subject in need thereof comprising administering to the subject a therapeutically effective amount of a combination therapy comprising one or more agents targeting the expression, activity, substrate or products of ATRX and SMARCAL1.

16. The method of claim 15, wherein the cancer is Acute myeloid leukemia (AML).

17. A method for treating cancer in a subject in need thereof comprising administering to the subject a therapeutically effective amount of a combination therapy comprising one or more agents targeting the expression, activity, substrate or products of MTA1 and MTA2.

18. The method of claim 17, wherein the cancer comprises Acute myeloid leukemia (AML).

19. The method of claim 17, wherein the cancer comprises a rearrangement in TEL or MLL.

20. A method for treating cancer in a subject in need thereof comprising administering to the subject a therapeutically effective amount of a combination therapy comprising one or more agents targeting the expression, activity, substrate or products of HDAC1 and HDAC2.

21. The method of claim 20, wherein the cancer comprises Acute myeloid leukemia (AML).

22. The method of claim 20, wherein the cancer comprises a rearrangement in TEL or MLL.

23. A method for treating cancer in a subject in need thereof comprising administering to the subject a therapeutically effective amount of a combination therapy comprising one or more agents targeting the expression, activity, substrate or products of CHD3 and HDAC2.

24. The method of claim 23, wherein the cancer comprises Acute myeloid leukemia (AML).

25. The method of claim 23, wherein the cancer does not comprise a rearrangement in TEL or MLL.

26. A method for treating cancer in a subject in need thereof comprising administering to the subject a therapeutically effective amount of a combination therapy comprising one or more agents targeting the expression, activity, substrate or products of ING1 and ING2.

27. The method of claim 26, wherein the cancer comprises Acute myeloid leukemia (AML).

28. The method of claim 26, wherein the cancer comprises a rearrangement in TEL or MLL.

29. A method for treating cancer in a subject in need thereof comprising administering to the subject a therapeutically effective amount of a combination therapy comprising one or more agents targeting the expression, activity, substrate or products of ASF1B and ASF1A.

30. The method of claim 29, wherein the cancer comprises Acute myeloid leukemia (AML).

31. The method of claim 29, wherein the cancer comprises a rearrangement in TEL or MLL.

32. A method for treating cancer in a subject in need thereof comprising administering to the subject a therapeutically effective amount of a combination therapy comprising one or more agents targeting the expression, activity, substrate or products of ING4 and ING5.

33. The method of claim 32, wherein the cancer comprises Acute myeloid leukemia (AML).

34. The method of claim 32, wherein the cancer comprises a rearrangement in TEL or MLL.

35. A personalized method for treating cancer comprising administering to a subject suffering from a cancer having a deficiency in function or expression or a mutation in either gene in a pair of genes selected from the group consisting of MTA1 and MTA2, HDAC1 and HDAC2, CHD3 and HDAC2, ING1 and ING2, ING4 and ING5, ASF1B and ASF1A, ARID4A and JADE2, ARID4A and SMYD1, ARID4A and SETD9, ATRX and HIRA, SLBP and HIRA, CREBBP and CARM1, ARID3A and RAD54L2, JMJD6 and WDR5, DPF2 and SMYD5, JMJD6 and MBD2, MSL3 and SRCAP, KMT2C and KMT2D, HDAC3 and SETD1B, KMT2A and KMT2B, KDM3B and KMT2D, SMARCA4 and SMARCA2, BRD8 and SMARCA1, WDR77 and BRD4, SETD6 and INO80, SMARCAL1 and ATRX, KAT6B and CHD8, ARID1B and ARID1A, WDR77 and HDAC6, WDR77 and KAT6B, KDM3B and ARID1A, KDM3B and CHD3, SETD2 and NSD1, MTA1 and DOT1L, KDM3B and BRD1, KDM4A and KAT6A, IN080 and CBX1, HDAC6 and EZH2, SMARCAL1 and HDAC8, KAT5 and CHAF1B, SUV39H1 and HDAC6, KDM3B and BRD4, KMT2B and BRD8, PRMT5 and KAT5, SIRT4 and CBX1, KAT6A and CHD6, WDR77 and DOT1L, KAT2B and EHMT1, KMT2E and KAT6A, KDM3B and DOT1L, KDM3B and KDM3A, CHD8 and BRD1, HIRA and ATRX, KDM5C and KDM3B, PRDM6 and KDM3B, KAT6B and KAT6A, SMARCB1 and KDM6A, MECP2 and KDM4B, KAT2A and HDAC5, SETD2 and KDM3B, RFWD2 and CHD6, SMARCB1 and ARID3C, SETMAR and BRD1, HDAC2 and DIDO1, HDAC2 and DNMT3B, KDM4D and BRD1, PRDM1 and HDAC8, SMARCA5 and KAT6A, and KMT2D and ARID1A a therapeutically effective amount of one or more agents targeting the expression, activity, substrate or products of the gene not having the deficiency or mutation.

36. The method of claim 35, wherein the cancer comprises Acute myeloid leukemia (AML).

37. The method of claim 35, wherein the cancer comprises a rearrangement in TEL or MLL.

38. A method for treating cancer in a subject in need thereof comprising administering to the subject a therapeutically effective amount of one or more agents targeting a first gene and one or more agents targeting a second gene for one or more gene pairs, wherein said one or more gene pairs are selected from the group consisting of MTA1 and MTA2, HDAC1 and HDAC2, CHD3 and HDAC2, ING1 and ING2, ING4 and ING5, ASF1B and ASF1A, ARID4A and JADE2, ARID4A and SMYD1, ARID4A and SETD9, ATRX and HIRA, SLBP and HIRA, CREBBP and CARM1, ARID3A and RAD54L2, JMJD6 and WDR5, DPF2 and SMYD5, JMJD6 and MBD2, MSL3 and SRCAP, KMT2C and KMT2D, HDAC3 and SETD1B, KMT2A and KMT2B, KDM3B and KMT2D, SMARCA4 and SMARCA2, BRD8 and SMARCA1, WDR77 and BRD4, SETD6 and INO80, SMARCAL1 and ATRX, KAT6B and CHD8, ARID1B and ARID1A, WDR77 and HDAC6, WDR77 and KAT6B, KDM3B and ARID1A, KDM3B and CHD3, SETD2 and NSD1, MTA1 and DOT1L, KDM3B and BRD1, KDM4A and KAT6A, INO80 and CBX1, HDAC6 and EZH2, SMARCAL1 and HDAC8, KAT5and CHAF1B, SUV39H1 and HDAC6, KDM3B and BRD4, KMT2B and BRD8, PRMT5 and KAT5, SIRT4 and CBX1, KAT6A and CHD6, WDR77 and DOT1L, KAT2B and EHMT1, KMT2E and KAT6A, KDM3B and DOT1L, KDM3B and KDM3A, CHD8 and BRD1, HIRA and ATRX, KDM5C and KDM3B, PRDM6 and KDM3B, KAT6B and KAT6A, SMARCB1 and KDM6A, MECP2 and KDM4B, KAT2A and HDAC5, SETD2 and KDM3B, RFWD2 and CHD6, SMARCB1 and ARID3C, SETMAR and BRD1, HDAC2 and DIDO1, HDAC2 and DNMT3B, KDM4D and BRD1, PRDM1 and HDAC8, SMARCA5 and KAT6A, and KMT2D and ARID1A, and
wherein the one or more agents target the expression, activity, substrate or products of said first and second genes.

39. A method for treating cancer in a subject in need thereof comprising administering to the subject a therapeutically effective amount of one or more agents targeting a gene selected from the group consisting of:
a) MEAF6, SRCAP, WDR77, CHAF1B, TAF5, CSTF1, WDHD1, BRD4, DNMT1, WDR61, GTF3C2, PRMT5, RBBP5, HDAC3, TRIM24, CHD7, HIRA and SMC1A; or
b) HDAC3, PRMT5, DNMT1 and TAF3; or
c) BRD4, KMT2A and CHD7; or
d) SMC2, SMC3, TAF1, WDR92, KDM2B and HUWE1,
wherein the one or more agents target the expression, activity, substrate or products of said gene.

40. The method of claim any of claims 1 to 39, wherein the one or more agents comprise a small molecule inhibitor, small molecule degrader, genetic modifying agent, antibody, antibody fragment, antibody-like protein scaffold, aptamer, protein, or any combination thereof.

41. The method of claim 40, wherein the one or more agents comprise a histone acetylation inhibitor, histone deacetylase (HDAC) inhibitor, histone lysine methylation inhibitor, histone lysine demethylation inhibitor, DNA methyltransferase (DNMT) inhibitor, inhibitor of acetylated histone binding proteins, inhibitor of methylated histone binding proteins, sirtuin inhibitor, protein arginine methyltransferase inhibitor or kinase inhibitor.

42. The method of claim 41, wherein the DNA methyltransferase (DNMT) inhibitor is selected from the group consisting of azacitidine (5-azacytidine), decitabine (5-aza-2′-deoxycytidine), EGCG (epigallocatechin-3-gallate), zebularine, hydralazine, and procainamide.

43. The method of claim 41, wherein the histone acetylation inhibitor is C646.

44. The method of claim 41, wherein the histone deacetylase (HDAC) inhibitor is selected from the group consisting of vorinostat, givinostat, panobinostat, belinostat, entinostat, CG-1521, romidepsin, ITF-A, ITF-B, valproic acid, OSU-HDAC-44, HC-toxin, magnesium valproate, plitidepsin, tasquinimod, sodium butyrate, mocetinostat, carbamazepine, SB939, CHR-2845, CHR-3996, JNJ-26481585, sodium phenylbutyrate, pivanex, abexinostat, resminostat, dacinostat, droxinostat, RGFP966, and trichostatin A (TSA).

45. The method of claim 41, wherein the histone lysine demethylation inhibitor is selected from the group consisting of pargyline, clorgyline, bizine, GSK2879552, GSK-J4, KDM5-C70, JIB-04, and tranylcypromine.

46. The method of claim 41, wherein the histone lysine methylation inhibitor is selected from the group consisting of EPZ004777, EPZ-6438, GSK126, CPI-360, CPI-1205, CPI-0209, DZNep, GSK343, EI1, BIX-01294, UNC0638, GSK343, UNC1999 and UNC0224.

47. The method of claim 41, wherein the inhibitor of acetylated histone binding proteins is selected from the group consisting of AZD5153, PFI-1, CPI-203, CPI-0610, RVX-208, OTX015, I-BET151, I-BET762, I-BET-726, dBET1, ARV-771, ARV-825, BETd-260/ZBC260 and MZ1.

48. The method of claim 41, wherein the inhibitor of methylated histone binding proteins is selected from the group consisting of UNC669 and UNC1215.

49. The method of claim 41, wherein the sirtuin inhibitor comprises nicotinamide.

50. The method of claim 40, wherein the genetic modifying agent comprises a CRISPR system, shRNA, a zinc finger nuclease system, a TALEN, or a meganuclease.

51. The method of claim 50, wherein the CRISPR system comprises a Cas13 system.

52. The method of claim 51, wherein the Cas13 system comprises Cas13-ADAR.

53. The method of claim 40, wherein the one or more agents target an active site.

54. The method of any of claims 35 to 39, wherein the cancer is Acute lymphoblastic leukemia (ALL) or Acute myeloid leukemia (AML).

55. The method of any of claims 1 to 54, wherein the agents are administered concurrently or sequentially.

56. The method of any of claims 1 to 55, wherein an additional cancer therapy is administered.

57. A DNA construct comprising a sequence encoding two CRISPR guide sequences positioned in an inverted orientation to each other and flanked by convergent regulatory sequences, wherein each guide sequence is operably linked to the regulatory sequence flanking the guide sequence, wherein each guide sequences is specific for an orthogonal CRISPR enzyme, and wherein the regulatory sequences do not have 100% sequence identity to one another.

58. The DNA construct according to claim 57, wherein each regulatory sequence is a RNA polymerase III (RNAP III) promoter.

59. The DNA construct according to claim 58, wherein one RNAP III promoter comprises the U6 promoter and one RNAP III promoter comprises the H1 promoter.

60. The DNA construct according to any of claims 57 to 59, wherein the orthogonal CRISPR enzymes comprise S. aureus Cas9 and S. pyogenes Cas9.

61. The DNA construct according to any of claims 57 to 60, further comprising a sequence encoding a CRISPR enzyme operably linked to a separate regulatory sequence.

62. The DNA construct according to claim 61, wherein the CRISPR enzyme is S. aureus Cas9.

63. The DNA construct according to any of claims 57 to 62, further comprising a sequence encoding at least one selectable marker.

64. The DNA construct according to claim 63, wherein the at least one selectable marker is an antibiotic resistance gene.

65. The DNA construct according to claim 63, wherein the at least one selectable marker is a fluorescent gene.

66. The DNA construct according to any of claims 57 to 65, wherein each guide sequence further comprises a barcode sequence.

67. The DNA construct according to any of claims 57 to 66, wherein one or more of the regulatory sequences are inducible.

68. The DNA construct according to any of claims 57 to 67, wherein one or both of the guide sequences comprise an aptamer sequence.

69. The DNA construct according to claim 68, wherein the aptamer sequence comprises an MS2 aptamer.

70. The DNA construct according to any of claims 57 to 69, further comprising primer binding sequences flanking the guide sequences.

71. A vector comprising a DNA construct according to any of claims 57 to 70.

72. The vector according to claim 71, wherein the vector is a viral vector.

73. The vector according to claim 72, wherein the viral vector is a lentivirus, adeno associated virus (AAV) or adenovirus vector.

74. A library for the combinatorial screening of phenotypic interactions between a set of target sequences comprising a plurality of vectors according to any of claims 71 to 73, wherein the library comprises vectors comprising all possible pairwise combinations of guide sequences specific for the set of target sequences.

75. The library according to claim 74, wherein the set of target sequences comprises sequences targeting expression of at least two protein coding genes.

76. The library according to claim 75, wherein at least one protein coding gene is selected from the group consisting of:
a) genes in Table 1; or
b) DNMT1, KDM5A, KDM5B, KDM5C, KDM5D, SETDB1, SETDB2, BAZ2A, BAZ2B, ASH1L, KMT2A, KMT2B, SUV39H1, SUV39H2, JARID2, KAT2A, KAT2B, CHD3, CHD4, CHD5, CHAF1A, ZMYND8, BRPF1, BRPF3, BRD1, MBD2, MBD3, MBD1, HDAC4, HDAC5, HDAC9, BRWD1, BRWD3, KDM2A, PHIP, PBRM1, CXXC1, SETMAR, EHMT1, EHMT2, ATAD2, ATAD2B, KMT2C, KMT2D, KMT2E, MGMT, WBSCR22, CARM1, KDM4A, KDM4B, KDM4C, KDM4D, KDM4E, ARID4A, ARID4B, PHF2, PHF8, SP140L, BPTF, BAZ1A, BAZ1B, KDM7A, TRIM24, TRIM33, TRIM66, KAT5, KAT6A, KAT6B, KATE, CHD1, CHD2, CHD6, CHD7, CHD8, CHD9, SMARCA2, SMARCA4, SMARCA1, SMARCA5, EPC1, EPC2, KDM1A, KDM1B, DNMT3A, DNMT3B, WHSC1, WHSC1L1, NSD1, ZMYND11, SHPRH, MBD4, MBD3L1, MBD3L2, MECP2, ASF1A, ASF1B, ELP3, ING1, ING2, ING3, ING4, ING5, SLBP, SAP30L, SAP30, HAT1, HDAC1, HDAC10, HDAC11, HDAC2, HDAC3, HDAC6, HDAC7, HDAC8, DOT1L, MEAF6, FBXW9, FBXL19, TAF5L, TAF5, WDHD1, WDR48, WDR5, WDR61, WDR77, WDR82, WDR92, CHAF1B, CSTF1, CORO2A, DDB2, ELP2, EED, GTF3C2, HIRA, KDM2B, MTA2, MTA3, MTA1, RBBP4, RBBP5, RBBP7, RFWD2, TET1, TET3, CBX1, CBX2, CBX3, CBX4, CBX5, CBX6, CBX7, CBX8, CDYL2, CDYL, CDY1, CDY1B, CDY2A, CDY2B, SIRT1, SIRT2, SIRT3, SIRT4, SIRT5, SIRT6, SIRT7, SMC1A, SMC1B, SMC2, SMC3, SMC4, PRDM1, PRDM11, PRDM14, PRDM16, PRDM2, PRDM6, PRDM9, SMYD1, SMYD2, SMYD3, SMYD4, SETD1A, SETD1B, SETD2, SETD3, SETD4, SETD5, SETD6, SETD9, SETD7, SMYD5, EZH1, EZH2, ARID1A, ARID1B, ARID2, ARID3A, ARID3B, ARID3C, ARID5A, ARID5B, CREBBP, EP300, SP100, SP140, TAF1L, TAF1, BRD2, BRD3, BRD4, BRD7, BRD8, BRD9, BRDT, CECR2, HR, JMJD1C, JMJD4, JMJD6, KDM3A, KDM3B, KDM6A, KDM6B, UTY, PHRF1, PHF1, PHF10, PHF12, PHF13, PHF14, PHF19, PHF21A, PHF21B, PHF23, PHF3, TAF3, AIRE, DIDO1, DPF1, DPF2, DPF3, INTS12, KAT7, MSL3, MTF2, METTL13, MORF4L1, PRMT1, PRMT2, PRMT5, PYGO1, PYGO2, RSF1, TRIM28, UHRF1, UHRF2, EP400, INO80, RAD54L, RAD54L2, SET, SMARCAL1, SMARCB1, SMARCAD1, SRCAP, TBP, TSPYL2, ATRX, CHD1L, IL4I1, JADE1, JADE2 and JADE3; or
c) DOT1L, EZH2, EHMT1, EHMT2, SETD7, SMYD2, DNMT1, PRMT1, PRMT3, PRMT5, PRMT4, PRMT6, PRMT8, KDM1A, KDM6A, KDM6B, HDAC1, HDAC2, HDAC3, HDAC6, HDAC8, SIRT1, SIRT2, SIRT6, BAZ2A, BAZ2B, BRD4, BRD9/7, EP300, CECR2, SMARCA4, P300, CDK7, EED, SMYD3, BRPF1, KDM4A, KDM4B, KDM4C, KDM4D, KDM4E, KDM5A, KDM5B, KDM5C and KDM5D.

77. The library according to claim 75, wherein at least one protein coding gene comprises a protein domain selected from the group consisting of PF00439:Bromodomain, PF00145:C-5 cytosine-specific DNA methylase, PF02373:JmjC domain, hydroxylase, PF00385:Chromo (CHRromatin Organisation MOdifier) domain, PF00850:Histone deacetylase domain, PF01388:ARID/BRIGHT DNA binding domain, PF02375:jmjN domain, PF00856:SET domain, PF13508:Acetyltransferase (GNAT) domain, PF06466:PCAF (P300/CBP-associated factor)N-terminal domain, PF01853:MOZ/SAS family, PF11717:RNA binding activity-knot of a chromodomain, PF08241:Methyltransferase domain, PF13847:Methyltransferase domain, PF05185:PRMT5 arginine-N-methyltransferase, PF12047:Cytosine specific DNA methyltransferase replication foci domain, PF11531:Coactivator-associated arginine methyltransferase 1 N terminal, PF12589:Methyltransferase involved in Williams-Beuren syndrome, PF01035:6-O-methylguanine DNA methyltransferase, DNA binding domain, PF02870:6-O-methylguanine DNA methyltransferase, ribonuclease-like domain, PF00628:PHD-finger, PF05033:Pre-SET motif, PF00004:ATPase family associated with various cellular activities (AAA), PF02463:RecF/RecN/SMC N terminal domain, PF02146:Sir2 family, PF01426:BAH domain, PF02008:CXXC zinc finger domain, PF06464:DMAP1-binding Domain, PF00400:WD domain, G-beta repeat, PF08123:Histone methylation protein DOT1, PF09340:Histone acetyltransferase subunit NuA4, PF10394:Histone acetyl transferase HAT1 N-terminus, PF13867:Sin3 binding region of histone deacetylase complex subunit SAP30, PF12203:Glutamine rich N terminal domain of histone deacetylase 4, PF04729:ASF1 like histone chaperone, PF12998:Inhibitor of growth proteins N-terminal histone-binding, PF15247:Histone RNA hairpin-binding protein RNA-binding domain, PF00583:Acetyltransferase (GNAT) family, PF01429:Methyl-CpG binding domain, PF14048:C-terminal domain of methyl-CpG binding protein 2 and 3, PF00956:Nucleosome assembly protein (NAP), PF01593:Flavin containing amine oxidoreductase, PF06752:Enhancer of Polycomb C-terminus, PF10513:Enhancer of polycomb-like, PF12253:Chromatin assembly factor 1 subunit A, PF15539:CAF1 complex subunit p150, region binding to CAF1-p60 at C-term, PF15557:CAF1 complex subunit p150, region binding to PCNA, PF00176:SNF2 family N-terminal domain, PF09110:HAND and PF04855: SNF5/SMARCB1/INI1.

78. The library according to claim 75, wherein each pairwise combination of guide sequences comprises a guide sequence selected from SEQ ID NOS: 1-552 and a guide sequence selected from SEQ ID NOS: 553-1104.

79. The library according to claim 75, wherein each pairwise combination of guide sequences comprises a guide sequence selected from the group consisting of SEQ ID NOS: 1105-23903 and a guide sequence selected from the group consisting of SEQ ID NOS: 23904-45515.

80. A method of combinatorial screening of phenotypic interactions between a set of target sequences in a population of cells comprising:
a) introducing a library according to any of claims 74 to 79 to a population of cells, wherein two orthogonal CRISPR enzymes are expressed in said cells;
b) selecting for cells comprising a vector of the library;
c) selecting for cells having a desired phenotype; and
d) determining in the cells having the desired phenotype the enrichment or depletion of combinations of guide sequences as compared to the representation in the library introduced.

81. The method according to claim 80, wherein the phenotypic interaction is lethality, wherein combinations of guide sequences depleted in viable cells indicate lethal combinations.

82. The method according to claim 80, further comprising treating the population of cells with a drug, wherein the phenotypic interaction is sensitivity or resistance to the drug.

83. The method according to claim 80, wherein the phenotypic interaction is differentiation, wherein combinations of guide sequences are detected in cells expressing a differentiation marker.

84. The method according to claim 80, wherein the phenotypic interaction is modulation of a cell state, wherein combinations of guide sequences are detected in cells expressing a marker of the cell state.

85. The method according to any of claims 80 to 84, wherein selecting for cells comprising a vector of the library comprises treating the population of cells with an antibiotic.

86. The method according to any of claims 80 to 85, wherein the population of cells is a population of cancer cells.

87. The method according to any of claims 80 to 85, wherein the population of cells is a population of stem cells.

88. The method according to any of claims 80 to 85, wherein the population of cells is a population of immune cells.

89. The method according to claim 88, wherein the method comprises screening for combinations of targets capable of altering the cell state in the immune cells.

90. The method according to claim 89, wherein the cell state is an effector or suppressive cell state.

91. The method according to claim 89, wherein the combinations of targets identified are used to treat autoimmunity.

92. The method according to claim 89, wherein the combinations of targets are used to treat cancer.

93. The method according to claim 89, wherein the combinations of targets are used to modulate cells for adoptive cell transfer (ACT).

94. The method according to claim 81, further comprising prioritizing candidate drug targets comprising determining epistatic genes, pseudo-essential genes, essential genes, pseudo-synthetic lethal genes and synthetic lethal genes, wherein candidate drug targets comprise synthetic lethal gene pairs.

95. The method according to claim 94, wherein determining epistatic genes, pseudo-essential genes, essential genes, pseudo-synthetic lethal genes and synthetic lethal genes comprises applying an algorithm to the pair wise combinations identified.

96. The method according to any of claims 80 to 95, wherein the orthogonal CRISPR enzymes comprise a Cas9, dCas9, Cas12, dCas12, or dCas13.

97. The method according to claim 96, wherein the dCas9 or dCas12 are fusion proteins comprising an activation or repression domain.

98. The method according to any of claims 80 to 97, wherein one CRISPR enzyme activates a gene and one CRISPR enzyme inactivates a gene.

99. A method for generating a library for the combinatorial screening of phenotypic interactions between a set of target sequences comprising:
a) synthesizing a first set of oligonucleotides, each oligonucleotide comprising a guide sequence specific for a target sequence in the set of target sequences and specific for a first orthogonal CRISPR enzyme, wherein the oligonucleotides comprise a first non-palindromic hybridization sequence at the 3′ end and a site for cloning into a vector at the 5′end;
b) synthesizing a second set oligonucleotides, each oligonucleotide comprising a guide sequence specific for a target sequence in the set of target sequences and specific for a second orthogonal CRISPR enzyme, wherein the oligonucleotides comprise a second hybridization sequence at the 3′ end of the sequence that is complementary to the first hybridization sequence and a site for cloning into a vector at the 5′end;
c) hybridizing the first and second set of oligonucleotides;
d) performing DNA extension using the hybridization region as priming sequences to generate a pool of dsDNA oligonucleotides comprising pairs of inverted guide sequences specific for orthogonal CRISPR enzymes, wherein all pairwise combinations of guide sequences from the first and second set of oligonucleotides is represented in the pool;
e) joining the oligonucleotides from the pool of dsDNA oligonucleotides into a vector comprising two convergent regulatory sequences flanking a cloning site, wherein the two convergent regulatory sequences do not have 100% sequence identity to one another, and wherein the oligonucleotides are joined between the convergent regulatory sequences.

100. The method according to claim 99, wherein the ends of the oligonucleotides comprise restriction enzyme sites and the vector comprises compatible restriction enzyme site(s) between the convergent regulatory sequences, whereby joining is by ligation of compatible restriction enzyme digested ends on the oligonucleotides and the vector.

101. The method according to claim 99, wherein the ends of the oligonucleotides comprise homologous sequences configured for recombination and the vector comprises compatible homologous sequences between the convergent regulatory sequences, whereby joining is by recombination of the oligonucleotides into the vector.

102. The method according to any of claims 99 to 101, wherein the convergent regulatory sequences are RNA polymerase III (RNAP III) promoters.

103. The method according to claim 102, wherein one RNAP III promoter comprises the U6 promoter and one RNAP III promoter comprises the H1 promoter.

104. The method according to any of claims 99 to 103, wherein the orthogonal CRISPR enzymes comprise S. aureus Cas9 and S. pyogenes Cas9.

105. The method according to any of claims 99 to 104, wherein the vector further comprises a sequence encoding a CRISPR enzyme operably linked to a regulatory sequence.

106. The method according to claim 105, wherein the CRISPR enzyme is S. aureus Cas9.

107. A method for treating cancer comprising a mutation in the MAPK pathway in a subject in need thereof, said method comprising administering to the subject a pharmaceutical composition capable of inhibiting the expression or activity of MAPK1 and MAPK3.

108. A method for treating cancer comprising a mutation in the MAPK pathway in a subject in need thereof, said method comprising administering to the subject a pharmaceutical composition capable of inhibiting the expression or activity of ERK1 and ERK2.

109. The method according to claim 107 or 108, wherein the mutation in the MAPK pathway comprises BRAF V600E, KRAS G12S or NRAS Q61L.

110. A method for treating cancer comprising a mutation in PIK3CA in a subject in need thereof, said method comprising administering to the subject a pharmaceutical composition capable of inhibiting the expression or activity of AKT1 and AKT2.

111. A kit comprising vectors according to any of claims 71 to 73 or a library according to any of claims 74 to 79 and instructions for use.

112. A system for generating a library for combinatorial screening, comprising a vector comprising convergent RNA polymerase III (RNAP III) promoters flanking a cloning site configured for accepting an oligonucleotide comprising inverted CRISPR guide sequences, optionally, a restriction enzyme and buffers specific to the cloning site.

113. A combination of one or more agents targeting a first gene and one or more agents targeting a second gene for use as a medicament, wherein said first and second genes are selected from the group consisting of MTA1 and MTA2, HDAC1 and HDAC2, CHD3 and HDAC2, ING1 and ING2, ING4 and ING5, ASF1B and ASF1A, ARID4A and JADE2, ARID4A and SMYD1, ARID4A and SETD9, ATRX and HIRA, SLBP and HIRA, CREBBP and CARM1, ARID3A and RAD54L2, JMJD6 and WDR5, DPF2 and SMYD5, JMJD6 and MBD2, MSL3 and SRCAP, KMT2C and KMT2D, HDAC3 and SETD1B, KMT2A and KMT2B, KDM3B and KMT2D, SMARCA4 and SMARCA2, BRD8 and SMARCA1, WDR77 and BRD4, SETD6 and INO80, SMARCAL1 and ATRX, KAT6B and CHD8, ARID and ARID1A, WDR77 and HDAC6, WDR77 and KAT6B, KDM3B and ARID1A, KDM3B and CHD3, SETD2 and NSD1, MTA1 and DOT1L, KDM3B and BRD1, KDM4A and KAT6A, INO80 and CBX1, HDAC6 and EZH2, SMARCAL1 and HDAC8, KAT5 and CHAF1B, SUV39H1 and HDAC6, KDM3B and BRD4, KMT2B and BRD8, PRMT5 and KAT5, SIRT4 and CBX1, KAT6A and CHD6, WDR77 and DOT1L, KAT2B and EHMT1, KMT2E and KAT6A, KDM3B and DOT1L, KDM3B and KDM3A, CHD8 and BRD1, HIRA and ATRX, KDM5C and KDM3B, PRDM6 and KDM3B, KAT6B and KAT6A, SMARCB1 and KDM6A, MECP2 and KDM4B, KAT2A and HDAC5, SETD2 and KDM3B, RFWD2 and CHD6, SMARCB1 and ARID3C, SETMAR and BRD1, HDAC2 and DIDO1, HDAC2 and DNMT3B, KDM4D and BRD1, PRDM1 and HDAC8, SMARCA5 and KAT6A, and KMT2D and ARID1A.

114. A personalized method for selecting a cancer treatment comprising determining in a subject suffering from cancer a deficiency in function or expression or a mutation in one or more pairs of genes selected from the group consisting of MTA1 and MTA2, HDAC1 and HDAC2, CHD3 and HDAC2, ING1 and ING2, ING4 and ING5, ASF1B and ASF1A, ARID4A and JADE2, ARID4A and SMYD1, ARID4A and SETD9, ATRX and HIRA, SLBP and HIRA, CREBBP and CARM1, ARID3A and RAD54L2, JMJD6 and WDR5, DPF2 and SMYD5, JMJD6 and MBD2, MSL3 and SRCAP, KMT2C and KMT2D, HDAC3 and SETD1B, KMT2A and KMT2B, KDM3B and KMT2D, SMARCA4 and SMARCA2, BRD8 and SMARCA1, WDR77 and BRD4, SETD6 and INO80, SMARCAL1 and ATRX, KAT6B and CHD8, ARID1B and ARID1A, WDR77 and HDAC6, WDR77 and KAT6B, KDM3B and ARID1A, KDM3B and CHD3, SETD2 and NSD1, MTA1 and DOT1L, KDM3B and BRD1, KDM4A and KAT6A, INO80 and CBX1, HDAC6 and EZH2, SMARCAL1 and HDAC8, KAT5 and CHAF1B, SUV39H1 and HDAC6, KDM3B and BRD4, KMT2B and BRD8, PRMT5 and KAT5, SIRT4 and CBX1, KAT6A and CHD6, WDR77 and DOT1L, KAT2B and EHMT1, KMT2E and KAT6A, KDM3B and DOT1L, KDM3B and KDM3A, CHD8 and BRD1, HIRA and ATRX, KDM5C and KDM3B, PRDM6 and KDM3B, KAT6B and KAT6A, SMARCB1 and KDM6A, MECP2 and KDM4B, KAT2A and HDAC5, SETD2 and KDM3B, RFWD2 and CHD6, SMARCB1 and ARID3C, SETMAR and BRD1, HDAC2 and DIDO1, HDAC2 and DNMT3B, KDM4D and BRD1, PRDM1 and HDAC8, SMARCA5 and KAT6A, and KMT2D and ARID1A; and
selecting a treatment targeting the gene without a deficiency in function or expression or a mutation if a gene pair has a deficiency in function or expression or a mutation in only one gene in the pair.

115. The method of claim 114, wherein the cancer comprises Acute myeloid leukemia (AML).

116. The method of claim 114, wherein the cancer comprises a rearrangement in TEL or MLL.
